SEQUENCE LISTING (290 sequences)

SEQ ID NO: 1 - protein, 8 aa, Artificial Sequence
  Synthetic
GFNIKDDY

SEQ ID NO: 2 - protein, 8 aa, Artificial Sequence
  Synthetic
IDPENGDT

SEQ ID NO: 3 - protein, 10 aa, Artificial Sequence
  Synthetic
TLWLRRGLDY

SEQ ID NO: 4 - protein, 11 aa, Artificial Sequence
  Synthetic
KSLLHSNGYTY

SEQ ID NO: 5 - protein, 3 aa, Artificial Sequence
  Synthetic
RMS

SEQ ID NO: 6 - protein, 9 aa, Artificial Sequence
  Synthetic
MQHLEYPFT

SEQ ID NO: 7 - protein, 117 aa, Artificial Sequence
  Synthetic
EVQLQQSGAELVRPGASVKLSCTASGFNIKDDYMYWVKQRPEQGLEWIGWIDPENGDTEYASKFQDKATVTADTSSNTAYLQLSSLTSEDTAVYYCTLWLRRGLDYWGQGTSVTVSS

SEQ ID NO: 8 - protein, 112 aa, Artificial Sequence
  Synthetic
DIVMTQAAPSVPVTPGESVSISCRSSKSLLHSNGYTYLFWFLQRPGQSPQLLIYRMSNLASGVPDRFSGSGSGTAFTLRISRVEAEDVGVYYCMQHLEYPFTFGGGTKLEIK

SEQ ID NO: 9 - protein, 9 aa, Artificial Sequence
  Synthetic
GYSITSGYY

SEQ ID NO: 10 - protein, 7 aa, Artificial Sequence
  Synthetic
ITFDGAN

SEQ ID NO: 11 - protein, 12 aa, Artificial Sequence
  Synthetic
TRSSYDYDVLDY

SEQ ID NO: 12 - protein, 6 aa, Artificial Sequence
  Synthetic
QDISNF

SEQ ID NO: 13 - protein, 3 aa, Artificial Sequence
  Synthetic
YTS

SEQ ID NO: 14 - protein, 9 aa, Artificial Sequence
  Synthetic
QQGHTLPYT

SEQ ID NO: 15 - protein, 119 aa, Artificial Sequence
  Synthetic
DVQLQESGPGLVKPSQSLSLTCSVTGYSITSGYYWNWIRQFPGNKLEWMGYITFDGANNYNPSLKNRISITRDTSKNQFFLKLTSVTTEDTATYYCTRSSYDYDVLDYWGQGTTLTVSS

SEQ ID NO: 16 - protein, 107 aa, Artificial Sequence
  Synthetic
DIQMTQTTSSLSASLGDRVTISCRASQDISNFLNWYQQRPDGTVKLLIYYTSRLHSGVPSRFSGSGSGTDFSLTVSNLEQEDIATYFCQQGHTLPYTFGGGTKLEIK

SEQ ID NO: 17 - protein, 8 aa, Artificial Sequence
  Synthetic
GYSFTDYC

SEQ ID NO: 18 - protein, 8 aa, Artificial Sequence
  Synthetic
IYPGSGNT

SEQ ID NO: 19 - protein, 13 aa, Artificial Sequence
  Synthetic
AREDYYPYHGMDY

SEQ ID NO: 20 - protein, 10 aa, Artificial Sequence
  Synthetic
ESVDGYDNSF

SEQ ID NO: 21 - protein, 3 aa, Artificial Sequence
  Synthetic
RAS

SEQ ID NO: 22 - protein, 9 aa, Artificial Sequence
  Synthetic
QQSSEDPWT

SEQ ID NO: 23 - protein, 120 aa, Artificial Sequence
  Synthetic
QIQLQQSGPELVRPGASVKISCKASGYSFTDYCINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 24 - protein, 111 aa, Artificial Sequence
  Synthetic
DIVLTQSPTSLAVSLGQRATISCRASESVDGYDNSFMHWYQQKPGQPPKLLIFRASNLESGIPARFSGSGSRTDFTLTINPVEAADVATYYCQQSSEDPWTFGGGTKLEIK

SEQ ID NO: 25 - protein, 8 aa, Artificial Sequence
  Synthetic
GYTFTSYW

SEQ ID NO: 26 - protein, 8 aa, Artificial Sequence
  Synthetic
IFPNSGRT

SEQ ID NO: 27 - protein, 11 aa, Artificial Sequence
  Synthetic
AREGNFGSLDY

SEQ ID NO: 28 - protein, 5 aa, Artificial Sequence
  Synthetic
SNLNY

SEQ ID NO: 29 - protein, 3 aa, Artificial Sequence
  Synthetic
DTS

SEQ ID NO: 30 - protein, 9 aa, Artificial Sequence
  Synthetic
QQWSRNPLT

SEQ ID NO: 31 - protein, 118 aa, Artificial Sequence
  Synthetic
QVHLQQPGAELVKPGASVKMSCKASGYTFTSYWITWVKQRPGQGLEWIGDIFPNSGRTNYDEKFKSKATLTVDTSSSTAYMQLSSLTSEDSAVYFCAREGNFGSLDYWGQGTTLTVSS

SEQ ID NO: 32 - protein, 106 aa, Artificial Sequence
  Synthetic
QIVLTQSPAIMSASPGEKVTMTCSANSNLNYMNWYHQKSGTSPKRWIYDTSKLASGVPARFSASGSGTSYSLTISSMEAEDAATYYCQQWSRNPLTFGAGTRLELK

SEQ ID NO: 33 - protein, 10 aa, Artificial Sequence
  Synthetic
GFSLNTYDVG

SEQ ID NO: 34 - protein, 7 aa, Artificial Sequence
  Synthetic
IWWNDDK

SEQ ID NO: 35 - protein, 11 aa, Artificial Sequence
  Synthetic
TLYSYDGGFAY

SEQ ID NO: 36 - protein, 7 aa, Artificial Sequence
  Synthetic
SSVSSSY

SEQ ID NO: 37 - protein, 3 aa, Artificial Sequence
  Synthetic
STS

SEQ ID NO: 38 - protein, 9 aa, Artificial Sequence
  Synthetic
HQYHRSPYT

SEQ ID NO: 39 - protein, 119 aa, Artificial Sequence
  Synthetic
QVTLKESGPGMLQPSQTLSLTCSFSGFSLNTYDVGVGWIRQPSGKGLEWLANIWWNDDKYYNSALKSRLTISKDTSNNQVFLKISSVDTADTATYYCTLYSYDGGFAYWGQGTLVTVSA

SEQ ID NO: 40 - protein, 108 aa, Artificial Sequence
  Synthetic
QIVLTQSPAIMSASLGERVTMTCTASSSVSSSYLHWYQQKPGSSPKLWIYSTSNLASGVPARFSGSGSGTSYSLTISSMEAEDAATYYCHQYHRSPYTFGGGTKLEIK

SEQ ID NO: 41 - protein, 8 aa, Artificial Sequence
  Synthetic
GYSFTGYN

SEQ ID NO: 42 - protein, 8 aa, Artificial Sequence
  Synthetic
INPYYGST

SEQ ID NO: 43 - protein, 15 aa, Artificial Sequence
  Synthetic
ARGDYGYDEGTWFAY

SEQ ID NO: 44 - protein, 12 aa, Artificial Sequence
  Synthetic
QSLLNSRTRKNY

SEQ ID NO: 45 - protein, 3 aa, Artificial Sequence
  Synthetic
WAS

SEQ ID NO: 46 - protein, 9 aa, Artificial Sequence
  Synthetic
KQSYNLPFT

SEQ ID NO: 47 - protein, 122 aa, Artificial Sequence
  Synthetic
EIQMKQSGAELVKPGASVKISCKASGYSFTGYNMNWVKQSHGKSLEWIGNINPYYGSTGYNQKFKGKATLTVDKSSSTAYMQLNSLTSEDSAVYYCARGDYGYDEGTWFAYWGQGTLVTVSA

SEQ ID NO: 48 - protein, 113 aa, Artificial Sequence
  Synthetic
DIVMSQSPSSLAVSAGEKVTMSCKSSQSLLNSRTRKNYLAWYQQKPEQSPKLLIYWASTRESGVPDRFTGSGSGTDFTLTISSVQAEDLAVYYCKQSYNLPFTFGSGTKLEIK

SEQ ID NO: 49 - protein, 8 aa, Artificial Sequence
  Synthetic
GYTFTRHW

SEQ ID NO: 50 - protein, 8 aa, Artificial Sequence
  Synthetic
IYPGSGRT

SEQ ID NO: 51 - protein, 13 aa, Artificial Sequence
  Synthetic
ARDGYLYINYFDY

SEQ ID NO: 52 - protein, 5 aa, Artificial Sequence
  Synthetic
SSVSF

SEQ ID NO: 53 - protein, 9 aa, Artificial Sequence
  Synthetic
FQGSGYPYT

SEQ ID NO: 54 - protein, 120 aa, Artificial Sequence
  Synthetic
QVQLQQPGAELVKPGASVKMSCKASGYTFTRHWITWVKQRPGQGLEWIGDIYPGSGRTNYNEKFKSTATLTVDTSSSTAYMQLSSLTSEDSAVYYCARDGYLYINYFDYWGQGTTLTVSS

SEQ ID NO: 55 - protein, 106 aa, Artificial Sequence
  Synthetic
ENVLTQSPAIMSASPGEKVTMTCSASSSVSFMHWFQQKSSTSPKLWIYDTSKLASGVPGRFSGSGSGSSYSLTISSMAAEDVATYYCFQGSGYPYTFGGGTKLEIK

SEQ ID NO: 56 - protein, 8 aa, Artificial Sequence
  Synthetic
GFNIVDDY

SEQ ID NO: 57 - protein, 8 aa, Artificial Sequence
  Synthetic
IYPENADT

SEQ ID NO: 58 - protein, 11 aa, Artificial Sequence
  Synthetic
TTATGTGWFAY

SEQ ID NO: 59 - protein, 11 aa, Artificial Sequence
  Synthetic
QSLLDSDGKTY

SEQ ID NO: 60 - protein, 3 aa, Artificial Sequence
  Synthetic
LVS

SEQ ID NO: 61 - protein, 9 aa, Artificial Sequence
  Synthetic
WQGTHFPWT

SEQ ID NO: 62 - protein, 118 aa, Artificial Sequence
  Synthetic
EVQLQQSGAELVRPGASVKLSCTASGFNIVDDYMHWVKQRPEQGLEWIGWIYPENADTEYASKFQGKATITADTSSNTAYLQLSSLTSEDTAVYYCTTATGTGWFAYWGQGTLVTVSA

SEQ ID NO: 63 - protein, 112 aa, Artificial Sequence
  Synthetic
DVVMTQTPLTLSVTIGQPASISCKSSQSLLDSDGKTYLNWLFQRPGQSPKRLIYLVSKLDSGVPDRFTGSGSGTDFTLKISRVETEDLGVYYCWQGTHFPWTFGGGAKLEIK

SEQ ID NO: 64 - protein, 8 aa, Artificial Sequence
  Synthetic
GYTFSNYW

SEQ ID NO: 65 - protein, 8 aa, Artificial Sequence
  Synthetic
ILPGSGST

SEQ ID NO: 66 - protein, 11 aa, Artificial Sequence
  Synthetic
ARRGAYGNFHY

SEQ ID NO: 67 - protein, 7 aa, Artificial Sequence
  Synthetic
SSISSSN

SEQ ID NO: 68 - protein, 3 aa, Artificial Sequence
  Synthetic
GTS

SEQ ID NO: 69 - protein, 9 aa, Artificial Sequence
  Synthetic
QQWRSYPYT

SEQ ID NO: 70 - protein, 118 aa, Artificial Sequence
  Synthetic
QVQLQQSGAELMKPGASVKISCKATGYTFSNYWIEWVKQRPGHGLEWIGEILPGSGSTNYNENFKGKATFTADTSSNTAYMQLSSLTSEDSAVYYCARRGAYGNFHYWGQGTTLTVSS

SEQ ID NO: 71 - protein, 108 aa, Artificial Sequence
  Synthetic
EIVLTQSPALMAASPGEKVTITCSVSSSISSSNLHWYQQKSETSPKPWIYGTSNLASGVPVRFSGSGSGTSYSLTISSMEAEDAATYYCQQWRSYPYTFGGGTKLEIK

SEQ ID NO: 72 - protein, 8 aa, Artificial Sequence
  Synthetic
GYTFTDYN

SEQ ID NO: 73 - protein, 8 aa, Artificial Sequence
  Synthetic
INPNYDTT

SEQ ID NO: 74 - protein, 16 aa, Artificial Sequence
  Synthetic
ARSGYYGSSYYWHFDV

SEQ ID NO: 75 - protein, 12 aa, Artificial Sequence
  Synthetic
QSLLYSSNQKNY

SEQ ID NO: 76 - protein, 9 aa, Artificial Sequence
  Synthetic
QQYYNYPFT

SEQ ID NO: 77 - protein, 123 aa, Artificial Sequence
  Synthetic
EVQLQQFGAELVKPGASVKISCKASGYTFTDYNMAWVKESHGKSLEWIGDINPNYDTTSYNQKFKGKATLTVDKSSSTAHMELRSLTSEGTAVYYCARSGYYGSSYYWHFDVWGTGTTVTVSS

SEQ ID NO: 78 - protein, 113 aa, Artificial Sequence
  Synthetic
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYNYPFTFGSGTKLEIK

SEQ ID NO: 79 - protein, 8 aa, Artificial Sequence
  Synthetic
GFNIKDYY

SEQ ID NO: 80 - protein, 8 aa, Artificial Sequence
  Synthetic
IDPESGDT

SEQ ID NO: 81 - protein, 9 aa, Artificial Sequence
  Synthetic
YGHDYRVDC

SEQ ID NO: 82 - protein, 11 aa, Artificial Sequence
  Synthetic
QSLVHSNGNTY

SEQ ID NO: 83 - protein, 3 aa, Artificial Sequence
  Synthetic
KVS

SEQ ID NO: 84 - protein, 9 aa, Artificial Sequence
  Synthetic
SQSTHIPWT

SEQ ID NO: 85 - protein, 116 aa, Artificial Sequence
  Synthetic
EVQLQQSGAELVRSGASVKLSCTASGFNIKDYYMHWVKQRPEQGLEWIGWIDPESGDTEYAPKFQGRATMTADTSSNTAYMQLSSLTSEDTAVYYCYGHDYRVDCWGQGTSVTVSS

SEQ ID NO: 86 - protein, 112 aa, Artificial Sequence
  Synthetic
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHIPWTFGGGTKLEIK

SEQ ID NO: 87 - protein, 8 aa, Artificial Sequence
  Synthetic
INPNYDST

SEQ ID NO: 88 - protein, 9 aa, Artificial Sequence
  Synthetic
QQYYHYPFT

SEQ ID NO: 89 - protein, 123 aa, Artificial Sequence
  Synthetic
EVQLQQFGAELVKPGASVKISCKASGYTFTDYNMGWVKQSHGKSLEWIGDINPNYDSTSYTQKFKGKATLTVDKSSSTAYMELRSLTSEDTAVYYCARSGYYGSSYYWHFDVWGTGTTVTVSS

SEQ ID NO: 90 - protein, 113 aa, Artificial Sequence
  Synthetic
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYHYPFTFGSGTKLEIK

SEQ ID NO: 91 - protein, 8 aa, Artificial Sequence
  Synthetic
GFSLTNYG

SEQ ID NO: 92 - protein, 7 aa, Artificial Sequence
  Synthetic
IWNDGSA

SEQ ID NO: 93 - protein, 11 aa, Artificial Sequence
  Synthetic
ARHESSNPFAY

SEQ ID NO: 94 - protein, 6 aa, Artificial Sequence
  Synthetic
QSIGTS

SEQ ID NO: 95 - protein, 3 aa, Artificial Sequence
  Synthetic
SAS

SEQ ID NO: 96 - protein, 9 aa, Artificial Sequence
  Synthetic
QQNNRWPYT

SEQ ID NO: 97 - protein, 117 aa, Artificial Sequence
  Synthetic
QVQLKESGPGLVAPSQSLSITCTVSGFSLTNYGVHWVRQPPGKGLEWLVVIWNDGSATYNSALESRLSISKDNSKSQVFLKMNSLQTDDTAMYYCARHESSNPFAYWGQGTLVTVSA

SEQ ID NO: 98 - protein, 107 aa, Artificial Sequence
  Synthetic
DILLTQSPAILSVSPGERVSFSCRASQSIGTSIHWYQQRTNGSPRLLIKSASESIAGIPSRFSGSGSGTDFTLSINSVESEDIADYYCQQNNRWPYTFGGGTKLEIK

SEQ ID NO: 99 - protein, 8 aa, Artificial Sequence
  Synthetic
DFNIKDDY

SEQ ID NO: 100 - protein, 8 aa, Artificial Sequence
  Synthetic
IDPANGNT

SEQ ID NO: 101 - protein, 6 aa, Artificial Sequence
  Synthetic
ALGYTY

SEQ ID NO: 102 - protein, 11 aa, Artificial Sequence
  Synthetic
QSLLHSYGKTY

SEQ ID NO: 103 - protein, 9 aa, Artificial Sequence
  Synthetic
LQTTHFPQT

SEQ ID NO: 104 - protein, 113 aa, Artificial Sequence
  Synthetic
EVQLQQSGAELVRPGASVKLSCTASDFNIKDDYIHWVKQRPEQGLEWIGRIDPANGNTKYAPKFQDKATITADTSSNTAYLQLSSLTSEDTAVYYCALGYTYWGQGTTLTVSS

SEQ ID NO: 105 - protein, 112 aa, Artificial Sequence
  Synthetic
DVVMTQTPLTLSVTIGQPASISCKSSQSLLHSYGKTYLNWLLQRPGQSPKLLIYLVSKLESGVPDRFSGSGSGTDFTLKISRVEAEDLGVYYCLQTTHFPQTFGGGTKLEIK

SEQ ID NO: 106 - protein, 8 aa, Artificial Sequence
  Synthetic
GFTFSDYG

SEQ ID NO: 107 - protein, 8 aa, Artificial Sequence
  Synthetic
INSGSSTI

SEQ ID NO: 108 - protein, 13 aa, Artificial Sequence
  Synthetic
ARPGDYDNYAMDY

SEQ ID NO: 109 - protein, 6 aa, Artificial Sequence
  Synthetic
QDVSVA

SEQ ID NO: 110 - protein, 3 aa, Artificial Sequence
  Synthetic
WAY

SEQ ID NO: 111 - protein, 10 aa, Artificial Sequence
  Synthetic
QQHYNTPPWT

SEQ ID NO: 112 - protein, 120 aa, Artificial Sequence
  Synthetic
EVQLVESGGDLVKPGGSLKLSCAASGFTFSDYGMHWVRQGPEKGLEWVAYINSGSSTIYYADTVKGRFTISRDNAKNTLFLQMTSLRSEDTAMYYCARPGDYDNYAMDYWGQGTSVTVSS

SEQ ID NO: 113 - protein, 108 aa, Artificial Sequence
  Synthetic
DIVMTQSHKFLSTSVGDRVSITCKASQDVSVAVAWYQQKPGQSPKLLIYWAYTRHTGVPDRFTGSGSGTEYTLTISSVQAEDLALYYCQQHYNTPPWTFGGGTKLEIK

SEQ ID NO: 114 - protein, 8 aa, Artificial Sequence
  Synthetic
IDPENADT

SEQ ID NO: 115 - protein, 9 aa, Artificial Sequence
  Synthetic
YAWDYSMDY

SEQ ID NO: 116 - protein, 9 aa, Artificial Sequence
  Synthetic
SQNTHVPYT

SEQ ID NO: 117 - protein, 116 aa, Artificial Sequence
  Synthetic
EVQLQQSGAELVRSGASVKLSCTASGFNIKDYYIHWVKQRPEQGLEWIGWIDPENADTEYAPKFQGKATMTPDTSSNTAYLQLSSLTSEDTAVYYCYAWDYSMDYWGQGTSVTVSS

SEQ ID NO: 118 - protein, 112 aa, Artificial Sequence
  Synthetic
DVVMTQTPLSLSVSLGDQASISCRSSQSLVHSNGNTYLHWYLQKPGQSPKLLIYKVSNRFSGVPDRFSGSGSGTDFILKISRVEAEDLGVYFCSQNTHVPYTFGGGTRLEIK

SEQ ID NO: 119 - protein, 8 aa, Artificial Sequence
  Synthetic
GFTFTDYG

SEQ ID NO: 120 - protein, 8 aa, Artificial Sequence
  Synthetic
IYPSSGNS

SEQ ID NO: 121 - protein, 12 aa, Artificial Sequence
  Synthetic
ARSTYYGSPIDY

SEQ ID NO: 122 - protein, 6 aa, Artificial Sequence
  Synthetic
QDVDTT

SEQ ID NO: 123 - protein, 9 aa, Artificial Sequence
  Synthetic
QQYSTYPLT

SEQ ID NO: 124 - protein, 119 aa, Artificial Sequence
  Synthetic
QVQLQQSGTELARPGASVKLSCKASGFTFTDYGINWVKQRTGQGLEWIGEIYPSSGNSYYNEKFKAKATLTADKSSSTAYMELRSLTSEDSAVYFCARSTYYGSPIDYWGQGTTLTVSS

SEQ ID NO: 125 - protein, 107 aa, Artificial Sequence
  Synthetic
DIVMTQSHKFMSTPVGDRVSITCKASQDVDTTVAWYQQKPGQSPKLLIYWASTRQIGVPDRFTGSGSGTDFTLTISNVQSEDLADYFCQQYSTYPLTFGGGTKLEIK

SEQ ID NO: 126 - protein, 8 aa, Artificial Sequence
  Synthetic
GFSLTSYA

SEQ ID NO: 127 - protein, 7 aa, Artificial Sequence
  Synthetic
IWTGGGT

SEQ ID NO: 128 - protein, 13 aa, Artificial Sequence
  Synthetic
ARIYDGYYRYFDV

SEQ ID NO: 129 - protein, 6 aa, Artificial Sequence
  Synthetic
QSVSND

SEQ ID NO: 130 - protein, 3 aa, Artificial Sequence
  Synthetic
YAS

SEQ ID NO: 131 - protein, 9 aa, Artificial Sequence
  Synthetic
QQDYSSPWT

SEQ ID NO: 132 - protein, 119 aa, Artificial Sequence
  Synthetic
QVQLKESGPGLVAPSQSLSITCTVSGFSLTSYAITWVRQSPGKGLEWLGLIWTGGGTNYNSALKSRLSISKDNSKSQVFLKMNSLQTDDTARYYCARIYDGYYRYFDVWGTGTTVTVSS

SEQ ID NO: 133 - protein, 107 aa, Artificial Sequence
  Synthetic
RIVLTQTPKFLLVSAGDRVTMTCKASQSVSNDVAWYQQKPGQSPKLLIYYASNRYTGVPDRFTGSGYGTDFTFTISTVQAEDLAVYFCQQDYSSPWTFGGGTKLEIK

SEQ ID NO: 134 - protein, 9 aa, Artificial Sequence
  Synthetic
NVLTMPTAY

SEQ ID NO: 135 - protein, 9 aa, Artificial Sequence
  Synthetic
QQYYSYPYT

SEQ ID NO: 136 - protein, 116 aa, Artificial Sequence
  Synthetic
EVQLQQSGAELVRSGASVKLSCTASGFNIKDYYMHWVKQRPEQGLDWIGWIDPENGDTEYAPKFQGKATMTADTSSNTAYLQLSSLTSEDTAVYYCNVLTMPTAYWGQGTLVTVSA

SEQ ID NO: 137 - protein, 113 aa, Artificial Sequence
  Synthetic
DIVMSQSPSSLAVSVGEKVIMSCKSSQSLLYSSNQKNYLAWYQQKPGQSPKLLIYWASTRESGVPDRFTGSGSGTDFTLTISSVKAEDLAVYYCQQYYSYPYTFGGGTKLEIK

SEQ ID NO: 138 - protein, 8 aa, Artificial Sequence
  Synthetic
GFTFSSYG

SEQ ID NO: 139 - protein, 8 aa, Artificial Sequence
  Synthetic
ITSGGSYT

SEQ ID NO: 140 - protein, 7 aa, Artificial Sequence
  Synthetic
ALWSLDY

SEQ ID NO: 141 - protein, 5 aa, Artificial Sequence
  Synthetic
SSLSY

SEQ ID NO: 142 - protein, 9 aa, Artificial Sequence
  Synthetic
HQRRSYPWT

SEQ ID NO: 143 - protein, 114 aa, Artificial Sequence
  Synthetic
EVQLMESGGDLVKPGGSLKLSCAASGFTFSSYGLSWVRQTPDKRLEWVATITSGGSYTYYPDSVKGRFTISRDNARNTLYLQMFSLKSEDTAMYYCALWSLDYWGQGTTLTVSS

SEQ ID NO: 144 - protein, 106 aa, Artificial Sequence
  Synthetic
QIVLTQSPAIMSASPGEKVTMTCSANSSLSYMHWYQQKPGTSPKRWIYDTSELASGVPARFSGSGSGTSYSLTISSMEAEDAATYYCHQRRSYPWTFGGGTKLEIK

SEQ ID NO: 145 - protein, 5 aa, Artificial Sequence
  Synthetic
DDYMY

SEQ ID NO: 146 - protein, 17 aa, Artificial Sequence
  Synthetic
WIDPENGDTEYASKFQD

SEQ ID NO: 147 - protein, 8 aa, Artificial Sequence
  Synthetic
WLRRGLDY

SEQ ID NO: 148 - protein, 16 aa, Artificial Sequence
  Synthetic
RSSKSLLHSNGYTYLF

SEQ ID NO: 149 - protein, 7 aa, Artificial Sequence
  Synthetic
RMSNLAS

SEQ ID NO: 150 - protein, 7 aa, Artificial Sequence
  Synthetic
GFNIKDD

SEQ ID NO: 151 - protein, 3 aa, Artificial Sequence
  Synthetic
ENG

SEQ ID NO: 152 - protein, 6 aa, Artificial Sequence
  Synthetic
LRRGLD

SEQ ID NO: 153 - protein, 12 aa, Artificial Sequence
  Synthetic
SKSLLHSNGYTY

SEQ ID NO: 154 - protein, 6 aa, Artificial Sequence
  Synthetic
HLEYPF

SEQ ID NO: 155 - protein, 6 aa, Artificial Sequence
  Synthetic
SGYYWN

SEQ ID NO: 156 - protein, 16 aa, Artificial Sequence
  Synthetic
YITFDGANNYNPSLKN

SEQ ID NO: 157 - protein, 10 aa, Artificial Sequence
  Synthetic
SSYDYDVLDY

SEQ ID NO: 158 - protein, 11 aa, Artificial Sequence
  Synthetic
RASQDISNFLN

SEQ ID NO: 159 - protein, 7 aa, Artificial Sequence
  Synthetic
YTSRLHS

SEQ ID NO: 160 - protein, 8 aa, Artificial Sequence
  Synthetic
GYSITSGY

SEQ ID NO: 161 - protein, 3 aa, Artificial Sequence
  Synthetic
FDG

SEQ ID NO: 162 - protein, 8 aa, Artificial Sequence
  Synthetic
SYDYDVLD

SEQ ID NO: 163 - protein, 7 aa, Artificial Sequence
  Synthetic
SQDISNF

SEQ ID NO: 164 - protein, 6 aa, Artificial Sequence
  Synthetic
GHTLPY

SEQ ID NO: 165 - protein, 5 aa, Artificial Sequence
  Synthetic
DYCIN

SEQ ID NO: 166 - protein, 17 aa, Artificial Sequence
  Synthetic
WIYPGSGNTRYSERFKG

SEQ ID NO: 167 - protein, 11 aa, Artificial Sequence
  Synthetic
EDYYPYHGMDY

SEQ ID NO: 168 - protein, 15 aa, Artificial Sequence
  Synthetic
RASESVDGYDNSFMH

SEQ ID NO: 169 - protein, 7 aa, Artificial Sequence
  Synthetic
RASNLES

SEQ ID NO: 170 - protein, 7 aa, Artificial Sequence
  Synthetic
GYSFTDY

SEQ ID NO: 171 - protein, 3 aa, Artificial Sequence
  Synthetic
GSG

SEQ ID NO: 172 - protein, 9 aa, Artificial Sequence
  Synthetic
DYYPYHGMD

SEQ ID NO: 173 - protein, 11 aa, Artificial Sequence
  Synthetic
SESVDGYDNSF

SEQ ID NO: 174 - protein, 6 aa, Artificial Sequence
  Synthetic
SSEDPW

SEQ ID NO: 175 - protein, 330 aa, Homo sapiens [sequence omitted]

SEQ ID NO: 176 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 107 aa, Artificial Sequence [sequence omitted]

SEQ ID NO: 178 - protein, 447 aa, Artificial Sequence
  Synthetic
EVQLQQSGAELVRPGASVKLSCTASGFNIKDDYMYWVKQRPEQGLEWIGWIDPENGDTEYASKFQDKATVTADTSSNTAYLQLSSLTSEDTAVYYCTLWLRRGLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 219 aa, Artificial Sequence
  Synthetic
DIVMTQAAPSVPVTPGESVSISCRSSKSLLHSNGYTYLFWFLQRPGQSPQLLIYRMSNLASGVPDRFSGSGSGTAFTLRISRVEAEDVGVYYCMQHLEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 449 aa, Artificial Sequence
  Synthetic
DVQLQESGPGLVKPSQSLSLTCSVTGYSITSGYYWNWIRQFPGNKLEWMGYITFDGANNYNPSLKNRISITRDTSKNQFFLKLTSVTTEDTATYYCTRSSYDYDVLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 214 aa, Artificial Sequence
  Synthetic
DIQMTQTTSSLSASLGDRVTISCRASQDISNFLNWYQQRPDGTVKLLIYYTSRLHSGVPSRFSGSGSGTDFSLTVSNLEQEDIATYFCQQGHTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 450 aa, Artificial Sequence
  Synthetic
QIQLQQSGPELVRPGASVKISCKASGYSFTDYCINWVNQRPGQGLEWIGWIYPGSGNTRYSERFKGKATLTVDTSSNTAYMQLSSLTSEDSAVYFCAREDYYPYHGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 218 aa, Artificial Sequence
  Synthetic
DIVLTQSPTSLAVSLGQRATISCRASESVDGYDNSFMHWYQQKPGQPPKLLIFRASNLESGIPARFSGSGSRTDFTLTINPVEAADVATYYCQQSSEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 108 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 185 - protein, 225 aa, Artificial Sequence
  Synthetic
EVQLQQSGAELVRPGASVKLSCTASGFNIKDDYMYWVKQRPEQGLEWIGWIDPENGDTEYASKFQDKATVTADTSSNTAYLQLSSLTSEDTAVYYCTLWLRRGLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 186 - protein, 227 aa, Artificial Sequence
  Synthetic
DVQLQESGPGLVKPSQSLSLTCSVTGYSITSGYYWNWIRQFPGNKLEWMGYITFDGANNYNPSLKNRISITRDTSKNQFFLKLTSVTTEDTATYYCTRSSYDYDVLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 187 - protein, 228 aa, Artificial Sequence
  Synthetic
QIQLQQSGPELVRPGASVKISCKASGYSFTDYCINWVNQRPGQGLEWIGWIYPGSGNTRYSERFKGKATLTVDTSSNTAYMQLSSLTSEDSAVYFCAREDYYPYHGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 188 - DNA, 351 bp, Artificial Sequence
  Synthetic
gaggttcagctgcagcagtctggggctgagcttgtgaggccaggggcctcagtcaagttgtcctgcacagcttctggctttaacattaaagacgactatatgtactgggtgaagcagaggcctgagcagggcctggagtggattggatggattgatcctgagaatggtgatactgaatatgcctcgaagttccaggacaaggccactgtaacagcagacacatcctccaacacagcctacctgcagctcagcagcctgacatctgaggacactgccgtctattactgtactttatggttacgccgagggttggactactggggtcaaggaacctcagtcaccgtctcctca

SEQ ID NO: 189 - DNA, 336 bp, Artificial Sequence
  Synthetic
gatattgtgatgactcaggctgcaccctctgtacctgtcactcctggagagtcagtatccatctcctgcaggtctagtaagagtctcctgcatagtaatggctacacttacttgttttggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgttcacgttcggaggggggaccaagctggaaataaaa

SEQ ID NO: 190 - DNA, 357 bp, Artificial Sequence
  Synthetic
gatgtacagcttcaggagtcaggacctggcctcgtgaaaccttctcagtctctgtctctcacctgctctgtcactggctactccatcaccagtggttattactggaactggatccggcagtttccaggaaacaaactggaatggatgggctacataaccttcgatggtgccaataactacaacccatctctcaaaaatcgcatctccatcactcgtgacacatctaagaaccagtttttcctgaagttgacttctgtgactactgaggacacagccacatattactgtacaagatcctcctatgattacgacgtccttgactactggggccaaggcaccactctcacagtctcctca

SEQ ID NO: 191 - DNA, 321 bp, Artificial Sequence
  Synthetic
gatatccagatgacacagactacatcctccctgtctgcctctctgggagacagagtcaccatcagttgcagggcaagtcaggacattagcaattttttaaactggtatcagcagagaccagatggaactgttaaactcctgatctactacacatcaagattacactcaggagtcccatcaaggttcagtggcagtgggtctgggacagatttttctctcaccgttagcaacctggagcaagaagatattgccacttacttttgccaacagggtcatacgcttccgtacacgttcggaggggggaccaagctggaaataaaa

SEQ ID NO: 192 - DNA, 360 bp, Artificial Sequence
  Synthetic
cagatccagctgcagcagtctggacctgagttggtgaggcctggggcttcagtgaagatatcctgcaaggcttctggctactccttcactgactactgtataaattgggtgaatcagaggcctggacagggacttgagtggattggatggatttatcctggaagcggtaatactaggtacagtgagaggttcaagggcaaggccacattgactgtagacacatcttccaacacagcctacatgcagctcagcagcctgacctctgaggactctgcggtctatttctgtgcaagagaggattactatccgtaccatggtatggactactggggtcaaggcacctcagtcaccgtctcctca

SEQ ID NO: 193 - DNA, 333 bp, Artificial Sequence
  Synthetic
gacattgtgctgacccaatctccaacttctttggctgtgtctctagggcagagggccaccatatcctgcagagccagtgaaagtgttgatggttatgacaatagttttatgcactggtaccagcagaaaccaggacagccacccaaactcctcatctttcgtgcatccaacctagaatctgggatccctgccaggttcagtggcagtgggtctaggacagacttcaccctcaccattaatcctgtggaggctgctgatgttgcaacctattactgtcagcaaagtagtgaggatccgtggacgttcggtggaggcaccaagctggaaatcaaa

SEQ ID NO: 194 - DNA, 354 bp, Artificial Sequence
  Synthetic
caggtccacctgcagcagcctggggctgagcttgtgaagcctggggcttcagtgaagatgtcctgcaaggcttctggctacaccttcaccagctactggataacctgggtgaagcagaggcctggacaaggccttgagtggattggagatatttttcctaatagtggtagaactaactacgatgagaagttcaagagcaaggccacactgactgtagacacatcctccagcaccgcctacatgcagctcagcagcctgacatctgaggactctgcggtctatttctgtgcaagggagggaaacttcggtagtcttgactactggggccaaggcaccactctcacagtctcctca

SEQ ID NO: 195 - DNA, 318 bp, Artificial Sequence
  Synthetic
caaattgttctcacccagtctccagcaatcatgtctgcatctccaggggagaaggtcaccatgacctgcagtgccaactcaaatttaaattacatgaactggtaccaccagaagtcaggcacctcccccaaaagatggatttatgacacatccaaactggcttctggagtccctgctcgcttcagtgccagtgggtctgggacctcttactctctcacaatcagcagcatggaggctgaagatgctgccacttattactgccagcagtggagtagaaaccctctcacgttcggtgctgggaccaggctggagctgaaa

SEQ ID NO: 196 - DNA, 357 bp, Artificial Sequence
  Synthetic
caggtcactctgaaagagtctggccctgggatgttgcagccctcccagaccctcagtctgacttgttctttctctgggttttcactgaacacttatgatgtgggtgtaggttggattcgtcagccttcagggaagggtctggagtggctggccaacatttggtggaatgatgataagtactataactcagccctgaagagccggctcacaatctccaaggatacctccaacaaccaggtcttcctcaagatctccagtgtggacactgcagatactgccacatactactgtaccctatatagttacgacggagggtttgcttactggggccaagggactctggtcactgtctctgca

SEQ ID NO: 197 - DNA, 324 bp, Artificial Sequence
  Synthetic
caaattgttctcacccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccgtacacgttcggaggggggaccaagctggaaataaaa

SEQ ID NO: 198 - DNA, 366 bp, Artificial Sequence
  Synthetic
gagatccagatgaagcagtctggagctgagctggtgaagcctggggcttcagtgaagatatcctgcaaggcttctggttactcattcactggctacaatatgaactgggtgaagcagagccatggaaagagccttgagtggattggaaatattaatccttactatggtagtactggctacaatcagaaattcaagggcaaggccacattgactgtagacaaatcttccagcacagcctacatgcagctcaacagcctgacatctgaggactctgcagtctattactgtgcaagaggggactatggttacgacgaggggacctggtttgcttactggggccaagggactctggtcactgtctctgca

SEQ ID NO: 199 - DNA, 339 bp, Artificial Sequence
  Synthetic
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagagcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttataatcttccattcacgttcggctcggggacaaaattggaaataaaa

SEQ ID NO: 200 - DNA, 360 bp, Artificial Sequence
  Synthetic
caggtccaactgcagcagcctggggctgagcttgtgaagcctggggcttcagtgaagatgtcctgcaaggcttctggctacaccttcaccaggcactggataacctgggtgaagcagaggcctggacaaggccttgagtggattggagatatttatcctggtagtggtagaactaactacaatgagaagttcaagagcacggccacactgactgtagacacatcctccagcacagcctacatgcagctcagcagcctgacatctgaggactctgcggtctattactgtgcacgagacggttacttatatataaactactttgactactggggccaaggaaccactctcacagtctcctca

SEQ ID NO: 201 - DNA, 318 bp, Artificial Sequence
  Synthetic
gaaaatgttctcacccagtctccagcaatcatgtctgcatctccaggggaaaaggtcaccatgacctgcagtgccagctcaagtgtaagtttcatgcactggttccagcagaagtcaagcacctcccccaaactctggatttatgacacatccaaactggcttctggagtcccaggtcgcttcagtggcagtgggtctggaagctcttactctctcacgatcagcagcatggcggctgaagatgttgccacttattactgttttcaggggagtgggtacccgtacacgttcggaggggggaccaagctggaaataaaa

SEQ ID NO: 202 - DNA, 354 bp, Artificial Sequence
  Synthetic
gaggttcagctgcagcagtctggggctgagcttgtgaggccaggggcctcagtcaagttgtcctgcacagcttctggctttaacattgtagacgactatatgcactgggtgaagcagaggcctgaacagggcctggagtggattggatggatttatcctgagaatgctgatactgaatatgcctcgaagttccagggcaaggccactataacagcagacacatcctccaacacagcctacctgcagctcagcagcctaacatctgaggacactgccgtctattactgtactaccgccactgggacgggctggtttgcttactggggccaagggactctggtcactgtctctgca

SEQ ID NO: 203 - DNA, 336 bp, Artificial Sequence
  Synthetic
gatgttgtgatgacccagactccactcactttgtcggttaccattggacaaccagcctccatctcttgcaagtcaagtcagagcctcttagatagtgatggaaagacatatttgaattggttgtttcagaggccaggccagtctccaaagcgcctaatctatctggtgtctaaactggactctggagtccctgacaggttcactggcagtggatcagggacagatttcacactgaaaatcagcagagtggagactgaggatttgggagtttattattgctggcaaggtacacattttccgtggacgttcggtggaggcgccaagctggaaatcaaa

SEQ ID NO: 204 - DNA, 354 bp, Artificial Sequence
  Synthetic
caggttcagctgcagcagtctggagctgagctgatgaagcctggggcctcagtgaagatatcctgcaaggctactggctacacattcagtaactactggatagagtgggtaaagcagaggcctggacatggccttgagtggattggagagattttacctggaagtggtagtactaactataatgagaacttcaagggcaaggccacattcactgcagatacatcctccaacacagcctacatgcaactcagcagcctgacatctgaggactctgccgtctattactgtgcacgaaggggggcctatggtaactttcactactggggccaaggcaccactctcacagtctcctca

SEQ ID NO: 205 - DNA, 324 bp, Artificial Sequence
  Synthetic
gaaattgtgctcacccagtctccagcactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggcgtagttacccgtacacgttcggaggggggaccaagctggaaataaaa

SEQ ID NO: 206 - DNA, 369 bp, Artificial Sequence
  Synthetic
gaggtccagctgcaacagtttggagctgagctggtgaagcctggggcttcagtgaagatatcctgcaaggcttctggctacacattcactgactacaacatggcctgggtgaaggagagccatggaaagagccttgagtggattggagatattaatcctaactatgatactactagctacaaccagaagttcaagggaaaggccacattgactgtagacaagtcctccagcacagcccacatggagctccgcagcctgacatctgagggcactgcagtctattactgtgcaagatcgggttactacggtagtagctactactggcacttcgatgtctggggcacagggaccacggtcaccgtctcctca

SEQ ID NO: 207 - DNA, 339 bp, Artificial Sequence
  Synthetic
gacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattataactatccattcacgttcggctcggggacaaagttggaaataaaa

SEQ ID NO: 208 - DNA, 348 bp, Artificial Sequence
  Synthetic
gaggttcagctgcagcagtctggggcagagctagtgaggtcaggggcctcagtcaagttgtcctgcacagcttctggcttcaacattaaagactactatatgcactgggtgaaacagaggcctgaacagggcctggagtggattggatggattgatcctgagagtggtgatactgaatatgccccgaagttccagggcagggccactatgactgcagacacatcctccaacacagcctacatgcagctcagcagcctgacatctgaggacactgccgtctattactgttatggacatgattatagggtggactgctggggtcaaggaacctcagtcactgtctcctca

SEQ ID NO: 209 - DNA, 336 bp, Artificial Sequence
  Synthetic
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatattccgtggacgttcggtggaggcaccaagctggaaatcaaa

SEQ ID NO: 210 - DNA, 369 bp, Artificial Sequence
  Synthetic
gaggtccagttgcaacagtttggagctgagctggtgaagcctggggcttcagtgaagatatcctgcaaggcttctggctacacattcactgactacaacatgggctgggtgaagcagagccatggaaagagccttgagtggattggagatattaatcctaactatgatagtactagctacacccagaagttcaagggaaaggccacattgactgtagacaagtcctccagcacagcctacatggaactccgcagcctgacatctgaggacactgcagtctattactgtgcaagatcgggttactacggtagtagctactactggcacttcgatgtctggggcacagggaccacggtcaccgtctcctca

SEQ ID NO: 211 - DNA, 339 bp, Artificial Sequence
  Synthetic
gacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcaacaatattatcactatccattcacgttcggctcggggacaaagttggaaataaaa

SEQ ID NO: 212 - DNA, 351 bp, Artificial Sequence
  Synthetic
caggtgcagctgaaggagtcaggacctggcctggtggcgccctcacagagcctgtccatcacatgcaccgtctcagggttctcattaaccaactatggtgttcactgggttcgccagcctccagggaagggtctggaatggctggtcgtgatatggaatgatggaagcgcaacctataattcagctctcgaatccagactgagcatcagcaaggacaactccaagagccaagttttcttaaaaatgaacagtctccaaactgatgacacagccatgtactactgtgccagacatgaatctagtaacccgtttgcttactggggccaagggactctggtcactgtctctgca

SEQ ID NO: 213 - DNA, 321 bp, Artificial Sequence
  Synthetic
gacatcttgctgactcagtctccagccatcctgtctgtgagtccaggagaaagagtcagtttctcctgcagggccagtcagagcattggcacaagcatccactggtatcagcaaagaacaaatggctctccaaggcttctcataaagtctgcttctgagtctatcgctgggattccttccaggtttagtggcagtggatcagggacagattttactcttagcatcaacagtgtggagtctgaagatattgcagattattactgtcaacaaaataataggtggccgtacacgttcggaggggggaccaagctggaaataaaa

SEQ ID NO: 214 - DNA, 339 bp, Artificial Sequence
  Synthetic
gaggttcagctgcagcagtctggggctgagcttgtgaggccaggggcctcagtcaagttgtcctgcacagcttctgactttaacattaaagacgactatatacattgggtgaaacagaggcctgaacagggcctggagtggattggaaggattgatcctgcgaatggtaatactaaatatgccccgaagttccaggacaaggccactataactgcagacacatcctccaacacagcctacctgcagctcagcagcctgacttctgaggacactgccgtctattactgtgctctgggctacacctactggggccaaggcaccactctcacagtctcctca

SEQ ID NO: 215 - DNA, 336 bp, Artificial Sequence
  Synthetic
gatgttgtgatgactcagaccccactcactttgtcggttaccattggacaaccagcctccatctcttgcaagtcaagtcagagcctcttacatagttatggaaagacatatttgaattggttattacagaggccaggccagtctccaaagctcctaatctatctggtgtctaaactggaatctggagtccctgacaggttcagtggcagtggatcagggacagatttcacactgaaaatcagcagagtggaggctgaggatttgggagtttattactgcttgcaaactacacattttcctcagacgttcggtggaggcaccaagctggaaatcaag

SEQ ID NO: 216 - DNA, 360 bp, Artificial Sequence
  Synthetic
gaggtgcagctggtggagtctgggggagacttagtgaagcctggagggtccctgaaactctcctgtgcagcctctggattcactttcagtgactatggaatgcactgggttcgtcagggtccagagaaggggctggagtgggttgcatacattaatagtggcagtagtaccatctactatgcagacacagtgaagggccgattcaccatctccagagacaatgccaagaacaccctgttcctgcaaatgaccagtctgaggtctgaggacacggccatgtattactgtgcaaggccgggggattacgacaactatgctatggactactggggtcaaggaacctcagtcaccgtctcctca

SEQ ID NO: 217 - DNA, 324 bp, Artificial Sequence
  Synthetic
gacattgtgatgacccagtctcacaaattcctgtccacatcagtaggagacagggtcagcatcacctgcaaggccagtcaggatgtgagtgttgctgtagcctggtatcaacaaaaaccagggcaatctcctaaactactgatttactgggcatacacccggcacactggagtccctgatcgcttcacaggcagtggatctgggacagaatatactctcaccatcagcagtgtgcaggctgaagacctggcactttattactgtcagcaacattataacactcctccgtggacgttcggtggaggcaccaagctggaaatcaaa

SEQ ID NO: 218 - DNA, 348 bp, Artificial Sequence
  Synthetic
gaggttcagctgcaacagtctggggcagagcttgtgaggtcaggggcctcagtcaagttgtcctgcacagcttctggcttcaacattaaagactactatatacactgggtgaagcagaggcctgaacagggcctggagtggattggatggattgatcctgagaatgctgatactgaatatgccccgaagttccagggcaaggccactatgactccagacacatcctccaacacagcctacctgcagctcagcagcctgacatctgaggacactgccgtctattactgttatgcctgggactattctatggactactggggtcaaggaacctcagtcaccgtctcctca

SEQ ID NO: 219 - DNA, 336 bp, Artificial Sequence
  Synthetic
gatgttgtgatgacccaaactccactctccctgtctgtcagtcttggagatcaagcctccatctcttgcagatctagtcagagccttgtacacagtaatggaaacacctatttacattggtacctgcagaagccgggccagtctccaaagctcctgatctacaaagtttccaaccgattttctggggtcccagacaggttcagtggcagtggatcagggacagatttcatactcaagatcagtagagtggaggctgaggatctgggagtttatttctgctctcaaaatacacatgttccgtacacgttcggaggggggaccaggctggaaataaaa

SEQ ID NO: 220 - DNA, 357 bp, Artificial Sequence
  Synthetic
caggttcagctgcagcagtctggaactgagctggcgaggcctggggcttcagtgaagctgtcctgcaaggcttctggcttcaccttcacagactatggtataaactgggtgaaacagagaactggacagggccttgagtggattggagagatttatcctagcagtggtaattcttactacaatgagaaattcaaggccaaggccacactgactgcagacaaatcctccagcacagcgtacatggagctccgcagtctgacatctgaggactctgcggtctatttctgtgcaagatcgacttactacggtagcccaattgactactggggccaaggcaccactctcacagtctcctca

SEQ ID NO: 221 - DNA, 321 bp, Artificial Sequence
  Synthetic
gacattgtgatgacccagtctcacaaattcatgtccacaccagtaggagacagggtcagcatcacctgcaaggccagtcaggatgtggatactactgtagcctggtatcaacagaaaccagggcaatctccgaaactactgatttactgggcatccacccggcaaattggagtccctgatcgcttcacaggcagtggatctgggacagatttcactctcaccattagtaatgtgcagtctgaagacttggcagattatttctgtcagcaatatagcacctatcctctgacgttcggtggaggcaccaagctggaaatcaaa

SEQ ID NO: 222 - DNA, 357 bp, Artificial Sequence
  Synthetic
caggtgcagctgaaggagtcaggacctggcctggtggcgccctcacagagcctgtccattacatgcactgtctctgggttctcattgaccagctatgctataacctgggttcgccagtcaccaggaaagggtctggagtggcttggattaatatggactggtggaggcacaaattataattcagcgctcaaatccagactgagcatcagcaaagacaactccaagagtcaagttttcttaaaaatgaacagtctgcaaactgatgacacagccaggtactactgtgccagaatctatgatggttactaccggtacttcgatgtctggggcacagggaccacggtcaccgtctcctca

SEQ ID NO: 223 - DNA, 321 bp, Artificial Sequence
  Synthetic
cgtattgtgttgacccagactcccaaattcctgcttgtatcagcaggagacagggttaccatgacctgcaaggccagtcagagtgtgagtaatgatgtagcttggtaccaacagaagccagggcagtctcctaaactgctgatatactatgcatccaatcgttacactggagtccctgatcgcttcactggcagtggatatgggacggatttcactttcaccatcagcactgtgcaggctgaagacctggcagtttatttctgtcagcaggattatagctctccgtggacgttcggtggaggcaccaagctggaaatcaaa

SEQ ID NO: 224 - DNA, 348 bp, Artificial Sequence
  Synthetic
gaggttcagctgcagcagtctggggcagagcttgtgaggtcaggggcctcagtcaagttgtcctgcacagcttctggcttcaacattaaggactactatatgcactgggtgaagcagaggcctgaacagggcctggactggattggatggattgatcctgagaatggtgatactgaatatgccccgaaattccagggcaaggccactatgactgcagacacatcctccaacacagcctacctgcagctcagcagcctgacatctgaggacactgccgtctattactgtaatgtccttactatgcctacggcttactggggccaagggactctggtcactgtctctgca

SEQ ID NO: 225 - DNA, 339 bp, Artificial Sequence
  Synthetic
gacattgtgatgtcacagtctccatcctccctagctgtgtcagttggagagaaggttattatgagctgcaagtccagtcagagccttttatatagtagcaatcaaaagaactatttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccgtacacgttcggaggggggaccaagctggaaataaaa

SEQ ID NO: 226 - DNA, 342 bp, Artificial Sequence
  Synthetic
gaggtgcaactgatggaatctgggggagacttagtgaagcctggagggtccctgaaactctcctgtgcagcctctggattcactttcagtagctatggcttgtcttgggttcgccagactccagacaagaggctggagtgggtcgcaaccattactagtggtggtagttatacctactatccagacagtgtgaaggggcgattcaccatctccagagacaatgccaggaacaccctgtacctgcaaatgttcagtctgaagtctgaggacacagccatgtattactgtgcactatggtcccttgactactggggccaaggcaccactctcacagtctcctca

SEQ ID NO: 227 - DNA, 318 bp, Artificial Sequence
  Synthetic
caaattgttctcacccagtctccagcaatcatgtctgcatctccaggggagaaggtcaccatgacctgcagtgccaattcaagtttaagttacatgcactggtaccagcagaagccaggcacctcccccaaaagatggatttatgacacatccgaactggcttctggagtccctgctcgcttcagtggcagtgggtctgggacctcttattctctcacaatcagcagcatggaggctgaagatgctgccacttattactgccatcagcggaggagttacccatggacgttcggtggaggcaccaagctggaaatcaaa

SEQ ID NO: 228 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 229 - protein, 760 aa, Macaca mulatta
MMDQARSAFSNLFGGEPLSYTRFSLARQVDGDNSHVEMKLGVDEEENTDNNTKPNGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 230 - protein, 760 aa, Macaca fascicularis
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 231 - protein, 763 aa, Mus musculus
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 232 - protein, 19 aa, Artificial Sequence
  Synthetic
MGWSCIILFLVATATGVHS

SEQ ID NO: 233 - protein, 8 aa, Artificial Sequence
  Synthetic
IDPETGDT

SEQ ID NO: 234 - protein, 17 aa, Artificial Sequence
  Synthetic
WIDPETGDTEYASKFQD

SEQ ID NO: 235 - protein, 5 aa, Artificial Sequence
  Synthetic
  misc_feature (3)..(3): Xaa can be any naturally occurring amino acid
LPXTG

SEQ ID NO: 236 - protein, 17 aa, Artificial Sequence
  Synthetic
WIDPESGDTEYASKFQD

SEQ ID NO: 237 - protein, 8 aa, Artificial Sequence
  Synthetic
GYSFTDYY

SEQ ID NO: 238 - protein, 5 aa, Artificial Sequence
  Synthetic
DYYIN

SEQ ID NO: 239 - protein, 8 aa, Artificial Sequence
  Synthetic
GYSFTDYD

SEQ ID NO: 240 - protein, 5 aa, Artificial Sequence
  Synthetic
DYDIN

SEQ ID NO: 241 - protein, 25 aa, Artificial Sequence [sequence omitted]

SEQ ID NO: 242 - protein, 13 aa, Artificial Sequence
  Synthetic
WVRQAPGKGLEWV

SEQ ID NO: 243 - protein, 30 aa, Artificial Sequence
  Synthetic
RFTISRDNSKNTLYLQMNSLRAEDTAVYYC

SEQ ID NO: 244 - protein, 11 aa, Artificial Sequence
  Synthetic
WGQGTLVTVSS

SEQ ID NO: 245 - protein, 25 aa, Artificial Sequence [sequence omitted]

SEQ ID NO: 246 - protein, 13 aa, Artificial Sequence
  Synthetic
WVRQAPGQGLEWM

SEQ ID NO: 247 - protein, 30 aa, Artificial Sequence
  Synthetic
RVTITADTSTSTAYMELSSLRSEDTAVYYC

SEQ ID NO: 248 - protein, 10 aa, Artificial Sequence
  Synthetic
FGQGTKVEIK

SEQ ID NO: 249 - protein, 25 aa, Artificial Sequence
  Synthetic
QVQLQESGPGLVKPSQTLSLTCTVS

SEQ ID NO: 250 - protein, 13 aa, Artificial Sequence
  Synthetic
WIRQPPGKGLEWI

SEQ ID NO: 251 - protein, 30 aa, Artificial Sequence
  Synthetic
RVTISVDTSKNQFSLKLSSVTAADTAVYYC

SEQ ID NO: 252 - protein, 23 aa, Artificial Sequence
  Synthetic
DIVMTQSPDSLAVSLGERATINC

SEQ ID NO: 253 - protein, 23 aa, Artificial Sequence
  Synthetic
DIQMTQSPSSLSASVGDRVTITC

SEQ ID NO: 254 - protein, 15 aa, Artificial Sequence
  Synthetic
WYQQKPGKAPKLLIY

SEQ ID NO: 255 - protein, 15 aa, Artificial Sequence
  Synthetic
WYQQKPGQPPKLLIY

SEQ ID NO: 256 - protein, 32 aa, Artificial Sequence
  Synthetic
GVPSRFSGSGSGTDFTLTISSLQPEDFATYYC

SEQ ID NO: 257 - protein, 23 aa, Artificial Sequence
  Synthetic
DIVMTQSPLSLPVTPGEPASISC

SEQ ID NO: 258 - protein, 15 aa, Artificial Sequence
  Synthetic
WYLQKPGQSPQLLIY

SEQ ID NO: 259 - protein, 32 aa, Artificial Sequence
  Synthetic
GVPDRFSGSGSGTDFTLKISRVEAEDVGVYYC

SEQ ID NO: 260 - protein, 32 aa, Artificial Sequence
  Synthetic
GVPDRFSGSGSGTDFTLTISSLQAEDFAVYYC

SEQ ID NO: 261 - protein, 8 aa, Artificial Sequence
  Synthetic
GYTFTDYG

SEQ ID NO: 262 - protein, 9 aa, Artificial Sequence
  Synthetic
GFSITSGYY

SEQ ID NO: 263 - protein, 8 aa, Artificial Sequence
  Synthetic
IYPSSGNT

SEQ ID NO: 264 - protein, 7 aa, Artificial Sequence
  Synthetic
INFDGNN

SEQ ID NO: 265 - protein, 7 aa, Artificial Sequence
  Synthetic
INFDGNS

SEQ ID NO: 266 - protein, 12 aa, Artificial Sequence
  Synthetic
ARSNYYGSPIDF

SEQ ID NO: 267 - protein, 12 aa, Artificial Sequence
  Synthetic
ARFYYDYDYFDY

SEQ ID NO: 268 - protein, 12 aa, Artificial Sequence
  Synthetic
ARIYYDYDYFDY

SEQ ID NO: 269 - protein, 447 aa, Artificial Sequence
  Synthetic
EVQLQQSGAELVRPGASVKLSCTASGFNIKDDYMYWVKQRPEQGLEWIGWIDPETGDTEYASKFQDKATVTADTSSNTAYLQLSSLTSEDTAVYYCTLWLRRGLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 270 - protein, 447 aa, Artificial Sequence
  Synthetic
EVQLQQSGAELVRPGASVKLSCTASGFNIKDDYMYWVKQRPEQGLEWIGWIDPESGDTEYASKFQDKATVTADTSSNTAYLQLSSLTSEDTAVYYCTLWLRRGLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 271 - protein, 450 aa, Artificial Sequence
  Synthetic
QIQLQQSGPELVRPGASVKISCKASGYSFTDYYINWVNQRPGQGLEWIGWIYPGSGNTRYSERFKGKATLTVDTSSNTAYMQLSSLTSEDSAVYFCAREDYYPYHGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 272 - protein, 450 aa, Artificial Sequence
  Synthetic
QIQLQQSGPELVRPGASVKISCKASGYSFTDYDINWVNQRPGQGLEWIGWIYPGSGNTRYSERFKGKATLTVDTSSNTAYMQLSSLTSEDSAVYFCAREDYYPYHGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 273 - protein, 225 aa, Artificial Sequence
  Synthetic
EVQLQQSGAELVRPGASVKLSCTASGFNIKDDYMYWVKQRPEQGLEWIGWIDPETGDTEYASKFQDKATVTADTSSNTAYLQLSSLTSEDTAVYYCTLWLRRGLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 274 - protein, 225 aa, Artificial Sequence
  Synthetic
EVQLQQSGAELVRPGASVKLSCTASGFNIKDDYMYWVKQRPEQGLEWIGWIDPESGDTEYASKFQDKATVTADTSSNTAYLQLSSLTSEDTAVYYCTLWLRRGLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 275 - protein, 228 aa, Artificial Sequence
  Synthetic
QIQLQQSGPELVRPGASVKISCKASGYSFTDYYINWVNQRPGQGLEWIGWIYPGSGNTRYSERFKGKATLTVDTSSNTAYMQLSSLTSEDSAVYFCAREDYYPYHGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 276 - protein, 228 aa, Artificial Sequence
  Synthetic
QIQLQQSGPELVRPGASVKISCKASGYSFTDYDINWVNQRPGQGLEWIGWIYPGSGNTRYSERFKGKATLTVDTSSNTAYMQLSSLTSEDSAVYFCAREDYYPYHGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 277 - protein, 12 aa, Artificial Sequence
  Synthetic
ARSFYDYDVLDY

SEQ ID NO: 278 - protein, 6 aa, Artificial Sequence
  Synthetic
QDISNC

SEQ ID NO: 279 - protein, 6 aa, Artificial Sequence
  Synthetic
QDISNS

SEQ ID NO: 280 - protein, 6 aa, Artificial Sequence
  Synthetic
QDITNF

SEQ ID NO: 281 - protein, 6 aa, Artificial Sequence
  Synthetic
QDISNY

SEQ ID NO: 282 - protein, 9 aa, Artificial Sequence
  Synthetic
QQGNTLPWT

SEQ ID NO: 283 - protein, 9 aa, Artificial Sequence
  Synthetic
QQGNTLPFT

SEQ ID NO: 284 - protein, 9 aa, Artificial Sequence
  Synthetic
QQGNTLPYT

SEQ ID NO: 285 - protein, 9 aa, Artificial Sequence
  Synthetic
QQGHAYPYT

SEQ ID NO: 286 - protein, 9 aa, Artificial Sequence
  Synthetic
  SITE (9)..(9): Xaa may be Y or G
GYSITSGYX

SEQ ID NO: 287 - protein, 7 aa, Artificial Sequence
  Synthetic
  SITE (2)..(2): Xaa may be T or N
  SITE (6)..(6): Xaa may be A or N
  SITE (7)..(7): Xaa may be N, T, or S
IXFDGXX

SEQ ID NO: 288 - protein, 12 aa, Artificial Sequence
  Synthetic
  SITE (1)..(1): Xaa may be T or A
  SITE (3)..(3): Xaa may be S, F, or I
  SITE (4)..(4): Xaa may be S, N, or Y
  SITE (9)..(9): Xaa may be V, P, or Y
  SITE (10)..(10): Xaa may be L, I, or F
  SITE (12)..(12): Xaa may be Y or F
XRXXYDYDXXDX

SEQ ID NO: 289 - protein, 6 aa, Artificial Sequence
  Synthetic
  SITE (4)..(4): Xaa may be S or T
  SITE (6)..(6): Xaa may be F, C, S, or Y
QDIXNX

SEQ ID NO: 290 - protein, 9 aa, Artificial Sequence
  Synthetic
  SITE (4)..(4): Xaa may be H or N
  SITE (5)..(5): Xaa may be T or A
  SITE (6)..(6): Xaa may be L or Y
  SITE (8)..(8): Xaa may be Y, W, or F
QQGXXXPXT

(19) United States
(12) Patent Application Publication
(10) Pub. No.: US 2023/0113823 A1
(43) Pub. Date: Apr. 13, 2023

(54) ANTI-TRANSFERRIN RECEPTOR (TFR) ANTIBODY AND USES THEREOF

(71) Applicant: Dyne Therapeutics, Inc., Waltham, MA (US)

(72) Inventors: Romesh R. Subramanian, Framingham, MA (US); Mohammed T. Qatanani, Waltham, MA (US); Timothy Weeden, Waltham, MA (US); Cody A. Desjardins, Waltham, MA (US); Brendan Quinn, Boston, MA (US)

(73) Assignee: Dyne Therapeutics, Inc., Waltham, MA (US)

(21) Appl. No.: 17/796,418

(22) PCT Filed: Jan. 8, 2021
(86) PCT No.: PCT/US2021/012666

§ 371 Date: Jul. 29, 2022

Related U.S. Application Data
(60) Provisional application No. 63/055,405, filed on Jul. 23, 2020.
(60) Provisional application No. 62/968,252, filed on Jan. 31, 2020.

(51) Int. Cl. C07K 16/28; A61K 47/68

(52) U.S. Cl. CPC C07K 16/2881; A61K 47/6807; A61K 47/6849; A61K 2039/505

(57) ABSTRACT

Aspects of the disclosure relate to antibodies that bind to transferrin receptor (e.g., transferrin receptor 1) and complexes comprising the antibody covalently linked to a molecular payload. Methods of making and using the antibodies are also provided.

### RELATED APPLICATIONS

This application claims the benefit under 35 U.S.C § 119(e) of the filing date of U.S. Provisional Application No. 63/055,405, entitled “ANTI-TRANSFERRIN RECEPTOR (TFR) ANTIBODY AND USES THEREOF”, filed Jul. 23, 2020, and U.S. Provisional Application No. 62/968,252, entitled “ANTI-TRANSFERRIN RECEPTOR (TFR) ANTIBODY AND USES THEREOF”, filed Jan. 31, 2020; the contents of each of which are incorporated herein by reference in their entirety.

### FIELD OF THE INVENTION

The present application relates to novel anti-transferrin receptor (TfR) antibodies and the use of the antibodies.

### REFERENCE TO SEQUENCE LISTING SUBMITTED AS A TEXT FILE VIA EFS-WEB

The instant application contains a sequence listing which has been submitted in ASCII format via EFS-Web and is hereby incorporated by reference in its entirety. Said ASCII copy, created on Jan. 8, 2021, is named D082470023WO00-SEQ-ZJG and is 180 kilobytes in size.

### BACKGROUND

Transferrin Receptor (TfR) is a dimeric transmembrane glycoprotein receptor involved in iron transport. Two transferrin receptors have been characterized in humans, transferrin receptor 1 (TfR1) and transferrin receptor 2 (TfR2). It has been shown that TfR is overexpressed in cancer cells with higher metastatic potential. TfR1 has been shown to express on the endothelial cells of the blood brain barrier can be used to allow the delivery of large molecules into the brain.

### SUMMARY

The present disclosure is based, at least in part, on the development of new antibodies that bind transferrin receptor (anti-TfR antibodies). In some embodiments, anti-TfR antibodies described herein selectively bind to human or non-human primate (NHP) transferrin receptor with high specificity and affinity (e.g., subnanomolar to nanomolar range). In some embodiments, the anti-TfR antibodies described herein are useful for targeting tissues and/or (e.g., and) cells that express TfR. In some embodiments, the anti-TfR antibodies provided herein are used for detection of TfR in a cell or a tissue. In some embodiments, the anti-TfR antibodies provided herein are used in diagnostic, therapeutic, or research applications. In some embodiments, the anti-TfR antibodies described herein are used to deliver a molecular payload to a target cell or tissue (e.g., a cell or tissue that expresses TfR).

As such, in some aspects, complexes comprising the anti-TfR antibodies conjugated (e.g., covalently conjugated) to a molecular payload (e.g., a diagnostic agent or a therapeutic agent) are provided. In some embodiments, the anti-TfR antibodies is used to deliver the conjugated molecular payload to a cell or a tissue that expresses TfR1 (e.g., muscle or the brain) for diagnosing and/or (e.g., and) treating a disease (e.g., a muscle disease or a neurological disease). In some aspects, the present disclosure provides data demonstrating that the anti-TfR antibodies described herein has superior activity in delivering molecular payload into a target cell (e.g., a muscle cell), compared with other known anti-TfR antibodies.

Some aspects of the present disclosure provide antibodies that bind to human transferrin receptor (TfR), wherein the antibody comprises: a CDR-H1, a CDR-H2, and a CDR-H3 of any one of the antibodies listed in Table 1; and/or wherein the antibody comprises a CDR-L1, a CDR-L2, and a CDR-L3, of any one of the antibodies listed in Table 1 or Table 3.

Some aspects of the present disclosure provide antibodies (e.g., isolated antibodies) that bind to human transferrin receptor (TfR), wherein the antibody comprises: (i) a heavy chain complementary determining region 1 (CDR-H1), a heavy chain complementary determining region 2 (CDR-H2), and a heavy chain complementary determining region 3 (CDR-H3) of a heavy chain variable region (VH) comprising the amino acid sequence of SEQ ID NO: 7; and/or (e.g., and) (ii) a light chain complementary determining region 1 (CDR-L1), a light chain complementary determining region 2 (CDR-L2), and a light chain complementary determining region 3 (CDR-L3) of a light chain variable region (VL) comprising the amino acid sequence of SEQ ID NO: 8.

In some embodiments, the antibody comprises CDR-H1 as set forth in SEQ ID NO: 1, a CDR-H2 as set forth in SEQ ID NO: 2, a CDR-H3 as set forth in SEQ ID NO: 3; and/or (e.g., and) a CDR-L1 as set forth in SEQ ID NO: 4, a CDR-L2 as set forth in SEQ ID NO: 5, and a CDR-L3 as set forth in SEQ ID NO: 6. In some embodiments, the antibody comprises a CDR-H1 as set forth in SEQ ID NO: 145, a CDR-H2 as set forth in SEQ ID NO: 146, a CDR-H3 as set forth in SEQ ID NO: 147; and/or (e.g., and) a CDR-L1 as set forth in SEQ ID NO: 148, a CDR-L2 as set forth in SEQ ID NO: 149, and a CDR-L3 as set forth in SEQ ID NO: 6. In some embodiments, the antibody comprises a CDR-H1 as set forth in SEQ ID NO: 150, a CDR-H2 as set forth in SEQ ID NO: 151, a CDR-H3 as set forth in SEQ ID NO: 152; and/or (e.g., and) a CDR-L1 as set forth in SEQ ID NO: 153, a CDR-L2 as set forth in SEQ ID NO: 5, and a CDR-L3 as set forth in SEQ ID NO: 154.

In some embodiments, the antibody comprises a CDR-H1 as set forth in SEQ ID NO: 1, a CDR-H2 as set forth in SEQ ID NO: 233 or SEQ ID NO: 80, a CDR-H3 as set forth in SEQ ID NO: 3; and/or a CDR-L1 as set forth in SEQ ID NO: 4, a CDR-L2 as set forth in SEQ ID NO: 5, and a CDR-L3 as set forth in SEQ ID NO: 6. In some embodiments, the antibody comprises CDR-H1 as set forth in SEQ ID NO: 145, a CDR-H2 as set forth in SEQ ID NO: 234 or SEQ ID NO: 236, a CDR-H3 as set forth in SEQ ID NO: 147; and/or a CDR-L1 as set forth in SEQ ID NO: 148, a CDR-L2 as set forth in SEQ ID NO: 149, and a CDR-L3 as set forth in SEQ ID NO: 6. In some embodiments, the antibody comprises a CDR-H1 as set forth in SEQ ID NO: 150, a CDR-H2 as set forth in SEQ ID NO: 277 or SEQ ID NO: 278, a CDR-H3 as set forth in SEQ ID NO: 152; and/or a CDR-L1 as set forth in SEQ ID NO: 153, a CDR-L2 as set forth in SEQ ID NO: 5, and a CDR-L3 as set forth in SEQ ID NO: 154.

In some embodiments, the antibody comprises a VH comprising an amino acid sequence at least 85% identical to SEQ ID NO: 7, and/or (e.g., and) a VL comprising an amino acid sequence at least 85% identical to SEQ ID NO: 8.

Other aspects of the present disclosure provide antibodies (e.g., isolated antibodies) that bind to human transferrin receptor (TfR), wherein the antibody comprises: (i) a heavy chain complementary determining region 1 (CDR-H1), a heavy chain complementary determining region 2 (CDR-H2), and a heavy chain complementary determining region 3 (CDR-H3) of a heavy chain variable region (VH) comprising the amino acid sequence of SEQ ID NO: 15; and/or (e.g., and) (ii) a light chain complementary determining region 1 (CDR-L1), a light chain complementary determining region 2 (CDR-L2), and a light chain complementary determining region 3 (CDR-L3) of a light chain variable region (VL) comprising the amino acid sequence of SEQ ID NO: 16.

In some embodiments, the antibody comprises a CDR-H1 as set forth in SEQ ID NO: 9, a CDR-H2 as set forth in SEQ ID NO: 10, a CDR-H3 as set forth in SEQ ID NO: 11; and/or (e.g., and) a CDR-L1 as set forth in SEQ ID NO: 12, a CDR-L2 as set forth in SEQ ID NO: 13, and a CDR-L3 as set forth in SEQ ID NO: 14. In some embodiments, the antibody comprises a CDR-H1 as set forth in SEQ ID NO: 155, a CDR-H2 as set forth in SEQ ID NO: 156, a CDR-H3 as set forth in SEQ ID NO: 157; and/or (e.g., and) a CDR-L1 as set forth in SEQ ID NO: 158, a CDR-L2 as set forth in SEQ ID NO: 159, and a CDR-L3 as set forth in SEQ ID NO: 14. In some embodiments, the antibody comprises a CDR-H1 as set forth in SEQ ID NO: 160, a CDR-H2 as set forth in SEQ ID NO: 161, a CDR-H3 as set forth in SEQ ID NO: 162; and/or (e.g., and) a CDR-L1 as set forth in SEQ ID NO: 163, a CDR-L2 as set forth in SEQ ID NO: 13, and a CDR-L3 as set forth in SEQ ID NO: 164.

In some embodiments, the antibody comprises a CDR-H1 as set forth in SEQ ID NO: 237 or SEQ ID NO: 239, a CDR-H2 as set forth in SEQ ID NO: 18, a CDR-H3 as set forth in SEQ ID NO: 19; and/or a CDR-L1 as set forth in SEQ ID NO: 20, a CDR-L2 as set forth in SEQ ID NO: 21, and a CDR-L3 as set forth in SEQ ID NO: 22. In some embodiments, the antibody comprises a CDR-H1 as set forth in SEQ ID NO: 238 or SEQ ID NO: 240, a CDR-H2 as set forth in SEQ ID NO: 166, a CDR-H3 as set forth in SEQ ID NO: 167; and/or a CDR-L1 as set forth in SEQ ID NO: 168, a CDR-L2 as set forth in SEQ ID NO: 169, and a CDR-L3 as set forth in SEQ ID NO: 22.

In some embodiments, the antibody comprises a VH comprising an amino acid sequence at least 85% identical to SEQ ID NO: 15, and/or (e.g., and) a VL comprising an amino acid sequence at least 85% identical to SEQ ID NO: 16.

Other aspects of the present disclosure provide antibodies (e.g., isolated antibodies) that bind to human transferrin receptor (TfR). In some embodiments, the antibody comprises: (i) a heavy chain complementary determining region 1 (CDR-H1), a heavy chain complementary determining region 2 (CDR-H2), and a heavy chain complementary determining region 3 (CDR-H3) of a heavy chain variable region (VH) comprising the amino acid sequence of SEQ ID NO: 23; and/or (e.g., and) (ii) a light chain complementary determining region 1 (CDR-L1), a light chain complementary determining region 2 (CDR-L2), and a light chain complementary determining region 3 (CDR-L3) of a light chain variable region (VL) comprising the amino acid sequence of SEQ ID NO: 24.

In some embodiments, the antibody comprises a CDR-H1 as set forth in SEQ ID NO: 17, a CDR-H2 as set forth in SEQ ID NO: 18, a CDR-H3 as set forth in SEQ ID NO: 19; and/or (e.g., and) a CDR-L1 as set forth in SEQ ID NO: 20, a CDR-L2 as set forth in SEQ ID NO: 21, and a CDR-L3 as set forth in SEQ ID NO: 22. In some embodiments, the antibody comprises a CDR-H1 as set forth in SEQ ID NO: 165, a CDR-H2 as set forth in SEQ ID NO: 166, a CDR-H3 as set forth in SEQ ID NO: 167; and/or (e.g., and) a CDR-L1 as set forth in SEQ ID NO: 168, a CDR-L2 as set forth in SEQ ID NO: 169, and a CDR-L3 as set forth in SEQ ID NO: 22. In some embodiments, the antibody comprises a CDR-H1 as set forth in SEQ ID NO: 170, a CDR-H2 as set forth in SEQ ID NO: 171, a CDR-H3 as set forth in SEQ ID NO: 172; and/or (e.g., and) a CDR-L1 as set forth in SEQ ID NO: 173, a CDR-L2 as set forth in SEQ ID NO: 21, and a CDR-L3 as set forth in SEQ ID NO: 174.

In some embodiments, the antibody comprises a VH comprising an amino acid sequence at least 85% identical to SEQ ID NO: 23, and/or (e.g., and) a VL comprising an amino acid sequence at least 85% identical to SEQ ID NO: 24.

In some embodiments, the antibody comprises human or humanized framework regions with the CDR-H1, the CDR-H2, the CDR-H3 of a VH as set forth in SEQ ID NO: 15, and the CDR-L1, the CDR-L2, the CDR-L3 of a VL as set forth in SEQ ID NO: 16. In some embodiments, the antibody comprises human or humanized framework regions with the CDR-H1, the CDR-H2, the CDR-H3 of a VH as set forth in SEQ ID NO: 7, and the CDR-L1, the CDR-L2, the CDR-L3 of a VL as set forth in SEQ ID NO: 8. In some embodiments, the antibody comprises human or humanized framework regions with the CDR-H1, the CDR-H2, the CDR-H3 of a VH as set forth in SEQ ID NO: 23, and the CDR-L1, the CDR-L2, the CDR-L3 of a VL as set forth in SEQ ID NO: 24.

In some embodiments, the antibody is a humanized antibody. In some embodiments, the humanized antibody comprises a humanized VH and/or (e.g., and) a humanized VL. In some embodiments, the antibody is selected from the group consisting of a full-length IgG, a Fab fragment, a F(ab′) fragment, a F(ab′)2 fragment, a scFv, and a Fv.

In some embodiments, the antibody is a full-length IgG. In some embodiments, the antibody comprises a heavy chain constant region of the isotype IgG1, IgG2, IgG3, or IgG4. In some embodiments, the antibody comprises a heavy chain constant region of the isotype IgG1 set forth in SEQ ID NO: 175 or SEQ ID NO: 176. In some embodiments, the antibody comprises a heavy chain comprising an amino acid sequence at least 85% identical to SEQ ID NO: 178, and/or (e.g., and) a light chain comprising an amino acid sequence at least 85% identical to SEQ ID NO: 179. In some embodiments, the antibody comprises a heavy chain comprising an amino acid sequence at least 85% identical to SEQ ID NO: 180, and/or (e.g., and) a light chain comprising an amino acid sequence at least 85% identical to SEQ ID NO: 181. In some embodiments, a heavy chain comprising an amino acid sequence at least 85% identical to SEQ ID NO: 182, and/or (e.g., and) a light chain comprising an amino acid sequence at least 85% identical to SEQ ID NO: 183.

In some embodiments, the antibody is a F(ab′) fragment. In some embodiments, the antibody comprises a heavy chain comprising an amino acid sequence at least 85% identical to SEQ ID NO: 185, and/or (e.g., and) a light chain comprising an amino acid sequence at least 85% identical to SEQ ID NO: 179. In some embodiments, the antibody comprises a heavy chain comprising an amino acid sequence at least 85% identical to SEQ ID NO: 186, and/or (e.g., and) a light chain comprising an amino acid sequence at least 85% identical to SEQ ID NO: 181. In some embodiments, the antibody comprises a heavy chain comprising an amino acid sequence at least 85% identical to SEQ ID NO: 187, and/or (e.g., and) a light chain comprising an amino acid sequence at least 85% identical to SEQ ID NO: 183.

Further provided herein are antibodies (e.g., isolated antibodies) that bind to human transferrin receptor (TfR). In some embodiments, the antibody comprises: a CDR-H1, a CDR-H2, and a CDR-H3, which collectively contains no more than 10 amino acid variations, preferably no more than 8 amino acid variations, and more preferably no more than 5 amino acid variations, as compared with the CDR-H1, a CDR-H2, and a CDR-H3 of any one of the antibodies listed in Table 1; and/or (e.g., and) wherein the antibody comprises a CDR-L1, a CDR-L2, and a CDR-L3, which collectively contains no more than 10 amino acid variations, preferably no more than 8 amino acids variations, as compared with the a CDR-L1, a CDR-L2, and a CDR-L3 of any one of the antibodies listed in Table 1. In some embodiments, the antibody comprises: a CDR-H1, a CDR-H2, and a CDR-H3 of any one of the antibodies listed in Table 1; and/or (e.g., and) wherein the antibody comprises a CDR-L1, a CDR-L2, and a CDR-L3, of any one of the antibodies listed in Table 1.

In some embodiments, antibodies (e.g., isolated antibodies) that bind to human transferrin receptor (TfR) comprise: a CDR-H1 set forth as GYSITSGYX₁ (SEQ ID NO: 286), in which X₁ can be Y or G; a CDR-H2 set forth as IX₂FDGX₃X₄ (SEQ ID NO: 287), in which X₂ can be T or N, X₃ can be A or N, and X₄ can be N, T, or S; a CDR-H3 set forth as X₅RX₆X₇YDYDX₈X₉DX₁₀ (SEQ ID NO: 288), wherein X₅ is T or A, X₆ is S, F, or I, X₇ is S, N, or Y, X₈ is P, Y, or V, X₉ is I, F, or L, and X₁₀ is Y or F; and/or (e.g., and) a CDR-L1 set forth as QDIX₁₁NX₁₂ (SEQ ID NO: 289), in which X₁₁ is S or T and X₁₂ is F, C, S, or Y; a CDR-L2 set forth as YTS (SEQ ID NO: 13); and a CDR-L3 set forth as QQGX₁₃X₁₄X₁₅PX₁₆T (SEQ ID NO: 290), in which X₁₃ is H or N, X₁₄ is T or A, X₁₅ is L or Y, and X₁₆ is Y, W, or F.

In some embodiments, any one of the anti-TfR antibodies described herein binds transferrin receptor 1 (TfR1) with a K_{D} of less than 10⁻⁸ M.

Nucleic acids encoding any of the antibodies described herein, vectors comprising such nucleic acids, cells comprising such vectors are also provided.

Other aspects of the present disclosure provide methods of producing an anti-TfR1 antibody. In some embodiments, the methods comprise culturing the cells comprising nucleic acids encoding any one of the antibodies described herein under conditions suitable for the expression of the antibody.

Other aspects of the present disclosure provide complexes comprising any one of the antibodies described herein covalently linked to a molecular payload. In some embodiments, the molecular payload is a diagnostic agent or a therapeutic agent. In some embodiments, the molecular payload is an oligonucleotide, a polypeptide, or a small molecule. In some embodiments, the antibody and the molecular payload are linked via a linker. In some embodiments, the linker is a reversible linker. In some embodiments, the linker is a val-Cit linker.

Composition comprising any one of the antibodies described herein, any one of the nucleic acids described herein, any one of the vectors described herein, or any one of the complexes described herein are also provided. In some embodiments, the composition comprises a pharmaceutically acceptable carrier.

Also provided herein are methods of detecting a transferrin receptor in a biological sample. In some embodiments, the methods comprise contacting any one of the antibodies described herein with the biological sample and measuring binding of the antibody to the biological sample. In some embodiments, the antibody is covalently linked to a diagnostic agent. In some embodiments, the biological sample is obtained from a human subject suspected of having or at risk for a disease associated with transferrin receptor. In some embodiments, the contacting step is performed by administering the subject an effective amount of the anti-TfR antibody.

Method of delivering a molecular payload to a cell are also provided. In some embodiments, the methods comprise contacting any one of the complexes described herein with a cell. In some embodiments, the cell is a muscle cell. In some embodiments, the cell is in vitro. In some embodiments, the cell is in a subject. In some embodiments, the subject is human.

In some aspects, method of delivering a molecular payload to the brain or the muscle of a subject are provided. In some embodiments, the method comprises administering to the subject an effective amount of any one of the complexes described herein. In some embodiments, the administration is intravenous.

In some aspects, methods of treating a disease are provided. In some embodiments, the method comprises administering to the subject an effective amount of any one of the complexes described herein, e.g., in which the molecular payload is a therapeutic agent. In some embodiments, the disease is a neurological disease and the molecular payload is a drug for treating a neurological disease. In some embodiments, the disease is a muscle disease and, e.g., the molecular payload is a drug for treating a muscle disease. In some embodiments, the muscle disease is a rare muscle disease or muscle atrophy.

### BRIEF DESCRIPTION OF THE DRAWINGS

FIG. 1 depicts the screen process of the anti-TfR antibodies.

FIGS. 2A and 2B are graphs showing the expression of TfR1 in tissues. FIG. 2A: mouse TfR1; FIG. 2B: Cyno TfR1.

FIG. 3 is a graph showing DMPK knock down (KD) efficiency in non-human primate (NHP) cells or cells from human DM1 patients (DM1) of conjugates containing selected anti-TfR1 antibodies covalently conjugated to a control antisense oligonucleotide targeting DMPK.

FIG. 4 shows the serum stability of the linker used for linking an anti-TfR antibody and a molecular payload (e.g., an oligonucleotide) in various species over time after intravenous administration.

### DETAILED DESCRIPTION

The present disclosure, at least in part, is based on the development of anti-TfR antibodies, e.g., antibodies listed in Table 1 and their variants, which showed high binding affinity and specificity to human TfR. Also provided are the use of the anti-TfR antibodies and their variants in research, diagnostic/detection, and therapeutic applications. In some embodiments, the anti-TfR antibodies described herein are used for delivering molecular payloads (e.g., oligonucleotides, peptides, small molecules) to a target cell or tissue that expresses TfR. In some embodiments, the molecular payload to be delivered is conjugated the anti-TfR antibodies and delivered to a target cell or tissue that expresses TfR via receptor internationalization. Exemplary tissues that express TfR and can be targeted using the anti-TfR antibodies described herein include, without limitation: brain, muscle, adrenal, appendix, bone marrow, colon, duodenum, endometrium, esophagus, fat, gall bladder, heart, kidney, liver, lung, lymph node, ovary, pancreas, placenta, prostate, salivary gland, skin, small intestine, spleen, stomach, testis, thyroid, urinary bladder. In some embodiments, such approach has beneficial effects in muscle cells and for delivering across the blood brain barrier, which have been proven challenging. In some aspects, the present disclosure provides data demonstrating that the anti-TfR antibodies described herein has superior activity in delivering molecular payload into a target cell (e.g., a muscle cell), compared with other known anti-TfR antibodies.

As such, the present disclosure also provides complexes comprising any one of the anti-TfR1 antibodies covalently linked to molecular payloads. In some embodiments, the complexes are particularly useful for delivering molecular payloads that inhibit the expression or activity of target genes in muscle cells, e.g., in a subject having or suspected of having a rare muscle disease or muscle atrophy (e.g., as listed in Table 7). In some embodiments, the complexes are particularly useful for delivering drugs to the brain for treating a neurological disease (e.g., as listed in Table 8).

Further aspects of the disclosure, including a description of defined terms, are provided below.

### I. DEFINITIONS

Administering: As used herein, the terms “administering” or “administration” means to provide a complex to a subject in a manner that is physiologically and/or (e.g., and) pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a F(ab′) fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (a), delta (A), epsilon (ε), gamma (γ) or mu (μ) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information System® http://www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). ee also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

CDR-grafted antibody: The term “CDR-grafted antibody” refers to antibodies which comprise heavy and light chain variable region sequences from one species but in which the sequences of one or more of the CDR regions of VH and/or (e.g., and) VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human transferrin receptor and non-human primate transferrin receptor) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti-TfR antibodies and antigen binding portions are provided. Such antibodies may be generated by obtaining murine anti-transferrin receptor monoclonal antibodies using traditional hybridoma technology followed by humanization using in vitro genetic engineering, such as those disclosed in Kasaian et al PCT publication No. WO 2005/123126 A2.

Isolated antibody: An “isolated antibody”, as used herein, is intended to refer to an antibody that is substantially free of other antibodies having different antigenic specificities (e.g., an isolated antibody that specifically binds transferrin receptor is substantially free of antibodies that specifically bind antigens other than transferrin receptor). An isolated antibody that specifically binds transferrin receptor complex may, however, have cross-reactivity to other antigens, such as transferrin receptor molecules from other species. Moreover, an isolated antibody may be substantially free of other cellular material and/or (e.g., and) chemicals.

Molecular payload: As used herein, the term “molecular payload” refers to a molecule or species that functions to modulate a biological outcome. In some embodiments, a molecular payload is linked to, or otherwise associated with an anti-TfR antibody. In some embodiments, the molecular payload is a small molecule, a protein, a peptide, a nucleic acid, or an oligonucleotide. In some embodiments, the molecular payload functions to modulate the transcription of a DNA sequence, to modulate the expression of a protein, or to modulate the activity of a protein. In some embodiments, the molecular payload is an oligonucleotide that comprises a strand having a region of complementarity to a target gene.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidite morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleotides (e.g. 2′-O-methyl sugar modifications, purine or pyrimidine modifications). In some embodiments, an oligonucleotide may comprise one or more modified internucleotide linkage. In some embodiments, an oligonucleotide may comprise one or more phosphorothioate linkages, which may be in the Rp or Sp stereochemical conformation.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human transferrin receptor which can be generated using techniques well known in the art, such as, but not limited to, using human Ig phage libraries such as those disclosed in Jermutus et al., PCT publication No. WO 2005/007699 A2.

Region of complementarity: As used herein, the term “region of complementarity” refers to a nucleotide sequence, e.g., of a oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a disease resulting from a disease-associated-repeat expansion, e.g., in a DMPK allele.

Transferrin receptor: As used herein, the term, “transferrin receptor” (also known as TFRC, CD71, p90, TfR, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

An example human transferrin receptor amino acid sequence, corresponding to NCBI sequence NP_003225.2 (transferrin receptor protein 1 isoform 1, Homo sapiens) is as follows:

(SEQ ID NO: 228)
MMDQARSAFSNLFGGEPLSYTRFSLARQVDGDNSHVEMKLAVDEEENAD
NNTKANVTKPKRCSGSICYGTIAVIVFFLIGFMIGYLGYCKGVEPKTEC
ERLAGTESPVREEPGEDFPAARRLYWDDLKRKLSEKLDSTDFTGTIKLL
NENSYVPREAGSQKDENLALYVENQFREFKLSKVWRDQHFVKIQVKDSA
QNSVIIVDKNGRLVYLVENPGGYVAYSKAATVTGKLVHANFGTKKDFED
LYTPVNGSIVIVRAGKITFAEKVANAESLNAIGVLIYMDQTKFPIVNAE
LSFFGHAHLGTGDPYTPGFPSFNHTQFPPSRSSGLPNIPVQTISRAAAE
KLFGNMEGDCPSDWKTDSTCRMVTSESKNVKLTVSNVLKEIKILNIFGV
IKGFVEPDHYVVVGAQRDAWGPGAAKSGVGTALLLKLAQMFSDMVLKDG
FQPSRSIIFASWSAGDFGSVGATEWLEGYLSSLHLKAFTYINLDKAVLG
TSNFKVSASPLLYTLIEKTMQNVKHPVTGQFLYQDSNWASKVEKLTLDN
AAFPFLAYSGIPAVSFCFCEDTDYPYLGTTMDTYKELIERIPELNKVAR
AAAEVAGQFVIKLTHDVELNLDYERYNSQLLSFVRDLNQYRADIKEMGL
SLQWLYSARGDFFRATSRLTTDFGNAEKTDRFVMKKLNDRVMRVEYHFL
SPYVSPKESPFRHVFWGSGSHTLPALLENLKLRKQNNGAFNETLFRNQL
ALATWTIQGAANALSGDVWDIDNEF.

An example non-human primate transferrin receptor amino acid sequence, corresponding to NCBI sequence NP_001244232.1 (transferrin receptor protein 1, Macaca mulatta) is as follows:

(SEQ ID NO: 229)
MMDQARSAFSNLFGGEPLSYTRFSLARQVDGDNSHVEMKLGVDEEENTD
NNTKPNGTKPKRCGGNICYGTIAVIIFFLIGFMIGYLGYCKGVEPKTEC
ERLAGTESPAREEPEEDFPAAPRLYWDDLKRKLSEKLDTTDFTSTIKLL
NENLYVPREAGSQKDENLALYIENQFREFKLSKVWRDQHFVKIQVKDSA
QNSVIIVDKNGGLVYLVENPGGYVAYSKAATVTGKLVHANFGTKKDFED
LDSPVNGSIVIVRAGKITFAEKVANAESLNAIGVLIYMDQTKFPIVKAD
LSFFGHAHLGTGDPYTPGFPSFNHTQFPPSQSSGLPNIPVQTISRAAAE
KLFGNMEGDCPSDWKTDSTCKMVTSENKSVKLTVSNVLKETKILNIFGV
IKGFVEPDHYVVVGAQRDAWGPGAAKSSVGTALLLKLAQMFSDMVLKDG
FQPSRSIIFASWSAGDFGSVGATEWLEGYLSSLHLKAFTYINLDKAVLG
TSNFKVSASPLLYTLIEKTMQDVKHPVTGRSLYQDSNWASKVEKLTLDN
AAFPFLAYSGIPAVSFCFCEDTDYPYLGTTMDTYKELVERIPELNKVAR
AAAEVAGQFVIKLTHDTELNLDYERYNSQLLLFLRDLNQYRADVKEMGL
SLQWLYSARGDFFRATSRLTTDFRNAEKRDKFVMKKLNDRVMRVEYYFL
SPYVSPKESPFRHVFWGSGSHTLSALLESLKLRRQNNSAFNETLFRNQL
ALATWTIQGAANALSGDVWDIDNEF

An example non-human primate transferrin receptor amino acid sequence, corresponding to NCBI sequence XP_005545315.1 (transferrin receptor protein 1, Macaca fascicularis) is as follows:

(SEQ ID NO: 230)
MMDQARSAFSNLFGGEPLSYTRFSLARQVDGDNSHVEMKLGVDEEENTD
NNTKANGTKPKRCGGNICYGTIAVIIFFLIGFMIGYLGYCKGVEPKTEC
ERLAGTESPAREEPEEDFPAAPRLYWDDLKRKLSEKLDTTDFTSTIKLL
NENLYVPREAGSQKDENLALYIENQFREFKLSKVWRDQHFVKIQVKDSA
QNSVIIVDKNGGLVYLVENPGGYVAYSKAATVTGKLVHANFGTKKDFED
LDSPVNGSIVIVRAGKITFAEKVANAESLNAIGVLIYMDQTKFPIVKAD
LSFFGHAHLGTGDPYTPGFPSFNHTQFPPSQSSGLPNIPVQTISRAAAE
KLFGNMEGDCPSDWKTDSTCKMVTSENKSVKLTVSNVLKETKILNIFGV
IKGFVEPDHYVVVGAQRDAWGPGAAKSSVGTALLLKLAQMFSDMVLKDG
FQPSRSIIFASWSAGDFGSVGATEWLEGYLSSLHLKAFTYINLDKAVLG
TSNFKVSASPLLYTLIEKTMQDVKHPVTGRSLYQDSNWASKVEKLTLDN
AAFPFLAYSGIPAVSFCFCEDTDYPYLGTTMDTYKELVERIPELNKVAR
AAAEVAGQFVIKLTHDTELNLDYERYNSQLLLFLRDLNQYRADVKEMGL
SLQWLYSARGDFFRATSRLTTDFRNAEKRDKFVMKKLNDRVMRVEYYFL
SPYVSPKESPFRHVFWGSGSHTLSALLESLKLRRQNNSAFNETLFRNQL
ALATWTIQGAANALSGDVWDIDNEF.

An example mouse transferrin receptor amino acid sequence, corresponding to NCBI sequence NP_001344227.1 (transferrin receptor protein 1, Mus musculus) is as follows:

(SEQ ID NO: 231)
MMDQARSAFSNLFGGEPLSYTRFSLARQVDGDNSHVEMKLAADEEENAD
NNMKASVRKPKRFNGRLCFAAIALVIFFLIGFMSGYLGYCKRVEQKEEC
VKLAETEETDKSETMETEDVPTSSRLYWADLKTLLSEKLNSIEFADTIK
QLSQNTYTPREAGSQKDESLAYYIENQFHEFKFSKVWRDEHYVKIQVKS
SIGQNMVTIVQSNGNLDPVESPEGYVAFSKPTEVSGKLVHANFGTKKDF
EELSYSVNGSLVIVRAGEITFAEKVANAQSFNAIGVLIYMDKNKFPVVE
ADLALFGHAHLGTGDPYTPGFPSFNHTQFPPSQSSGLPNIPVQTISRAA
AEKLFGKMEGSCPARWNIDSSCKLELSQNQNVKLIVKNVLKERRILNIF
GVIKGYEEPDRYVVVGAQRDALGAGVAAKSSVGTGLLLKLAQVFSDMIS
KDGFRPSRSIIFASWTAGDFGAVGATEWLEGYLSSLHLKAFTYINLDKV
VLGTSNFKVSASPLLYTLMGKIMQDVKHPVDGKSLYRDSNWISKVEKLS
FDNAAYPFLAYSGIPAVSFCFCEDADYPYLGTRLDTYEALTQKVPQLNQ
MVRTAAEVAGQLIIKLTHDVELNLDYEMYNSKLLSFMKDLNQFKTDIRD
MGLSLQWLYSARGDYFRATSRLTTDFHNAEKTNRFVMREINDRIMKVEY
HFLSPYVSPRESPFRHIFWGSGSHTLSALVENLKLRQKNITAFNETLFR
NQLALATWTIQGVANALSGDIWNIDNEF.

2′-modified nucleoside: As used herein, the terms “2′-modified nucleoside” and “2′-modified ribonucleoside” are used interchangeably and refer to a nucleoside having a sugar moiety modified at the 2′ position. In some embodiments, the 2′-modified nucleoside is a 2′-4′ bicyclic nucleoside, where the 2′ and 4′ positions of the sugar are bridged (e.g., via a methylene, an ethylene, or a (S)-constrained ethyl bridge). In some embodiments, the 2′-modified nucleoside is a non-bicyclic 2′-modified nucleoside, e.g., where the 2′ position of the sugar moiety is substituted. Non-limiting examples of 2′-modified nucleosides include: 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA, methylene-bridged nucleic acid), ethylene-bridged nucleic acid (ENA), and (S)-constrained ethyl-bridged nucleic acid (cEt). In some embodiments, the 2′-modified nucleosides described herein are high-affinity modified nucleotides and oligonucleotides comprising the 2′-modified nucleotides have increased affinity to a target sequences, relative to an unmodified oligonucleotide. Examples of structures of 2′-modified nucleosides are provided below:

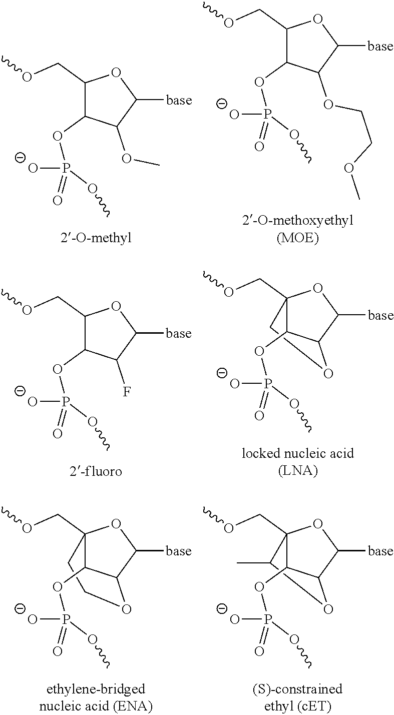

### II. ANTI-TFR ANTIBODIES

In some embodiments, agents binding to transferrin receptor, e.g., anti-TfR antibodies, are capable of targeting muscle cell and/or (e.g., and) mediate the transportation of an agent across the blood brain barrier. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Antibodies that bind, e.g. specifically bind, to a transferrin receptor may be internalized into the cell, e.g. through receptor-mediated endocytosis, upon binding to a transferrin receptor.

Provided herein, in some aspects, are antibodies that bind to transferrin receptor with high specificity and affinity. In some embodiments, the anti-TfR antibody described herein specifically binds to any extracellular epitope of a transferrin receptor or an epitope that becomes exposed to an antibody. In some embodiments, anti-TfR antibodies provided herein bind specifically to transferrin receptor from human, non-human primates, mouse, rat, etc. In some embodiments, anti-TfR antibodies provided herein bind to human transferrin receptor. In some embodiments, the anti-TfR antibody described herein binds to an amino acid segment of a human or non-human primate transferrin receptor, as provided in SEQ ID NOs: 228-231. In some embodiments, the anti-TfR antibody described herein binds to an amino acid segment corresponding to amino acids 90-96 of a human transferrin receptor as set forth in SEQ ID NO: 228, which is not in the apical domain of the transferrin receptor.

In some embodiments, an anti-TFR antibody specifically binds a TfR1 (e.g., a human or non-human primate TfR1) with binding affinity (e.g., as indicated by Kd) of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, the anti-TfR antibodies described herein binds to TfR1 with a KD of sub-nanomolar range. In some embodiments, the anti-TfR antibodies described herein selectively binds to transferrin receptor 1 (TfR1) but does not binding to transferrin receptor 2 (TfR2). In some embodiments, the anti-TfR antibodies described herein binds to human TfR1 and cyno TfR1 (e.g., with a Kd of 10⁻⁷ M, 10⁻⁸ M, 10⁻⁹ M, 10⁻¹⁰ M, 10⁻¹¹ M, 10⁻¹² M, 10⁻¹³ M, or less), but does not bind to a mouse TfR1. The affinity and binding kinetics of the anti-TfR antibody can be tested using any suitable method including but not limited to biosensor technology (e.g., OCTET or BIACORE). In some embodiments, binding of any one of the anti-TfR antibody described herein does not complete with or inhibit transferrin binding to the TfR1. In some embodiments, binding of any one of the anti-TfR antibody described herein does not complete with or inhibit HFE-beta-2-microglobulin binding to the TfR1.

The heavy chain and light chain variable domain and CDR sequences of non-limiting examples of anti-TfR antibodies are provided in Table 1.

TABLE 1
Examples of anti-TfR1 antibodies (CDRs according to the IMGT ® definition)

| Ab | CDRs | Variable domains |
|---|---|---|
| 3-A4 | CDR-H1: | VH |
| | GFNIKDDY (SEQ ID NO: 1) | EVQLQQSGAELVRPGASVKLSCTASGFNIKDDYMYWVKQ |
| | CDR-H2: | RPEQGLEWIGWIDPENGDTEYASKFQDKATVTADTSSNTA |
| | IDPENGDT (SEQ ID NO: 2) | YLQLSSLTSEDTAVYYCTLWLRRGLDYWGQGTSVTVSS |
| | CDR-H3: | (SEQ ID NO: 7) |
| | TLWLRRGLDY (SEQ ID NO: 3) | |
| | CDR-L1: | VL |
| | KSLLHSNGYTY (SEQ ID NO: 4) | DIVMTQAAPSVPVTPGESVSISCRSSKSLLHSNGYTYLFWF |
| | CDR-L2: | LQRPGQSPQLLIYRMSNLASGVPDRFSGSGSGTAFTLRISR |
| | RMS (SEQ ID NO: 5) | VEAEDVGVYYCMQHLEYPFTFGGGTKLEIK (SEQ ID NO: |
| | CDR-L3: | 8) |
| | MQHLEYPFT (SEQ ID NO: 6) | |
| 3-M12 | CDR-H1: | VH |
| | GYSITSGYY (SEQ ID NO: 9) | DVQLQESGPGLVKPSQSLSLTCSVTGYSITSGYYWNWIRQ |
| | CDR-H2: | FPGNKLEWMGYITFDGANNYNPSLKNRISITRDTSKNQFFL |
| | ITFDGAN (SEQ ID NO: 10) | KLTSVTTEDTATYYCTRSSYDYDVLDYWGQGTTLTVSS |
| | CDR-H3: | (SEQ ID NO: 15) |
| | TRSSYDYDVLDY (SEQ ID NO: 11) | |
| | CDR-L1: | VL |
| | QDISNF (SEQ ID NO: 12) | DIQMTQTTSSLSASLGDRVTISCRASQDISNFLNWYQQRPD |
| | CDR-L2: | GTVKLLIYYTSRLHSGVPSRFSGSGSGTDFSLTVSNLEQEDI |
| | YTS (SEQ ID NO: 13) | ATYFCQQGHTLPYTFGGGTKLEIK (SEQ ID NO: 16) |
| | CDR-L3: | |
| | QQGHTLPYT (SEQ ID NO: 14) | |
| 5-H12 | CDR-H1: | VH |
| | GYSFTDYC (SEQ ID NO: 17) | QIQLQQSGPELVRPGASVKISCKASGYSFTDYCINWVNQR |
| | CDR-H2: | PGQGLEWIGWIYPGSGNTRYSERFKGKATLTVDTSSNTAY |
| | IYPGSGNT (SEQ ID NO: 18) | MQLSSLTSEDSAVYFCAREDYYPYHGMDYWGQGTSVTV |
| | CDR-H3: | SS (SEQ ID NO: 23) |
| | AREDYYPYHGMDY (SEQ ID NO: 19) | |
| | CDR-L1: | VL |
| | ESVDGYDNSF (SEQ ID NO: 20) | DIVLTQSPTSLAVSLGQRATISCRASESVDGYDNSFMHWY |
| | CDR-L2: | QQKPGQPPKLLIFRASNLESGIPARFSGSGSRTDFTLTINPV |
| | RAS (SEQ ID NO: 21) | EAADVATYYCQQSSEDPWTFGGGTKLEIK (SEQ ID NO: |
| | CDR-L3: | 24) |
| | QQSSEDPWT (SEQ ID NO: 22) | |
| 8-K6 | CDR-H1: | VH |
| | GYTFTSYW (SEQ ID NO: 25) | QVHLQQPGAELVKPGASVKMSCKASGYTFTSYWITWVK |
| | CDR-H2: | QRPGQGLEWIGDIFPNSGRTNYDEKFKSKATLTVDTSSSTA |
| | IFPNSGRT (SEQ ID NO: 26) | YMQLSSLTSEDSAVYFCAREGNFGSLDYWGQGTTLTVSS |
| | CDR-H3: | (SEQ ID NO: 31) |
| | AREGNFGSLDY (SEQ ID NO: 27) | |
| | CDR-L1: | VL |
| | SNLNY (SEQ ID NO: 28) | QIVLTQSPAIMSASPGEKVTMTCSANSNLNYMNWYHQKS |
| | CDR-L2: | GTSPKRWIYDTSKLASGVPARFSASGSGTSYSLTISSMEAE |
| | DTS (SEQ ID NO: 29) | DAATYYCQQWSRNPLTFGAGTRLELK (SEQ ID NO: 32) |
| | CDR-L3: | |
| | QQWSRNPLT (SEQ ID NO: 30) | |
| 9-K23 | CDR-H1: | VH |
| | GFSLNTYDVG (SEQ ID NO: 33) | QPSGKGLEWLANIWWNDDKYYNSALKSRLTISKDTSNNQ |
| | CDR-H2: | VFLKISSVDTADTATYYCTLYSYDGGFAYWGQGTLVTVS |
| | IWWNDDK (SEQ ID NO: 34) | QVTLKESGPGMLQPSQTLSLTCSFSGFSLNTYDVGVGWIR |
| | CDR-H3: | A (SEQ ID NO: 39) |
| | TLYSYDGGFAY (SEQ ID NO: 35) | |
| | CDR-L1: | VL |
| | SSVSSSY (SEQ ID NO: 36) | QIVLTQSPAIMSASLGERVTMTCTASSSVSSSYLHWYQQK |
| | CDR-L2: | PGSSPKLWIYSTSNLASGVPARFSGSGSGTSYSLTISSMEAE |
| | STS (SEQ ID NO: 37) | DAATYYCHQYHRSPYTFGGGTKLEIK (SEQ ID NO: 40) |
| | CDR-L3: | |
| | HQYHRSPYT (SEQ ID NO: 38) | |
| 3-E5 | CDR-H1: | VHEIQMKQSGAELVKPGASVKISCKASGYSFTGYNMNWVKQ |
| | GYSFTGYN (SEQ ID NO: 41) | SHGKSLEWIGNINPYYGSTGYNQKFKGKATLTVDKSSSTA |
| | CDR-H2: | YMQLNSLTSEDSAVYYCARGDYGYDEGTWFAYWGQGTL |
| | INPYYGST (SEQ ID NO: 42) | VTVSA (SEQ ID NO: 47) |
| | CDR-H3: | |
| | ARGDYGYDEGTWFAY (SEQ ID NO: 43) | |
| | CDR-L1: | VL |
| | QSLLNSRTRKNY (SEQ ID NO: 44) | DIVMSQSPSSLAVSAGEKVTMSCKSSQSLLNSRTRKNYLA |
| | CDR-L2: | WYQQKPEQSPKLLIYWASTRESGVPDRFTGSGSGTDFTLTI |
| | WAS (SEQ ID NO: 45) | SSVQAEDLAVYYCKQSYNLPFTFGSGTKLEIK (SEQ ID |
| | CDR-L3: | NO: 48) |
| | KQSYNLPFT (SEQ ID NO: 46) | |
| 6-D3 | CDR-H1: | VH |
| | GYTFTRHW (SEQ ID NO: 49) | QVQLQQPGAELVKPGASVKMSCKASGYTFTRHWITWVK |
| | CDR-H2: | QRPGQGLEWIGDIYPGSGRTNYNEKFKSTATLTVDTSSST |
| | IYPGSGRT (SEQ ID NO: 50) | AYMQLSSLTSEDSAVYYCARDGYLYINYFDYWGQGTTLT |
| | CDR-H3: | VSS (SEQ ID NO: 54) |
| | ARDGYLYINYFDY (SEQ ID NO: 51) | |
| | CDR-L1: | VL |
| | SSVSF (SEQ ID NO: 52) | ENVLTQSPAIMSASPGEKVTMTCSASSSVSFMHWFQQKSS |
| | CDR-L2: | TSPKLWIYDTSKLASGVPGRFSGSGSGSSYSLTISSMAAED |
| | DTS (SEQ ID NO: 29) | VATYYCFQGSGYPYTFGGGTKLEIK (SEQ ID NO: 55) |
| | CDR-L3: | |
| | FQGSGYPYT (SEQ ID NO: 53) | |
| 4-O12 | CDR-H1: | VH |
| | GFNIVDDY (SEQ ID NO: 56) | EVQLQQSGAELVRPGASVKLSCTASGFNIVDDYMHWVKQ |
| | CDR-H2: | RPEQGLEWIGWIYPENADTEYASKFQGKATITADTSSNTA |
| | IYPENADT (SEQ ID NO: 57) | YLQLSSLTSEDTAVYYCTTATGTGWFAYWGQGTLVTVSA |
| | CDR-H3: | (SEQ ID NO: 62) |
| | TTATGTGWFAY (SEQ ID NO: 58) | |
| | CDR-L1: | VL |
| | QSLLDSDGKTY (SEQ ID NO: 59) | DVVMTQTPLTLSVTIGQPASISCKSSQSLLDSDGKTYLNWL |
| | CDR-L2: | FQRPGQSPKRLIYLVSKLDSGVPDRFTGSGSGTDFTLKISR |
| | LVS (SEQ ID NO: 60) | VETEDLGVYYCWQGTHFPWTFGGGAKLEIK (SEQ ID NO: |
| | CDR-L3: | 63) |
| | WQGTHFPWT (SEQ ID NO: 61) | |
| 4-C5 | CDR-H1: | VH |
| | GYTFSNYW (SEQ ID NO: 64) | QVQLQQSGAELMKPGASVKISCKATGYTFSNYWIEWVKQ |
| | CDR-H2: | RPGHGLEWIGEILPGSGSTNYNENFKGKATFTADTSSNTA |
| | ILPGSGST (SEQ ID NO: 65) | YMQLSSLTSEDSAVYYCARRGAYGNFHYWGQGTTLTVSS |
| | CDR-H3: | (SEQ ID NO: 70) |
| | ARRGAYGNFHY (SEQ ID NO: 66) | |
| | CDR-L1: | VL |
| | SSISSSN (SEQ ID NO: 67) | EIVLTQSPALMAASPGEKVTITCSVSSSISSSNLHWYQQKS |
| | CDR-L2: | ETSPKPWIYGTSNLASGVPVRFSGSGSGTSYSLTISSMEAE |
| | GTS (SEQ ID NO: 68) | DAATYYCQQWRSYPYTFGGGTKLEIK (SEQ ID NO: 71) |
| | CDR-L3: | |
| | QQWRSYPYT (SEQ ID NO: 69) | |
| 10-P5 | CDR-H1: | VH |
| | GYTFTDYN (SEQ ID NO: 72) | EVQLQQFGAELVKPGASVKISCKASGYTFTDYNMAWVKE |
| | CDR-H2: | SHGKSLEWIGDINPNYDTTSYNQKFKGKATLTVDKSSSTA |
| | INPNYDTT (SEQ ID NO: 73) | HMELRSLTSEGTAVYYCARSGYYGSSYYWHFDVWGTGT |
| | CDR-H3: | TVTVSS (SEQ ID NO: 77) |
| | ARSGYYGSSYYWHFDV (SEQ ID NO: 74) | |
| | CDR-L1: | VL |
| | QSLLYSSNQKNY (SEQ ID NO: 75) | DIVMSQSPSSLAVSVGEKVTMSCKSSQSLLYSSNQKNYLA |
| | CDR-L2: | WYQQKPGQSPKLLIYWASTRESGVPDRFTGSGSGTDFTLT |
| | WAS (SEQ ID NO: 45) | ISSVKAEDLAVYYCQQYYNYPFTFGSGTKLEIK (SEQ ID |
| | CDR-L3: | NO: 78) |
| | QQYYNYPFT (SEQ ID NO: 76) | |
| 2-H19 | CDR-H1: | VH |
| | GFNIKDYY (SEQ ID NO: 79) | EVQLQQSGAELVRSGASVKLSCTASGFNIKDYYMHWVKQ |
| | CDR-H2: | RPEQGLEWIGWIDPESGDTEYAPKFQGRATMTADTSSNTA |
| | IDPESGDT (SEQ ID NO: 80) | YMQLSSLTSEDTAVYYCYGHDYRVDCWGQGTSVTVSS |
| | CDR-H3: | (SEQ ID NO: 85) |
| | YGHDYRVDC (SEQ ID NO: 81) | |
| | CDR-L1: | VL |
| | QSLVHSNGNTY (SEQ ID NO: 82) | DVVMTQTPLSLPVSLGDQASISCRSSQSLVHSNGNTYLHW |
| | CDR-L2: | YLQKPGQSPKLLIYKVSNRFSGVPDRFSGSGSGTDFTLKIS |
| | KVS (SEQ ID NO: 83) | RVEAEDLGVYFCSQSTHIPWTFGGGTKLEIK (SEQ ID NO: |
| | CDR-L3: | 86) |
| | SQSTHIPWT (SEQ ID NO: 84) | |
| 3-F3 | CDR-H1: | VH |
| | GYTFTDYN (SEQ ID NO: 72) | EVQLQQFGAELVKPGASVKISCKASGYTFTDYNMGWVKQ |
| | CDR-H2: | SHGKSLEWIGDINPNYDSTSYTQKFKGKATLTVDKSSSTA |
| | INPNYDST (SEQ ID NO: 87) | YMELRSLTSEDTAVYYCARSGYYGSSYYWHFDVWGTGT |
| | CDR-H3: | TVTVSS (SEQ ID NO: 89) |
| | ARSGYYGSSYYWHFDV (SEQ ID NO: 74) | |
| | CDR-L1: | VL |
| | QSLLYSSNQKNY (SEQ ID NO: 75) | DIVMSQSPSSLAVSVGEKVTMSCKSSQSLLYSSNQKNYLA |
| | CDR-L2: | WYQQKPGQSPKLLIYWASTRESGVPDRFTGSGSGTDFTLT |
| | WAS (SEQ ID NO: 45) | ISSVKAEDLAVYYCQQYYHYPFTFGSGTKLEIK (SEQ ID |
| | CDR-L3: | NO: 90) |
| | QQYYHYPFT (SEQ ID NO: 88) | |
| 8-017 | CDR-H1: | VH |
| | GFSLTNYG (SEQ ID NO: 91) | QVQLKESGPGLVAPSQSLSITCTVSGFSLTNYGVHWVRQP |
| | CDR-H2: | PGKGLEWLVVIWNDGSATYNSALESRLSISKDNSKSQVFL |
| | IWNDGSA (SEQ ID NO: 92) | KMNSLQTDDTAMYYCARHESSNPFAYWGQGTLVTVSA |
| | CDR-H3: | (SEQ ID NO: 97) |
| | ARHESSNPFAY (SEQ ID NO: 93) | |
| | CDR-L1: | VL |
| | QSIGTS (SEQ ID NO: 94) | DILLTQSPAILSVSPGERVSFSCRASQSIGTSIHWYQQRTNG |
| | CDR-L2: | SPRLLIKSASESIAGIPSRFSGSGSGTDFTLSINSVESEDIADY |
| | SAS (SEQ ID NO: 95) | YCQQNNRWPYTFGGGTKLEIK (SEQ ID NO: 98) |
| | CDR-L3: | |
| | QQNNRWPYT (SEQ ID NO: 96) | |
| 3-M9 | CDR-H1: | VH |
| | DFNIKDDY (SEQ ID NO: 99) | EVQLQQSGAELVRPGASVKLSCTASDFNIKDDYIHWVKQ |
| | CDR-H2: | RPEQGLEWIGRIDPANGNTKYAPKFQDKATITADTSSNTA |
| | IDPANGNT (SEQ ID NO: 100) | YLQLSSLTSEDTAVYYCALGYTYWGQGTTLTVSS (SEQ ID |
| | CDR-H3: | NO: 104) |
| | ALGYTY (SEQ ID NO: 101) | |
| | CDR-L1: | VL |
| | QSLLHSYGKTY (SEQ ID NO: 102) | DVVMTQTPLTLSVTIGQPASISCKSSQSLLHSYGKTYLNWL |
| | CDR-L2: | LQRPGQSPKLLIYLVSKLESGVPDRFSGSGSGTDFTLKISRV |
| | LVS (SEQ ID NO: 60) | EAEDLGVYYCLQTTHFPQTFGGGTKLEIK (SEQ ID NO: |
| | CDR-L3: | 105) |
| | LQTTHFPQT (SEQ ID NO: 103) | |
| 10-H2 | CDR-H1: | VH |
| | GFTFSDYG (SEQ ID NO: 106) | EVQLVESGGDLVKPGGSLKLSCAASGFTFSDYGMHWVRQ |
| | CDR-H2: | GPEKGLEWVAYINSGSSTIYYADTVKGRFTISRDNAKNTL |
| | INSGSSTI (SEQ ID NO: 107) | FLQMTSLRSEDTAMYYCARPGDYDNYAMDYWGQGTSVT |
| | CDR-H3: | VSS (SEQ ID NO: 112) |
| | ARPGDYDNYAMDY (SEQ ID NO: 108) | |
| | CDR-L1: | VL |
| | QDVSVA (SEQ ID NO: 109) | DIVMTQSHKFLSTSVGDRVSITCKASQDVSVAVAWYQQK |
| | CDR-L2: | PGQSPKLLIYWAYTRHTGVPDRFTGSGSGTEYTLTISSVQA |
| | WAY (SEQ ID NO: 110) | EDLALYYCQQHYNTPPWTFGGGTKLEIK (SEQ ID NO: 113) |
| | CDR-L3: | |
| | QQHYNTPPWT (SEQ ID NO: 111) | |
| 4-J22 | CDR-H1: | VH |
| | GFNIKDYY (SEQ ID NO: 79) | EVQLQQSGAELVRSGASVKLSCTASGFNIKDYYIHWVKQ |
| | CDR-H2: | RPEQGLEWIGWIDPENADTEYAPKFQGKATMTPDTSSNTA |
| | IDPENADT (SEQ ID NO: 114) | YLQLSSLTSEDTAVYYCYAWDYSMDYWGQGTSVTVSS |
| | CDR-H3: | (SEQ ID NO: 117) |
| | YAWDYSMDY (SEQ ID NO: 115) | |
| | CDR-L1: | VL |
| | QSLVHSNGNTY (SEQ ID NO: 82) | DVVMTQTPLSLSVSLGDQASISCRSSQSLVHSNGNTYLHW |
| | CDR-L2: | YLQKPGQSPKLLIYKVSNRFSGVPDRFSGSGSGTDFILKISR |
| | KVS (SEQ ID NO: 83) | VEAEDLGVYFCSQNTHVPYTFGGGTRLEIK (SEQ ID NO: |
| | CDR-L3: | 118) |
| | SQNTHVPYT (SEQ ID NO: 116) | |
| 9-D4 | CDR-H1: | VH |
| | GFTFTDYG (SEQ ID NO: 119) | QVQLQQSGTELARPGASVKLSCKASGFTFTDYGINWVKQ |
| | CDR-H2: | RTGQGLEWIGEIYPSSGNSYYNEKFKAKATLTADKSSSTA |
| | IYPSSGNS (SEQ ID NO: 120) | YMELRSLTSEDSAVYFCARSTYYGSPIDYWGQGTTLTVSS |
| | CDR-H3: | (SEQ ID NO: 124) |
| | ARSTYYGSPIDY (SEQ ID NO: 121) | |
| | CDR-L1: | VL |
| | QDVDTT (SEQ ID NO: 122) | DIVMTQSHKFMSTPVGDRVSITCKASQDVDTTVAWYQQK |
| | CDR-L2: | PGQSPKLLIYWASTRQIGVPDRFTGSGSGTDFTLTISNVQSE |
| | WAS (SEQ ID NO: 45) | DLADYFCQQYSTYPLTFGGGTKLEIK (SEQ ID NO: 125) |
| | CDR-L3: | |
| | QQYSTYPLT (SEQ ID NO: 123) | |
| 8-D15 | CDR-H1: | VH |
| | GFSLTSYA (SEQ ID NO: 126) | QVQLKESGPGLVAPSQSLSITCTVSGFSLTSYAITWVRQSP |
| | CDR-H2: | GKGLEWLGLIWTGGGTNYNSALKSRLSISKDNSKSQVFLK |
| | IWTGGGT (SEQ ID NO: 127) | MNSLQTDDTARYYCARIYDGYYRYFDVWGTGTTVTVSS |
| | CDR-H3: | (SEQ ID NO: 132) |
| | ARIYDGYYRYFDV (SEQ ID NO: 128) | |
| | CDR-L1: | VL |
| | QSVSND (SEQ ID NO: 129) | RIVLTQTPKFLLVSAGDRVTMTCKASQSVSNDVAWYQQK |
| | CDR-L2: | PGQSPKLLIYYASNRYTGVPDRFTGSGYGTDFTFTISTVQA |
| | YAS (SEQ ID NO: 130) | EDLAVYFCQQDYSSPWTFGGGTKLEIK (SEQ ID NO: 133) |
| | CDR-L3: | |
| | QQDYSSPWT (SEQ ID NO: 131) | |
| 4-H4 | CDR-H1: | VH |
| | GFNIKDYY (SEQ ID NO: 79) | EVQLQQSGAELVRSGASVKLSCTASGFNIKDYYMHWVKQ |
| | CDR-H2: | RPEQGLDWIGWIDPENGDTEYAPKFQGKATMTADTSSNT |
| | IDPENGDT (SEQ ID NO: 2) | AYLQLSSLTSEDTAVYYCNVLTMPTAYWGQGTLVTVSA |
| | CDR-H3: | (SEQ ID NO: 136) |
| | NVLTMPTAY (SEQ ID NO: 134) | |
| | CDR-L1: | VL |
| | QSLLYSSNQKNY (SEQ ID NO: 75) | DIVMSQSPSSLAVSVGEKVIMSCKSSQSLLYSSNQKNYLA |
| | CDR-L2: | WYQQKPGQSPKLLIYWASTRESGVPDRFTGSGSGTDFTLT |
| | WAS (SEQ ID NO: 45) | ISSVKAEDLAVYYCQQYYSYPYTFGGGTKLEIK (SEQ ID |
| | CDR-L3: | NO: 137) |
| | QQYYSYPYT (SEQ ID NO: 135) | |
| 9-C4 | CDR-H1: | VH |
| | GFTFSSYG (SEQ ID NO: 138) | EVQLMESGGDLVKPGGSLKLSCAASGFTFSSYGLSWVRQ |
| | CDR-H2: | TPDKRLEWVATITSGGSYTYYPDSVKGRFTISRDNARNTL |
| | ITSGGSYT (SEQ ID NO: 139) | YLQMFSLKSEDTAMYYCALWSLDYWGQGTTLTVSS (SEQ |
| | CDR-H3: | ID NO: 143) |
| | ALWSLDY (SEQ ID NO: 140) | |
| | CDR-L1: | VL |
| | SSLSY (SEQ ID NO: 141) | QIVLTQSPAIMSASPGEKVTMTCSANSSLSYMHWYQQKPG |
| | CDR-L2: | TSPKRWIYDTSELASGVPARFSGSGSGTSYSLTISSMEAED |
| | DTS (SEQ ID NO: 29) | AATYYCHQRRSYPWTFGGGTKLEIK (SEQ ID NO: 144) |
| | CDR-L3: | |
| | HQRRSYPWT (SEQ ID NO: 142) | |

In some embodiments, the anti-TfR antibodies of the present disclosure comprises one or more of the CDR-H (e.g., CDR-H1, CDR-H2, and CDR-H3) amino acid sequences from any one of the anti-TfR antibodies selected from Table 1. In some embodiments, the anti-TfR antibodies of the present disclosure comprise the CDR-H1, CDR-H2, and CDR-H3 as provided for any one of the antibodies elected from Table 1. In some embodiments, the anti-TfR antibodies of the present disclosure comprises one or more of the CDR-L (e.g., CDR-L1, CDR-L2, and CDR-L3) amino acid sequences from any one of the anti-TfR antibodies selected from Table 1. In some embodiments, the anti-TfR antibodies of the present disclosure comprise the CDR-L1, CDR-L2, and CDR-L3 as provided for any one of the anti-TfR antibodies selected from Table 1.

In some embodiments, the anti-TfR antibodies of the present disclosure comprises the CDR-H1, CDR-H2, CDR-H3, CDR-L1, CDR-L2, and CDR-L3 as provided for any one of the anti-TfR antibodies selected from Table 1. In some embodiments, antibody heavy and light chain CDR3 domains may play a particularly important role in the binding specificity/affinity of an antibody for an antigen. Accordingly, the anti-TfR antibodies of the disclosure may include at least the heavy and/or (e.g., and) light chain CDR3s of any one of the anti-TfR antibodies selected from Table 1.

In some examples, any of the anti-TfR antibodies of the disclosure have one or more CDR (e.g., CDR-H or CDR-L) sequences substantially similar to any of the CDR-H1, CDR-H2, CDR-H3, CDR-L1, CDR-L2, and/or (e.g., and) CDR-L3 sequences from one of the anti-TfR antibodies selected from Table 1. In some embodiments, the position of one or more CDRs along the VH (e.g., CDR-H1, CDR-H2, or CDR-H3) and/or (e.g., and) VL (e.g., CDR-L1, CDR-L2, or CDR-L3) region of an antibody described herein can vary by one, two, three, four, five, or six amino acid positions so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or (e.g., and) C-terminal boundary of the CDR by one, two, three, four, five, or six amino acids, relative to the CDR position of any one of the antibodies described herein, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., CDR-H1, CDR-H2, or CDR-H3) and/or (e.g., and) VL (e.g., CDR-L1, CDR-L2, or CDR-L3) region of an antibody described herein can vary (e.g., be shorter or longer) by one, two, three, four, five, or more amino acids, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived).

Accordingly, in some embodiments, a CDR-L1, CDR-L2, CDR-L3, CDR-H1, CDR-H2, and/or (e.g., and) CDR-H3 described herein may be one, two, three, four, five or more amino acids shorter than one or more of the CDRs described herein (e.g., CDRS from any of the anti-TfR antibodies selected from Table 1) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, a CDR-L1, CDR-L2, CDR-L3, CDR-H1, CDR-H2, and/or (e.g., and) CDR-H3 described herein may be one, two, three, four, five or more amino acids longer than one or more of the CDRs described herein (e.g., CDRS from any of the anti-TfR antibodies selected from Table 1) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or (e.g., and) CDR-H3 described herein can be extended by one, two, three, four, five or more amino acids compared to one or more of the CDRs described herein (e.g., CDRS from any of the anti-TfR antibodies selected from Table 1) so long as immunospecific binding to transferrin receptor (e.g., human transferrin receptor is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or (e.g., and) CDR-H3 described herein can be extended by one, two, three, four, five or more amino acids compared to one or more of the CDRs described herein (e.g., CDRS from any of the anti-TfR antibodies selected from Table 1) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or (e.g., and) CDR-H3 described herein can be shortened by one, two, three, four, five or more amino acids compared to one or more of the CDRs described herein (e.g., CDRS from any of the anti-TfR antibodies selected from Table 1) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or (e.g., and) CDR-H3 described herein can be shortened by one, two, three, four, five or more amino acids compared to one or more of the CDRs described herein (e.g., CDRS from any of the anti-TfR antibodies selected from Table 1) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). Any method can be used to ascertain whether immunospecific binding to transferrin receptor (e.g., human transferrin receptor) is maintained, for example, using binding assays and conditions described in the art.

In some examples, any of the anti-TfR antibodies of the disclosure have one or more CDR (e.g., CDR-H or CDR-L) sequences substantially similar to any one of the anti-TfR antibodies selected from Table 1. For example, the antibodies may include one or more CDR sequence(s) from any of the anti-TfR antibodies selected from Table 1 containing up to 5, 4, 3, 2, or 1 amino acid residue variations as compared to the corresponding CDR region in any one of the CDRs provided herein (e.g., CDRs from any of the anti-TfR antibodies selected from Table 1) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a transferrin receptor protein (e.g., a human transferrin receptor protein), for example, as determined based on a crystal structure. Some aspects of the disclosure provide anti-TfR antibodies that comprise one or more of the heavy chain variable (VH) and/or (e.g., and) light chain variable (VL) domains provided herein. In some embodiments, any of the VH domains provided herein include one or more of the CDR-H sequences (e.g., CDR-H1, CDR-H2, and CDR-H3) provided herein, for example, any of the CDR-H sequences provided in any one of the anti-TfR selected from Table 1. In some embodiments, any of the VL domains provided herein include one or more of the CDR-L sequences (e.g., CDR-L1, CDR-L2, and CDR-L3) provided herein, for example, any of the CDR-L sequences provided in any one of the anti-TfR antibodies selected from Table 1.

In some embodiments, the anti-TfR antibodies of the disclosure include any antibody that includes a heavy chain variable domain and/or (e.g., and) a light chain variable domain of any one of the anti-TfR antibodies selected from Table 1, and variants thereof. In some embodiments, anti-TfR antibodies of the disclosure include any antibody that includes the heavy chain variable and light chain variable pairs of any anti-TfR antibodies selected from Table 1.

Aspects of the disclosure provide anti-TfR antibodies having a heavy chain variable (VH) and/or (e.g., and) a light chain variable (VL) domain amino acid sequence homologous to any of those described herein. In some embodiments, the anti-TfR antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any one of the anti-TfR antibodies selected from Table 1. In some embodiments, the homologous heavy chain variable and/or (e.g., and) a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or (e.g., and) a light chain variable sequence excluding any of the CDR sequences provided herein. In some embodiments, any of the anti-TfR antibodies provided herein comprise a heavy chain variable sequence and a light chain variable sequence that comprises a framework sequence that is at least 75%, 80%, 85%, 90%, 95%, 98%, or 99% identical to the framework sequence of any anti-TfR antibodies selected from Table 1.

In some embodiments, the anti-TfR antibodies of the present disclosure can bind to a target antigen (e.g., transferrin receptor) with relatively high affinity, e.g., with a K_{D} less than 10⁻⁶ M, 10⁻⁷ M, 10⁻⁸M, 10⁻⁹M, 10⁻¹⁰ M, 10⁻¹¹ M or lower. For example, the anti-TfR antibodies of the present disclosure can bind to a transferrin receptor protein (e.g., human transferrin receptor) with an affinity between 5 pM and 500 nM, e.g., between 50 pM and 100 nM, e.g., between 500 pM and 50 nM. The disclosure also includes antibodies that compete with any of the antibodies described herein for binding to a transferrin receptor protein (e.g., human transferrin receptor) and that have an affinity of 50 nM or lower (e.g., 20 nM or lower, 10 nM or lower, 500 pM or lower, 50 pM or lower, or 5 pM or lower). The affinity and binding kinetics of the anti-TfR antibody can be tested using any suitable method including but not limited to biosensor technology (e.g., OCTET or BIACORE).

In some embodiments, the anti-TfR antibody of the present disclosure comprises a VL domain and/or (e.g., and) VH domain of any one of the anti-TfR antibodies selected from Table 1, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra.

In some embodiments, the anti-TfR antibody of the present disclosure comprises a CDR-H1, a CDR-H2, and a CDR-H3 of a heavy chain variable domain having the amino acid sequence of SEQ ID NO: 7. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 8.

In some embodiments, the anti-TfR antibody of the present disclosure comprises a CDR-H1 having the amino acid sequence of SEQ ID NO: 1 (according to the IMGT definition system), a CDR-H2 having the amino acid sequence of SEQ ID NO: 2 (according to the IMGT definition system), a CDR-H3 having the amino acid sequence of SEQ ID NO: 3 (according to the IMGT definition system), a CDR-L1 having the amino acid sequence of SEQ ID NO: 4 (according to the IMGT definition system), a CDR-L2 having the amino acid sequence of SEQ ID NO: 5 (according to the IMGT definition system), and a CDR-L3 having the amino acid sequence of SEQ ID NO: 6 (according to the IMGT definition system).

In some embodiments, the anti-TfR antibody of the present disclosure comprises: a CDR-H1 having the amino acid sequence of SEQ ID NO: 1; a CDR-H2 having the amino acid sequence of SEQ ID NO: 2 with an amino acid substitution at position 5 (e.g., the asparagine at position 5 is substituted, e.g., with any one of Arg (R), Lys (K), Asp (D), Glu (E), Gln (Q), His (H), Ser (S), Thr (T), Tyr (Y), Cys (C), Trp (W), Met (M), Ala (A), Ile (I), Leu (L), Phe (F), Val (V), Pro (P), Gly (G)); and a CDR-H3 having the amino acid sequence of SEQ ID NO: 3. Alternatively or in addition (e.g., in addition), the anti-TfR antibody of the present disclosure comprises: a CDR-L1 having the amino acid sequence of SEQ ID NO: 4; a CDR-L2 having the amino acid sequence of SEQ ID NO: 5; and a CDR-L3 having the amino acid sequence of SEQ ID NO: 6. In some embodiments, the amino acid substitution at position 5 of the CDR-H2 as set forth in SEQ ID NO: 2 is N5T or N5S.

In some embodiments, the anti-TfR antibody of the present disclosure comprises: a CDR-H1 having the amino acid sequence of SEQ ID NO: 1; a CDR-H2 having the amino acid sequence of SEQ ID NO: 233 or SEQ ID NO: 80; and a CDR-H3 having the amino acid sequence of SEQ ID NO: 3. Alternatively or in addition (e.g., in addition), the anti-TfR antibody of the present disclosure comprises: a CDR-L1 having the amino acid sequence of SEQ ID NO: 4; a CDR-L2 having the amino acid sequence of SEQ ID NO: 5; and a CDR-L3 having the amino acid sequence of SEQ ID NO: 6.

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 1, CDR-H2 having the amino acid sequence of SEQ ID NO: 2, SEQ ID NO: 233 or SEQ ID NO: 80, and CDR-H3 having the amino acid sequence of SEQ ID NO: 3. “Collectively,” as used anywhere in the present disclosure, means that the total number of amino acid variations in all of the three heavy chain CDRs is within the defined range. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 4, CDR-L2 having the amino acid sequence of SEQ ID NO: 5, and CDR-L3 having the amino acid sequence of SEQ ID NO: 6.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 1, CDR-H2 having the amino acid sequence of SEQ ID NO: 2, SEQ ID NO: 233 or SEQ ID NO: 80, and CDR-H3 having the amino acid sequence of SEQ ID NO: 3. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 4, CDR-L2 having the amino acid sequence of SEQ ID NO: 5, and CDR-L3 having the amino acid sequence of SEQ ID NO: 6.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 1; a CDR-H2 having no more than 3 amino acid variations (e.g., no more than 3, 2, or 1 amino acid variation) as compared with the CDR-H2 having the amino acid sequence of SEQ ID NO: 2, SEQ ID NO: 233 or SEQ ID NO: 80; and/or (e.g., and) a CDR-H3 having no more than 3 amino acid variations (e.g., no more than 3, 2, or 1 amino acid variation) as compared with the CDR-H3 having the amino acid sequence of SEQ ID NO: 3. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 4; a CDR-L2 having no more than 3 amino acid variations (e.g., no more than 3, 2, or 1 amino acid variation) as compared with the CDR-L2 having the amino acid sequence of SEQ ID NO: 5; and/or (e.g., and) a CDR-L3 having no more than 3 amino acid variations (e.g., no more than 3, 2, or 1 amino acid variation) as compared with the CDR-L3 having the amino acid sequence of SEQ ID NO: 6.

In some embodiments, the anti-TfR antibody of the present disclosure comprises a VH comprising the amino acid sequence of SEQ ID NO: 7. Alternatively or in addition (e.g., in addition), the anti-TfR antibody of the present disclosure comprises a VL comprising the amino acid sequence of SEQ ID NO: 8.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 8.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 7. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 8.

In some embodiments, the anti-TfR antibody of the present disclosure comprises a VH as set forth in SEQ ID NO: 7 with an amino acid substitution at position 55 (e.g., the asparagine at position 55 is substituted, e.g., with any one of Arg (R), Lys (K), Asp (D), Glu (E), Gln (Q), His (H), Ser (S), Thr (T), Tyr (Y), Cys (C), Trp (W), Met (M), Ala (A), Ile (I), Leu (L), Phe (F), Val (V), Pro (P), Gly (G)). Alternatively or in addition (e.g., in addition), the anti-TfR antibody of the present disclosure comprises a VL as set forth in SEQ ID NO: 8. In some embodiments, the amino acid substitution at position 55 of the VH as set forth in SEQ ID NO: 7 is N55T or N55S. Amino acid position 55 in SEQ ID NO: 7 is assigned a number 54 when the VH set forth in SEQ ID NO: 7 is annotated using the Kabat numbering system. When N54T or N54S is referred to herein, it is referring to the mutations using the Kabat numbering system.

In some embodiments, the anti-TfR antibody of the present disclosure comprises a VH comprising an amino acid substitution at position 64 relative to SEQ ID NO: 7. In some embodiments, the anti-TfR antibody of the present disclosure comprises a VH comprising a Met at a position corresponding to position 64 of SEQ ID NO: 7. Alternatively or in addition (e.g., in addition), the anti-TfR antibody of the present disclosure comprises a VL comprising an amino acid sequence that is at least 80% (e.g., 80%, 85%, 90%, 95%, 98%, 99%, or 100%) identical to the VL as set forth in SEQ ID NO: 8.

In some embodiments, the anti-TfR antibody of the present disclosure comprises a CDR-H1 set forth as GYSITSGYX₁ (SEQ ID NO: 286), in which X₁ can be Y or G; a CDR-H2 set forth as IX₂FDGX₃X₄ (SEQ ID NO: 287), in which X₂ can be T or N, X₃ can be A or N, and X₄ can be N, T, or S; and a CDR-H3 set forth as X₅RX₆X₇YDYDX₈X₉DX₁₀ (SEQ ID NO: 288), wherein X₅ is T or A, X₆ is S, F, or I, X₇ is S, N, or Y, X₈ is P, Y, or V, X₉ is I, F, or L, and X₁₀ is Y or F. Alternatively or in addition (e.g., in addition), the anti-TfR antibody of the present disclosure comprises a CDR-L1 set forth as QDIX₁₁NX₁₂ (SEQ ID NO: 289), in which X₁₁ is S or T and X₁₂ is F, C, S, or Y; a CDR-L2 set forth as YTS (SEQ ID NO: 13), and a CDR-L3 set forth as QQGX₁₃X₁₄X₁₅PX₁₆T (SEQ ID NO: 290), in which X₁₃ is H or N, X₁₄ is T or A, X₁₅ is L or Y, and X₁₆ is Y, W, or F.

In some embodiments, the anti-TfR antibody of the present disclosure comprises a CDR-H1, a CDR-H2, and a CDR-H3 as the CDR-H1, CDR-H2, and CDR-H3 listed in Table 11. Alternatively or in addition (e.g., in addition), the anti-TfR antibody of the present disclosure comprises a CDR-L1, a CDR-L2, and a CDR-L3 as the CDR-L1, CDR-L2, and CDR-L3 listed in Table 11.

In some embodiments, the anti-TfR antibody of the present disclosure comprises a CDR-H1, a CDR-H2, and a CDR-H3 of a heavy chain variable domain having the amino acid sequence of SEQ ID NO: 15. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 16.

In some embodiments, the anti-TfR antibody of the present disclosure comprises a CDR-H1 having the amino acid sequence of SEQ ID NO: 9 (according to the IMGT definition system), a CDR-H2 having the amino acid sequence of SEQ ID NO: 10 (according to the IMGT definition system), a CDR-H3 having the amino acid sequence of SEQ ID NO: 11 (according to the IMGT definition system), a CDR-L1 having the amino acid sequence of SEQ ID NO: 12 (according to the IMGT definition system), a CDR-L2 having the amino acid sequence of SEQ ID NO: 13 (according to the IMGT definition system), and a CDR-L3 having the amino acid sequence of SEQ ID NO: 14 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 9, CDR-H2 having the amino acid sequence of SEQ ID NO: 10, and CDR-H3 having the amino acid sequence of SEQ ID NO: 11. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 12, CDR-L2 having the amino acid sequence of SEQ ID NO: 13, and CDR-L3 having the amino acid sequence of SEQ ID NO: 14.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 9, CDR-H2 having the amino acid sequence of SEQ ID NO: 10, and CDR-H3 having the amino acid sequence of SEQ ID NO: 11. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 12, CDR-L2 having the amino acid sequence of SEQ ID NO: 13, and CDR-L3 having the amino acid sequence of SEQ ID NO: 14.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 9; a CDR-H2 having no more than 3 amino acid variations (e.g., no more than 3, 2, or 1 amino acid variation) as compared with the CDR-H2 having the amino acid sequence of SEQ ID NO: 10; and/or (e.g., and) a CDR-H3 having no more than 3 amino acid variations (e.g., no more than 3, 2, or 1 amino acid variation) as compared with the CDR-H3 having the amino acid sequence of SEQ ID NO: 11. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 12; a CDR-L2 having no more than 3 amino acid variations (e.g., no more than 3, 2, or 1 amino acid variation) as compared with the CDR-L2 having the amino acid sequence of SEQ ID NO: 13; and/or (e.g., and) a CDR-L3 having no more than 3 amino acid variations (e.g., no more than 3, 2, or 1 amino acid variation) as compared with the CDR-L3 having the amino acid sequence of SEQ ID NO: 14.

In some embodiments, the anti-TfR antibody of the present disclosure comprises a VH comprising the amino acid sequence of SEQ ID NO: 15. Alternatively or in addition (e.g., in addition), the anti-TfR antibody of the present disclosure comprises a VL comprising the amino acid sequence of SEQ ID NO: 16.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 15. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 16.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 15. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 16.

In some embodiments, the anti-TfR antibody of the present disclosure comprises a CDR-H1, a CDR-H2, and a CDR-H3 of a heavy chain variable domain having the amino acid sequence of SEQ ID NO: 23. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 24.

In some embodiments, the anti-TfR antibody of the present disclosure comprises a CDR-H1 having the amino acid sequence of SEQ ID NO: 17 (according to the IMGT definition system), a CDR-H2 having the amino acid sequence of SEQ ID NO: 18 (according to the IMGT definition system), a CDR-H3 having the amino acid sequence of SEQ ID NO: 19 (according to the IMGT definition system), a CDR-L1 having the amino acid sequence of SEQ ID NO: 20 (according to the IMGT definition system), a CDR-L2 having the amino acid sequence of SEQ ID NO: 21 (according to the IMGT definition system), and a CDR-L3 having the amino acid sequence of SEQ ID NO: 22 (according to the IMGT definition system).

In some embodiments, the anti-TfR antibody of the present disclosure comprises: a CDR-H1 having the amino acid sequence of SEQ ID NO: 17 with an amino acid substitution at position 8 (e.g., the cysteine at position 8 is substituted, e.g., with any one of Arg (R), Lys (K), Asp (D), Glu (E), Gln (Q), His (H), Ser (S), Thr (T), Tyr (Y), Asn (N), Trp (W), Met (M), Ala (A), Ile (I), Leu (L), Phe (F), Val (V), Pro (P), Gly (G)); a CDR-H2 having the amino acid sequence of SEQ ID NO: 18; and a CDR-H3 having the amino acid sequence of SEQ ID NO: 19. Alternatively or in addition (e.g., in addition), the anti-TfR antibody of the present disclosure comprises: a CDR-L1 having the amino acid sequence of SEQ ID NO: 20; a CDR-L2 having the amino acid sequence of SEQ ID NO: 21; and a CDR-L3 having the amino acid sequence of SEQ ID NO: 22. In some embodiments, the amino acid substitution at position 8 of the CDR-H1 as set forth in SEQ ID NO: 17 is C8D or C8Y.

In some embodiments, the anti-TfR antibody of the present disclosure comprises: a CDR-H1 having the amino acid sequence of SEQ ID NO: 237 or SEQ ID NO: 239; a CDR-H2 having the amino acid sequence of SEQ ID NO: 18; and a CDR-H3 having the amino acid sequence of SEQ ID NO: 19. Alternatively or in addition (e.g., in addition), the anti-TfR antibody of the present disclosure comprises: a CDR-L1 having the amino acid sequence of SEQ ID NO: 20; a CDR-L2 having the amino acid sequence of SEQ ID NO: 21; and a CDR-L3 having the amino acid sequence of SEQ ID NO: 22.

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 17, SEQ ID NO: 237, or SEQ ID NO: 239, CDR-H2 having the amino acid sequence of SEQ ID NO: 18, and CDR-H3 having the amino acid sequence of SEQ ID NO: 19. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 20, CDR-L2 having the amino acid sequence of SEQ ID NO: 21, and CDR-L3 having the amino acid sequence of SEQ ID NO: 22.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 17, SEQ ID NO: 237, or SEQ ID NO: 239, CDR-H2 having the amino acid sequence of SEQ ID NO: 18, and CDR-H3 having the amino acid sequence of SEQ ID NO: 19. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 20, CDR-L2 having the amino acid sequence of SEQ ID NO: 21, and CDR-L3 having the amino acid sequence of SEQ ID NO: 22.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 17, SEQ ID NO: 237, or SEQ ID NO: 239; a CDR-H2 having no more than 3 amino acid variations (e.g., no more than 3, 2, or 1 amino acid variation) as compared with the CDR-H2 having the amino acid sequence of SEQ ID NO: 18; and/or (e.g., and) a CDR-H3 having no more than 3 amino acid variations (e.g., no more than 3, 2, or 1 amino acid variation) as compared with the CDR-H3 having the amino acid sequence of SEQ ID NO: 19. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 20; a CDR-L2 having no more than 3 amino acid variations (e.g., no more than 3, 2, or 1 amino acid variation) as compared with the CDR-L2 having the amino acid sequence of SEQ ID NO: 21; and/or (e.g., and) a CDR-L3 having no more than 3 amino acid variations (e.g., no more than 3, 2, or 1 amino acid variation) as compared with the CDR-L3 having the amino acid sequence of SEQ ID NO: 22.

In some embodiments, the anti-TfR antibody of the present disclosure comprises a VH comprising the amino acid sequence of SEQ ID NO: 23. Alternatively or in addition (e.g., in addition), the anti-TfR antibody of the present disclosure comprises a VL comprising the amino acid sequence of SEQ ID NO: 24.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 23. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 24.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 23. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 24.

In some embodiments, the anti-TfR antibody of the present disclosure comprises a VH as set forth in SEQ ID NO: 23 with an amino acid substitution at position 33 (e.g., the cysteine at position 33 is substituted, e.g., with any one of Arg (R), Lys (K), Asp (D), Glu (E), Gln (Q), His (H), Ser (S), Thr (T), Tyr (Y), Asn (N), Trp (W), Met (M), Ala (A), Ile (I), Leu (L), Phe (F), Val (V), Pro (P), Gly (G)). Alternatively or in addition (e.g., in addition), the anti-TfR antibody of the present disclosure comprises a VL as set forth in SEQ ID NO: 24. In some embodiments, the amino acid substitution at position 33 of the VH as set forth in SEQ ID NO: 23 is C33D or C33Y. Amino acid 33 in SEQ ID NO: 23 is assigned a number 33 when the VH set forth in SEQ ID NO: 23 is annotated with the Kabat numbering system.

In some embodiments, the anti-TfR antibody of the present disclosure comprises a CDR-H1, a CDR-H2, and a CDR-H3 of a heavy chain variable domain having the amino acid sequence of SEQ ID NO: 31. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 32.

In some embodiments, the anti-TfR antibody of the present disclosure comprises a CDR-H1 having the amino acid sequence of SEQ ID NO: 25 (according to the IMGT definition system), a CDR-H2 having the amino acid sequence of SEQ ID NO: 26 (according to the IMGT definition system), a CDR-H3 having the amino acid sequence of SEQ ID NO: 27 (according to the IMGT definition system), a CDR-L1 having the amino acid sequence of SEQ ID NO: 28 (according to the IMGT definition system), a CDR-L2 having the amino acid sequence of SEQ ID NO: 29 (according to the IMGT definition system), and a CDR-L3 having the amino acid sequence of SEQ ID NO: 30 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 25, CDR-H2 having the amino acid sequence of SEQ ID NO: 26, and CDR-H3 having the amino acid sequence of SEQ ID NO: 27. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 28, CDR-L2 having the amino acid sequence of SEQ ID NO: 29, and CDR-L3 having the amino acid sequence of SEQ ID NO: 30.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 25, CDR-H2 having the amino acid sequence of SEQ ID NO: 26, and CDR-H3 having the amino acid sequence of SEQ ID NO: 27. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 28, CDR-L2 having the amino acid sequence of SEQ ID NO: 29, and CDR-L3 having the amino acid sequence of SEQ ID NO: 30.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 25; a CDR-H2 having no more than 3 amino acid variations (e.g., no more than 3, 2, or 1 amino acid variation) as compared with the CDR-H2 having the amino acid sequence of SEQ ID NO: 26; and/or (e.g., and) a CDR-H3 having no more than 3 amino acid variations (e.g., no more than 3, 2, or 1 amino acid variation) as compared with the CDR-H3 having the amino acid sequence of SEQ ID NO: 27. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 28; a CDR-L2 having no more than 3 amino acid variations (e.g., no more than 3, 2, or 1 amino acid variation) as compared with the CDR-L2 having the amino acid sequence of SEQ ID NO: 29; and/or (e.g., and) a CDR-L3 having no more than 3 amino acid variations (e.g., no more than 3, 2, or 1 amino acid variation) as compared with the CDR-L3 having the amino acid sequence of SEQ ID NO: 30.

In some embodiments, the anti-TfR antibody of the present disclosure comprises a VH comprising the amino acid sequence of SEQ ID NO: 31. Alternatively or in addition (e.g., in addition), the anti-TfR antibody of the present disclosure comprises a VL comprising the amino acid sequence of SEQ ID NO: 32.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 31. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 32.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 31. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 32.

In some embodiments, the anti-TfR antibody of the present disclosure comprises a CDR-H1, a CDR-H2, and a CDR-H3 of a heavy chain variable domain having the amino acid sequence of SEQ ID NO: 39. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 40.

In some embodiments, the anti-TfR antibody of the present disclosure comprises a CDR-H1 having the amino acid sequence of SEQ ID NO: 33 (according to the IMGT definition system), a CDR-H2 having the amino acid sequence of SEQ ID NO: 34 (according to the IMGT definition system), a CDR-H3 having the amino acid sequence of SEQ ID NO: 35 (according to the IMGT definition system), a CDR-L1 having the amino acid sequence of SEQ ID NO: 36 (according to the IMGT definition system), a CDR-L2 having the amino acid sequence of SEQ ID NO: 37 (according to the IMGT definition system), and a CDR-L3 having the amino acid sequence of SEQ ID NO: 38 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 33, CDR-H2 having the amino acid sequence of SEQ ID NO: 34, and CDR-H3 having the amino acid sequence of SEQ ID NO: 35. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 36, CDR-L2 having the amino acid sequence of SEQ ID NO: 37, and CDR-L3 having the amino acid sequence of SEQ ID NO: 38.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 33, CDR-H2 having the amino acid sequence of SEQ ID NO: 34, and CDR-H3 having the amino acid sequence of SEQ ID NO: 35. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 36, CDR-L2 having the amino acid sequence of SEQ ID NO: 37, and CDR-L3 having the amino acid sequence of SEQ ID NO: 38.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 33; a CDR-H2 having no more than 3 amino acid variations (e.g., no more than 3, 2, or 1 amino acid variation) as compared with the CDR-H2 having the amino acid sequence of SEQ ID NO: 34; and/or (e.g., and) a CDR-H3 having no more than 3 amino acid variations (e.g., no more than 3, 2, or 1 amino acid variation) as compared with the CDR-H3 having the amino acid sequence of SEQ ID NO: 35. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 36; a CDR-L2 having no more than 3 amino acid variations (e.g., no more than 3, 2, or 1 amino acid variation) as compared with the CDR-L2 having the amino acid sequence of SEQ ID NO: 37; and/or (e.g., and) a CDR-L3 having no more than 3 amino acid variations (e.g., no more than 3, 2, or 1 amino acid variation) as compared with the CDR-L3 having the amino acid sequence of SEQ ID NO: 38.

In some embodiments, the anti-TfR antibody of the present disclosure comprises a VH comprising the amino acid sequence of SEQ ID NO: 39. Alternatively or in addition (e.g., in addition), the anti-TfR antibody of the present disclosure comprises a VL comprising the amino acid sequence of SEQ ID NO: 40.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 39. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 40.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 39. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 40.

In some embodiments, the anti-TfR antibody of the present disclosure comprises a CDR-H1, a CDR-H2, and a CDR-H3 of a heavy chain variable domain having the amino acid sequence of SEQ ID NO: 47. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 48.

In some embodiments, the anti-TfR antibody of the present disclosure comprises a CDR-H1 having the amino acid sequence of SEQ ID NO: 41 (according to the IMGT definition system), a CDR-H2 having the amino acid sequence of SEQ ID NO: 42 (according to the IMGT definition system), a CDR-H3 having the amino acid sequence of SEQ ID NO: 43 (according to the IMGT definition system), a CDR-L1 having the amino acid sequence of SEQ ID NO: 44 (according to the IMGT definition system), a CDR-L2 having the amino acid sequence of SEQ ID NO: 45 (according to the IMGT definition system), and a CDR-L3 having the amino acid sequence of SEQ ID NO: 46 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 41, CDR-H2 having the amino acid sequence of SEQ ID NO: 42, and CDR-H3 having the amino acid sequence of SEQ ID NO: 43. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 44, CDR-L2 having the amino acid sequence of SEQ ID NO: 45, and CDR-L3 having the amino acid sequence of SEQ ID NO: 46.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 41, CDR-H2 having the amino acid sequence of SEQ ID NO: 42, and CDR-H3 having the amino acid sequence of SEQ ID NO: 43. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 44, CDR-L2 having the amino acid sequence of SEQ ID NO: 45, and CDR-L3 having the amino acid sequence of SEQ ID NO: 46.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 41; a CDR-H2 having no more than 3 amino acid variations (e.g., no more than 3, 2, or 1 amino acid variation) as compared with the CDR-H2 having the amino acid sequence of SEQ ID NO: 42; and/or (e.g., and) a CDR-H3 having no more than 3 amino acid variations (e.g., no more than 3, 2, or 1 amino acid variation) as compared with the CDR-H3 having the amino acid sequence of SEQ ID NO: 43. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 44; a CDR-L2 having no more than 3 amino acid variations (e.g., no more than 3, 2, or 1 amino acid variation) as compared with the CDR-L2 having the amino acid sequence of SEQ ID NO: 45; and/or (e.g., and) a CDR-L3 having no more than 3 amino acid variations (e.g., no more than 3, 2, or 1 amino acid variation) as compared with the CDR-L3 having the amino acid sequence of SEQ ID NO: 46.

In some embodiments, the anti-TfR antibody of the present disclosure comprises a VH comprising the amino acid sequence of SEQ ID NO: 47. Alternatively or in addition (e.g., in addition), the anti-TfR antibody of the present disclosure comprises a VL comprising the amino acid sequence of SEQ ID NO: 48.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 47. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 48.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 47. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 48.

In some embodiments, the anti-TfR antibody of the present disclosure comprises a CDR-H1, a CDR-H2, and a CDR-H3 of a heavy chain variable domain having the amino acid sequence of SEQ ID NO: 54. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 55.

In some embodiments, the anti-TfR antibody of the present disclosure comprises a CDR-H1 having the amino acid sequence of SEQ ID NO: 49 (according to the IMGT definition system), a CDR-H2 having the amino acid sequence of SEQ ID NO: 50 (according to the IMGT definition system), a CDR-H3 having the amino acid sequence of SEQ ID NO: 51 (according to the IMGT definition system), a CDR-L1 having the amino acid sequence of SEQ ID NO: 52 (according to the IMGT definition system), a CDR-L2 having the amino acid sequence of SEQ ID NO: 29 (according to the IMGT definition system), and a CDR-L3 having the amino acid sequence of SEQ ID NO: 53 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 49, CDR-H2 having the amino acid sequence of SEQ ID NO: 50, and CDR-H3 having the amino acid sequence of SEQ ID NO: 51. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 52, CDR-L2 having the amino acid sequence of SEQ ID NO: 29, and CDR-L3 having the amino acid sequence of SEQ ID NO: 53.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 49, CDR-H2 having the amino acid sequence of SEQ ID NO: 50, and CDR-H3 having the amino acid sequence of SEQ ID NO: 51. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 52, CDR-L2 having the amino acid sequence of SEQ ID NO: 29, and CDR-L3 having the amino acid sequence of SEQ ID NO: 53.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 49; a CDR-H2 having no more than 3 amino acid variations (e.g., no more than 3, 2, or 1 amino acid variation) as compared with the CDR-H2 having the amino acid sequence of SEQ ID NO: 50; and/or (e.g., and) a CDR-H3 having no more than 3 amino acid variations (e.g., no more than 3, 2, or 1 amino acid variation) as compared with the CDR-H3 having the amino acid sequence of SEQ ID NO: 51. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 52; a CDR-L2 having no more than 3 amino acid variations (e.g., no more than 3, 2, or 1 amino acid variation) as compared with the CDR-L2 having the amino acid sequence of SEQ ID NO: 29; and/or (e.g., and) a CDR-L3 having no more than 3 amino acid variations (e.g., no more than 3, 2, or 1 amino acid variation) as compared with the CDR-L3 having the amino acid sequence of SEQ ID NO: 53.

In some embodiments, the anti-TfR antibody of the present disclosure comprises a VH comprising the amino acid sequence of SEQ ID NO: 54. Alternatively or in addition (e.g., in addition), the anti-TfR antibody of the present disclosure comprises a VL comprising the amino acid sequence of SEQ ID NO: 55.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 54. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 55.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 54. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 55.

In some embodiments, the anti-TfR antibody of the present disclosure comprises a CDR-H1, a CDR-H2, and a CDR-H3 of a heavy chain variable domain having the amino acid sequence of SEQ ID NO: 62. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 63.

In some embodiments, the anti-TfR antibody of the present disclosure comprises a CDR-H1 having the amino acid sequence of SEQ ID NO: 56 (according to the IMGT definition system), a CDR-H2 having the amino acid sequence of SEQ ID NO: 57 (according to the IMGT definition system), a CDR-H3 having the amino acid sequence of SEQ ID NO: 58 (according to the IMGT definition system), a CDR-L1 having the amino acid sequence of SEQ ID NO: 59 (according to the IMGT definition system), a CDR-L2 having the amino acid sequence of SEQ ID NO: 60 (according to the IMGT definition system), and a CDR-L3 having the amino acid sequence of SEQ ID NO: 61 (according to the IMGT definition system).

In some embodiments, anti-TfR antibody of the present disclosure comprises a CDR-H1, a CDR-H2, and a CDR-H3, which collectively contains no more than 5 amino acid H1 having the amino acid sequence of SEQ ID NO: 56, CDR-H2 having the amino acid sequence of SEQ ID NO: 57, and CDR-H3 having the amino acid sequence of SEQ ID NO: 58. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 59, CDR-L2 having the amino acid sequence of SEQ ID NO: 60, and CDR-L3 having the amino acid sequence of SEQ ID NO: 61.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 56, CDR-H2 having the amino acid sequence of SEQ ID NO: 57, and CDR-H3 having the amino acid sequence of SEQ ID NO: 58. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 59, CDR-L2 having the amino acid sequence of SEQ ID NO: 60, and CDR-L3 having the amino acid sequence of SEQ ID NO: 61.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 56; a CDR-H2 having no more than 3 amino acid variations (e.g., no more than 3, 2, or 1 amino acid variation) as compared with the CDR-H2 having the amino acid sequence of SEQ ID NO: 57; and/or (e.g., and) a CDR-H3 having no more than 3 amino acid variations (e.g., no more than 3, 2, or 1 amino acid variation) as compared with the CDR-H3 having the amino acid sequence of SEQ ID NO: 58. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 59; a CDR-L2 having no more than 3 amino acid variations (e.g., no more than 3, 2, or 1 amino acid variation) as compared with the CDR-L2 having the amino acid sequence of SEQ ID NO: 60; and/or (e.g., and) a CDR-L3 having no more than 3 amino acid variations (e.g., no more than 3, 2, or 1 amino acid variation) as compared with the CDR-L3 having the amino acid sequence of SEQ ID NO: 61.

In some embodiments, the anti-TfR antibody of the present disclosure comprises a VH comprising the amino acid sequence of SEQ ID NO: 62. Alternatively or in addition (e.g., in addition), the anti-TfR antibody of the present disclosure comprises a VL comprising the amino acid sequence of SEQ ID NO: 63.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 62. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 63.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 62. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 63.

In some embodiments, the anti-TfR antibody of the present disclosure comprises a CDR-H1, a CDR-H2, and a CDR-H3 of a heavy chain variable domain having the amino acid sequence of SEQ ID NO: 70. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 71.

In some embodiments, the anti-TfR antibody of the present disclosure comprises a CDR-H1 having the amino acid sequence of SEQ ID NO: 64 (according to the IMGT definition system), a CDR-H2 having the amino acid sequence of SEQ ID NO: 65 (according to the IMGT definition system), a CDR-H3 having the amino acid sequence of SEQ ID NO: 66 (according to the IMGT definition system), a CDR-L1 having the amino acid sequence of SEQ ID NO: 67 (according to the IMGT definition system), a CDR-L2 having the amino acid sequence of SEQ ID NO: 68 (according to the IMGT definition system), and a CDR-L3 having the amino acid sequence of SEQ ID NO: 69 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 64, CDR-H2 having the amino acid sequence of SEQ ID NO: 65, and CDR-H3 having the amino acid sequence of SEQ ID NO: 66. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 67, CDR-L2 having the amino acid sequence of SEQ ID NO: 68, and CDR-L3 having the amino acid sequence of SEQ ID NO: 69.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 64, CDR-H2 having the amino acid sequence of SEQ ID NO: 65, and CDR-H3 having the amino acid sequence of SEQ ID NO: 66. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 67, CDR-L2 having the amino acid sequence of SEQ ID NO: 68, and CDR-L3 having the amino acid sequence of SEQ ID NO: 69.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 64; a CDR-H2 having no more than 3 amino acid variations (e.g., no more than 3, 2, or 1 amino acid variation) as compared with the CDR-H2 having the amino acid sequence of SEQ ID NO: 65; and/or (e.g., and) a CDR-H3 having no more than 3 amino acid variations (e.g., no more than 3, 2, or 1 amino acid variation) as compared with the CDR-H3 having the amino acid sequence of SEQ ID NO: 66. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 67; a CDR-L2 having no more than 3 amino acid variations (e.g., no more than 3, 2, or 1 amino acid variation) as compared with the CDR-L2 having the amino acid sequence of SEQ ID NO: 68; and/or (e.g., and) a CDR-L3 having no more than 3 amino acid variations (e.g., no more than 3, 2, or 1 amino acid variation) as compared with the CDR-L3 having the amino acid sequence of SEQ ID NO: 69.

In some embodiments, the anti-TfR antibody of the present disclosure comprises a VH comprising the amino acid sequence of SEQ ID NO: 70. Alternatively or in addition (e.g., in addition), the anti-TfR antibody of the present disclosure comprises a VL comprising the amino acid sequence of SEQ ID NO: 71.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 70. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 71.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 70. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 71.

In some embodiments, the anti-TfR antibody of the present disclosure comprises a CDR-H1, a CDR-H2, and a CDR-H3 of a heavy chain variable domain having the amino acid sequence of SEQ ID NO: 77. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 78.

In some embodiments, the anti-TfR antibody of the present disclosure comprises a CDR-H1 having the amino acid sequence of SEQ ID NO: 72 (according to the IMGT definition system), a CDR-H2 having the amino acid sequence of SEQ ID NO: 73 (according to the IMGT definition system), a CDR-H3 having the amino acid sequence of SEQ ID NO: 74 (according to the IMGT definition system), a CDR-L1 having the amino acid sequence of SEQ ID NO: 75 (according to the IMGT definition system), a CDR-L2 having the amino acid sequence of SEQ ID NO: 45 (according to the IMGT definition system), and a CDR-L3 having the amino acid sequence of SEQ ID NO: 76 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 72, CDR-H2 having the amino acid sequence of SEQ ID NO: 73, and CDR-H3 having the amino acid sequence of SEQ ID NO: 74. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 75, CDR-L2 having the amino acid sequence of SEQ ID NO: 45, and CDR-L3 having the amino acid sequence of SEQ ID NO: 76.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 72, CDR-H2 having the amino acid sequence of SEQ ID NO: 73, and CDR-H3 having the amino acid sequence of SEQ ID NO: 74. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 75, CDR-L2 having the amino acid sequence of SEQ ID NO: 45, and CDR-L3 having the amino acid sequence of SEQ ID NO: 76.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 72; a CDR-H2 having no more than 3 amino acid variations (e.g., no more than 3, 2, or 1 amino acid variation) as compared with the CDR-H2 having the amino acid sequence of SEQ ID NO: 73; and/or (e.g., and) a CDR-H3 having no more than 3 amino acid variations (e.g., no more than 3, 2, or 1 amino acid variation) as compared with the CDR-H3 having the amino acid sequence of SEQ ID NO: 74. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 75; a CDR-L2 having no more than 3 amino acid variations (e.g., no more than 3, 2, or 1 amino acid variation) as compared with the CDR-L2 having the amino acid sequence of SEQ ID NO: 45; and/or (e.g., and) a CDR-L3 having no more than 3 amino acid variations (e.g., no more than 3, 2, or 1 amino acid variation) as compared with the CDR-L3 having the amino acid sequence of SEQ ID NO: 76.

In some embodiments, the anti-TfR antibody of the present disclosure comprises a VH comprising the amino acid sequence of SEQ ID NO: 77. Alternatively or in addition (e.g., in addition), the anti-TfR antibody of the present disclosure comprises a VL comprising the amino acid sequence of SEQ ID NO: 78.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 77. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 78.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 77. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 78.

In some embodiments, the anti-TfR antibody of the present disclosure comprises a CDR-H1, a CDR-H2, and a CDR-H3 of a heavy chain variable domain having the amino acid sequence of SEQ ID NO: 85. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 86.

In some embodiments, the anti-TfR antibody of the present disclosure comprises a CDR-H1 having the amino acid sequence of SEQ ID NO: 79 (according to the IMGT definition system), a CDR-H2 having the amino acid sequence of SEQ ID NO: 80 (according to the IMGT definition system), a CDR-H3 having the amino acid sequence of SEQ ID NO: 81 (according to the IMGT definition system), a CDR-L1 having the amino acid sequence of SEQ ID NO: 82 (according to the IMGT definition system), a CDR-L2 having the amino acid sequence of SEQ ID NO: 83 (according to the IMGT definition system), and a CDR-L3 having the amino acid sequence of SEQ ID NO: 84 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 79, CDR-H2 having the amino acid sequence of SEQ ID NO: 80, and CDR-H3 having the amino acid sequence of SEQ ID NO: 81. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 82, CDR-L2 having the amino acid sequence of SEQ ID NO: 83, and CDR-L3 having the amino acid sequence of SEQ ID NO: 84.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 79, CDR-H2 having the amino acid sequence of SEQ ID NO: 80, and CDR-H3 having the amino acid sequence of SEQ ID NO: 81. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 82, CDR-L2 having the amino acid sequence of SEQ ID NO: 83, and CDR-L3 having the amino acid sequence of SEQ ID NO: 84.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 79; a CDR-H2 having no more than 3 amino acid variations (e.g., no more than 3, 2, or 1 amino acid variation) as compared with the CDR-H2 having the amino acid sequence of SEQ ID NO: 80; and/or (e.g., and) a CDR-H3 having no more than 3 amino acid variations (e.g., no more than 3, 2, or 1 amino acid variation) as compared with the CDR-H3 having the amino acid sequence of SEQ ID NO: 81. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 82; a CDR-L2 having no more than 3 amino acid variations (e.g., no more than 3, 2, or 1 amino acid variation) as compared with the CDR-L2 having the amino acid sequence of SEQ ID NO: 83; and/or (e.g., and) a CDR-L3 having no more than 3 amino acid variations (e.g., no more than 3, 2, or 1 amino acid variation) as compared with the CDR-L3 having the amino acid sequence of SEQ ID NO: 84.

In some embodiments, the anti-TfR antibody of the present disclosure comprises a VH comprising the amino acid sequence of SEQ ID NO: 85. Alternatively or in addition (e.g., in addition), the anti-TfR antibody of the present disclosure comprises a VL comprising the amino acid sequence of SEQ ID NO: 86.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 85. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 86.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 85. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 86.

In some embodiments, the anti-TfR antibody of the present disclosure comprises a CDR-H1, a CDR-H2, and a CDR-H3 of a heavy chain variable domain having the amino acid sequence of SEQ ID NO: 89. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 90.

In some embodiments, the anti-TfR antibody of the present disclosure comprises a CDR-H1 having the amino acid sequence of SEQ ID NO: 72 (according to the IMGT definition system), a CDR-H2 having the amino acid sequence of SEQ ID NO: 87 (according to the IMGT definition system), a CDR-H3 having the amino acid sequence of SEQ ID NO: 74 (according to the IMGT definition system), a CDR-L1 having the amino acid sequence of SEQ ID NO: 75 (according to the IMGT definition system), a CDR-L2 having the amino acid sequence of SEQ ID NO: 45 (according to the IMGT definition system), and a CDR-L3 having the amino acid sequence of SEQ ID NO: 88 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 72, CDR-H2 having the amino acid sequence of SEQ ID NO: 87, and CDR-H3 having the amino acid sequence of SEQ ID NO: 74. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 75, CDR-L2 having the amino acid sequence of SEQ ID NO: 45, and CDR-L3 having the amino acid sequence of SEQ ID NO: 88.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 72, CDR-H2 having the amino acid sequence of SEQ ID NO: 87, and CDR-H3 having the amino acid sequence of SEQ ID NO: 74. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 75, CDR-L2 having the amino acid sequence of SEQ ID NO: 45, and CDR-L3 having the amino acid sequence of SEQ ID NO: 88.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 72; a CDR-H2 having no more than 3 amino acid variations (e.g., no more than 3, 2, or 1 amino acid variation) as compared with the CDR-H2 having the amino acid sequence of SEQ ID NO: 87; and/or (e.g., and) a CDR-H3 having no more than 3 amino acid variations (e.g., no more than 3, 2, or 1 amino acid variation) as compared with the CDR-H3 having the amino acid sequence of SEQ ID NO: 74. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 75; a CDR-L2 having no more than 3 amino acid variations (e.g., no more than 3, 2, or 1 amino acid variation) as compared with the CDR-L2 having the amino acid sequence of SEQ ID NO: 45; and/or (e.g., and) a CDR-L3 having no more than 3 amino acid variations (e.g., no more than 3, 2, or 1 amino acid variation) as compared with the CDR-L3 having the amino acid sequence of SEQ ID NO: 88.

In some embodiments, the anti-TfR antibody of the present disclosure comprises a VH comprising the amino acid sequence of SEQ ID NO: 89. Alternatively or in addition (e.g., in addition), the anti-TfR antibody of the present disclosure comprises a VL comprising the amino acid sequence of SEQ ID NO: 90.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 89. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 90.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 89. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 90.

In some embodiments, the anti-TfR antibody of the present disclosure comprises a CDR-H1, a CDR-H2, and a CDR-H3 of a heavy chain variable domain having the amino acid sequence of SEQ ID NO: 97. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 98.

In some embodiments, the anti-TfR antibody of the present disclosure comprises a CDR-H1 having the amino acid sequence of SEQ ID NO: 91 (according to the IMGT definition system), a CDR-H2 having the amino acid sequence of SEQ ID NO: 92 (according to the IMGT definition system), a CDR-H3 having the amino acid sequence of SEQ ID NO: 93 (according to the IMGT definition system), a CDR-L1 having the amino acid sequence of SEQ ID NO: 94 (according to the IMGT definition system), a CDR-L2 having the amino acid sequence of SEQ ID NO: 95 (according to the IMGT definition system), and a CDR-L3 having the amino acid sequence of SEQ ID NO: 96 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 91, CDR-H2 having the amino acid sequence of SEQ ID NO: 92, and CDR-H3 having the amino acid sequence of SEQ ID NO: 93. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 94, CDR-L2 having the amino acid sequence of SEQ ID NO: 95, and CDR-L3 having the amino acid sequence of SEQ ID NO: 96.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 91, CDR-H2 having the amino acid sequence of SEQ ID NO: 92, and CDR-H3 having the amino acid sequence of SEQ ID NO: 93. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 94, CDR-L2 having the amino acid sequence of SEQ ID NO: 95, and CDR-L3 having the amino acid sequence of SEQ ID NO: 96.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 91; a CDR-H2 having no more than 3 amino acid variations (e.g., no more than 3, 2, or 1 amino acid variation) as compared with the CDR-H2 having the amino acid sequence of SEQ ID NO: 92; and/or (e.g., and) a CDR-H3 having no more than 3 amino acid variations (e.g., no more than 3, 2, or 1 amino acid variation) as compared with the CDR-H3 having the amino acid sequence of SEQ ID NO: 93. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 94; a CDR-L2 having no more than 3 amino acid variations (e.g., no more than 3, 2, or 1 amino acid variation) as compared with the CDR-L2 having the amino acid sequence of SEQ ID NO: 95; and/or (e.g., and) a CDR-L3 having no more than 3 amino acid variations (e.g., no more than 3, 2, or 1 amino acid variation) as compared with the CDR-L3 having the amino acid sequence of SEQ ID NO: 96.

In some embodiments, the anti-TfR antibody of the present disclosure comprises a VH comprising the amino acid sequence of SEQ ID NO: 97. Alternatively or in addition (e.g., in addition), the anti-TfR antibody of the present disclosure comprises a VL comprising the amino acid sequence of SEQ ID NO: 98.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 97. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 98.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 97. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 98.

In some embodiments, the anti-TfR antibody of the present disclosure comprises a CDR-H1, a CDR-H2, and a CDR-H3 of a heavy chain variable domain having the amino acid sequence of SEQ ID NO: 104. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 105.

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 99, CDR-H2 having the amino acid sequence of SEQ ID NO: 100, and CDR-H3 having the amino acid sequence of SEQ ID NO: 101. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 102, CDR-L2 having the amino acid sequence of SEQ ID NO: 60, and CDR-L3 having the amino acid sequence of SEQ ID NO: 103.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 99, CDR-H2 having the amino acid sequence of SEQ ID NO: 100, and CDR-H3 having the amino acid sequence of SEQ ID NO: 101. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 102, CDR-L2 having the amino acid sequence of SEQ ID NO: 60, and CDR-L3 having the amino acid sequence of SEQ ID NO: 103.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 99; a CDR-H2 having no more than 3 amino acid variations (e.g., no more than 3, 2, or 1 amino acid variation) as compared with the CDR-H2 having the amino acid sequence of SEQ ID NO: 100; and/or (e.g., and) a CDR-H3 having no more than 3 amino acid variations (e.g., no more than 3, 2, or 1 amino acid variation) as compared with the CDR-H3 having the amino acid sequence of SEQ ID NO: 101. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 102; a CDR-L2 having no more than 3 amino acid variations (e.g., no more than 3, 2, or 1 amino acid variation) as compared with the CDR-L2 having the amino acid sequence of SEQ ID NO: 60; and/or (e.g., and) a CDR-L3 having no more than 3 amino acid variations (e.g., no more than 3, 2, or 1 amino acid variation) as compared with the CDR-L3 having the amino acid sequence of SEQ ID NO: 103.

In some embodiments, the anti-TfR antibody of the present disclosure comprises a VH comprising the amino acid sequence of SEQ ID NO: 104. Alternatively or in addition (e.g., in addition), the anti-TfR antibody of the present disclosure comprises a VL comprising the amino acid sequence of SEQ ID NO: 105.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 104. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 105.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 104. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 105.

In some embodiments, the anti-TfR antibody of the present disclosure comprises a CDR-H1, a CDR-H2, and a CDR-H3 of a heavy chain variable domain having the amino acid sequence of SEQ ID NO: 112. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 113.

In some embodiments, the anti-TfR antibody of the present disclosure comprises a CDR-H1 having the amino acid sequence of SEQ ID NO: 106 (according to the IMGT definition system), a CDR-H2 having the amino acid sequence of SEQ ID NO: 107 (according to the IMGT definition system), a CDR-H3 having the amino acid sequence of SEQ ID NO: 108 (according to the IMGT definition system), a CDR-L1 having the amino acid sequence of SEQ ID NO: 109 (according to the IMGT definition system), a CDR-L2 having the amino acid sequence of SEQ ID NO: 110 (according to the IMGT definition system), and a CDR-L3 having the amino acid sequence of SEQ ID NO: 111 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 106, CDR-H2 having the amino acid sequence of SEQ ID NO: 107, and CDR-H3 having the amino acid sequence of SEQ ID NO: 108. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 109, CDR-L2 having the amino acid sequence of SEQ ID NO: 110, and CDR-L3 having the amino acid sequence of SEQ ID NO: 111.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 106, CDR-H2 having the amino acid sequence of SEQ ID NO: 107, and CDR-H3 having the amino acid sequence of SEQ ID NO: 108. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 109, CDR-L2 having the amino acid sequence of SEQ ID NO: 110, and CDR-L3 having the amino acid sequence of SEQ ID NO: 111.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 106; a CDR-H2 having no more than 3 amino acid variations (e.g., no more than 3, 2, or 1 amino acid variation) as compared with the CDR-H2 having the amino acid sequence of SEQ ID NO: 107; and/or (e.g., and) a CDR-H3 having no more than 3 amino acid variations (e.g., no more than 3, 2, or 1 amino acid variation) as compared with the CDR-H3 having the amino acid sequence of SEQ ID NO: 108. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 109; a CDR-L2 having no more than 3 amino acid variations (e.g., no more than 3, 2, or 1 amino acid variation) as compared with the CDR-L2 having the amino acid sequence of SEQ ID NO: 110; and/or (e.g., and) a CDR-L3 having no more than 3 amino acid variations (e.g., no more than 3, 2, or 1 amino acid variation) as compared with the CDR-L3 having the amino acid sequence of SEQ ID NO: 111.

In some embodiments, the anti-TfR antibody of the present disclosure comprises a VH comprising the amino acid sequence of SEQ ID NO: 112. Alternatively or in addition (e.g., in addition), the anti-TfR antibody of the present disclosure comprises a VL comprising the amino acid sequence of SEQ ID NO: 113.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 112. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 113.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 112. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 113.

In some embodiments, the anti-TfR antibody of the present disclosure comprises a CDR-H1, a CDR-H2, and a CDR-H3 of a heavy chain variable domain having the amino acid sequence of SEQ ID NO: 117. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 118.

In some embodiments, the anti-TfR antibody of the present disclosure comprises a CDR-H1 having the amino acid sequence of SEQ ID NO: 79 (according to the IMGT definition system), a CDR-H2 having the amino acid sequence of SEQ ID NO: 114 (according to the IMGT definition system), a CDR-H3 having the amino acid sequence of SEQ ID NO: 115 (according to the IMGT definition system), a CDR-L1 having the amino acid sequence of SEQ ID NO: 82 (according to the IMGT definition system), a CDR-L2 having the amino acid sequence of SEQ ID NO: 83 (according to the IMGT definition system), and a CDR-L3 having the amino acid sequence of SEQ ID NO: 116 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 79, CDR-H2 having the amino acid sequence of SEQ ID NO: 114, and CDR-H3 having the amino acid sequence of SEQ ID NO: 115. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 82, CDR-L2 having the amino acid sequence of SEQ ID NO: 83, and CDR-L3 having the amino acid sequence of SEQ ID NO: 116.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 79, CDR-H2 having the amino acid sequence of SEQ ID NO: 114, and CDR-H3 having the amino acid sequence of SEQ ID NO: 115. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 82, CDR-L2 having the amino acid sequence of SEQ ID NO: 83, and CDR-L3 having the amino acid sequence of SEQ ID NO: 116.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 79; a CDR-H2 having no more than 3 amino acid variations (e.g., no more than 3, 2, or 1 amino acid variation) as compared with the CDR-H2 having the amino acid sequence of SEQ ID NO: 114; and/or (e.g., and) a CDR-H3 having no more than 3 amino acid variations (e.g., no more than 3, 2, or 1 amino acid variation) as compared with the CDR-H3 having the amino acid sequence of SEQ ID NO: 115. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 82; a CDR-L2 having no more than 3 amino acid variations (e.g., no more than 3, 2, or 1 amino acid variation) as compared with the CDR-L2 having the amino acid sequence of SEQ ID NO: 83; and/or (e.g., and) a CDR-L3 having no more than 3 amino acid variations (e.g., no more than 3, 2, or 1 amino acid variation) as compared with the CDR-L3 having the amino acid sequence of SEQ ID NO: 116.

In some embodiments, the anti-TfR antibody of the present disclosure comprises a VH comprising the amino acid sequence of SEQ ID NO: 117. Alternatively or in addition (e.g., in addition), the anti-TfR antibody of the present disclosure comprises a VL comprising the amino acid sequence of SEQ ID NO: 118.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 117. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 118.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 117. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 118.

In some embodiments, the anti-TfR antibody of the present disclosure comprises a CDR-H1, a CDR-H2, and a CDR-H3 of a heavy chain variable domain having the amino acid sequence of SEQ ID NO: 124. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 125.

In some embodiments, the anti-TfR antibody of the present disclosure comprises a CDR-H1 having the amino acid sequence of SEQ ID NO: 119 (according to the IMGT definition system), a CDR-H2 having the amino acid sequence of SEQ ID NO: 120 (according to the IMGT definition system), a CDR-H3 having the amino acid sequence of SEQ ID NO: 121 (according to the IMGT definition system), a CDR-L1 having the amino acid sequence of SEQ ID NO: 122 (according to the IMGT definition system), a CDR-L2 having the amino acid sequence of SEQ ID NO: 45 (according to the IMGT definition system), and a CDR-L3 having the amino acid sequence of SEQ ID NO: 123 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 119, CDR-H2 having the amino acid sequence of SEQ ID NO: 120, and CDR-H3 having the amino acid sequence of SEQ ID NO: 121. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 122, CDR-L2 having the amino acid sequence of SEQ ID NO: 45, and CDR-L3 having the amino acid sequence of SEQ ID NO: 123.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 119, CDR-H2 having the amino acid sequence of SEQ ID NO: 120, and CDR-H3 having the amino acid sequence of SEQ ID NO: 121. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 122, CDR-L2 having the amino acid sequence of SEQ ID NO: 45, and CDR-L3 having the amino acid sequence of SEQ ID NO: 123.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 119; a CDR-H2 having no more than 3 amino acid variations (e.g., no more than 3, 2, or 1 amino acid variation) as compared with the CDR-H2 having the amino acid sequence of SEQ ID NO: 120; and/or (e.g., and) a CDR-H3 having no more than 3 amino acid variations (e.g., no more than 3, 2, or 1 amino acid variation) as compared with the CDR-H3 having the amino acid sequence of SEQ ID NO: 121. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 122; a CDR-L2 having no more than 3 amino acid variations (e.g., no more than 3, 2, or 1 amino acid variation) as compared with the CDR-L2 having the amino acid sequence of SEQ ID NO: 45; and/or (e.g., and) a CDR-L3 having no more than 3 amino acid variations (e.g., no more than 3, 2, or 1 amino acid variation) as compared with the CDR-L3 having the amino acid sequence of SEQ ID NO: 123.

In some embodiments, the anti-TfR antibody of the present disclosure comprises a VH comprising the amino acid sequence of SEQ ID NO: 124. Alternatively or in addition (e.g., in addition), the anti-TfR antibody of the present disclosure comprises a VL comprising the amino acid sequence of SEQ ID NO: 125.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 124. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 125.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 124. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 125.

In some embodiments, the anti-TfR antibody of the present disclosure comprises a CDR-H1, a CDR-H2, and a CDR-H3 of a heavy chain variable domain having the amino acid sequence of SEQ ID NO: 132. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 133.

In some embodiments, the anti-TfR antibody of the present disclosure comprises a CDR-H1 having the amino acid sequence of SEQ ID NO: 126 (according to the IMGT definition system), a CDR-H2 having the amino acid sequence of SEQ ID NO: 127 (according to the IMGT definition system), a CDR-H3 having the amino acid sequence of SEQ ID NO: 128 (according to the IMGT definition system), a CDR-L1 having the amino acid sequence of SEQ ID NO: 129 (according to the IMGT definition system), a CDR-L2 having the amino acid sequence of SEQ ID NO: 130 (according to the IMGT definition system), and a CDR-L3 having the amino acid sequence of SEQ ID NO: 131 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 126, CDR-H2 having the amino acid sequence of SEQ ID NO: 127, and CDR-H3 having the amino acid sequence of SEQ ID NO: 128. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 129, CDR-L2 having the amino acid sequence of SEQ ID NO: 130, and CDR-L3 having the amino acid sequence of SEQ ID NO: 131.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 126, CDR-H2 having the amino acid sequence of SEQ ID NO: 127, and CDR-H3 having the amino acid sequence of SEQ ID NO: 128. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 129, CDR-L2 having the amino acid sequence of SEQ ID NO: 130, and CDR-L3 having the amino acid sequence of SEQ ID NO: 131.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 126; a CDR-H2 having no more than 3 amino acid variations (e.g., no more than 3, 2, or 1 amino acid variation) as compared with the CDR-H2 having the amino acid sequence of SEQ ID NO: 127; and/or (e.g., and) a CDR-H3 having no more than 3 amino acid variations (e.g., no more than 3, 2, or 1 amino acid variation) as compared with the CDR-H3 having the amino acid sequence of SEQ ID NO: 128. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 129; a CDR-L2 having no more than 3 amino acid variations (e.g., no more than 3, 2, or 1 amino acid variation) as compared with the CDR-L2 having the amino acid sequence of SEQ ID NO: 130; and/or (e.g., and) a CDR-L3 having no more than 3 amino acid variations (e.g., no more than 3, 2, or 1 amino acid variation) as compared with the CDR-L3 having the amino acid sequence of SEQ ID NO: 131.

In some embodiments, the anti-TfR antibody of the present disclosure comprises a VH comprising the amino acid sequence of SEQ ID NO: 132. Alternatively or in addition (e.g., in addition), the anti-TfR antibody of the present disclosure comprises a VL comprising the amino acid sequence of SEQ ID NO: 133.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 132. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 133.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 132. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 133.

In some embodiments, the anti-TfR antibody of the present disclosure comprises a CDR-H1, a CDR-H2, and a CDR-H3 of a heavy chain variable domain having the amino acid sequence of SEQ ID NO: 136. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 137.

In some embodiments, the anti-TfR antibody of the present disclosure comprises a CDR-H1 having the amino acid sequence of SEQ ID NO: 79 (according to the IMGT definition system), a CDR-H2 having the amino acid sequence of SEQ ID NO: 2 (according to the IMGT definition system), a CDR-H3 having the amino acid sequence of SEQ ID NO: 134 (according to the IMGT definition system), a CDR-L1 having the amino acid sequence of SEQ ID NO: 75 (according to the IMGT definition system), a CDR-L2 having the amino acid sequence of SEQ ID NO: 45 (according to the IMGT definition system), and a CDR-L3 having the amino acid sequence of SEQ ID NO: 135 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 79, CDR-H2 having the amino acid sequence of SEQ ID NO: 2, and CDR-H3 having the amino acid sequence of SEQ ID NO: 134. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 75, CDR-L2 having the amino acid sequence of SEQ ID NO: 45, and CDR-L3 having the amino acid sequence of SEQ ID NO: 135.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 79, CDR-H2 having the amino acid sequence of SEQ ID NO: 2, and CDR-H3 having the amino acid sequence of SEQ ID NO: 134. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 75, CDR-L2 having the amino acid sequence of SEQ ID NO: 45, and CDR-L3 having the amino acid sequence of SEQ ID NO: 135.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 79; a CDR-H2 having no more than 3 amino acid variations (e.g., no more than 3, 2, or 1 amino acid variation) as compared with the CDR-H2 having the amino acid sequence of SEQ ID NO: 2; and/or (e.g., and) a CDR-H3 having no more than 3 amino acid variations (e.g., no more than 3, 2, or 1 amino acid variation) as compared with the CDR-H3 having the amino acid sequence of SEQ ID NO: 134. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 75; a CDR-L2 having no more than 3 amino acid variations (e.g., no more than 3, 2, or 1 amino acid variation) as compared with the CDR-L2 having the amino acid sequence of SEQ ID NO: 45; and/or (e.g., and) a CDR-L3 having no more than 3 amino acid variations (e.g., no more than 3, 2, or 1 amino acid variation) as compared with the CDR-L3 having the amino acid sequence of SEQ ID NO: 135.

In some embodiments, the anti-TfR antibody of the present disclosure comprises a VH comprising the amino acid sequence of SEQ ID NO: 136. Alternatively or in addition (e.g., in addition), the anti-TfR antibody of the present disclosure comprises a VL comprising the amino acid sequence of SEQ ID NO: 137.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 136. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 137.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 136. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 137.

In some embodiments, the anti-TfR antibody of the present disclosure comprises a CDR-H1, a CDR-H2, and a CDR-H3 of a heavy chain variable domain having the amino acid sequence of SEQ ID NO: 143. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 144.

In some embodiments, the anti-TfR antibody of the present disclosure comprises a CDR-H1 having the amino acid sequence of SEQ ID NO: 138 (according to the IMGT definition system), a CDR-H2 having the amino acid sequence of SEQ ID NO: 139 (according to the IMGT definition system), a CDR-H3 having the amino acid sequence of SEQ ID NO: 140 (according to the IMGT definition system), a CDR-L1 having the amino acid sequence of SEQ ID NO: 141 (according to the IMGT definition system), a CDR-L2 having the amino acid sequence of SEQ ID NO: 29 (according to the IMGT definition system), and a CDR-L3 having the amino acid sequence of SEQ ID NO: 142 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 138, CDR-H2 having the amino acid sequence of SEQ ID NO: 139, and CDR-H3 having the amino acid sequence of SEQ ID NO: 140. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 141, CDR-L2 having the amino acid sequence of SEQ ID NO: 29, and CDR-L3 having the amino acid sequence of SEQ ID NO: 142.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 138, CDR-H2 having the amino acid sequence of SEQ ID NO: 139, and CDR-H3 having the amino acid sequence of SEQ ID NO: 140. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 141, CDR-L2 having the amino acid sequence of SEQ ID NO: 29, and CDR-L3 having the amino acid sequence of SEQ ID NO: 142.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 138; a CDR-H2 having no more than 3 amino acid variations (e.g., no more than 3, 2, or 1 amino acid variation) as compared with the CDR-H2 having the amino acid sequence of SEQ ID NO: 139; and/or (e.g., and) a CDR-H3 having no more than 3 amino acid variations (e.g., no more than 3, 2, or 1 amino acid variation) as compared with the CDR-H3 having the amino acid sequence of SEQ ID NO: 140. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 141; a CDR-L2 having no more than 3 amino acid variations (e.g., no more than 3, 2, or 1 amino acid variation) as compared with the CDR-L2 having the amino acid sequence of SEQ ID NO: 29; and/or (e.g., and) a CDR-L3 having no more than 3 amino acid variations (e.g., no more than 3, 2, or 1 amino acid variation) as compared with the CDR-L3 having the amino acid sequence of SEQ ID NO: 142.

In some embodiments, the anti-TfR antibody of the present disclosure comprises a VH comprising the amino acid sequence of SEQ ID NO: 143. Alternatively or in addition (e.g., in addition), the anti-TfR antibody of the present disclosure comprises a VL comprising the amino acid sequence of SEQ ID NO: 144.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 143. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 144.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 143. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 144.

The CDRs of an antibody may have different amino acid sequences when different definition systems are used (e.g., the IMGT definition, the Kabat definition, or the Chothia definition). A definition system annotates each amino acid in a given antibody sequence (e.g., VH or VL sequence) with a number, and numbers corresponding to the heavy chain and light chain CDRs are provided in Table 2. The CDRs listed in Table 1 are defined in accordance with the IMGT definition. CDR sequences of examples of anti-TfR antibodies according to the different definition systems are provided in Table 3. One skilled in the art is able to derive the CDR sequences using the different numbering systems for the anti-TfR antibodies provided in Table 1.

TABLE 2
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M.-P. et al., Nucleic Acids Res., 27:209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196:901-917 (1987))

TABLE 3
CDR sequences of examples of anti-TfR antibodies according to different definition
systems

| | No. | | | |
|---|---|---|---|---|
| | system | IMGT | Kabat | Chothia |
| 3-A4 | CDR-H1 | GFNIKDDY | DDYMY (SEQ ID NO: | GFNIKDD (SEQ ID NO: |
| | | (SEQ ID NO: 1) | 145) | 150) |
| | CDR-H2 | IDPENGDT (SEQ ID NO: 2) | WIDPENGDTEYASKFQD | ENG (SEQ ID NO: 151) |
| | | | (SEQ ID NO: 146) | |
| | CDR-H3 | TLWLRRGLDY (SEQ ID | WLRRGLDY (SEQ ID | LRRGLD (SEQ ID NO: |
| | | NO: 3) | NO: 147) | 152) |
| | CDR-L1 | KSLLHSNGYTY (SEQ ID | RSSKSLLHSNGYTYLF | SKSLLHSNGYTY(SEQ |
| | | NO: 4) | (SEQ ID NO: 148) | ID NO: 153) |
| | CDR-L2 | RMS (SEQ ID NO: 5) | RMSNLAS (SEQ ID NO: | RMS(SEQ ID NO: 5) |
| | | | 149) | |
| | CDR-L3 | MQHLEYPFT (SEQ ID NO: | MQHLEYPFT (SEQ ID | HLEYPF (SEQ ID NO: |
| | | 6) | NO: 6) | 154) |
| 3-A4 | CDR-H1 | GFNIKDDY (SEQ ID NO: 1) | DDYMY (SEQ ID NO: | GFNIKDD (SEQ ID NO: |
| Variant | | | 145) | 150) |
| 1 | CDR-H2 | IDPETGDT (SEQ ID NO: | WIDPETGDTEYASKFQD | ETG (SEQ ID NO: 277) |
| | | 233) | (SEQ ID NO: 234) | |
| | CDR-H3 | TLWLRRGLDY (SEQ ID | WLRRGLDY (SEQ ID | LRRGLD (SEQ ID NO: |
| | | NO: 3) | NO: 147) | 152) |
| | CDR-L1 | KSLLHSNGYTY (SEQ ID | RSSKSLLHSNGYTYLF | SKSLLHSNGYTY(SEQ |
| | | NO: 4) | (SEQ ID NO: 148) | ID NO: 153) |
| | CDR-L2 | RMS (SEQ ID NO: 5) | RMSNLAS (SEQ ID NO: | RMS(SEQ ID NO: 5) |
| | | | 149) | |
| | CDR-L3 | MQHLEYPFT (SEQ ID NO: | MQHLEYPFT (SEQ ID | HLEYPF (SEQ ID NO: |
| | | 6) | NO: 6) | 154) |
| 3-A4 | CDR-H1 | GFNIKDDY (SEQ ID NO: 1) | DDYMY (SEQ ID NO: | GFNIKDD (SEQ ID NO: |
| Variant | | | 145) | 150) |
| 2 | CDR-H2 | IDPESGDT (SEQ ID NO: | WIDPESGDTEYASKFQD | ESG (SEQ ID NO: 278) |
| | | 80) | (SEQ ID NO: 236) | |
| | CDR-H3 | TLWLRRGLDY (SEQ ID | WLRRGLDY (SEQ ID | LRRGLD (SEQ ID NO: |
| | | NO: 3) | NO: 147) | 152) |
| | CDR-L1 | KSLLHSNGYTY (SEQ ID | RSSKSLLHSNGYTYLF | SKSLLHSNGYTY(SEQ |
| | | NO: 4) | (SEQ ID NO: 148) | ID NO: 153) |
| | CDR-L2 | RMS (SEQ ID NO: 5) | RMSNLAS (SEQ ID NO: | RMS(SEQ ID NO: 5) |
| | | | 149) | |
| | CDR-L3 | MQHLEYPFT (SEQ ID NO: | MQHLEYPFT (SEQ ID | HLEYPF (SEQ ID NO: |
| | | 6) | NO: 6) | 154) |
| 3-M12 | CDR-H1 | GYSITSGYY (SEQ ID NO: | SGYYWN (SEQ ID NO: | GYSITSGY (SEQ ID NO: |
| | | 9) | 155) | 160) |
| | CDR-H2 | ITFDGAN (SEQ ID NO: 10) | YITFDGANNYNPSLKN | FDG (SEQ ID NO: 161) |
| | | | (SEQ ID NO: 156) | |
| | CDR-H3 | TRSSYDYDVLDY (SEQ ID | SSYDYDVLDY (SEQ ID | SYDYDVLD (SEQ ID |
| | | NO: 11) | NO: 157) | NO: 162) |
| | CDR-L1 | QDISNF (SEQ ID NO: 12) | RASQDISNFLN (SEQ ID | SQDISNF (SEQ ID NO: |
| | | | NO: 158) | 163) |
| | CDR-L2 | YTS (SEQ ID NO: 13) | YTSRLHS (SEQ ID NO: | YTS (SEQ ID NO: 13) |
| | | | 159) | |
| | CDR-L3 | QQGHTLPYT (SEQ ID NO: | QQGHTLPYT (SEQ ID | GHTLPY (SEQ ID NO: |
| | | 14) | NO: 14) | 164) |
| 5-H12 | CDR-H1 | GYSFTDYC (SEQ ID NO: | DYCIN (SEQ ID NO: 165) | GYSFTDY (SEQ ID NO: |
| | | 17) | | 170) |
| | CDR-H2 | IYPGSGNT (SEQ ID NO: | WIYPGSGNTRYSERFKG | GSG (SEQ ID NO: 171) |
| | | 18) | (SEQ ID NO: 166) | |
| | CDR-H3 | AREDYYPYHGMDY (SEQ | EDYYPYHGMDY (SEQ | DYYPYHGMD (SEQ ID |
| | | ID NO: 19) | ID NO: 167) | NO: 172) |
| | CDR-L1 | ESVDGYDNSF (SEQ ID | RASES VDGYDNSFMH | SESVDGYDNSF (SEQ ID |
| | | NO: 20) | (SEQ ID NO: 168) | NO: 173) |
| | CDR-L2 | RAS (SEQ ID NO: 21) | RASNLES (SEQ ID NO: | RAS (SEQ ID NO: 21) |
| | | | 169) | |
| | CDR-L3 | QQSSEDPWT (SEQ ID NO: | QQSSEDPWT (SEQ ID | SSEDPW (SEQ ID NO: |
| | | 22) | NO: 22) | 174) |
| 5-H12 | CDR-H1 | GYSFTDYY (SEQ ID NO: | DYYIN (SEQ ID NO: 238) | GYSFTDY (SEQ ID NO: |
| Variant | | 237) | | 170) |
| 1 | CDR-H2 | IYPGSGNT (SEQ ID NO: | WIYPGSGNTRYSERFKG | GSG (SEQ ID NO: 171) |
| | | 18) | (SEQ ID NO: 166) | |
| | CDR-H3 | AREDYYPYHGMDY (SEQ | EDYYPYHGMDY (SEQ | DYYPYHGMD (SEQ ID |
| | | ID NO: 19) | ID NO: 167) | NO: 172) |
| | CDR-L1 | ESVDGYDNSF (SEQ ID | RASES VDGYDNSFMH | SESVDGYDNSF (SEQ ID |
| | | NO: 20) | (SEQ ID NO: 168) | NO: 173) |
| | CDR-L2 | RAS (SEQ ID NO: 21) | RASNLES (SEQ ID NO: | RAS (SEQ ID NO: 21) |
| | | | 169) | |
| | CDR-L3 | QQSSEDPWT (SEQ ID NO: | QQSSEDPWT (SEQ ID | SSEDPW (SEQ ID NO: |
| | | 22) | NO: 22) | 174) |
| 5-H12 | CDR-H1 | GYSFTDYD (SEQ ID NO: | DYDIN (SEQ ID NO: 240) | GYSFTDY (SEQ ID NO: |
| Variant | | 239) | | 170) |
| 2 | CDR-H2 | IYPGSGNT (SEQ ID NO: | WIYPGSGNTRYSERFKG | GSG (SEQ ID NO: 171) |
| | | 18) | (SEQ ID NO: 166) | |
| | CDR-H3 | AREDYYPYHGMDY (SEQ | EDYYPYHGMDY (SEQ | DYYPYHGMD (SEQ ID |
| | | ID NO: 19) | ID NO: 167) | NO: 172) |
| | CDR-L1 | ESVDGYDNSF (SEQ ID | RASES VDGYDNSFMH | SESVDGYDNSF (SEQ ID |
| | | NO: 20) | (SEQ ID NO: 168) | NO: 173) |
| | CDR-L2 | RAS (SEQ ID NO: 21) | RASNLES (SEQ ID NO: | RAS (SEQ ID NO: 21) |
| | | | 169) | |
| | CDR-L3 | QQSSEDPWT (SEQ ID NO: | QQSSEDPWT (SEQ ID | SSEDPW (SEQ ID NO: |
| | | 22) | NO: 22) | 174) |

In some embodiments, the anti-TfR antibody of the present disclosure comprises a CDR-H1 having the amino acid sequence of SEQ ID NO: 145 (according to the Kabat definition system), a CDR-H2 having the amino acid sequence of SEQ ID NO: 146, SEQ ID NO: 234, or SEQ ID NO: 236 (according to the Kabat definition system), a CDR-H3 having the amino acid sequence of SEQ ID NO: 147 (according to the Kabat definition system), a CDR-L1 having the amino acid sequence of SEQ ID NO: 148 (according to the Kabat definition system), a CDR-L2 having the amino acid sequence of SEQ ID NO: 149 (according to the Kabat definition system), and a CDR-L3 having the amino acid sequence of SEQ ID NO: 6 (according to the Kabat definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 145, CDR-H2 having the amino acid sequence of SEQ ID NO: 146, SEQ ID NO: 234, or SEQ ID NO: 236, and CDR-H3 having the amino acid sequence of SEQ ID NO: 147. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 148, CDR-L2 having the amino acid sequence of SEQ ID NO: 149, and CDR-L3 having the amino acid sequence of SEQ ID NO: 6.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 145, CDR-H2 having the amino acid sequence of SEQ ID NO: 146, SEQ ID NO: 234, or SEQ ID NO: 236, and CDR-H3 having the amino acid sequence of SEQ ID NO: 147. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 148, CDR-L2 having the amino acid sequence of SEQ ID NO: 149, and CDR-L3 having the amino acid sequence of SEQ ID NO: 6.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 145; a CDR-H2 having no more than 3 amino acid variations (e.g., no more than 3, 2, or 1 amino acid variation) as compared with the CDR-H2 having the amino acid sequence of SEQ ID NO: 146, SEQ ID NO: 234, or SEQ ID NO: 236; and/or (e.g., and) a CDR-H3 having no more than 3 amino acid variations (e.g., no more than 3, 2, or 1 amino acid variation) as compared with the CDR-H3 having the amino acid sequence of SEQ ID NO: 147. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 148; a CDR-L2 having no more than 3 amino acid variations (e.g., no more than 3, 2, or 1 amino acid variation) as compared with the CDR-L2 having the amino acid sequence of SEQ ID NO: 149; and/or (e.g., and) a CDR-L3 having no more than 3 amino acid variations (e.g., no more than 3, 2, or 1 amino acid variation) as compared with the CDR-L3 having the amino acid sequence of SEQ ID NO: 6.

In some embodiments, the anti-TfR antibody of the present disclosure comprises a CDR-H1 having the amino acid sequence of SEQ ID NO: 150 (according to the Chothia definition system), a CDR-H2 having the amino acid sequence of SEQ ID NO: 151, SEQ ID NO: 277, or SEQ ID NO: 278 (according to the Chothia definition system), a CDR-H3 having the amino acid sequence of SEQ ID NO: 152 (according to the Chothia definition system), a CDR-L1 having the amino acid sequence of SEQ ID NO: 153 (according to the Chothia definition system), a CDR-L2 having the amino acid sequence of SEQ ID NO: 5 (according to the Chothia definition system), and a CDR-L3 having the amino acid sequence of SEQ ID NO: 154 (according to the Chothia definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 150, CDR-H2 having the amino acid sequence of SEQ ID NO: 151, SEQ ID NO: 277, or SEQ ID NO: 278, and CDR-H3 having the amino acid sequence of SEQ ID NO: 152. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 153, CDR-L2 having the amino acid sequence of SEQ ID NO: 5, and CDR-L3 having the amino acid sequence of SEQ ID NO: 154.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 150, CDR-H2 having the amino acid sequence of SEQ ID NO: 151, SEQ ID NO: 277, or SEQ ID NO: 278, and CDR-H3 having the amino acid sequence of SEQ ID NO: 152. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 153, CDR-L2 having the amino acid sequence of SEQ ID NO: 5, and CDR-L3 having the amino acid sequence of SEQ ID NO: 154.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 150; a CDR-H2 having no more than 3 amino acid variations (e.g., no more than 3, 2, or 1 amino acid variation) as compared with the CDR-H2 having the amino acid sequence of SEQ ID NO: 151, SEQ ID NO: 277, or SEQ ID NO: 278; and/or (e.g., and) a CDR-H3 having no more than 3 amino acid variations (e.g., no more than 3, 2, or 1 amino acid variation) as compared with the CDR-H3 having the amino acid sequence of SEQ ID NO: 152. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 153; a CDR-L2 having no more than 3 amino acid variations (e.g., no more than 3, 2, or 1 amino acid variation) as compared with the CDR-L2 having the amino acid sequence of SEQ ID NO: 5; and/or (e.g., and) a CDR-L3 having no more than 3 amino acid variations (e.g., no more than 3, 2, or 1 amino acid variation) as compared with the CDR-L3 having the amino acid sequence of SEQ ID NO: 154.

In some embodiments, the anti-TfR antibody of the present disclosure comprises a CDR-H1 having the amino acid sequence of SEQ ID NO: 155 (according to the Kabat definition system), a CDR-H2 having the amino acid sequence of SEQ ID NO: 156 (according to the Kabat definition system), a CDR-H3 having the amino acid sequence of SEQ ID NO: 157 (according to the Kabat definition system), a CDR-L1 having the amino acid sequence of SEQ ID NO: 158 (according to the Kabat definition system), a CDR-L2 having the amino acid sequence of SEQ ID NO: 159 (according to the Kabat definition system), and a CDR-L3 having the amino acid sequence of SEQ ID NO: 14 (according to the Kabat definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 155, CDR-H2 having the amino acid sequence of SEQ ID NO: 156, and CDR-H3 having the amino acid sequence of SEQ ID NO: 157. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 158, CDR-L2 having the amino acid sequence of SEQ ID NO: 159, and CDR-L3 having the amino acid sequence of SEQ ID NO: 14.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 155, CDR-H2 having the amino acid sequence of SEQ ID NO: 156, and CDR-H3 having the amino acid sequence of SEQ ID NO: 157. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 158, CDR-L2 having the amino acid sequence of SEQ ID NO: 159, and CDR-L3 having the amino acid sequence of SEQ ID NO: 14.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 155; a CDR-H2 having no more than 3 amino acid variations (e.g., no more than 3, 2, or 1 amino acid variation) as compared with the CDR-H2 having the amino acid sequence of SEQ ID NO: 156; and/or (e.g., and) a CDR-H3 having no more than 3 amino acid variations (e.g., no more than 3, 2, or 1 amino acid variation) as compared with the CDR-H3 having the amino acid sequence of SEQ ID NO: 157. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 158; a CDR-L2 having no more than 3 amino acid variations (e.g., no more than 3, 2, or 1 amino acid variation) as compared with the CDR-L2 having the amino acid sequence of SEQ ID NO: 159; and/or (e.g., and) a CDR-L3 having no more than 3 amino acid variations (e.g., no more than 3, 2, or 1 amino acid variation) as compared with the CDR-L3 having the amino acid sequence of SEQ ID NO: 14.

In some embodiments, the anti-TfR antibody of the present disclosure comprises a CDR-H1 having the amino acid sequence of SEQ ID NO: 160 (according to the Chothia definition system), a CDR-H2 having the amino acid sequence of SEQ ID NO: 161 (according to the Chothia definition system), a CDR-H3 having the amino acid sequence of SEQ ID NO: 162 (according to the Chothia definition system), a CDR-L1 having the amino acid sequence of SEQ ID NO: 163 (according to the Chothia definition system), a CDR-L2 having the amino acid sequence of SEQ ID NO: 13 (according to the Chothia definition system), and a CDR-L3 having the amino acid sequence of SEQ ID NO: 164 (according to the Chothia definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 160, CDR-H2 having the amino acid sequence of SEQ ID NO: 161, and CDR-H3 having the amino acid sequence of SEQ ID NO: 162. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 163, CDR-L2 having the amino acid sequence of SEQ ID NO: 13, and CDR-L3 having the amino acid sequence of SEQ ID NO: 164.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 160, CDR-H2 having the amino acid sequence of SEQ ID NO: 161, and CDR-H3 having the amino acid sequence of SEQ ID NO: 162. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 163, CDR-L2 having the amino acid sequence of SEQ ID NO: 13, and CDR-L3 having the amino acid sequence of SEQ ID NO: 164.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 160; a CDR-H2 having no more than 3 amino acid variations (e.g., no more than 3, 2, or 1 amino acid variation) as compared with the CDR-H2 having the amino acid sequence of SEQ ID NO: 161; and/or (e.g., and) a CDR-H3 having no more than 3 amino acid variations (e.g., no more than 3, 2, or 1 amino acid variation) as compared with the CDR-H3 having the amino acid sequence of SEQ ID NO: 162. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 163; a CDR-L2 having no more than 3 amino acid variations (e.g., no more than 3, 2, or 1 amino acid variation) as compared with the CDR-L2 having the amino acid sequence of SEQ ID NO: 13; and/or (e.g., and) a CDR-L3 having no more than 3 amino acid variations (e.g., no more than 3, 2, or 1 amino acid variation) as compared with the CDR-L3 having the amino acid sequence of SEQ ID NO: 164.

In some embodiments, the anti-TfR antibody of the present disclosure comprises a CDR-H1 having the amino acid sequence of SEQ ID NO: 165, SEQ ID NO: 238, or SEQ ID NO: 240 (according to the Kabat definition system), a CDR-H2 having the amino acid sequence of SEQ ID NO: 166 (according to the Kabat definition system), a CDR-H3 having the amino acid sequence of SEQ ID NO: 167 (according to the Kabat definition system), a CDR-L1 having the amino acid sequence of SEQ ID NO: 168 (according to the Kabat definition system), a CDR-L2 having the amino acid sequence of SEQ ID NO: 169 (according to the Kabat definition system), and a CDR-L3 having the amino acid sequence of SEQ ID NO: 22 (according to the Kabat definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 165, SEQ ID NO: 238, or SEQ ID NO: 240, CDR-H2 having the amino acid sequence of SEQ ID NO: 166, and CDR-H3 having the amino acid sequence of SEQ ID NO: 167. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 168, CDR-L2 having the amino acid sequence of SEQ ID NO: 169, and CDR-L3 having the amino acid sequence of SEQ ID NO: 22.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 165, SEQ ID NO: 238, or SEQ ID NO: 240, CDR-H2 having the amino acid sequence of SEQ ID NO: 166, and CDR-H3 having the amino acid sequence of SEQ ID NO: 167. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 168, CDR-L2 having the amino acid sequence of SEQ ID NO: 169, and CDR-L3 having the amino acid sequence of SEQ ID NO: 22.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 165, SEQ ID NO: 238, or SEQ ID NO: 240; a CDR-H2 having no more than 3 amino acid variations (e.g., no more than 3, 2, or 1 amino acid variation) as compared with the CDR-H2 having the amino acid sequence of SEQ ID NO: 166; and/or (e.g., and) a CDR-H3 having no more than 3 amino acid variations (e.g., no more than 3, 2, or 1 amino acid variation) as compared with the CDR-H3 having the amino acid sequence of SEQ ID NO: 167. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 168; a CDR-L2 having no more than 3 amino acid variations (e.g., no more than 3, 2, or 1 amino acid variation) as compared with the CDR-L2 having the amino acid sequence of SEQ ID NO: 169; and/or (e.g., and) a CDR-L3 having no more than 3 amino acid variations (e.g., no more than 3, 2, or 1 amino acid variation) as compared with the CDR-L3 having the amino acid sequence of SEQ ID NO: 22.

In some embodiments, the anti-TfR antibody of the present disclosure comprises a CDR-H1 having the amino acid sequence of SEQ ID NO: 170 (according to the Chothia definition system), a CDR-H2 having the amino acid sequence of SEQ ID NO: 171 (according to the Chothia definition system), a CDR-H3 having the amino acid sequence of SEQ ID NO: 172 (according to the Chothia definition system), a CDR-L1 having the amino acid sequence of SEQ ID NO: 173 (according to the Chothia definition system), a CDR-L2 having the amino acid sequence of SEQ ID NO: 21 (according to the Chothia definition system), and a CDR-L3 having the amino acid sequence of SEQ ID NO: 174 (according to the Chothia definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 170, CDR-H2 having the amino acid sequence of SEQ ID NO: 171, and CDR-H3 having the amino acid sequence of SEQ ID NO: 172. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 173, CDR-L2 having the amino acid sequence of SEQ ID NO: 21, and CDR-L3 having the amino acid sequence of SEQ ID NO: 174.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 170, CDR-H2 having the amino acid sequence of SEQ ID NO: 171, and CDR-H3 having the amino acid sequence of SEQ ID NO: 172. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 173, CDR-L2 having the amino acid sequence of SEQ ID NO: 21, and CDR-L3 having the amino acid sequence of SEQ ID NO: 174.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 170; a CDR-H2 having no more than 3 amino acid variations (e.g., no more than 3, 2, or 1 amino acid variation) as compared with the CDR-H2 having the amino acid sequence of SEQ ID NO: 171; and/or (e.g., and) a CDR-H3 having no more than 3 amino acid variations (e.g., no more than 3, 2, or 1 amino acid variation) as compared with the CDR-H3 having the amino acid sequence of SEQ ID NO: 172. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 173; a CDR-L2 having no more than 3 amino acid variations (e.g., no more than 3, 2, or 1 amino acid variation) as compared with the CDR-L2 having the amino acid sequence of SEQ ID NO: 21; and/or (e.g., and) a CDR-L3 having no more than 3 amino acid variations (e.g., no more than 3, 2, or 1 amino acid variation) as compared with the CDR-L3 having the amino acid sequence of SEQ ID NO: 174.

In some embodiments, the anti-TfR antibody of the present disclosure is a humanized antibody (e.g., a humanized variant containing one or more CDRs of Table 1 or Table 3). In some embodiments, the anti-TfR antibody of the present disclosure comprises a CDR-H1, a CDR-H2, a CDR-H3, a CDR-L1, a CDR-L2, and a CDR-L3 that are the same as the CDR-H1, CDR-H2, and CDR-H3 shown in Table 1 or Table 3, and comprises a humanized heavy chain variable region and/or (e.g., and) a humanized light chain variable region.

Humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some embodiments, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs derived from one or more CDRs from the original antibody. Humanized antibodies may also involve affinity maturation.

Humanized antibodies and methods of making them are known, e.g., as described in Almagro et al., Front. Biosci. 13:1619-1633 (2008); Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005); Padlan et al., Mol. Immunol. 28:489-498 (1991); Dall'Acqua et al., Methods 36:43-60 (2005); Osbourn et al., Methods 36:61-68 (2005); and Klimka et al., Br. J. Cancer, 83:252-260 (2000), the contents of all of which are incorporated herein by reference. Human framework regions that may be used for humanization are described in e.g., Sims et al. J. Immunol. 151:2296 (1993); Carter et al. Proc. Natl. Acad. Sci. USA, 89:4285 (1992); Presta et al. J. Immunol., 151:2623 (1993); Almagro et al., Front. Biosci. 13:1619-1633 (2008)); Baca et al., J. Biol. Chem. 272:10678-10684 (1997); and Rosok et al., J Biol. Chem. 271:22611-22618 (1996), the contents of all of which are incorporated herein by reference. In some embodiments, humanization is achieved by grafting the CDRs (e.g., as shown in Table 1 or Table 3) into the IGKV1-NL1*01 and IGHV1-3*01 human variable domains.

In some embodiments, a humanized VH framework or VL framework is a consensus human framework. In some embodiments, a consensus humanized framework can represent the most commonly occurring amino acid residue in a selection of human immunoglobulin VL or VH framework sequences.

In some embodiments, the consensus human VH framework regions suitable for use with heavy chain CDRs in the humanized anti-TfR antibodies described herein include (subgroup III consensus):

a) VH FR1:
(SEQ ID NO: 241)
EVQLVESGGGLVQPGGSLRLSCAAS;
b) VH FR2:
(SEQ ID NO: 242)
WVRQAPGKGLEWV;
c) VH FR3:
(SEQ ID NO: 243)
RFTISRDNSKNTLYLQMNSLRAEDTAVYYC;
and
d) VH FR4:
(SEQ ID NO: 244)
WGQGTLVTVSS.

In some embodiments, the consensus human VH framework regions suitable for use with heavy chain CDRs in the humanized anti-TfR antibodies described herein include (subgroup I consensus):

a) VH FR1:
(SEQ ID NO: 245)
QVQLVQSGAEVKKPGASVKVSCKAS;
b) VH FR2:
(SEQ ID NO: 246)
WVRQAPGQGLEWM;
c) VH FR3:
(SEQ ID NO: 247)
RVTITADTSTSTAYMELSSLRSEDTAVYYC;
and
d) VH FR4:
(SEQ ID NO: 244)
WGQGTLVTVSS.

In some embodiments, the consensus human VH framework regions suitable for use with heavy chain CDRs in the humanized anti-TfR antibodies described herein include (subgroup II consensus):

a) VH FR1:
(SEQ ID NO: 249)
QVQLQESGPGLVKPSQTLSLTCTVS;
b) VH FR2:
(SEQ ID NO: 250)
WIRQPPGKGLEWI;
c) VH FR3:
(SEQ ID NO: 251)
RVTISVDTSKNQFSLKLSSVTAADTAVYYC;
and
d) VH FR4:
(SEQ ID NO: 244)
WGQGTLVTVSS.

In some embodiments, the consensus human VL framework regions suitable for use with light chain CDRs in the humanized anti-TfR antibodies described herein include (subgroup I consensus):

a) VL FR1:
(SEQ ID NO: 253)
DIQMTQSPSSLSASVGDRVTITC;
b) VL FR2:
(SEQ ID NO: 254)
WYQQKPGKAPKLLIY;
c) VL FR3:
(SEQ ID NO: 256)
GVPSRFSGSGSGTDFTLTISSLQPEDFATYYC;
and
d) VL FR4:
(SEQ ID NO: 248)
FGQGTKVEIK.

In some embodiments, the consensus human VL framework regions suitable for use with light chain CDRs in the humanized anti-TfR antibodies described herein include (subgroup II consensus):

a) VL FR1:
(SEQ ID NO: 257)
DIVMTQSPLSLPVTPGEPASISC;
b) VL FR2:
(SEQ ID NO: 258)
WYLQKPGQSPQLLIY;
c) VL FR3:
(SEQ ID NO: 259)
GVPDRFSGSGSGTDFTLKISRVEAEDVGVYYC;
and
d) VL FR4:
(SEQ ID NO: 248)
FGQGTKVEIK.

In some embodiments, the consensus human VL framework regions suitable for use with light chain CDRs in the humanized anti-TfR antibodies described herein include (subgroup III consensus):

a) VL FR1:
(SEQ ID NO: 252)
DIVMTQSPDSLAVSLGERATINC;
b) VL FR2:
(SEQ ID NO: 255)
WYQQKPGQPPKLLIY;
c) VL FR3:
(SEQ ID NO: 263)
GVPDRFSGSGSGTDFTLTISSLQAEDFAVYYC;
and
d) VL FR4:
(SEQ ID NO: 248)
FGQGTKVEIK.

In some embodiments, the consensus human VL framework regions suitable for use with light chain CDRs in the humanized anti-TfR antibodies described herein include (subgroup IV consensus):

a) VL FR1:
(SEQ ID NO: 252)
DIVMTQSPDSLAVSLGERATINC;
b) VL FR2:
(SEQ ID NO: 255)
WYQQKPGQPPKLLIY;
c) VL FR3:
(SEQ ID NO: 260)
GVPDRFSGSGSGTDFTLTISSLQAEDFAVYYC;
and
d) VL FR4:
(SEQ ID NO: 248)
FGQGTKVEIK.

In some embodiments, the humanized anti-TfR antibody of the present disclosure comprises humanized VH framework regions that collectively contain no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with any one of the consensus human VH framework region subgroups described herein. Alternatively or in addition (e.g., in addition), the humanized anti-TfR antibody of the present disclosure comprises humanized VL framework regions that collectively contain no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with any one of the consensus human VL framework region subgroups described herein.

In some embodiments, the humanized anti-TfR antibody of the present disclosure comprises humanized VH framework regions that collectively are at least 75% (e.g., 75%, 80%, 85%, 90%, 95%, 98%, or 99%) identical to any one of the consensus human VH framework region subgroups described herein. Alternatively or in addition (e.g., in addition), the humanized anti-TfR antibody of the present disclosure comprises humanized VL framework regions that are at least 75% (e.g., 75%, 80%, 85%, 90%, 95%, 98%, or 99%) identical to any one of the consensus human VL framework region subgroups described herein.

In some embodiments, the anti-TfR antibody of the present disclosure is a humanized variant comprising one or more amino acid variations (e.g., in the VH framework region) as compared with any one of the VHs listed in Table 1 or Table 3, and/or (e.g., and) one or more amino acid variations (e.g., in the VL framework region) as compared with any one of the VLs listed in Table 1 or Table 3.

In some embodiments, the anti-TfR antibody of the present disclosure is a humanized antibody comprising a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH of any of the anti-TfR antibodies listed in Table 1. Alternatively or in addition (e.g., in addition), the anti-TfR antibody of the present disclosure is a humanized antibody comprising a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL of any one of the anti-TfR antibodies listed in Table 1.

In some embodiments, the anti-TfR antibody of the present disclosure is a humanized antibody comprising a VH comprising an amino acid sequence that is at least 75% (e.g., 75%, 80%, 85%, 90%, 95%, 98%, or 99%) identical to the VH as set forth in any one of SEQ ID NOs: 7, 15, and 23. Alternatively or in addition (e.g., in addition), the anti-TfR antibody of the present disclosure is a humanized antibody comprising a VL comprising an amino acid sequence that is at least 75% (e.g., 75%, 80%, 85%, 90%, 95%, 98%, or 99%) identical to the VL as set forth in any one of SEQ ID NOs: 8, 16, and 24.

In some embodiments, the anti-TfR antibody of the present disclosure is a humanized antibody comprising a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in any one of SEQ ID NOs: 7, 15, and 23. Alternatively or in addition (e.g., in addition), the anti-TfR antibody of the present disclosure is a humanized antibody comprising a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in any one of SEQ ID NOs: 8, 16, and 24.

In some embodiments, the anti-TfR antibody of the present disclosure is a humanized antibody comprising a VH comprising an amino acid sequence that is at least 75% (e.g., 75%, 80%, 85%, 90%, 95%, 98%, or 99%) identical to the VH as set forth in any one of SEQ ID NOs: 7, 15, and 23. Alternatively or in addition (e.g., in addition), the anti-TfR antibody of the present disclosure is a humanized antibody comprising a VL comprising an amino acid sequence that is at least 75% (e.g., 75%, 80%, 85%, 90%, 95%, 98%, or 99%) identical to the VL as set forth in any one of SEQ ID NOs: 8, 16, and 24.

In some embodiments, the anti-TfR antibody of the present disclosure is a humanized antibody comprising a VH having one or more (e.g., 10-25) amino acid variations at positions 1, 2, 5, 9, 11, 12, 13, 17, 20, 23, 33, 38, 40, 41, 42, 43, 44, 45, 48, 49, 55, 67, 68, 70, 71, 72, 76, 77, 80, 81, 82, 84, 87, 88, 91, 95, 112, or 115 relative to the VH as set forth in any one of SEQ ID NOs: 7, 15, and 23. Alternatively or in addition (e.g., in addition), the anti-TfR antibody of the present disclosure is a humanized antibody comprising a VL having one or more (e.g., 10-20) amino acid variations at positions 4, 7, 8, 9, 11, 15, 17, 18, 19, 22, 39, 41, 42, 43, 50, 62, 64, 72, 75, 77, 79, 80, 81, 82, 83, 85, 87, 89, 100, 104, or 109 relative to the VL as set forth in any one of SEQ ID NOs: 8, 16, and 24.

In some embodiments, the anti-TfR antibody of the present disclosure comprises a humanized VH comprising a CDR-H1 having the amino acid sequence of SEQ ID NO: 1 (according to the IMGT definition system), a CDR-H2 having the amino acid sequence of SEQ ID NO: 2, SEQ ID NO: 233, or SEQ ID NO: 80 (according to the IMGT definition system), a CDR-H3 having the amino acid sequence of SEQ ID NO: 3 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 7. Alternatively or in addition (e.g., in addition), the anti-TfR antibody of the present disclosure comprises a humanized VL comprising a CDR-L1 having the amino acid sequence of SEQ ID NO: 4 (according to the IMGT definition system), a CDR-L2 having the amino acid sequence of SEQ ID NO: 5 (according to the IMGT definition system), and a CDR-L3 having the amino acid sequence of SEQ ID NO: 6 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 8.

In some embodiments, the anti-TfR antibody of the present disclosure comprises a humanized VH comprising a CDR-H1 having the amino acid sequence of SEQ ID NO: 1 (according to the IMGT definition system), a CDR-H2 having the amino acid sequence of SEQ ID NO: 2, SEQ ID NO: 233, or SEQ ID NO: 80 (according to the IMGT definition system), a CDR-H3 having the amino acid sequence of SEQ ID NO: 3 (according to the IMGT definition system), and is at least 75% (e.g., 75%, 80%, 85%, 90%, 95%, 98%, or 99%) identical in the framework regions to the VH as set forth in SEQ ID NO: 7. Alternatively or in addition (e.g., in addition), the anti-TfR antibody of the present disclosure comprises a humanized VL comprising a CDR-L1 having the amino acid sequence of SEQ ID NO: 4 (according to the IMGT definition system), a CDR-L2 having the amino acid sequence of SEQ ID NO: 5 (according to the IMGT definition system), and a CDR-L3 having the amino acid sequence of SEQ ID NO: 6 (according to the IMGT definition system), and is at least 75% (e.g., 75%, 80%, 85%, 90%, 95%, 98%, or 99%) identical in the framework regions to the VL as set forth in SEQ ID NO: 8.

In some embodiments, the anti-TfR antibody of the present disclosure comprises a humanized VH comprising a CDR-H1 having the amino acid sequence of SEQ ID NO: 145 (according to the Kabat definition system), a CDR-H2 having the amino acid sequence of SEQ ID NO: 146, SEQ ID NO: 234, or SEQ ID NO: 236 (according to the Kabat definition system), a CDR-H3 having the amino acid sequence of SEQ ID NO: 147 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 7. Alternatively or in addition (e.g., in addition), the anti-TfR antibody of the present disclosure comprises a humanized VL comprising a CDR-L1 having the amino acid sequence of SEQ ID NO: 148 (according to the Kabat definition system), a CDR-L2 having the amino acid sequence of SEQ ID NO: 149 (according to the Kabat definition system), and a CDR-L3 having the amino acid sequence of SEQ ID NO: 6 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 8.

In some embodiments, the anti-TfR antibody of the present disclosure comprises a humanized VH comprising a CDR-H1 having the amino acid sequence of SEQ ID NO: 145 (according to the Kabat definition system), a CDR-H2 having the amino acid sequence of SEQ ID NO: 146, SEQ ID NO: 234, or SEQ ID NO: 236 (according to the Kabat definition system), a CDR-H3 having the amino acid sequence of SEQ ID NO: 147 (according to the Kabat definition system), and is at least 75% (e.g., 75%, 80%, 85%, 90%, 95%, 98%, or 99%) identical in the framework regions to the VH as set forth in SEQ ID NO: 7. Alternatively or in addition (e.g., in addition), the anti-TfR antibody of the present disclosure comprises a humanized VL comprising a CDR-L1 having the amino acid sequence of SEQ ID NO: 148 (according to the Kabat definition system), a CDR-L2 having the amino acid sequence of SEQ ID NO: 149 (according to the Kabat definition system), and a CDR-L3 having the amino acid sequence of SEQ ID NO: 6 (according to the Kabat definition system), and is at least 75% (e.g., 75%, 80%, 85%, 90%, 95%, 98%, or 99%) identical in the framework regions to the VL as set forth in SEQ ID NO: 8.

In some embodiments, the anti-TfR antibody of the present disclosure comprises a humanized VH comprising a CDR-H1 having the amino acid sequence of SEQ ID NO: 150 (according to the Chothia definition system), a CDR-H2 having the amino acid sequence of SEQ ID NO: 151, SEQ ID NO: 277, or SEQ ID NO: 278 (according to the Chothia definition system), a CDR-H3 having the amino acid sequence of SEQ ID NO: 152 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 7. Alternatively or in addition (e.g., in addition), the anti-TfR antibody of the present disclosure comprises a humanized VL comprising a CDR-L1 having the amino acid sequence of SEQ ID NO: 153 (according to the Chothia definition system), a CDR-L2 having the amino acid sequence of SEQ ID NO: 5 (according to the Chothia definition system), and a CDR-L3 having the amino acid sequence of SEQ ID NO: 154 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 8.

In some embodiments, the anti-TfR antibody of the present disclosure comprises a humanized VH comprising a CDR-H1 having the amino acid sequence of SEQ ID NO: 150 (according to the Chothia definition system), a CDR-H2 having the amino acid sequence of SEQ ID NO: 151, SEQ ID NO: 277, or SEQ ID NO: 278 (according to the Chothia definition system), a CDR-H3 having the amino acid sequence of SEQ ID NO: 152 (according to the Chothia definition system), and is at least 75% (e.g., 75%, 80%, 85%, 90%, 95%, 98%, or 99%) identical in the framework regions to the VH as set forth in SEQ ID NO: 7. Alternatively or in addition (e.g., in addition), the anti-TfR antibody of the present disclosure comprises a humanized VL comprising a CDR-L1 having the amino acid sequence of SEQ ID NO: 153 (according to the Chothia definition system), a CDR-L2 having the amino acid sequence of SEQ ID NO: 5 (according to the Chothia definition system), and a CDR-L3 having the amino acid sequence of SEQ ID NO: 154 (according to the Chothia definition system), and is at least 75% (e.g., 75%, 80%, 85%, 90%, 95%, 98%, or 99%) identical in the framework regions to the VL as set forth in SEQ ID NO: 8.

In some embodiments, the anti-TfR antibody of the present disclosure comprises a humanized VH comprising a CDR-H1 having the amino acid sequence of SEQ ID NO: 9 (according to the IMGT definition system), a CDR-H2 having the amino acid sequence of SEQ ID NO: 10 (according to the IMGT definition system), a CDR-H3 having the amino acid sequence of SEQ ID NO: 11 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 15. Alternatively or in addition (e.g., in addition), the anti-TfR antibody of the present disclosure comprises a humanized VL comprising a CDR-L1 having the amino acid sequence of SEQ ID NO: 12 (according to the IMGT definition system), a CDR-L2 having the amino acid sequence of SEQ ID NO: 13 (according to the IMGT definition system), and a CDR-L3 having the amino acid sequence of SEQ ID NO: 14 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 16.

In some embodiments, the anti-TfR antibody of the present disclosure comprises a humanized VH comprising a CDR-H1 having the amino acid sequence of SEQ ID NO: 9 (according to the IMGT definition system), a CDR-H2 having the amino acid sequence of SEQ ID NO: 10 (according to the IMGT definition system), a CDR-H3 having the amino acid sequence of SEQ ID NO: 11 (according to the IMGT definition system), and is at least 75% (e.g., 75%, 80%, 85%, 90%, 95%, 98%, or 99%) identical in the framework regions to the VH as set forth in SEQ ID NO: 15. Alternatively or in addition (e.g., in addition), the anti-TfR antibody of the present disclosure comprises a humanized VL comprising a CDR-L1 having the amino acid sequence of SEQ ID NO: 12 (according to the IMGT definition system), a CDR-L2 having the amino acid sequence of SEQ ID NO: 13 (according to the IMGT definition system), and a CDR-L3 having the amino acid sequence of SEQ ID NO: 14 (according to the IMGT definition system), and is at least 75% (e.g., 75%, 80%, 85%, 90%, 95%, 98%, or 99%) identical in the framework regions to the VL as set forth SEQ ID NO: 16.

In some embodiments, the anti-TfR antibody of the present disclosure comprises a humanized VH comprising a CDR-H1 having the amino acid sequence of SEQ ID NO: 155 (according to the Kabat definition system), a CDR-H2 having the amino acid sequence of SEQ ID NO: 156 (according to the Kabat definition system), a CDR-H3 having the amino acid sequence of SEQ ID NO: 157 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 15. Alternatively or in addition (e.g., in addition), the anti-TfR antibody of the present disclosure comprises a humanized VL comprising a CDR-L1 having the amino acid sequence of SEQ ID NO: 158 (according to the Kabat definition system), a CDR-L2 having the amino acid sequence of SEQ ID NO: 159 (according to the Kabat definition system), and a CDR-L3 having the amino acid sequence of SEQ ID NO: 14 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 16.

In some embodiments, the anti-TfR antibody of the present disclosure comprises a humanized VH comprising a CDR-H1 having the amino acid sequence of SEQ ID NO: 155 (according to the Kabat definition system), a CDR-H2 having the amino acid sequence of SEQ ID NO: 156 (according to the Kabat definition system), a CDR-H3 having the amino acid sequence of SEQ ID NO: 157 (according to the Kabat definition system), and is at least 75% (e.g., 75%, 80%, 85%, 90%, 95%, 98%, or 99%) identical in the framework regions to the VH as set forth in SEQ ID NO: 15. Alternatively or in addition (e.g., in addition), the anti-TfR antibody of the present disclosure comprises a humanized VL comprising a CDR-L1 having the amino acid sequence of SEQ ID NO: 158 (according to the Kabat definition system), a CDR-L2 having the amino acid sequence of SEQ ID NO: 159 (according to the Kabat definition system), and a CDR-L3 having the amino acid sequence of SEQ ID NO: 14 (according to the Kabat definition system), and is at least 75% (e.g., 75%, 80%, 85%, 90%, 95%, 98%, or 99%) identical in the framework regions to the VL as set forth in SEQ ID NO: 16.

In some embodiments, the anti-TfR antibody of the present disclosure comprises a humanized VH comprising a CDR-H1 having the amino acid sequence of SEQ ID NO: 160 (according to the Chothia definition system), a CDR-H2 having the amino acid sequence of SEQ ID NO: 161 (according to the Chothia definition system), a CDR-H3 having the amino acid sequence of SEQ ID NO: 162 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 15. Alternatively or in addition (e.g., in addition), the anti-TfR antibody of the present disclosure comprises a humanized VL comprising a CDR-L1 having the amino acid sequence of SEQ ID NO: 163 (according to the Chothia definition system), a CDR-L2 having the amino acid sequence of SEQ ID NO: 13 (according to the Chothia definition system), and a CDR-L3 having the amino acid sequence of SEQ ID NO: 164 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 16.

In some embodiments, the anti-TfR antibody of the present disclosure comprises a humanized VH comprising a CDR-H1 having the amino acid sequence of SEQ ID NO: 160 (according to the Chothia definition system), a CDR-H2 having the amino acid sequence of SEQ ID NO: 161 (according to the Chothia definition system), a CDR-H3 having the amino acid sequence of SEQ ID NO: 162 (according to the Chothia definition system), and is at least 75% (e.g., 75%, 80%, 85%, 90%, 95%, 98%, or 99%) identical in the framework regions to the VH as set forth in SEQ ID NO: 15. Alternatively or in addition (e.g., in addition), the anti-TfR antibody of the present disclosure comprises a humanized VL comprising a CDR-L1 having the amino acid sequence of SEQ ID NO: 163 (according to the Chothia definition system), a CDR-L2 having the amino acid sequence of SEQ ID NO: 13 (according to the Chothia definition system), and a CDR-L3 having the amino acid sequence of SEQ ID NO: 164 (according to the Chothia definition system), and is at least 75% (e.g., 75%, 80%, 85%, 90%, 95%, 98%, or 99%) identical in the framework regions to the VL as set forth in SEQ ID NO: 16.

In some embodiments, the anti-TfR antibody of the present disclosure comprises a humanized VH comprising a CDR-H1 having the amino acid sequence of SEQ ID NO: 17, SEQ ID NO: 237, or SEQ ID NO: 239 (according to the IMGT definition system), a CDR-H2 having the amino acid sequence of SEQ ID NO: 18 (according to the IMGT definition system), a CDR-H3 having the amino acid sequence of SEQ ID NO: 19 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 23. Alternatively or in addition (e.g., in addition), the anti-TfR antibody of the present disclosure comprises a humanized VL comprising a CDR-L1 having the amino acid sequence of SEQ ID NO: 20 (according to the IMGT definition system), a CDR-L2 having the amino acid sequence of SEQ ID NO: 21 (according to the IMGT definition system), and a CDR-L3 having the amino acid sequence of SEQ ID NO: 22 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 24.

In some embodiments, the anti-TfR antibody of the present disclosure comprises a humanized VH comprising a CDR-H1 having the amino acid sequence of SEQ ID NO: 17, SEQ ID NO: 237, or SEQ ID NO: 239 (according to the IMGT definition system), a CDR-H2 having the amino acid sequence of SEQ ID NO: 18 (according to the IMGT definition system), a CDR-H3 having the amino acid sequence of SEQ ID NO: 19 (according to the IMGT definition system), and is at least 75% (e.g., 75%, 80%, 85%, 90%, 95%, 98%, or 99%) identical in the framework regions to the VH as set forth in SEQ ID NO: 23. Alternatively or in addition (e.g., in addition), the anti-TfR antibody of the present disclosure comprises a humanized VL comprising a CDR-L1 having the amino acid sequence of SEQ ID NO: 20 (according to the IMGT definition system), a CDR-L2 having the amino acid sequence of SEQ ID NO: 21 (according to the IMGT definition system), and a CDR-L3 having the amino acid sequence of SEQ ID NO: 22 (according to the IMGT definition system), and is at least 75% (e.g., 75%, 80%, 85%, 90%, 95%, 98%, or 99%) identical in the framework regions to the VL as set forth in SEQ ID NO: 24.

In some embodiments, the anti-TfR antibody of the present disclosure comprises a humanized VH comprising a CDR-H1 having the amino acid sequence of SEQ ID NO: 165, SEQ ID NO: 238, or SEQ ID NO: 240 (according to the Kabat definition system), a CDR-H2 having the amino acid sequence of SEQ ID NO: 166 (according to the Kabat definition system), a CDR-H3 having the amino acid sequence of SEQ ID NO: 167 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 23. Alternatively or in addition (e.g., in addition), the anti-TfR antibody of the present disclosure comprises a humanized VL comprising a CDR-L1 having the amino acid sequence of SEQ ID NO: 168 (according to the Kabat definition system), a CDR-L2 having the amino acid sequence of SEQ ID NO: 169 (according to the Kabat definition system), and a CDR-L3 having the amino acid sequence of SEQ ID NO: 22 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 24.

In some embodiments, the anti-TfR antibody of the present disclosure comprises a humanized VH comprising a CDR-H1 having the amino acid sequence of SEQ ID NO: 165, SEQ ID NO: 238, or SEQ ID NO: 240 (according to the Kabat definition system), a CDR-H2 having the amino acid sequence of SEQ ID NO: 166 (according to the Kabat definition system), a CDR-H3 having the amino acid sequence of SEQ ID NO: 167 (according to the Kabat definition system), and is at least 75% (e.g., 75%, 80%, 85%, 90%, 95%, 98%, or 99%) identical in the framework regions to the VH as set forth in SEQ ID NO: 23. Alternatively or in addition (e.g., in addition), the anti-TfR antibody of the present disclosure comprises a humanized VL comprising a CDR-L1 having the amino acid sequence of SEQ ID NO: 168 (according to the Kabat definition system), a CDR-L2 having the amino acid sequence of SEQ ID NO: 169 (according to the Kabat definition system), and a CDR-L3 having the amino acid sequence of SEQ ID NO: 22 (according to the Kabat definition system), and is at least 75% (e.g., 75%, 80%, 85%, 90%, 95%, 98%, or 99%) identical in the framework regions to the VL as set forth in SEQ ID NO: 24.

In some embodiments, the anti-TfR antibody of the present disclosure comprises a humanized VH comprising a CDR-H1 having the amino acid sequence of SEQ ID NO: 170 (according to the Chothia definition system), a CDR-H2 having the amino acid sequence of SEQ ID NO: 171 (according to the Chothia definition system), a CDR-H3 having the amino acid sequence of SEQ ID NO: 172 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 23. Alternatively or in addition (e.g., in addition), the anti-TfR antibody of the present disclosure comprises a humanized VL comprising a CDR-L1 having the amino acid sequence of SEQ ID NO: 173 (according to the Chothia definition system), a CDR-L2 having the amino acid sequence of SEQ ID NO: 21 (according to the Chothia definition system), and a CDR-L3 having the amino acid sequence of SEQ ID NO: 174 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 24.

In some embodiments, the anti-TfR antibody of the present disclosure comprises a humanized VH comprising a CDR-H1 having the amino acid sequence of SEQ ID NO: 170 (according to the Chothia definition system), a CDR-H2 having the amino acid sequence of SEQ ID NO: 171 (according to the Chothia definition system), a CDR-H3 having the amino acid sequence of SEQ ID NO: 172 (according to the Chothia definition system), and is at least 75% (e.g., 75%, 80%, 85%, 90%, 95%, 98%, or 99%) identical in the framework regions to the VH as set forth in SEQ ID NO: 23. Alternatively or in addition (e.g., in addition), the anti-TfR antibody of the present disclosure comprises a humanized VL comprising a CDR-L1 having the amino acid sequence of SEQ ID NO: 173 (according to the Chothia definition system), a CDR-L2 having the amino acid sequence of SEQ ID NO: 21 (according to the Chothia definition system), and a CDR-L3 having the amino acid sequence of SEQ ID NO: 174 (according to the Chothia definition system), and is at least 75% (e.g., 75%, 80%, 85%, 90%, 95%, 98%, or 99%) identical in the framework regions to the VL as set forth in SEQ ID NO: 24.

In some embodiments, the anti-TfR antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or (e.g., and) the constant region.

In some embodiments, the anti-TfR antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or (e.g., and) the constant region.

In some embodiments, the heavy chain of any of the anti-TfR antibodies as described herein may comprises a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain), e.g., IgG1, IgG2, or IgG4. An example of a human IgG1 constant region is given below:

(SEQ ID NO: 175)
ASTKGPSVFPLAPSSKSTSGGTAALGCLVKDYFPEPVT
VSWNSGALTSGVHTFPAVLQSSGLYSLSSVVTVPSSSL
GTQTYICNVNHKPSNTKVDKKVEPKSCDKTHTCPPCPA
PELLGGPSVFLFPPKPKDTLMISRTPEVTCVVVDVSHE
DPEVKFNWYVDGVEVHNAKTKPREEQYNSTYRVVSVLT
VLHQDWLNGKEYKCKVSNKALPAPIEKTISKAKGQPRE
PQVYTLPPSRDELTKNQVSLTCLVKGFYPSDIAVEWES
NGQPENNYKTTPPVLDSDGSFFLYSKLTVDKSRWQQGN
VFSCSVMHEALHNHYTQKSLSLSPGK

In some embodiments, the heavy chain of any of the anti-TfR antibodies described herein comprises a mutant human IgG1 constant region. For example, the introduction of LALA mutations (a mutant derived from mAb b12 that has been mutated to replace the lower hinge residues Leu234 Leu235 with Ala234 and Ala235) in the CH2 domain of human IgG1 is known to reduce Fcg receptor binding (Bruhns, P., et al. (2009) and Xu, D. et al. (2000)). The mutant human IgG1 constant region is provided below (mutations bonded and underlined):

A (SEQ ID NO: 176)
STKGPSVFPLAPSSKSTSGGTAALGCLVKDYFPEPVT
VSWNSGALTSGVHTFPAVLQSSGLYSLSSVVTVPSSSL
GTQTYICNVNHKPSNTKVDKKVEPKSCDKTHTCPPCPA
PEAAGGPSVFLFPPKPKDTLMISRTPEVTCVVVDVSHE
DPEVKFNWYVDGVEVHNAKTKPREEQYNSTYRVVSVLT
VLHQDWLNGKEYKCKVSNKALPAPIEKTISKAKGQPRE
PQVYTLPPSRDELTKNQVSLTCLVKGFYPSDIAVEWES
NGQPENNYKTTPPVLDSDGSFFLYSKLTVDKSRWQQGN
VFSCSVMHEALHNHYTQKSLSLSPGK

In some embodiments, the light chain of any of the anti-TfR antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 177)
RTVAAPSVFIFPPSDEQLKSGTASVVCLLNNFYPREAK
VQWKVDNALQSGNSQESVTEQDSKDSTYSLSSTLTLSK
ADYEKHKVYACEVTHQGLSSPVTKSFNRGEC

Other antibody heavy and light chain constant regions are well known in the art, e.g., those provided in the IMGT database (www.imgt.org) or at www.vbase2.org/vbstat.php., both of which are incorporated by reference herein.

In some embodiments, the anti-TfR antibody described herein comprises a heavy chain comprising any one of the VH as listed in Table 1 or any variants thereof and a heavy chain constant region that is at least 80%, at least 85%, at least 90%, at least 95%, or at least 99% identical to SEQ ID NO: 175 or SEQ ID NO: 176. In some embodiments, the anti-TfR antibody described herein comprises a heavy chain comprising any one of the VH as listed in Table 1 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 175 or SEQ ID NO: 176. In some embodiments, the anti-TfR antibody described herein comprises a heavy chain comprising any one of the VH as listed in Table 1 or any variants thereof and a heavy chain constant region as set forth in SEQ ID NO: 175. In some embodiments, the anti-TfR antibody described herein comprises heavy chain comprising any one of the VH as listed in Table 1 or any variants thereof and a heavy chain constant region as set forth in SEQ ID NO: 176.

In some embodiments, the anti-TfR antibody described herein comprises a light chain comprising any one of the VL as listed in Table 1 or any variants thereof and a light chain constant region that is at least 80%, at least 85%, at least 90%, at least 95%, or at least 99% identical to SEQ ID NO: 177. In some embodiments, the anti-TfR antibody described herein comprises a light chain comprising any one of the VL as listed in Table 1 or any variants thereof and a light chain constant region contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 177. In some embodiments, the anti-TfR antibody described herein comprises a light chain comprising any one of the VL as listed in Table 1 or any variants thereof and a light chain constant region set forth in SEQ ID NO: 177.

Examples of IgG heavy chain and light chain amino acid sequences of the anti-TfR antibodies described are provided in Table 4 below.

TABLE 4
Heavy chain and light chain sequences of examples of anti-TfR IgGs

| Antibody | IgG Heavy Chain/Light Chain Sequences |
|---|---|
| 3-A4 | Heavy Chain (with wild type human IgG1 constant region) |
| | EVQLQQSGAELVRPGASVKLSCTASGFNIKDDYMYWVKQRPEQGLEWIGWIDPENGDTEYAS |
| | KFQDKATVTADTSSNTAYLQLSSLTSEDTAVYYCTLWLRRGLDYWGQGTSVTVSSASTKGPS |
| | VFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVSVLTVLHQDWL |
| | NGKEYKCKVSNKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEALHNHYTQKS |
| | LSLSPGK (SEQ ID NO: 178) |
| | Light Chain (with kappa light chain constant region) |
| | DIVMTQAAPSVPVTPGESVSISCRSSKSLLHSNGYTYLFWFLQRPGQSPQLLIYRMSNLASGVP |
| | DRFSGSGSGTAFTLRISRVEAEDVGVYYCMQHLEYPFTFGGGTKLEIKRTVAAPSVFIFPPSDE |
| | QLKSGTASVVCLLNNFYPREAKVQWKVDNALQSGNSQESVTEQDSKDSTYSLSSTLTLSKADYE |
| | KHKVYACEVTHQGLSSPVTKSFNRGEC (SEQ ID NO: 179) |
| 3-A4 | Heavy Chain (with wild type human IgG1 constant region) |
| Variant 1 | EVQLQQSGAELVRPGASVKLSCTASGFNIKDDYMYWVKQRPEQGLEWIGWIDPETGDTEYAS |
| | KFQDKATVTADTSSNTAYLQLSSLTSEDTAVYYCTLWLRRGLDYWGQGTSVTVSSASTKGPS |
| | VFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVSVLTVLHQDWL |
| | NGKEYKCKVSNKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEALHNHYTQKS |
| | LSLSPGK (SEQ ID NO: 269) |
| | Light Chain (with kappa light chain constant region) |
| | DIVMTQAAPSVPVTPGESVSISCRSSKSLLHSNGYTYLFWFLQRPGQSPQLLIYRMSNLASGVP |
| | DRFSGSGSGTAFTLRISRVEAEDVGVYYCMQHLEYPFTFGGGTKLEIKRTVAAPSVFIFPPSDEQ |
| | LKSGTASVVCLLNNFYPREAKVQWKVDNALQSGNSQESVTEQDSKDSTYSLSSTLTLSKADYE |
| | KHKVYACEVTHQGLSSPVTKSFNRGEC (SEQ ID NO: 179) |
| 3-A4 | Heavy Chain (with wild type human IgG1 constant region) |
| Variant 2 | EVQLQQSGAELVRPGASVKLSCTASGFNIKDDYMYWVKQRPEQGLEWIGWIDPESGDTEYAS |
| | KFQDKATVTADTSSNTAYLQLSSLTSEDTAVYYCTLWLRRGLDYWGQGTSVTVSSASTKGPS |
| | VFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVSVLTVLHQDWL |
| | NGKEYKCKVSNKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEALHNHYTQKS |
| | LSLSPGK (SEQ ID NO: 270) |
| | Light Chain (with kappa light chain constant region) |
| | DIVMTQAAPSVPVTPGESVSISCRSSKSLLHSNGYTYLFWFLQRPGQSPQLLIYRMSNLASGVP |
| | DRFSGSGSGTAFTLRISRVEAEDVGVYYCMQHLEYPFTFGGGTKLEIKRTVAAPSVFIFPPSDEQ |
| | LKSGTASVVCLLNNFYPREAKVQWKVDNALQSGNSQESVTEQDSKDSTYSLSSTLTLSKADYE |
| | KHKVYACEVTHQGLSSPVTKSFNRGEC (SEQ ID NO: 179) |
| 3-M12 | Heavy Chain (with wild type human IgG1 constant region) |
| | DVQLQESGPGLVKPSQSLSLTCSVTGYSITSGYYWNWIRQFPGNKLEWMGYITFDGANNYNPS |
| | LKNRISITRDTSKNQFFLKLTSVTTEDTATYYCTRSSYDYDVLDYWGQGTTLTVSSASTKGPSV |
| | FPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVVSVLTVLHQDWLN |
| | GKEYKCKVSNKALPAPIEKTISKAKGQPREPQVYTLPPSRDELTKNQVSLTCLVKGFYPSDIAV |
| | EWESNGQPENNYKTTPPVLDSDGSFFLYSKLTVDKSRWQQGNVFSCSVMHEALHNHYTQKSL |
| | SLSPGK (SEQ ID NO: 180) |
| | Light Chain (with kappa light chain constant region) |
| | DIQMTQTTSSLSASLGDRVTISCRASQDISNFLNWYQQRPDGTVKLLIYYTSRLHSGVPSRFSGS |
| | GSGTDFSLTVSNLEQEDIATYFCQQGHTLPYTFGGGTKLEIKRTVAAPSVFIFPPSDEQLKSGTA |
| | SVVCLLNNFYPREAKVQWKVDNALQSGNSQESVTEQDSKDSTYSLSSTLTLSKADYEKHKVY |
| | ACEVTHQGLSSPVTKSFNRGEC (SEQ ID NO: 181) |
| 5-H12 | Heavy Chain (with wild type human IgG1 constant region) |
| | QIQLQQSGPELVRPGASVKISCKASGYSFTDYCINWVNQRPGQGLEWIGWIYPGSGNTRYSERF |
| | KGKATLTVDTSSNTAYMQLSSLTSEDSAVYFCAREDYYPYHGMDYWGQGTSVTVSSASTKGP |
| | SVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCSVMHEALHNHYT |
| | QKSLSLSPGK (SEQ ID NO: 182) |
| | Light Chain (with kappa light chain constant region) |
| | DIVLTQSPTSLAVSLGQRATISCRASESVDGYDNSFMHWYQQKPGQPPKLLIFRASNLESGIPAR |
| | FSGSGSRTDFTLTINPVEAADVATYYCQQSSEDPWTFGGGTKLEIKRTVAAPSVFIFPPSDEQLK |
| | SGTASVVCLLNNFYPREAKVQWKVDNALQSGNSQESVTEQDSKDSTYSLSSTLTLSKADYEK |
| | HKVYACEVTHQGLSSPVTKSFNRGEC (SEQ ID NO: 183) |
| 5-H12 | Heavy Chain (with wild type human IgG1 constant region) |
| Variant 1 | QIQLQQSGPELVRPGASVKISCKASGYSFTDYYINWVNQRPGQGLEWIGWIYPGSGNTRYSERF |
| | KGKATLTVDTSSNTAYMQLSSLTSEDSAVYFCAREDYYPYHGMDYWGQGTSVTVSSASTKGP |
| | SVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCSVMHEALHNHYT |
| | QKSLSLSPGK (SEQ ID NO: 271) |
| | Light Chain (with kappa light chain constant region) |
| | DIVLTQSPTSLAVSLGQRATISCRASESVDGYDNSFMHWYQQKPGQPPKLLIFRASNLESGIPAR |
| | FSGSGSRTDFTLTINPVEAADVATYYCQQSSEDPWTFGGGTKLEIKRTVAAPSVFIFPPSDEQLK |
| | SGTASVVCLLNNFYPREAKVQWKVDNALQSGNSQESVTEQDSKDSTYSLSSTLTLSKADYEK |
| | HKVYACEVTHQGLSSPVTKSFNRGEC (SEQ ID NO: 183) |
| 5-H12 | Heavy Chain (with wild type human IgG1 constant region) |
| Variant 2 | QIQLQQSGPELVRPGASVKISCKASGYSFTDYDINWVNQRPGQGLEWIGWIYPGSGNTRYSER |
| | FKGKATLTVDTSSNTAYMQLSSLTSEDSAVYFCAREDYYPYHGMDYWGQGTSVTVSSASTKGP |
| | SVFPLAPSSKSTSGGTAALGCLVKDYFPEPVTVSWNSGALTSGVHTFPAVLQSSGLYSLSSVV |
| | TVPSSSLGTQTYICNVNHKPSNTKVDKKVEPKSCDKTHTCPPCPAPELLGGPSVFLFPPKPKD |
| | TLMISRTPEVTCVVVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCSVMHEALHNHYT |
| | QKSLSLSPGK (SEQ ID NO: 272) |
| | Light Chain (with kappa light chain constant region) |
| | DIVLTQSPTSLAVSLGQRATISCRASESVDGYDNSFMHWYQQKPGQPPKLLIFRASNLESGIPA |
| | RFSGSGSRTDFTLTINPVEAADVATYYCQQSSEDPWTFGGGTKLEIKRTVAAPSVFIFPPSDEQ |
| | LKSGTASVVCLLNNFYPREAKVQWKVDNALQSGNSQESVTEQDSKDSTYSLSSTLTLSKADYEK |
| | HKVYACEVTHQGLSSPVTKSFNRGEC (SEQ ID NO: 183) |

* VH/VL sequences underlined

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 178, 180, 182, and 269-272. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 179, 181, and 183. In some embodiments, the anti-TfR antibody described herein comprises a heavy chain comprising an amino acid sequence that is at least 75% (e.g., 75%, 80%, 85%, 90%, 95%, 98%, or 99%) identical to any one of SEQ ID NOs: 178, 180, 182, and 269-272. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to any one of SEQ ID NOs: 179, 181, 183. In some embodiments, the anti-TfR antibody described herein comprises a heavy chain comprising the amino acid sequence of any one of SEQ ID NOs: 178, 180, 182, and 269-272. Alternatively or in addition (e.g., in addition), the anti-TfR antibody described herein comprises a light chain comprising the amino acid sequence of any one of SEQ ID NOs: 179, 181, and 183.

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 178, SEQ ID NO: 269, or SEQ ID NO: 270. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 10, 11, 9, 8, 7, 6, 5, 4, 3, 2, or 1 amino acid variation) as compared with the light chain as set forth in SEQ ID NO: 179. In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 178, SEQ ID NO: 269, or SEQ ID NO: 270. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to SEQ ID NO: 179. In some embodiments, the anti-TfR antibody described herein comprises a heavy chain comprising the amino acid sequence of SEQ ID NO: 178, SEQ ID NO: 269, or SEQ ID NO: 270. Alternatively or in addition (e.g., in addition), the anti-TfR antibody described herein comprises a light chain comprising the amino acid sequence of SEQ ID NO: 179.

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 180. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in SEQ ID NO: 181. In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 180. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to SEQ ID NO: 181. In some embodiments, the anti-TfR antibody described herein comprises a heavy chain comprising the amino acid sequence of SEQ ID NO: 180. Alternatively or in addition (e.g., in addition), the anti-TfR antibody described herein comprises a light chain comprising the amino acid sequence of SEQ ID NO: 181.

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 182, SEQ ID NO: 271 or SEQ ID NO: 272. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in SEQ ID NO: 183. In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 182, SEQ ID NO: 271 or SEQ ID NO: 272. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to SEQ ID NO: 183. In some embodiments, the anti-TfR antibody described herein comprises a heavy chain comprising the amino acid sequence of SEQ ID NO: 182, SEQ ID NO: 271 or SEQ ID NO: 272. Alternatively or in addition (e.g., in addition), the anti-TfR antibody described herein comprises a light chain comprising the amino acid sequence of SEQ ID NO: 183.

In some embodiments, the anti-TfR antibody is a FAB fragment, F(ab′) fragment, or F(ab′)₂ fragment of an intact antibody (full-length antibody). Antigen binding fragment of an intact antibody (full-length antibody) can be prepared via routine methods (e.g., recombinantly or by digesting the heavy chain constant region of a full length IgG using an enzyme such as papain). For example, F(ab′)₂ fragments can be produced by pepsin or papain digestion of an antibody molecule, and Fab fragments that can be generated by reducing the disulfide bridges of F(ab′)₂ fragments. In some embodiments, a heavy chain constant region in a F(ab′) fragment of the anti-TfR1 antibody described herein comprises the amino acid sequence of:

(SEQ ID NO: 184)
ASTKGPSVFPLAPSSKSTSGGTAALGCLVKDYFPEPVT
VSWNSGALTSGVHTFPAVLQSSGLYSLSSVVTVPSSSL
GTQTYICNVNHKPSNTKVDKKVEPKSCDKTHT

In some embodiments, the anti-TfR antibody described herein comprises a heavy chain comprising any one of the VH as listed in Table 1 or any variants thereof and a heavy chain constant region that is at least 80%, at least 85%, at least 90%, at least 95%, or at least 99% identical to SEQ ID NO: 184. In some embodiments, the anti-TfR antibody described herein comprises a heavy chain comprising any one of the VH as listed in Table 1 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 184. In some embodiments, the anti-TfR antibody described herein comprises a heavy chain comprising any one of the VH as listed in Table 1 or any variants thereof and a heavy chain constant region as set forth in SEQ ID NO: 184.

Examples of F(ab′) amino acid sequences of the anti-TfR antibodies described herein are provided in Table 5.

TABLE 5
Heavy chain and light chain sequences of examples of anti-TfR F(ab′)

| Antibody | F(ab′) Heavy Chain/Light Chain Sequences |
|---|---|
| 3-A4 | Heavy Chain (with partial human IgG1 constant region) |
| | EVQLQQSGAELVRPGASVKLSCTASGFNIKDDYMYWVKQRPEQGLEWIGWIDPENGDTEYAS |
| | KFQDKATVTADTSSNTAYLQLSSLTSEDTAVYYCTLWLRRGLDYWGQGTSVTVSSASTKGPS |
| | VFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHT (SEQ ID NO: 185) |
| | Light Chain (with kappa light chain constant region) |
| | DIVMTQAAPSVPVTPGESVSISCRSSKSLLHSNGYTYLFWFLQRPGQSPQLLIYRMSNLASGVP |
| | DRFSGSGSGTAFTLRISRVEAEDVGVYYCMQHLEYPFTFGGGTKLEIKRTVAAPSVFIFPPSDEQ |
| | LKSGTASVVCLLNNFYPREAKVQWKVDNALQSGNSQESVTEQDSKDSTYSLSSTLTLSKADYE |
| | KHKVYACEVTHQGLSSPVTKSFNRGEC (SEQ ID NO: 179) |
| 3-A4 | Heavy Chain (with partial human IgG1 constant region) |
| Variant 1 | EVQLQQSGAELVRPGASVKLSCTASGFNIKDDYMYWVKQRPEQGLEWIGWIDPETGDTEYAS |
| | KFQDKATVTADTSSNTAYLQLSSLTSEDTAVYYCTLWLRRGLDYWGQGTSVTVSSASTKGPS |
| | VFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHT (SEQ ID NO: 273) |
| | Light Chain (with kappa light chain constant region) |
| | DIVMTQAAPSVPVTPGESVSISCRSSKSLLHSNGYTYLFWFLQRPGQSPQLLIYRMSNLASGVP |
| | DRFSGSGSGTAFTLRISRVEAEDVGVYYCMQHLEYPFTFGGGTKLEIKRTVAAPSVFIFPPSDEQ |
| | LKSGTASVVCLLNNFYPREAKVQWKVDNALQSGNSQESVTEQDSKDSTYSLSSTLTLSKADYE |
| | KHKVYACEVTHQGLSSPVTKSFNRGEC (SEQ ID NO: 179) |
| 3-A4 | Heavy Chain (with partial human IgG1 constant region) |
| Variant 2 | EVQLQQSGAELVRPGASVKLSCTASGFNIKDDYMYWVKQRPEQGLEWIGWIDPESGDTEYAS |
| | KFQDKATVTADTSSNTAYLQLSSLTSEDTAVYYCTLWLRRGLDYWGQGTSVTVSSASTKGPS |
| | VFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHT (SEQ ID NO: 274) |
| | Light Chain (with kappa light chain constant region) |
| | DIVMTQAAPSVPVTPGESVSISCRSSKSLLHSNGYTYLFWFLQRPGQSPQLLIYRMSNLASGVP |
| | DRFSGSGSGTAFTLRISRVEAEDVGVYYCMQHLEYPFTFGGGTKLEIKRTVAAPSVFIFPPSDEQ |
| | LKSGTASVVCLLNNFYPREAKVQWKVDNALQSGNSQESVTEQDSKDSTYSLSSTLTLSKADYE |
| | KHKVYACEVTHQGLSSPVTKSFNRGEC (SEQ ID NO: 179) |
| 3-M12 | Heavy Chain (with partial human IgG1 constant region) |
| | DVQLQESGPGLVKPSQSLSLTCSVTGYSITSGYYWNWIRQFPGNKLEWMGYITFDGANNYNPS |
| | LKNRISITRDTSKNQFFLKLTSVTTEDTATYYCTRSSYDYDVLDYWGQGTTLTVSSASTKGPSV |
| | FPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHT (SEQ ID NO: 186) |
| | Light Chain (with kappa light chain constant region) |
| | DIQMTQTTSSLSASLGDRVTISCRASQDISNFLNWYQQRPDGTVKLLIYYTSRLHSGVPSRFSGS |
| | GSGTDFSLTVSNLEQEDIATYFCQQGHTLPYTFGGGTKLEIKRTVAAPSVFIFPPSDEQLKSGTA |
| | SVVCLLNNFYPREAKVQWKVDNALQSGNSQESVTEQDSKDSTYSLSSTLTLSKADYEKHKVY |
| | ACEVTHQGLSSPVTKSFNRGEC (SEQ ID NO: 181) |
| 5-H12 | Heavy Chain (with partial human IgG1 constant region) |
| | QIQLQQSGPELVRPGASVKISCKASGYSFTDYCINWVNQRPGQGLEWIGWIYPGSGNTRYSERF |
| | KGKATLTVDTSSNTAYMQLSSLTSEDSAVYFCAREDYYPYHGMDYWGQGTSVTVSSASTKGP |
| | SVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHT (SEQ ID NO: 187) |
| | Light Chain (with kappa light chain constant region) |
| | DIVLTQSPTSLAVSLGQRATISCRASESVDGYDNSFMHWYQQKPGQPPKLLIFRASNLESGIPAR |
| | FSGSGSRTDFTLTINPVEAADVATYYCQQSSEDPWTFGGGTKLEIKRTVAAPSVFIFPPSDEQLK |
| | SGTASVVCLLNNFYPREAKVQWKVDNALQSGNSQESVTEQDSKDSTYSLSSTLTLSKADYEK |
| | HKVYACEVTHQGLSSPVTKSFNRGEC (SEQ ID NO: 183) |
| 5-H12 | Heavy Chain (with partial human IgG1 constant region) |
| Variant 1 | QIQLQQSGPELVRPGASVKISCKASGYSFTDYYINWVNQRPGQGLEWIGWIYPGSGNTRYSERF |
| | KGKATLTVDTSSNTAYMQLSSLTSEDSAVYFCAREDYYPYHGMDYWGQGTSVTVSSASTKGP |
| | SVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHT (SEQ ID NO: 275) |
| | Light Chain (with kappa light chain constant region) |
| | DIVLTQSPTSLAVSLGQRATISCRASESVDGYDNSFMHWYQQKPGQPPKLLIFRASNLESGIPAR |
| | FSGSGSRTDFTLTINPVEAADVATYYCQQSSEDPWTFGGGTKLEIKRTVAAPSVFIFPPSDEQLK |
| | SGTASVVCLLNNFYPREAKVQWKVDNALQSGNSQESVTEQDSKDSTYSLSSTLTLSKADYEK |
| | HKVYACEVTHQGLSSPVTKSFNRGEC (SEQ ID NO: 183) |
| 5-H12 | Heavy Chain (with partial human IgG1 constant region) |
| Variant 2 | QIQLQQSGPELVRPGASVKISCKASGYSFTDYDINWVNQRPGQGLEWIGWIYPGSGNTRYSERF |
| | KGKATLTVDTSSNTAYMQLSSLTSEDSAVYFCAREDYYPYHGMDYWGQGTSVTVSSASTKGP |
| | SVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHT (SEQ ID NO: 276) |
| | Light Chain (with kappa light chain constant region) |
| | DIVLTQSPTSLAVSLGQRATISCRASESVDGYDNSFMHWYQQKPGQPPKLLIFRASNLESGIPAR |
| | FSGSGSRTDFTLTINPVEAADVATYYCQQSSEDPWTFGGGTKLEIKRTVAAPSVFIFPPSDEQLK |
| | SGTASVVCLLNNFYPREAKVQWKVDNALQSGNSQESVTEQDSKDSTYSLSSTLTLSKADYEK |
| | HKVYACEVTHQGLSSPVTKSFNRGEC (SEQ ID NO: 183) |

* VH/VL sequences underlined

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 185, 186, 187, and 273-276. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 179, 181, and 183. In some embodiments, the anti-TfR antibody described herein comprises a heavy chain comprising an amino acid sequence that is at least 75% (e.g., 75%, 80%, 85%, 90%, 95%, 98%, or 99%) identical to any one of SEQ ID NOs: 185, 186, 187, and 273-276. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to any one of SEQ ID NOs: 179, 181, and 183. In some embodiments, the anti-TfR antibody described herein comprises a heavy chain comprising the amino acid sequence of any one of SEQ ID NOs: 185, 186, 187, and 273-276. Alternatively or in addition (e.g., in addition), the anti-TfR antibody described herein comprises a light chain comprising the amino acid sequence of any one of SEQ ID NOs: 179, 181, and 183.

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 185, SEQ ID NO: 273, or SEQ ID NO: 274. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in SEQ ID NO: 179. In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 185, SEQ ID NO: 273, or SEQ ID NO: 274. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to SEQ ID NO: 179. In some embodiments, the anti-TfR antibody described herein comprises a heavy chain comprising the amino acid sequence of SEQ ID NO: 185, SEQ ID NO: 273, or SEQ ID NO: 274. Alternatively or in addition (e.g., in addition), the anti-TfR antibody described herein comprises a light chain comprising the amino acid sequence of SEQ ID NO: 179.

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 186. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 10, 11, 9, 8, 7, 6, 5, 4, 3, 2, or 1 amino acid variation) as compared with the light chain as set forth in SEQ ID NO: 181. In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 186. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to SEQ ID NO: 181. In some embodiments, the anti-TfR antibody described herein comprises a heavy chain comprising the amino acid sequence of SEQ ID NO: 186. Alternatively or in addition (e.g., in addition), the anti-TfR antibody described herein comprises a light chain comprising the amino acid sequence of SEQ ID NO: 181.

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 187, SEQ ID NO: 275, or SEQ ID NO: 276. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in SEQ ID NO: 183. In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 187, SEQ ID NO: 275, or SEQ ID NO: 276. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to SEQ ID NO: 183. In some embodiments, the anti-TfR antibody described herein comprises a heavy chain comprising the amino acid sequence of SEQ ID NO: 187, SEQ ID NO: 275, or SEQ ID NO: 276. Alternatively or in addition (e.g., in addition), the anti-TfR antibody described herein comprises a light chain comprising the amino acid sequence of SEQ ID NO: 183.

The anti-TfR receptor antibodies described herein can be in any antibody form, including, but not limited to, intact (i.e., full-length) antibodies, antigen-binding fragments thereof (such as Fab, F(ab′), F(ab′)2, Fv), single chain antibodies, bi-specific antibodies, or nanobodies. In some embodiments, the anti-TfR antibody described herein is a scFv. In some embodiments, the anti-TfR antibody described herein is a scFv-Fab (e.g., scFv fused to a portion of a constant region). In some embodiments, the anti-TfR receptor antibody described herein is a scFv fused to a constant region (e.g., human IgG1 constant region as set forth in SEQ ID NO: 175 or SEQ ID NO: 176, or a portion thereof such as the Fc portion) at either the N-terminus of C-terminus.

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of an anti-TfR antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-anti-TfR antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-anti-TfR antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of an anti-TfR antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fey receptor. This approach is described further in International Publication No. WO 00/42072.

In some embodiments, the heavy and/or (e.g., and) light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, any one of the anti-TfR1 antibodies described herein may comprise a signal peptide in the heavy and/or (e.g., and) light chain sequence (e.g., a N-terminal signal peptide). In some embodiments, the anti-TfR1 antibody described herein comprises any one of the VH and VL sequences, any one of the IgG heavy chain and light chain sequences, or any one of the F(ab′) heavy chain and light chain sequences described herein, and further comprises a signal peptide (e.g., a N-terminal signal peptide). In some embodiments, the signal peptide comprises the amino acid sequence of MGWSCIILFLVATATGVHS (SEQ ID NO: 232).

In some embodiments, the anti-TfR of the present disclosure is a humanized antibody comprising human framework regions with the CDRs of a murine antibody listed in Table 1 or Table 3 (e.g., 3A4, 3M12, or 5H12). In some embodiments, the anti-TfR of the present disclosure is an IgG1 kappa that comprises human framework regions with the CDRs of a murine antibody listed in Table 1 or Table 3 (e.g., 3A4, 3M12, or 5H12). In some embodiments, the anti-TfR of the present disclosure is a Fab′ fragment of an IgG1 kappa that comprises human framework regions with the CDRs of a murine antibody listed in Table 1 or Table 3 (e.g., 3A4, 3M12, or 5H12).

In some embodiments, an antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. In some embodiments, pyroglutamate formation occurs in a heavy chain sequence. In some embodiments, pyroglutamate formation occurs in a light chain sequence.

### III PREPARATION OF THE ANTI-TFR ANTIBODIES

Antibodies capable of binding TfR as described herein can be made by any method known in the art. See, for example, Harlow and Lane, (1998) Antibodies: A Laboratory Manual, Cold Spring Harbor Laboratory, New York.

In some embodiments, antibodies specific to a target antigen (e.g., TfR) can be made by the conventional hybridoma technology. The full-length target antigen or a fragment thereof, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or (e.g., and) intradermally with an amount of immunogen, including as described herein.

If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are XenomouseR™ from Amgen, Inc. (Fremont, Calif.) and HuMAb-MouseR™ and TC Mouse™ from Medarex, Inc. (Princeton, N.J.) or H2L2 mice from Harbour Antibodies BV (Holland). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments. Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, human HEK293 cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

Alternatively, techniques described for the production of single chain antibodies (U.S. Pat. Nos. 4,946,778 and 4,704,692) can be adapted to produce a phage or yeast scFv library and scFv clones specific to TfR can be identified from the library following routine procedures. Positive clones can be subjected to further screening to identify those that has high TfR binding affinity.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In one example, epitope mapping can be accomplished use H/D-Ex (hydrogen deuterium exchange) coupled with proteolysis and mass spectrometry. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or (e.g., and) necessary for epitope binding. Alternatively, competition assays can be performed using other antibodies known to bind to the same antigen to determine whether an antibody binds to the same epitope as the other antibodies. Competition assays are well known to those of skill in the art.

In some examples, an anti-TfR antibody is prepared by recombinant technology as exemplified below. Nucleic acids encoding the heavy and light chain of an anti-TfR antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV promoter, and the herpes simplex tk virus promoter.

Regulatable promoters can also be used. Such regulatable promoters include those using the lac repressor from E. coli as a transcription modulator to regulate transcription from lac operator bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-555115 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad, among others.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551(1992)] combined the tetracycline repressor (tetR) with the transcription activator (VP 16) to create a tetR-mammalian cell transcription activator fusion protein, tTa (tetR-VP 16), with the tetO bearing minimal promoter derived from the human cytomegalovirus (hCMV) promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art. Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-TfR antibody, as also described herein. The recombinant expression vector can be introduced into a suitable host cell (e.g., a dhfr-CHO cell) by a conventional method, e.g., calcium phosphate mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody. In some embodiments, the host cell used for expressing the anti-TfR antibodies described herein are CHO-S cells (e.g., ThermoFisher Catalog #R80007).

In one example, two recombinant expression vectors are provided, one encoding the heavy chain of the anti-TfR antibody and the other encoding the light chain of the anti-TfR antibody. Both of the two recombinant expression vectors can be introduced into a suitable host cell (e.g., dhfr-CHO cell) by a conventional method, e.g., calcium phosphate-mediated transfection.

Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-TfR antibody as described herein (e.g., as provided in Table 6), vectors (e.g., expression vectors) containing such; and host cells comprising the vectors are within the scope of the present disclosure.

TABLE 6
Nucleic acids Sequences encoding the VH/VL of anti-TfR antibodies listed in Table 1

| Antibody | VH/VL Nucleic Acid Sequence |
|---|---|
| 3-A4 | VH |
| | GAGGTTCAGCTGCAGCAGTCTGGGGCTGAGCTTGTGAGGCCAGGGGCCTCAGTCAAGTTGTC |
| | CTGCACAGCTTCTGGCTTTAACATTAAAGACGACTATATGTACTGGGTGAAGCAGAGGCCTG |
| | AGCAGGGCCTGGAGTGGATTGGATGGATTGATCCTGAGAATGGTGATACTGAATATGCCTCG |
| | AAGTTCCAGGACAAGGCCACTGTAACAGCAGACACATCCTCCAACACAGCCTACCTGCAGCT |
| | CAGCAGCCTGACATCTGAGGACACTGCCGTCTATTACTGTACTTTATGGTTACGCCGAGGGT |
| | TGGACTACTGGGGTCAAGGAACCTCAGTCACCGTCTCCTCA (SEQ ID NO: 188) |
| | VL |
| | GATATTGTGATGACTCAGGCTGCACCCTCTGTACCTGTCACTCCTGGAGAGTCAGTATCCATC |
| | TCCTGCAGGTCTAGTAAGAGTCTCCTGCATAGTAATGGCTACACTTACTTGTTTTGGTTCCTG |
| | CAGAGGCCAGGCCAGTCTCCTCAGCTCCTGATATATCGGATGTCCAACCTTGCCTCAGGAGT |
| | CCCAGACAGGTTCAGTGGCAGTGGGTCAGGAACTGCTTTCACACTGAGAATCAGTAGAGTG |
| | GAGGCTGAGGATGTGGGTGTTTATTACTGTATGCAACATCTAGAATATCCGTTCACGTTCGG |
| | AGGGGGGACCAAGCTGGAAATAAAA (SEQ ID NO: 189) |
| 3-M12 | VH |
| | GATGTACAGCTTCAGGAGTCAGGACCTGGCCTCGTGAAACCTTCTCAGTCTCTGTCTCTCACC |
| | TGCTCTGTCACTGGCTACTCCATCACCAGTGGTTATTACTGGAACTGGATCCGGCAGTTTCCA |
| | GGAAACAAACTGGAATGGATGGGCTACATAACCTTCGATGGTGCCAATAACTACAACCCAT |
| | CTCTCAAAAATCGCATCTCCATCACTCGTGACACATCTAAGAACCAGTTTTTCCTGAAGTTGA |
| | CTTCTGTGACTACTGAGGACACAGCCACATATTACTGTACAAGATCCTCCTATGATTACGAC |
| | GTCCTTGACTACTGGGGCCAAGGCACCACTCTCACAGTCTCCTCA (SEQ ID NO: 190) |
| | VL |
| | GATATCCAGATGACACAGACTACATCCTCCCTGTCTGCCTCTCTGGGAGACAGAGTCACCAT |
| | CAGTTGCAGGGCAAGTCAGGACATTAGCAATTTTTTAAACTGGTATCAGCAGAGACCAGATG |
| | GAACTGTTAAACTCCTGATCTACTACACATCAAGATTACACTCAGGAGTCCCATCAAGGTTC |
| | AGTGGCAGTGGGTCTGGGACAGATTTTTCTCTCACCGTTAGCAACCTGGAGCAAGAAGATAT |
| | TGCCACTTACTTTTGCCAACAGGGTCATACGCTTCCGTACACGTTCGGAGGGGGGACCAAGC |
| | TGGAAATAAAA (SEQ ID NO: 191) |
| 5-H12 | VH |
| | CAGATCCAGCTGCAGCAGTCTGGACCTGAGTTGGTGAGGCCTGGGGCTTCAGTGAAGATATC |
| | CTGCAAGGCTTCTGGCTACTCCTTCACTGACTACTGTATAAATTGGGTGAATCAGAGGCCTG |
| | GACAGGGACTTGAGTGGATTGGATGGATTTATCCTGGAAGCGGTAATACTAGGTACAGTGA |
| | GAGGTTCAAGGGCAAGGCCACATTGACTGTAGACACATCTTCCAACACAGCCTACATGCAGC |
| | TCAGCAGCCTGACCTCTGAGGACTCTGCGGTCTATTTCTGTGCAAGAGAGGATTACTATCCG |
| | TACCATGGTATGGACTACTGGGGTCAAGGCACCTCAGTCACCGTCTCCTCA |
| | (SEQ ID NO: 192) |
| | VL |
| | GACATTGTGCTGACCCAATCTCCAACTTCTTTGGCTGTGTCTCTAGGGCAGAGGGCCACCAT |
| | ATCCTGCAGAGCCAGTGAAAGTGTTGATGGTTATGACAATAGTTTTATGCACTGGTACCAGC |
| | AGAAACCAGGACAGCCACCCAAACTCCTCATCTTTCGTGCATCCAACCTAGAATCTGGGATC |
| | CCTGCCAGGTTCAGTGGCAGTGGGTCTAGGACAGACTTCACCCTCACCATTAATCCTGTGGA |
| | GGCTGCTGATGTTGCAACCTATTACTGTCAGCAAAGTAGTGAGGATCCGTGGACGTTCGGTG |
| | GAGGCACCAAGCTGGAAATCAAA (SEQ ID NO: 193) |
| 8-K6 | VH |
| | CAGGTCCACCTGCAGCAGCCTGGGGCTGAGCTTGTGAAGCCTGGGGCTTCAGTGAAGATGTC |
| | CTGCAAGGCTTCTGGCTACACCTTCACCAGCTACTGGATAACCTGGGTGAAGCAGAGGCCTG |
| | GACAAGGCCTTGAGTGGATTGGAGATATTTTTCCTAATAGTGGTAGAACTAACTACGATGAG |
| | AAGTTCAAGAGCAAGGCCACACTGACTGTAGACACATCCTCCAGCACCGCCTACATGCAGCT |
| | CAGCAGCCTGACATCTGAGGACTCTGCGGTCTATTTCTGTGCAAGGGAGGGAAACTTCGGTA |
| | GTCTTGACTACTGGGGCCAAGGCACCACTCTCACAGTCTCCTCA (SEQ ID NO: 194) |
| | VL |
| | CAAATTGTTCTCACCCAGTCTCCAGCAATCATGTCTGCATCTCCAGGGGAGAAGGTCACCAT |
| | GACCTGCAGTGCCAACTCAAATTTAAATTACATGAACTGGTACCACCAGAAGTCAGGCACCT |
| | CCCCCAAAAGATGGATTTATGACACATCCAAACTGGCTTCTGGAGTCCCTGCTCGCTTCAGT |
| | GCCAGTGGGTCTGGGACCTCTTACTCTCTCACAATCAGCAGCATGGAGGCTGAAGATGCTGC |
| | CACTTATTACTGCCAGCAGTGGAGTAGAAACCCTCTCACGTTCGGTGCTGGGACCAGGCTGG |
| | AGCTGAAA (SEQ ID NO: 195) |
| 9-K23 | VH |
| | CAGGTCACTCTGAAAGAGTCTGGCCCTGGGATGTTGCAGCCCTCCCAGACCCTCAGTCTGAC |
| | TTGTTCTTTCTCTGGGTTTTCACTGAACACTTATGATGTGGGTGTAGGTTGGATTCGTCAGCC |
| | TTCAGGGAAGGGTCTGGAGTGGCTGGCCAACATTTGGTGGAATGATGATAAGTACTATAACT |
| | CAGCCCTGAAGAGCCGGCTCACAATCTCCAAGGATACCTCCAACAACCAGGTCTTCCTCAAG |
| | ATCTCCAGTGTGGACACTGCAGATACTGCCACATACTACTGTACCCTATATAGTTACGACGG |
| | AGGGTTTGCTTACTGGGGCCAAGGGACTCTGGTCACTGTCTCTGCA (SEQ ID NO: 196) |
| | VL |
| | CAAATTGTTCTCACCCAGTCTCCAGCAATCATGTCTGCATCTCTAGGGGAACGGGTCACCAT |
| | GACCTGCACTGCCAGCTCAAGTGTAAGTTCCAGTTACTTGCACTGGTACCAGCAGAAGCCAG |
| | GATCCTCCCCCAAACTCTGGATTTATAGCACATCCAACCTGGCTTCTGGAGTCCCAGCTCGCT |
| | TCAGTGGCAGTGGGTCTGGGACCTCTTACTCTCTCACAATCAGCAGCATGGAGGCTGAAGAT |
| | GCTGCCACTTATTACTGCCACCAGTATCATCGTTCCCCGTACACGTTCGGAGGGGGGACCAA |
| | GCTGGAAATAAAA (SEQ ID NO: 197) |
| 3-E5 | VH |
| | GAGATCCAGATGAAGCAGTCTGGAGCTGAGCTGGTGAAGCCTGGGGCTTCAGTGAAGATAT |
| | CCTGCAAGGCTTCTGGTTACTCATTCACTGGCTACAATATGAACTGGGTGAAGCAGAGCCAT |
| | GGAAAGAGCCTTGAGTGGATTGGAAATATTAATCCTTACTATGGTAGTACTGGCTACAATCA |
| | GAAATTCAAGGGCAAGGCCACATTGACTGTAGACAAATCTTCCAGCACAGCCTACATGCAG |
| | CTCAACAGCCTGACATCTGAGGACTCTGCAGTCTATTACTGTGCAAGAGGGGACTATGGTTA |
| | CGACGAGGGGACCTGGTTTGCTTACTGGGGCCAAGGGACTCTGGTCACTGTCTCTGCA (SEQ |
| | ID NO: 198) |
| | VL |
| | GACATTGTGATGTCACAGTCTCCATCCTCCCTGGCTGTGTCAGCAGGAGAGAAGGTCACTAT |
| | GAGCTGCAAATCCAGTCAGAGTCTGCTCAACAGTAGAACCCGAAAGAACTACTTGGCTTGGT |
| | ACCAGCAGAAACCAGAGCAGTCTCCTAAACTGCTGATCTACTGGGCATCCACTAGGGAATCT |
| | GGGGTCCCTGATCGCTTCACAGGCAGTGGATCTGGGACAGATTTCACTCTCACCATCAGCAG |
| | TGTGCAGGCTGAAGACCTGGCAGTTTATTACTGCAAGCAATCTTATAATCTTCCATTCACGTT |
| | CGGCTCGGGGACAAAATTGGAAATAAAA (SEQ ID NO: 199) |
| 6-D3 | VH |
| | CAGGTCCAACTGCAGCAGCCTGGGGCTGAGCTTGTGAAGCCTGGGGCTTCAGTGAAGATGTC |
| | CTGCAAGGCTTCTGGCTACACCTTCACCAGGCACTGGATAACCTGGGTGAAGCAGAGGCCTG |
| | GACAAGGCCTTGAGTGGATTGGAGATATTTATCCTGGTAGTGGTAGAACTAACTACAATGAG |
| | AAGTTCAAGAGCACGGCCACACTGACTGTAGACACATCCTCCAGCACAGCCTACATGCAGCT |
| | CAGCAGCCTGACATCTGAGGACTCTGCGGTCTATTACTGTGCACGAGACGGTTACTTATATA |
| | TAAACTACTTTGACTACTGGGGCCAAGGAACCACTCTCACAGTCTCCTCA |
| | (SEQ ID NO: 200) |
| | VL |
| | GAAAATGTTCTCACCCAGTCTCCAGCAATCATGTCTGCATCTCCAGGGGAAAAGGTCACCAT |
| | GACCTGCAGTGCCAGCTCAAGTGTAAGTTTCATGCACTGGTTCCAGCAGAAGTCAAGCACCT |
| | CCCCCAAACTCTGGATTTATGACACATCCAAACTGGCTTCTGGAGTCCCAGGTCGCTTCAGT |
| | GGCAGTGGGTCTGGAAGCTCTTACTCTCTCACGATCAGCAGCATGGCGGCTGAAGATGTTGC |
| | CACTTATTACTGTTTTCAGGGGAGTGGGTACCCGTACACGTTCGGAGGGGGGACCAAGCTGG |
| | AAATAAAA (SEQ ID NO: 201) |
| 4-012 | VH |
| | GAGGTTCAGCTGCAGCAGTCTGGGGCTGAGCTTGTGAGGCCAGGGGCCTCAGTCAAGTTGTC |
| | CTGCACAGCTTCTGGCTTTAACATTGTAGACGACTATATGCACTGGGTGAAGCAGAGGCCTG |
| | AACAGGGCCTGGAGTGGATTGGATGGATTTATCCTGAGAATGCTGATACTGAATATGCCTCG |
| | AAGTTCCAGGGCAAGGCCACTATAACAGCAGACACATCCTCCAACACAGCCTACCTGCAGCT |
| | CAGCAGCCTAACATCTGAGGACACTGCCGTCTATTACTGTACTACCGCCACTGGGACGGGCT |
| | GGTTTGCTTACTGGGGCCAAGGGACTCTGGTCACTGTCTCTGCA (SEQ ID NO: 202) |
| | VL |
| | GATGTTGTGATGACCCAGACTCCACTCACTTTGTCGGTTACCATTGGACAACCAGCCTCCATC |
| | TCTTGCAAGTCAAGTCAGAGCCTCTTAGATAGTGATGGAAAGACATATTTGAATTGGTTGTT |
| | TCAGAGGCCAGGCCAGTCTCCAAAGCGCCTAATCTATCTGGTGTCTAAACTGGACTCTGGAG |
| | TCCCTGACAGGTTCACTGGCAGTGGATCAGGGACAGATTTCACACTGAAAATCAGCAGAGTG |
| | GAGACTGAGGATTTGGGAGTTTATTATTGCTGGCAAGGTACACATTTTCCGTGGACGTTCGG |
| | TGGAGGCGCCAAGCTGGAAATCAAA (SEQ ID NO: 203) |
| 4-C5 | VH |
| | CAGGTTCAGCTGCAGCAGTCTGGAGCTGAGCTGATGAAGCCTGGGGCCTCAGTGAAGATATC |
| | CTGCAAGGCTACTGGCTACACATTCAGTAACTACTGGATAGAGTGGGTAAAGCAGAGGCCT |
| | GGACATGGCCTTGAGTGGATTGGAGAGATTTTACCTGGAAGTGGTAGTACTAACTATAATGA |
| | GAACTTCAAGGGCAAGGCCACATTCACTGCAGATACATCCTCCAACACAGCCTACATGCAAC |
| | TCAGCAGCCTGACATCTGAGGACTCTGCCGTCTATTACTGTGCACGAAGGGGGGCCTATGGT |
| | AACTTTCACTACTGGGGCCAAGGCACCACTCTCACAGTCTCCTCA (SEQ ID NO: 204) |
| | VL |
| | GAAATTGTGCTCACCCAGTCTCCAGCACTCATGGCTGCATCTCCAGGGGAGAAGGTCACCAT |
| | CACCTGCAGTGTCAGCTCAAGTATAAGTTCCAGCAACTTGCACTGGTACCAGCAGAAGTCAG |
| | AAACCTCCCCCAAACCCTGGATTTATGGCACATCCAACCTGGCTTCTGGAGTCCCTGTTCGCT |
| | TCAGTGGCAGTGGATCTGGGACCTCTTATTCTCTCACAATCAGCAGCATGGAGGCTGAAGAT |
| | GCTGCCACTTATTACTGTCAACAGTGGCGTAGTTACCCGTACACGTTCGGAGGGGGGACCAA |
| | GCTGGAAATAAAA (SEQ ID NO: 205) |
| 10-P5 | VH |
| | GAGGTCCAGCTGCAACAGTTTGGAGCTGAGCTGGTGAAGCCTGGGGCTTCAGTGAAGATATC |
| | CTGCAAGGCTTCTGGCTACACATTCACTGACTACAACATGGCCTGGGTGAAGGAGAGCCATG |
| | GAAAGAGCCTTGAGTGGATTGGAGATATTAATCCTAACTATGATACTACTAGCTACAACCAG |
| | AAGTTCAAGGGAAAGGCCACATTGACTGTAGACAAGTCCTCCAGCACAGCCCACATGGAGC |
| | TCCGCAGCCTGACATCTGAGGGCACTGCAGTCTATTACTGTGCAAGATCGGGTTACTACGGT |
| | AGTAGCTACTACTGGCACTTCGATGTCTGGGGCACAGGGACCACGGTCACCGTCTCCTCA |
| | (SEQ ID NO: 206) |
| | VL |
| | GACATTGTGATGTCACAGTCTCCATCCTCCCTAGCTGTGTCAGTTGGAGAGAAGGTTACTAT |
| | GAGCTGCAAGTCCAGTCAGAGCCTTTTATATAGTAGCAATCAAAAGAACTACTTGGCCTGGT |
| | ACCAGCAGAAACCAGGGCAGTCTCCTAAACTGCTGATTTACTGGGCATCCACTAGGGAATCT |
| | GGGGTCCCTGATCGCTTCACAGGCAGTGGATCTGGGACAGATTTCACTCTCACCATCAGCAG |
| | TGTGAAGGCTGAAGACCTGGCAGTTTATTACTGTCAGCAATATTATAACTATCCATTCACGTT |
| | CGGCTCGGGGACAAAGTTGGAAATAAAA (SEQ ID NO: 207) |
| 2-H19 | VH |
| | GAGGTTCAGCTGCAGCAGTCTGGGGCAGAGCTAGTGAGGTCAGGGGCCTCAGTCAAGTTGT |
| | CCTGCACAGCTTCTGGCTTCAACATTAAAGACTACTATATGCACTGGGTGAAACAGAGGCCT |
| | GAACAGGGCCTGGAGTGGATTGGATGGATTGATCCTGAGAGTGGTGATACTGAATATGCCCC |
| | GAAGTTCCAGGGCAGGGCCACTATGACTGCAGACACATCCTCCAACACAGCCTACATGCAG |
| | CTCAGCAGCCTGACATCTGAGGACACTGCCGTCTATTACTGTTATGGACATGATTATAGGGT |
| | GGACTGCTGGGGTCAAGGAACCTCAGTCACTGTCTCCTCA (SEQ ID NO: 208) |
| | VL |
| | GATGTTGTGATGACCCAAACTCCACTCTCCCTGCCTGTCAGTCTTGGAGATCAAGCCTCCATC |
| | TCTTGCAGATCTAGTCAGAGCCTTGTACACAGTAATGGAAACACCTATTTACATTGGTACCT |
| | GCAGAAGCCAGGCCAGTCTCCAAAGCTCCTGATCTACAAAGTTTCCAACCGATTTTCTGGGG |
| | TCCCAGACAGGTTCAGTGGCAGTGGATCAGGGACAGATTTCACACTCAAGATCAGCAGAGT |
| | GGAGGCTGAGGATCTGGGAGTTTATTTCTGCTCTCAAAGTACACATATTCCGTGGACGTTCG |
| | GTGGAGGCACCAAGCTGGAAATCAAA (SEQ ID NO: 209) |
| 3-F3 | VH |
| | GAGGTCCAGTTGCAACAGTTTGGAGCTGAGCTGGTGAAGCCTGGGGCTTCAGTGAAGATATC |
| | CTGCAAGGCTTCTGGCTACACATTCACTGACTACAACATGGGCTGGGTGAAGCAGAGCCATG |
| | GAAAGAGCCTTGAGTGGATTGGAGATATTAATCCTAACTATGATAGTACTAGCTACACCCAG |
| | AAGTTCAAGGGAAAGGCCACATTGACTGTAGACAAGTCCTCCAGCACAGCCTACATGGAAC |
| | TCCGCAGCCTGACATCTGAGGACACTGCAGTCTATTACTGTGCAAGATCGGGTTACTACGGT |
| | AGTAGCTACTACTGGCACTTCGATGTCTGGGGCACAGGGACCACGGTCACCGTCTCCTCA |
| | (SEQ ID NO: 210) |
| | VL |
| | GACATTGTGATGTCACAGTCTCCATCCTCCCTAGCTGTGTCAGTTGGAGAGAAGGTTACTAT |
| | GAGCTGCAAGTCCAGTCAGAGCCTTTTATATAGTAGCAATCAAAAGAACTACTTGGCCTGGT |
| | ACCAGCAGAAACCAGGGCAGTCTCCTAAACTGCTGATTTACTGGGCATCCACTAGGGAATCT |
| | GGGGTCCCTGATCGCTTCACAGGCAGTGGATCTGGGACAGATTTCACTCTCACCATCAGCAG |
| | TGTGAAGGCTGAAGACCTGGCAGTTTATTACTGTCAACAATATTATCACTATCCATTCACGTT |
| | CGGCTCGGGGACAAAGTTGGAAATAAAA (SEQ ID NO: 211) |
| 8-017 | VH |
| | CAGGTGCAGCTGAAGGAGTCAGGACCTGGCCTGGTGGCGCCCTCACAGAGCCTGTCCATCAC |
| | ATGCACCGTCTCAGGGTTCTCATTAACCAACTATGGTGTTCACTGGGTTCGCCAGCCTCCAGG |
| | GAAGGGTCTGGAATGGCTGGTCGTGATATGGAATGATGGAAGCGCAACCTATAATTCAGCTC |
| | TCGAATCCAGACTGAGCATCAGCAAGGACAACTCCAAGAGCCAAGTTTTCTTAAAAATGAA |
| | CAGTCTCCAAACTGATGACACAGCCATGTACTACTGTGCCAGACATGAATCTAGTAACCCGT |
| | TTGCTTACTGGGGCCAAGGGACTCTGGTCACTGTCTCTGCA (SEQ ID NO: 212) |
| | VL |
| | GACATCTTGCTGACTCAGTCTCCAGCCATCCTGTCTGTGAGTCCAGGAGAAAGAGTCAGTTT |
| | CTCCTGCAGGGCCAGTCAGAGCATTGGCACAAGCATCCACTGGTATCAGCAAAGAACAAAT |
| | GGCTCTCCAAGGCTTCTCATAAAGTCTGCTTCTGAGTCTATCGCTGGGATTCCTTCCAGGTTT |
| | AGTGGCAGTGGATCAGGGACAGATTTTACTCTTAGCATCAACAGTGTGGAGTCTGAAGATAT |
| | TGCAGATTATTACTGTCAACAAAATAATAGGTGGCCGTACACGTTCGGAGGGGGGACCAAG |
| | CTGGAAATAAAA (SEQ ID NO: 213) |
| 3-M9 | VH |
| | GAGGTTCAGCTGCAGCAGTCTGGGGCTGAGCTTGTGAGGCCAGGGGCCTCAGTCAAGTTGTC |
| | CTGCACAGCTTCTGACTTTAACATTAAAGACGACTATATACATTGGGTGAAACAGAGGCCTG |
| | AACAGGGCCTGGAGTGGATTGGAAGGATTGATCCTGCGAATGGTAATACTAAATATGCCCC |
| | GAAGTTCCAGGACAAGGCCACTATAACTGCAGACACATCCTCCAACACAGCCTACCTGCAGC |
| | TCAGCAGCCTGACTTCTGAGGACACTGCCGTCTATTACTGTGCTCTGGGCTACACCTACTGGG |
| | GCCAAGGCACCACTCTCACAGTCTCCTCA (SEQ ID NO: 214) |
| | VL |
| | GATGTTGTGATGACTCAGACCCCACTCACTTTGTCGGTTACCATTGGACAACCAGCCTCCATC |
| | TCTTGCAAGTCAAGTCAGAGCCTCTTACATAGTTATGGAAAGACATATTTGAATTGGTTATTA |
| | CAGAGGCCAGGCCAGTCTCCAAAGCTCCTAATCTATCTGGTGTCTAAACTGGAATCTGGAGT |
| | CCCTGACAGGTTCAGTGGCAGTGGATCAGGGACAGATTTCACACTGAAAATCAGCAGAGTG |
| | GAGGCTGAGGATTTGGGAGTTTATTACTGCTTGCAAACTACACATTTTCCTCAGACGTTCGGT |
| | GGAGGCACCAAGCTGGAAATCAAG (SEQ ID NO: 215) |
| 10-H2 | VH |
| | GAGGTGCAGCTGGTGGAGTCTGGGGGAGACTTAGTGAAGCCTGGAGGGTCCCTGAAACTCT |
| | CCTGTGCAGCCTCTGGATTCACTTTCAGTGACTATGGAATGCACTGGGTTCGTCAGGGTCCA |
| | GAGAAGGGGCTGGAGTGGGTTGCATACATTAATAGTGGCAGTAGTACCATCTACTATGCAG |
| | ACACAGTGAAGGGCCGATTCACCATCTCCAGAGACAATGCCAAGAACACCCTGTTCCTGCAA |
| | ATGACCAGTCTGAGGTCTGAGGACACGGCCATGTATTACTGTGCAAGGCCGGGGGATTACG |
| | ACAACTATGCTATGGACTACTGGGGTCAAGGAACCTCAGTCACCGTCTCCTCA |
| | (SEQ ID NO: 216) |
| | VL |
| | GACATTGTGATGACCCAGTCTCACAAATTCCTGTCCACATCAGTAGGAGACAGGGTCAGCAT |
| | CACCTGCAAGGCCAGTCAGGATGTGAGTGTTGCTGTAGCCTGGTATCAACAAAAACCAGGG |
| | CAATCTCCTAAACTACTGATTTACTGGGCATACACCCGGCACACTGGAGTCCCTGATCGCTTC |
| | ACAGGCAGTGGATCTGGGACAGAATATACTCTCACCATCAGCAGTGTGCAGGCTGAAGACC |
| | TGGCACTTTATTACTGTCAGCAACATTATAACACTCCTCCGTGGACGTTCGGTGGAGGCACC |
| | AAGCTGGAAATCAAA (SEQ ID NO: 217) |
| 4-J22 | VH |
| | GAGGTTCAGCTGCAACAGTCTGGGGCAGAGCTTGTGAGGTCAGGGGCCTCAGTCAAGTTGTC |
| | CTGCACAGCTTCTGGCTTCAACATTAAAGACTACTATATACACTGGGTGAAGCAGAGGCCTG |
| | AACAGGGCCTGGAGTGGATTGGATGGATTGATCCTGAGAATGCTGATACTGAATATGCCCCG |
| | AAGTTCCAGGGCAAGGCCACTATGACTCCAGACACATCCTCCAACACAGCCTACCTGCAGCT |
| | CAGCAGCCTGACATCTGAGGACACTGCCGTCTATTACTGTTATGCCTGGGACTATTCTATGG |
| | ACTACTGGGGTCAAGGAACCTCAGTCACCGTCTCCTCA |
| | (SEQ ID NO: 218) |
| | VL |
| | GATGTTGTGATGACCCAAACTCCACTCTCCCTGTCTGTCAGTCTTGGAGATCAAGCCTCCATC |
| | TCTTGCAGATCTAGTCAGAGCCTTGTACACAGTAATGGAAACACCTATTTACATTGGTACCT |
| | GCAGAAGCCGGGCCAGTCTCCAAAGCTCCTGATCTACAAAGTTTCCAACCGATTTTCTGGGG |
| | TCCCAGACAGGTTCAGTGGCAGTGGATCAGGGACAGATTTCATACTCAAGATCAGTAGAGTG |
| | GAGGCTGAGGATCTGGGAGTTTATTTCTGCTCTCAAAATACACATGTTCCGTACACGTTCGG |
| | AGGGGGGACCAGGCTGGAAATAAAA (SEQ ID NO: 219) |
| 9-D4 | VH |
| | CAGGTTCAGCTGCAGCAGTCTGGAACTGAGCTGGCGAGGCCTGGGGCTTCAGTGAAGCTGTC |
| | CTGCAAGGCTTCTGGCTTCACCTTCACAGACTATGGTATAAACTGGGTGAAACAGAGAACTG |
| | GACAGGGCCTTGAGTGGATTGGAGAGATTTATCCTAGCAGTGGTAATTCTTACTACAATGAG |
| | AAATTCAAGGCCAAGGCCACACTGACTGCAGACAAATCCTCCAGCACAGCGTACATGGAGC |
| | TCCGCAGTCTGACATCTGAGGACTCTGCGGTCTATTTCTGTGCAAGATCGACTTACTACGGTA |
| | GCCCAATTGACTACTGGGGCCAAGGCACCACTCTCACAGTCTCCTCA |
| | (SEQ ID NO: 220) |
| | VL |
| | GACATTGTGATGACCCAGTCTCACAAATTCATGTCCACACCAGTAGGAGACAGGGTCAGCAT |
| | CACCTGCAAGGCCAGTCAGGATGTGGATACTACTGTAGCCTGGTATCAACAGAAACCAGGG |
| | CAATCTCCGAAACTACTGATTTACTGGGCATCCACCCGGCAAATTGGAGTCCCTGATCGCTT |
| | CACAGGCAGTGGATCTGGGACAGATTTCACTCTCACCATTAGTAATGTGCAGTCTGAAGACT |
| | TGGCAGATTATTTCTGTCAGCAATATAGCACCTATCCTCTGACGTTCGGTGGAGGCACCAAG |
| | CTGGAAATCAAA (SEQ ID NO: 221) |
| 8-D15 | VH |
| | CAGGTGCAGCTGAAGGAGTCAGGACCTGGCCTGGTGGCGCCCTCACAGAGCCTGTCCATTAC |
| | ATGCACTGTCTCTGGGTTCTCATTGACCAGCTATGCTATAACCTGGGTTCGCCAGTCACCAGG |
| | AAAGGGTCTGGAGTGGCTTGGATTAATATGGACTGGTGGAGGCACAAATTATAATTCAGCGC |
| | TCAAATCCAGACTGAGCATCAGCAAAGACAACTCCAAGAGTCAAGTTTTCTTAAAAATGAAC |
| | AGTCTGCAAACTGATGACACAGCCAGGTACTACTGTGCCAGAATCTATGATGGTTACTACCG |
| | GTACTTCGATGTCTGGGGCACAGGGACCACGGTCACCGTCTCCTCA |
| | SEQ ID NO: 222) |
| | VL |
| | CGTATTGTGTTGACCCAGACTCCCAAATTCCTGCTTGTATCAGCAGGAGACAGGGTTACCAT |
| | GACCTGCAAGGCCAGTCAGAGTGTGAGTAATGATGTAGCTTGGTACCAACAGAAGCCAGGG |
| | CAGTCTCCTAAACTGCTGATATACTATGCATCCAATCGTTACACTGGAGTCCCTGATCGCTTC |
| | ACTGGCAGTGGATATGGGACGGATTTCACTTTCACCATCAGCACTGTGCAGGCTGAAGACCT |
| | GGCAGTTTATTTCTGTCAGCAGGATTATAGCTCTCCGTGGACGTTCGGTGGAGGCACCAAGC |
| | TGGAAATCAAA (SEQ ID NO: 223) |
| 4-H4 | VH |
| | GAGGTTCAGCTGCAGCAGTCTGGGGCAGAGCTTGTGAGGTCAGGGGCCTCAGTCAAGTTGTC |
| | CTGCACAGCTTCTGGCTTCAACATTAAGGACTACTATATGCACTGGGTGAAGCAGAGGCCTG |
| | AACAGGGCCTGGACTGGATTGGATGGATTGATCCTGAGAATGGTGATACTGAATATGCCCCG |
| | AAATTCCAGGGCAAGGCCACTATGACTGCAGACACATCCTCCAACACAGCCTACCTGCAGCT |
| | CAGCAGCCTGACATCTGAGGACACTGCCGTCTATTACTGTAATGTCCTTACTATGCCTACGGC |
| | TTACTGGGGCCAAGGGACTCTGGTCACTGTCTCTGCA (SEQ ID NO: 224) |
| | VL |
| | GACATTGTGATGTCACAGTCTCCATCCTCCCTAGCTGTGTCAGTTGGAGAGAAGGTTATTATG |
| | AGCTGCAAGTCCAGTCAGAGCCTTTTATATAGTAGCAATCAAAAGAACTATTTGGCCTGGTA |
| | CCAGCAGAAACCAGGGCAGTCTCCTAAACTGCTGATTTACTGGGCATCCACTAGGGAATCTG |
| | GGGTCCCTGATCGCTTCACAGGCAGTGGATCTGGGACAGATTTCACTCTCACCATCAGCAGT |
| | GTGAAGGCTGAAGACCTGGCAGTTTATTACTGTCAGCAATATTATAGCTATCCGTACACGTT |
| | CGGAGGGGGGACCAAGCTGGAAATAAAA (SEQ ID NO: 225) |
| 9-C4 | VH |
| | GAGGTGCAACTGATGGAATCTGGGGGAGACTTAGTGAAGCCTGGAGGGTCCCTGAAACTCT |
| | CCTGTGCAGCCTCTGGATTCACTTTCAGTAGCTATGGCTTGTCTTGGGTTCGCCAGACTCCAG |
| | ACAAGAGGCTGGAGTGGGTCGCAACCATTACTAGTGGTGGTAGTTATACCTACTATCCAGAC |
| | AGTGTGAAGGGGCGATTCACCATCTCCAGAGACAATGCCAGGAACACCCTGTACCTGCAAA |
| | TGTTCAGTCTGAAGTCTGAGGACACAGCCATGTATTACTGTGCACTATGGTCCCTTGACTACT |
| | GGGGCCAAGGCACCACTCTCACAGTCTCCTCA (SEQ ID NO: 226) |
| | VL |
| | CAAATTGTTCTCACCCAGTCTCCAGCAATCATGTCTGCATCTCCAGGGGAGAAGGTCACCAT |
| | GACCTGCAGTGCCAATTCAAGTTTAAGTTACATGCACTGGTACCAGCAGAAGCCAGGCACCT |
| | CCCCCAAAAGATGGATTTATGACACATCCGAACTGGCTTCTGGAGTCCCTGCTCGCTTCAGT |
| | GGCAGTGGGTCTGGGACCTCTTATTCTCTCACAATCAGCAGCATGGAGGCTGAAGATGCTGC |
| | CACTTATTACTGCCATCAGCGGAGGAGTTACCCATGGACGTTCGGTGGAGGCACCAAGCTGG |
| | AAATCAAA (SEQ ID NO: 227) |

In some embodiments, the anti-TfR antibodies is a humanized antibody that comprises a VH that contains human framework regions with the CDR-H1, CDR-H2, and CDR-H3 of a murine antibody listed in Table 1 or Table 3 (e.g., 3A4, 3M12, or 5H12), and a VL that contains human framework regions with the CDR-L1, CDR-L2, and CDR-L3 of a murine antibody listed in Table 1 or Table 3 (e.g., 3A4, 3M12, or 5H12), wherein the antibody is produced by recombinant DNA technology in Chinese hamster ovary (CHO) cell suspension culture, optionally in CHO-K1 cell (e.g., CHO-K1 cells derived from European Collection of Animal Cell Culture, Cat. No. 85051005) suspension culture.

In some embodiments, the anti-TfR antibodies is an IgG1 kappa that comprises human framework regions with the CDRs of a murine antibody listed in Table 1 or Table 3 (e.g., 3A4, 3M12, or 5H12), wherein the antibody is produced by recombinant DNA technology in Chinese hamster ovary (CHO) cell suspension culture, optionally in CHO-K1 cell (e.g., CHO-K1 cells derived from European Collection of Animal Cell Culture, Cat. No. 85051005) suspension culture.

In some embodiments, the anti-TfR antibodies is a Fab′ fragment of an IgG1 kappa that comprises human framework regions with the CDRs of a murine antibody listed in Table 1 or Table 3 (e.g., 3A4, 3M12, or 5H12), wherein the antibody is produced by recombinant DNA technology in Chinese hamster ovary (CHO) cell suspension culture, optionally in CHO-K1 cell (e.g., CHO-K1 cells derived from European Collection of Animal Cell Culture, Cat. No. 85051005) suspension culture.

### IV. COMPLEXES

In some embodiments, the anti-TfR antibodies described herein can be used for delivering a molecular payload to a target cell or a target tissue (e.g., a cell or tissue that expresses TfR). Accordingly, some aspects of the present disclosure provide complexes comprising any one of the anti-TfR antibody described herein (e.g., 3-A4, 3-M12, or 5-H12 in IgG or FAB form as provided in Table 4 and Table 5, and variants (e.g., humanized variants) thereof) to a molecular payload. The complexes described herein may be used in various applications, e.g., diagnostic or therapeutic applications.

In some embodiments, a complex comprises an anti-TfR antibody covalently linked to an oligonucleotide (e.g., an antisense oligonucleotide). In some embodiments, the complex described herein is used to modulate the activity or function of at least one gene, protein, and/or (e.g., and) nucleic acid. In some embodiments, the molecular payload present with a complex is responsible for the modulation of a gene, protein, and/or (e.g., and) nucleic acids. A molecular payload may be a small molecule, protein, nucleic acid, oligonucleotide, or any molecular entity capable of modulating the activity or function of a gene, protein, and/or (e.g., and) nucleic acid in a cell. In some embodiments, a molecular payload is an oligonucleotide that targets a disease-associated repeat in muscle cells.

A. Molecular Payloads

Some aspects of the disclosure provide molecular payloads, e.g., for modulating a biological outcome, e.g., the transcription of a DNA sequence, the expression of a protein, or the activity of a protein, that can be linked to any one of the anti-TfR antibodies described herein. In some embodiments, such molecular payloads are capable of targeting to a muscle cell, e.g., via specifically binding to a nucleic acid or protein in the muscle cell following delivery to the muscle cell by the linked anti-TfR antibody. It should be appreciated that various types of molecular payloads may be used in accordance with the disclosure. For example, the molecular payload may comprise, or consist of, an oligonucleotide (e.g., antisense oligonucleotide), a peptide (e.g., a peptide that binds a nucleic acid or protein associated with disease in a muscle cell), a protein (e.g., a protein that binds a nucleic acid or protein associated with disease in a muscle cell), or a small molecule (e.g., a small molecule that modulates the function of a nucleic acid or protein associated with disease in a muscle cell).

In some embodiments, the molecular payload is an oligonucleotide that comprises a strand having a region of complementarity to a gene provided in Table 7.

TABLE 7
List of muscle diseases and corresponding genes.
Rare Muscle Disease Target Genes

| | Gene | GenBank |
|---|---|---|
| Disease | Symbol | Accession No. |
| Adult Pompe | GAA | NM_000152; NM_001079803; |
| | | NM_001079804 |
| Adult Pompe | GYS1 | NM_001161587; NM_002103 |
| Centronuclear myopathy (CNM) | DNM2 | NM_001190716; NM_004945; |
| | | NM_001005362; |
| | | NM_001005360; |
| | | NM_001005361; NM_007871 |
| Duchenne muscular dystrophy | DMD | NM_004023; NM_004020; |
| | | NM_004018; NM_004012 |
| Facioscapulohumeral | DUX4 | NM_001306068; |
| muscular | | NM_001363820; |
| dystrophy (FSHD) | | NM_001205218; |
| | | NM_001293798 |
| Familial hypertrophic | MYBPC3 | NM_000256 |
| cardiomyopathy | | |
| Familial hypertrophic cardiomyopathy | MYH6 | NM_002471; NM_001164171; |
| | | NM_010856 |
| Familial hypertrophic | MYH7 | NM_000257; |
| cardiomyopathy | | NM_080728 |
| Familial hypertrophic | TNNI3 | NM_000363 |
| cardiomyopathy | | |
| Familial hypertrophic | TNNT2 | NM_001001432; |
| cardiomyopathy | | NM_001001431; NM_000364; |
| | | NM_001001430; |
| | | NM_001276347; |
| | | NM_001276346; NM_001276345 |
| Fibrodysplasia Ossificans | ACVR1 | NM_001105; NM_001347663; |
| Progressiva (FOP) | | NM_001347664; |
| | | NM_001347665; |
| | | NM_001347666; |
| | | NM_001347667; NM_001111067 |
| Friedreich's ataxia (FRDA) | FXN | NM_001161706; NM_181425; |
| | | NM_000144 |
| Inclusion body | GNE | NM_001190383; |
| myopathy 2 | | NM_001190384; |
| | | NM_001128227; NM_005476; |
| | | NM_001190388 |
| Laing distal myopathy | MYH7 | NM_000257; NM_080728 |
| Myofibrillar myopathy | BAG3 | NM_004281 |
| Myofibrillar myopathy | CRYAB | NM_001885; NM_001330379; |
| | | NM_001289807; NM_001289808 |
| Myofibrillar myopathy | DES | NM_001927 |
| Myofibrillar myopathy | DNAJB6 | NM_005494; NM_058246 |
| Myofibrillar | FHL1 | NM_001159701; |
| myopathy | | NM_001159699; |
| | | NM_001159702; |
| | | NM_001159703; |
| | | NM_001159704; NM_001159700; |
| | | NM_001167819; |
| | | NM_001330659; NM_001449; |
| | | NM_001077362 |
| Myofibrillar myopathy | FLNC | NM_001458; NM_001127487 |
| Myofibrillar myopathy | LDB3 | NM_007078; NM_001171611; |
| | | NM_001171610; |
| | | NM_001080114; |
| | | NM_001080115; NM_001080116 |
| Myofibrillar myopathy | MYOT | NM_001300911; NM_006790; |
| | | NM_001135940 |
| Myofibrillar myopathy | PLEC | NM_201378; NM_201379; |
| | | NM_201380; NM_201381; |
| | | NM_201382; NM_201383; |
| | | NM_201384; NM_000445 |
| Myofibrillar myopathy | TTN | NM_133432; NM_133379; |
| | | NM_133437; NM_003319; |
| | | NM_001256850; |
| | | NM_001267550; NM_133378 |
| Myotonia congenita (autosomal | CLCN1 | NM_000083; NM_013491 |
| dominant form, Thomsen Disease) | | NM_001081563; NM_004409; |
| | | NM_001081560; |
| Myotonic | DMPK | NM_001081562; |
| dystrophy | | NM_001288764; |
| type I | | NM_001288765; NM_001288766 |
| Myotonic dystrophy | CNBP | NM_001127192; |
| type II | | NM_001127193; |
| | | NM_001127194; |
| | | NM_001127195; |
| | | NM_001127196; NM_003418 |
| Myotubular myopathy | MTM1 | NM_000252 |
| Oculopharyngeal muscular dystrophy | PABPN1 | NM_004643 |
| Paramyotonia congenita | SCN4A | NM_000334 |

Muscle Atrophy Gene Targets

| | GenBank | |
|---|---|---|
| Gene Symbol | Accession No. | Related Publications* |
| INHBA | NM_002192; | Lee SJ, et al., Regulation of |
| (also known | XM_017012175.1; | muscle mass by follistatin and |
| as EDF; FRP) | XM_017012176.1; | activins., Mol Endocrinol. 2010 |
| | XM_017012174.1 | October; 24(10): 1998-2008. |
| | | doi: 10.1210/me.2010-0127. |
| | | Epub 2010 Sep. 1. |
| FBXO32 (also | NM_058229.3; | Bodine, S. C., et al., |
| known as Fbx32; | NM_001242463.1; | Identification |
| MAFbx) | NM_148177.2 | of ubiquitin ligases required |
| | | for skeletal muscle atrophy. |
| | | Science 294: 1704-1708, 2001. |
| | | Gomes, M. D., et al., Atrogin-1, a |
| | | muscle-specific F-box protein |
| | | highly expressed during muscle |
| | | atrophy. Proc. Nat. Acad. |
| | | Sci. 98: 14440-14445, 2001. |
| MSTN (also | NM_005259.2 | Saunders, M. A., et al., Human |
| known as GDF8; | | adaptive evolution of myostatin |
| MSLHP) | | (GDF8), a regulator of muscle |
| | | growth. Am. J. Hum. Genet. |
| | | 79:1089-1097, 2006. |
| | | Lin, J., et al., Myostatin knockout |
| | | in mice increases myogenesis and decreases |
| | | adipogenesis. Biochem. |
| | | Biophys. Res. Commun. 291: |
| | | 701-706, 2002. |
| | | Wei Y, et al., Prevention of |
| | | Muscle Wasting by |
| | | CRISPR/Cas9-mediated |
| | | Disruption of Myostatin In |
| | | Vivo Volume 24, Issue 11, |
| | | p1889-1891, November 2016 |
| TRIM63 | NM_032588.3; | Höllriegel R, et al. Anabolic |
| (also known as | XM_017002559.2 | effects of exercise training in |
| IRF; SMRZ; | | patients with advanced chronic |
| MURF1; MURF2; | | heart failure (NYHA IIIb): |
| RNF28) | | impact on ubiquitin-protein |
| | | ligases expression and |
| | | skeletal muscle size. Int J |
| | | Cardiol, 2013 Aug. 10. |
| | | Eddins MJ, et al. Targeting the |
| | | ubiquitin E3 ligase MuRF1 to |
| | | inhibit muscle atrophy. |
| | | Cell Biochem Biophys, 2011 June. |

*The contents of the cited references are incorporated herein by reference in their entireties.

In some embodiments, the molecular payload is an agent for the treatment of a neurological disorder. A “neurological disorder” as used herein refers to a disease or disorder which affects the CNS and/or (e.g., and) which has an etiology in the CNS. Examples of CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease. For the purposes of this application, the CNS will be understood to include the eye, which is normally sequestered from the rest of the body by the blood-retina barrier. Specific examples of neurological disorders include, but are not limited to, neurodegenerative diseases (including, but not limited to, Lewy body disease, postpoliomyelitis syndrome, Shy-Draeger syndrome, olivopontocerebellar atrophy, Parkinson's disease, multiple system atrophy, striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g. of the CNS, including brain metastases resulting from cancer elsewhere in the body). Non-limiting, examples of neurological disorder drugs that may be conjugated to any one of the anti-TfR antibodies described herein and the corresponding conditions they may treat are provided in Table 8.

TABLE 8
Examples of neurological disorder drugs and conditions treated

| Drug | Neurological disorder |
|---|---|
| Anti-BACE1 Antibody | Alzheimer’s, acute and chronic brain injury, |
| | stroke |
| Anti-Abeta Antibody | Alzheimer’s disease |
| Anti-Tau Antibody | Alzheimer’s disease, taupathies |
| Neurotrophin | Stroke, acute brain injury, spinal cord injury |
| Brain-derived neurotrophic factor (BDNF), | Chronic brain injury |
| Fibroblast growth factor 2 (FGF-2) | (Neurogenesis) |
| Anti-Epidermal Growth Factor | Brain Cancer |
| Receptor (EGFR)-antibody | |
| Glial cell-line derived neural factor (GDNF) | Parkinson’s disease |
| Brain derived neurotrophic factor (BDNF) | Amyotrophic lateral sclerosis, depression |
| Lysosomal enzyme | Lysosomal storage disorders of the brain |
| Ciliary neurotrophic factor (CNTF) | Amyotrophic lateral sclerosis |
| Neuregulin-1 | Schizophrenia |
| Anti-HER2 antibody (e.g. trastuzamab, | Brain metastasis from |
| pertuzumab, etc.) | HER2-positive cancer |
| Anti-VEGF antibody (e.g. bevacizumab) | Recurrent or newly diagnosed glioblastoma, |
| | recurrent malignant glioma, brain metastasis |

In some embodiments, at least one (e.g., at least 2, at least 3, at least 4, at least 5, at least 10) molecular payload (e.g., oligonucleotides) is linked to any one of the anti-TfR antibody described herein. In some embodiments, all molecular payloads attached to the anti-TfR antibody are the same, e.g. target the same gene. In some embodiments, all molecular payloads attached to the anti-TfR antibody are different, for example the molecular payloads may target different portions of the same target gene, or the molecular payloads may target at least two different target genes. In some embodiments, an anti-TfR antibody described herein may be attached to some molecular payloads that are the same and some molecular payloads that are different.

The present disclosure also provides a composition comprising a plurality of complexes, for which at least 80% (e.g., at least 85%, at least 90%, at least 91%, at least 92%, at least 93%, at least 94%, at least 95%, at least 96%, at least 97%, at least 98%, or at least 99%) of the complexes comprise an anti-TfR antibody linked to the same number of molecular payloads (e.g., oligonucleotides).

Exemplary molecular payloads are described in further detail herein, however, it should be appreciated that the exemplary molecular payloads provided herein are not meant to be limiting.

i. Oligonucleotides

Any suitable oligonucleotide may be used as a molecular payload, as described herein. In some embodiments, the oligonucleotide may be designed to cause degradation of an mRNA (e.g., the oligonucleotide may be a gapmer, an siRNA, a ribozyme or an aptamer that causes degradation). In some embodiments, the oligonucleotide may be designed to block translation of an mRNA (e.g., the oligonucleotide may be a mixmer, an siRNA or an aptamer that blocks translation). In some embodiments, an oligonucleotide may be designed to caused degradation and block translation of an mRNA. In some embodiments, an oligonucleotide may be a guide nucleic acid (e.g., guide RNA) for directing activity of an enzyme (e.g., a gene editing enzyme). Other examples of oligonucleotides are provided herein. It should be appreciated that, in some embodiments, oligonucleotides in one format (e.g., antisense oligonucleotides) may be suitably adapted to another format (e.g., siRNA oligonucleotides) by incorporating functional sequences (e.g., antisense strand sequences) from one format to the other format.

In some embodiments, an oligonucleotide may comprise a region of complementarity to a target gene provided in Table 7.

In some embodiments, the oligonucleotide may target lncRNA or mRNA, e.g., for degradation. In some embodiments, the oligonucleotide may target, e.g., for degradation, a nucleic acid encoding a protein involved in a mismatch repair pathway, e.g., MSH2, MutLalpha, MutSbeta, MutLalpha. Non-limiting examples of proteins involved in mismatch repair pathways, for which mRNAs encoding such proteins may be targeted by oligonucleotides described herein, are described in Iyer, R. R. et al., “DNA triplet repeat expansion and mismatch repair” Annu Rev Biochem. 2015; 84:199-226; and Schmidt M. H. and Pearson C. E., “Disease-associated repeat instability and mismatch repair” DNA Repair (Amst). 2016 February; 38:117-26.

In some embodiments, any one of the oligonucleotides can be in salt form, e.g., as sodium, potassium, or magnesium salts.

In some embodiments, the 5′ or 3′ nucleoside (e.g., terminal nucleoside) of any one of the oligonucleotides described herein is conjugated to an amine group, optionally via a spacer. In some embodiments, the spacer comprises an aliphatic moiety. In some embodiments, the spacer comprises a polyethylene glycol moiety. In some embodiments, a phosphodiester linkage is present between the spacer and the 5′ or 3′ nucleoside of the oligonucleotide. In some embodiments, the 5′ or 3′ nucleoside (e.g., terminal nucleoside) of any of the oligonucleotides described herein is conjugated to a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; each R^{A} is independently hydrogen or substituted or unsubstituted alkyl. In certain embodiments, the spacer is a substituted or unsubstituted alkylene, substituted or unsubstituted heterocyclylene, substituted or unsubstituted heteroarylene, —O—, —N(R^{A})—, or —C(═O)N(R^{A})₂, or a combination thereof.

In some embodiments, the 5′ or 3′ nucleoside of any one of the oligonucleotides described herein is conjugated to a compound of the formula —NH₂—(CH₂)ₙ—, wherein n is an integer from 1 to 12. In some embodiments, n is 6, 7, 8, 9, 10, 11, or 12. In some embodiments, a phosphodiester linkage is present between the compound of the formula NH₂—(CH₂)ₙ— and the 5′ or 3′ nucleoside of the oligonucleotide. In some embodiments, a compound of the formula NH₂—(CH₂)₆— is conjugated to the oligonucleotide via a reaction between 6-amino-1-hexanol (NH₂—(CH₂)₆—OH) and the 5′ phosphate of the oligonucleotide.

In some embodiments, the oligonucleotide is conjugated to a targeting agent, e.g., a muscle targeting agent such as an anti-TfR antibody, e.g., via the amine group.

a. Oligonucleotide Size/Sequence

Oligonucleotides may be of a variety of different lengths, e.g., depending on the format. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the oligonucleotide is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 21 to 23 nucleotides in lengths, etc.

In some embodiments, a complementary nucleic acid sequence of an oligonucleotide for purposes of the present disclosure is specifically hybridizable or specific for the target nucleic acid when binding of the sequence to the target molecule (e.g., mRNA) interferes with the normal function of the target (e.g., mRNA) to cause a loss of activity (e.g., inhibiting translation) or expression (e.g., degrading a target mRNA) and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency. Thus, in some embodiments, an oligonucleotide may be at least 80%, at least 85%, at least 90%, at least 91%, at least 92%, at least 93%, at least 94%, at least 95%, at least 96%, at least 97%, at least 98%, at least 99% or 100% complementary to the consecutive nucleotides of an target nucleic acid. In some embodiments a complementary nucleotide sequence need not be 100% complementary to that of its target to be specifically hybridizable or specific for a target nucleic acid.

In some embodiments, an oligonucleotide comprises region of complementarity to a target nucleic acid that is in the range of 8 to 15, 8 to 30, 8 to 40, or 10 to 50, or 5 to 50, or 5 to 40 nucleotides in length. In some embodiments, a region of complementarity of an oligonucleotide to a target nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 8 consecutive nucleotides of a target nucleic acid. In some embodiments, an oligonucleotide may contain 1, 2 or 3 base mismatches compared to the portion of the consecutive nucleotides of target nucleic acid. In some embodiments the oligonucleotide may have up to 3 mismatches over 15 bases, or up to 2 mismatches over 10 bases.

In some embodiments, the oligonucleotide is complementary (e.g., at least 85% at least 90%, at least 95%, or 100%) to a target sequence of any one of the oligonucleotides provided herein. In some embodiments, such target sequence is 100% complementary to the oligonucleotide provided herein.

In some embodiments, any one or more of the thymine bases (T's) in any one of the oligonucleotides provided herein may optionally be uracil bases (U's), and/or any one or more of the U's may optionally be T's.

b. Oligonucleotide Modifications:

The oligonucleotides described herein may be modified, e.g., comprise a modified sugar moiety, a modified internucleoside linkage, a modified nucleotide and/or (e.g., and) combinations thereof. In addition, in some embodiments, oligonucleotides may exhibit one or more of the following properties: do not mediate alternative splicing; are not immune stimulatory; are nuclease resistant; have improved cell uptake compared to unmodified oligonucleotides; are not toxic to cells or mammals; have improved endosomal exit internally in a cell; minimizes TLR stimulation; or avoid pattern recognition receptors. Any of the modified chemistries or formats of oligonucleotides described herein can be combined with each other. For example, one, two, three, four, five, or more different types of modifications can be included within the same oligonucleotide.

In some embodiments, certain nucleotide modifications may be used that make an oligonucleotide into which they are incorporated more resistant to nuclease digestion than the native oligodeoxynucleotide or oligoribonucleotide molecules; these modified oligonucleotides survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. Accordingly, oligonucleotides of the disclosure can be stabilized against nucleolytic degradation such as by the incorporation of a modification, e.g., a nucleotide modification.

In some embodiments, an oligonucleotide may be of up to 50 or up to 100 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are modified nucleotides. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are modified nucleotides. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are modified nucleotides. Optionally, the oligonucleotides may have every nucleotide except 1, 2, 3, 4, 5, 6, 7, 8, 9, or 10 nucleotides modified. Oligonucleotide modifications are described further herein.

c. Modified Nucleosides

In some embodiments, the oligonucleotide described herein comprises at least one nucleoside modified at the 2′ position of the sugar. In some embodiments, an oligonucleotide comprises at least one 2′-modified nucleoside. In some embodiments, all of the nucleosides in the oligonucleotide are 2′-modified nucleosides.

In some embodiments, the oligonucleotide described herein comprises one or more non-bicyclic 2′-modified nucleosides, e.g., 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleoside.

In some embodiments, the oligonucleotide described herein comprises one or more 2′-4′ bicyclic nucleosides in which the ribose ring comprises a bridge moiety connecting two atoms in the ring, e.g., connecting the 2′-O atom to the 4′-C atom via a methylene (LNA) bridge, an ethylene (ENA) bridge, or a (S)-constrained ethyl (cEt) bridge. Examples of LNAs are described in International Patent Application Publication WO/2008/043753, published on Apr. 17, 2008, and entitled “RNA Antagonist Compounds For The Modulation Of PCSK9”, the contents of which are incorporated herein by reference in its entirety. Examples of ENAs are provided in International Patent Publication No. WO 2005/042777, published on May 12, 2005, and entitled “APP/ENA Antisense”; Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties. Examples of cEt are provided in U.S. Pat. Nos. 7,101,993; 7,399,845 and 7,569,686, each of which is herein incorporated by reference in its entirety.

In some embodiments, the oligonucleotide comprises a modified nucleoside disclosed in one of the following U.S. patent or patent application Publications: U.S. Pat. No. 7,399,845, issued on Jul. 15, 2008, and entitled “6-Modified Bicyclic Nucleic Acid Analogs”; U.S. Pat. No. 7,741,457, issued on Jun. 22, 2010, and entitled “6-Modified Bicyclic Nucleic Acid Analogs”; U.S. Pat. No. 8,022,193, issued on Sep. 20, 2011, and entitled “6-Modified Bicyclic Nucleic Acid Analogs”; U.S. Pat. No. 7,569,686, issued on Aug. 4, 2009, and entitled “Compounds And Methods For Synthesis Of Bicyclic Nucleic Acid Analogs”; U.S. Pat. No. 7,335,765, issued on Feb. 26, 2008, and entitled “Novel Nucleoside And Oligonucleotide Analogues”; U.S. Pat. No. 7,314,923, issued on Jan. 1, 2008, and entitled “Novel Nucleoside And Oligonucleotide Analogues”; U.S. Pat. No. 7,816,333, issued on Oct. 19, 2010, and entitled “Oligonucleotide Analogues And Methods Utilizing The Same” and US Publication Number 2011/0009471 now U.S. Pat. No. 8,957,201, issued on Feb. 17, 2015, and entitled “Oligonucleotide Analogues And Methods Utilizing The Same”, the entire contents of each of which are incorporated herein by reference for all purposes.

In some embodiments, the oligonucleotide comprises at least one modified nucleoside that results in an increase in Tm of the oligonucleotide in a range of 1° C., 2° C., 3° C., 4° C., or 5° C. compared with an oligonucleotide that does not have the at least one modified nucleoside. The oligonucleotide may have a plurality of modified nucleosides that result in a total increase in Tm of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the modified nucleoside.

The oligonucleotide may comprise a mix of nucleosides of different kinds. For example, an oligonucleotide may comprise a mix of 2′-deoxyribonucleosides or ribonucleosides and 2′-fluoro modified nucleosides. An oligonucleotide may comprise a mix of deoxyribonucleosides or ribonucleosides and 2′-O-Me modified nucleosides. An oligonucleotide may comprise a mix of 2′-fluoro modified nucleosides and 2′-O-Me modified nucleosides. An oligonucleotide may comprise a mix of 2′-4′ bicyclic nucleosides and 2′-MOE, 2′-fluoro, or 2′-O-Me modified nucleosides. An oligonucleotide may comprise a mix of non-bicyclic 2′-modified nucleosides (e.g., 2′-MOE, 2′-fluoro, or 2′-O-Me) and 2′-4′ bicyclic nucleosides (e.g., LNA, ENA, cEt).

The oligonucleotide may comprise alternating nucleosides of different kinds. For example, an oligonucleotide may comprise alternating 2′-deoxyribonucleosides or ribonucleosides and 2′-fluoro modified nucleosides. An oligonucleotide may comprise alternating deoxyribonucleosides or ribonucleosides and 2′-O-Me modified nucleosides. An oligonucleotide may comprise alternating 2′-fluoro modified nucleosides and 2′-O-Me modified nucleosides. An oligonucleotide may comprise alternating 2′-4′ bicyclic nucleosides and 2′-MOE, 2′-fluoro, or 2′-O-Me modified nucleosides. An oligonucleotide may comprise alternating non-bicyclic 2′-modified nucleosides (e.g., 2′-MOE, 2′-fluoro, or 2′-O-Me) and 2′-4′ bicyclic nucleosides (e.g., LNA, ENA, cEt).

In some embodiments, an oligonucleotide described herein comprises a 5′-vinylphosphonate modification, one or more abasic residues, and/or one or more inverted abasic residues.

d. Internucleoside Linkages/Backbones

In some embodiments, oligonucleotide may contain a phosphorothioate or other modified internucleoside linkage. In some embodiments, the oligonucleotide comprises phosphorothioate internucleoside linkages. In some embodiments, the oligonucleotide comprises phosphorothioate internucleoside linkages between at least two nucleotides. In some embodiments, the oligonucleotide comprises phosphorothioate internucleoside linkages between all nucleotides. For example, in some embodiments, oligonucleotides comprise modified internucleoside linkages at the first, second, and/or (e.g., and) third internucleoside linkage at the 5′ or 3′ end of the nucleotide sequence.

Phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

In some embodiments, oligonucleotides may have heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497).

e. Stereospecific Oligonucleotides

In some embodiments, internucleotidic phosphorus atoms of oligonucleotides are chiral, and the properties of the oligonucleotides by adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43.) In some embodiments, phosphorothioate containing oligonucleotides comprise nucleoside units that are joined together by either substantially all Sp or substantially all Rp phosphorothioate intersugar linkages are provided. In some embodiments, such phosphorothioate oligonucleotides having substantially chirally pure intersugar linkages are prepared by enzymatic or chemical synthesis, as described, for example, in U.S. Pat. No. 5,587,261, issued on Dec. 12, 1996, the contents of which are incorporated herein by reference in their entirety. In some embodiments, chirally controlled oligonucleotides provide selective cleavage patterns of a target nucleic acid. For example, in some embodiments, a chirally controlled oligonucleotide provides single site cleavage within a complementary sequence of a nucleic acid, as described, for example, in US Patent Application Publication 20170037399 A1, published on Feb. 2, 2017, entitled “CHIRAL DESIGN”, the contents of which are incorporated herein by reference in their entirety.

f. Morpholinos

In some embodiments, the oligonucleotide may be a morpholino-based compounds. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

g. Peptide Nucleic Acids (PNAs)

In some embodiments, both a sugar and an internucleoside linkage (the backbone) of the nucleotide units of an oligonucleotide are replaced with novel groups. In some embodiments, the base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative publication that report the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

h. Gapmers

In some embodiments, an oligonucleotide described herein is a gapmer. A gapmer oligonucleotide generally has the formula 5′-X—Y—Z-3′, with X and Z as flanking regions around a gap region Y. In some embodiments, flanking region X of formula 5′-X—Y—Z-3′ is also referred to as X region, flanking sequence X, 5′ wing region X, or 5′ wing segment. In some embodiments, flanking region Z of formula 5′-X—Y—Z-3′ is also referred to as Z region, flanking sequence Z, 3′ wing region Z, or 3′ wing segment. In some embodiments, gap region Y of formula 5′-X—Y—Z-3′ is also referred to as Y region, Y segment, or gap-segment Y. In some embodiments, each nucleoside in the gap region Y is a 2′-deoxyribonucleoside, and neither the 5′ wing region X or the 3′ wing region Z contains any 2′-deoxyribonucleosides.

In some embodiments, the Y region is a contiguous stretch of nucleotides, e.g., a region of 6 or more DNA nucleotides, which are capable of recruiting an RNAse, such as RNAse H. In some embodiments, the gapmer binds to the target nucleic acid, at which point an RNAse is recruited and can then cleave the target nucleic acid. In some embodiments, the Y region is flanked both 5′ and 3′ by regions X and Z comprising high-affinity modified nucleosides, e.g., one to six high-affinity modified nucleosides. Examples of high affinity modified nucleosides include, but are not limited to, 2′-modified nucleosides (e.g., 2′-MOE, 2′O-Me, 2′-F) or 2′-4′ bicyclic nucleosides (e.g., LNA, cEt, ENA). In some embodiments, the flanking sequences X and Z may be of 1-20 nucleotides, 1-8 nucleotides, or 1-5 nucleotides in length. The flanking sequences X and Z may be of similar length or of dissimilar lengths. In some embodiments, the gap-segment Y may be a nucleotide sequence of 5-20 nucleotides, 5-15 twelve nucleotides, or 6-10 nucleotides in length.

In some embodiments, the gap region of the gapmer oligonucleotides may contain modified nucleotides known to be acceptable for efficient RNase H action in addition to DNA nucleotides, such as C4′-substituted nucleotides, acyclic nucleotides, and arabino-configured nucleotides. In some embodiments, the gap region comprises one or more unmodified internucleosides. In some embodiments, one or both flanking regions each independently comprise one or more phosphorothioate internucleoside linkages (e.g., phosphorothioate internucleoside linkages or other linkages) between at least two, at least three, at least four, at least five or more nucleotides. In some embodiments, the gap region and two flanking regions each independently comprise modified internucleoside linkages (e.g., phosphorothioate internucleoside linkages or other linkages) between at least two, at least three, at least four, at least five or more nucleotides.

A gapmer may be produced using appropriate methods. Representative U.S. patents, U.S. patent publications, and PCT publications that teach the preparation of gapmers include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; 5,898,031; 7,015,315; 7,101,993; 7,399,845; 7,432,250; 7,569,686; 7,683,036; 7,750,131; 8,580,756; 9,045,754; 9,428,534; 9,695,418; 10,017,764; 10,260,069; 9,428,534; 8,580,756; U.S. patent publication Nos. US20050074801, US20090221685; US20090286969, US20100197762, and US20110112170; PCT publication Nos. WO2004069991; WO2005023825; WO2008049085 and WO2009090182; and EP Patent No. EP2,149,605, each of which is herein incorporated by reference in its entirety.

In some embodiments, a gapmer is 10-40 nucleosides in length. For example, a gapmer may be 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35, or 35-40 nucleosides in length. In some embodiments, a gapmer is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleosides in length.

In some embodiments, the gap region Y in a gapmer is 5-20 nucleosides in length. For example, the gap region Y may be 5-20, 5-15, 5-10, 10-20, 10-15, or 15-20 nucleosides in length. In some embodiments, the gap region Y is 5, 6, 7, 8, 9, 10, 11, 12, 13, 14, 15, 16, 17, 18, 19, or 20 nucleosides in length. In some embodiments, each nucleoside in the gap region Y is a 2′-deoxyribonucleoside. In some embodiments, all nucleosides in the gap region Y are 2′-deoxyribonucleosides. In some embodiments, one or more of the nucleosides in the gap region Y is a modified nucleoside (e.g., a 2′ modified nucleoside such as those described herein). In some embodiments, one or more cytosines in the gap region Y are optionally 5-methyl-cytosines. In some embodiments, each cytosine in the gap region Y is a 5-methyl-cytosines.

In some embodiments, the 5′wing region of a gapmer (X in the 5′-X—Y—Z-3′ formula) and the 3′wing region of a gapmer (Z in the 5′-X—Y—Z-3′ formula) are independently 1-20 nucleosides long. For example, the 5′wing region of a gapmer (X in the 5′-X—Y—Z-3′ formula) and the 3′wing region of the gapmer (Z in the 5′-X—Y—Z-3′ formula) may be independently 1-20, 1-15, 1-10, 1-7, 1-5, 1-3, 1-2, 2-5, 2-7, 3-5, 3-7, 5-20, 5-15, 5-10, 10-20, 10-15, or 15-20 nucleosides long. In some embodiments, the 5′wing region of the gapmer (X in the 5′-X—Y—Z-3′ formula) and the 3′wing region of the gapmer (Z in the 5′-X—Y—Z-3′ formula) are independently 1, 2, 3, 4, 5, 6, 7, 8, 9, 10, 11, 12, 13, 14, 15, 16, 17, 18, 19, or 20 nucleosides long. In some embodiments, the 5′wing region of the gapmer (X in the 5′-X—Y—Z-3′ formula) and the 3′wing region of the gapmer (Z in the 5′-X—Y—Z-3′ formula) are of the same length. In some embodiments, the 5′wing region of the gapmer (X in the 5′-X—Y—Z-3′ formula) and the 3′wing region of the gapmer (Z in the 5′-X—Y—Z-3′ formula) are of different lengths. In some embodiments, the 5′wing region of the gapmer (X in the 5′-X—Y—Z-3′ formula) is longer than the 3′wing region of the gapmer (Z in the 5′-X—Y—Z-3′ formula). In some embodiments, the 5′wing region of the gapmer (X in the 5′-X—Y—Z-3′ formula) is shorter than the 3′wing region of the gapmer (Z in the 5′-X—Y—Z-3′ formula).

In some embodiments, a gapmer comprises a 5′-X—Y—Z-3′ of 5-10-5, 4-12-4, 3-14-3, 2-16-2, 1-18-1, 3-10-3, 2-10-2, 1-10-1, 2-8-2, 4-6-4, 3-6-3, 2-6-2, 4-7-4, 3-7-3, 2-7-2, 4-8-4, 3-8-3, 2-8-2, 1-8-1, 2-9-2, 1-9-1, 2-10-2, 1-10-1, 1-12-1, 1-16-1, 2-15-1, 1-15-2, 1-14-3, 3-14-1, 2-14-2, 1-13-4, 4-13-1, 2-13-3, 3-13-2, 1-12-5, 5-12-1, 2-12-4, 4-12-2, 3-12-3, 1-11-6, 6-11-1, 2-11-5, 5-11-2, 3-11-4, 4-11-3, 1-17-1, 2-16-1, 1-16-2, 1-15-3, 3-15-1, 2-15-2, 1-14-4, 4-14-1, 2-14-3, 3-14-2, 1-13-5, 5-13-1, 2-13-4, 4-13-2, 3-13-3, 1-12-6, 6-12-1, 2-12-5, 5-12-2, 3-12-4, 4-12-3, 1-11-7, 7-11-1, 2-11-6, 6-11-2, 3-11-5, 5-11-3, 4-11-4, 1-18-1, 1-17-2, 2-17-1, 1-16-3, 1-16-3, 2-16-2, 1-15-4, 4-15-1, 2-15-3, 3-15-2, 1-14-5, 5-14-1, 2-14-4, 4-14-2, 3-14-3, 1-13-6, 6-13-1, 2-13-5, 5-13-2, 3-13-4, 4-13-3, 1-12-7, 7-12-1, 2-12-6, 6-12-2, 3-12-5, 5-12-3, 1-11-8, 8-11-1, 2-11-7, 7-11-2, 3-11-6, 6-11-3, 4-11-5, 5-11-4, 1-18-1, 1-17-2, 2-17-1, 1-16-3, 3-16-1, 2-16-2, 1-15-4, 4-15-1, 2-15-3, 3-15-2, 1-14-5, 2-14-4, 4-14-2, 3-14-3, 1-13-6, 6-13-1, 2-13-5, 5-13-2, 3-13-4, 4-13-3, 1-12-7, 7-12-1, 2-12-6, 6-12-2, 3-12-5, 5-12-3, 1-11-8, 8-11-1, 2-11-7, 7-11-2, 3-11-6, 6-11-3, 4-11-5, 5-11-4, 1-19-1, 1-18-2, 2-18-1, 1-17-3, 3-17-1, 2-17-2, 1-16-4, 4-16-1, 2-16-3, 3-16-2, 1-15-5, 2-15-4, 4-15-2, 3-15-3, 1-14-6, 6-14-1, 2-14-5, 5-14-2, 3-14-4, 4-14-3, 1-13-7, 7-13-1, 2-13-6, 6-13-2, 3-13-5, 5-13-3, 4-13-4, 1-12-8, 8-12-1, 2-12-7, 7-12-2, 3-12-6, 6-12-3, 4-12-5, 5-12-4, 2-11-8, 8-11-2, 3-11-7, 7-11-3, 4-11-6, 6-11-4, 5-11-5, 1-20-1, 1-19-2, 2-19-1, 1-18-3, 3-18-1, 2-18-2, 1-17-4, 4-17-1, 2-17-3, 3-17-2, 1-16-5, 2-16-4, 4-16-2, 3-16-3, 1-15-6, 6-15-1, 2-15-5, 5-15-2, 3-15-4, 4-15-3, 1-14-7, 7-14-1, 2-14-6, 6-14-2, 3-14-5, 5-14-3, 4-14-4, 1-13-8, 8-13-1, 2-13-7, 7-13-2, 3-13-6, 6-13-3, 4-13-5, 5-13-4, 2-12-8, 8-12-2, 3-12-7, 7-12-3, 4-12-6, 6-12-4, 5-12-5, 3-11-8, 8-11-3, 4-11-7, 7-11-4, 5-11-6, 6-11-5, 1-21-1, 1-20-2, 2-20-1, 1-20-3, 3-19-1, 2-19-2, 1-18-4, 4-18-1, 2-18-3, 3-18-2, 1-17-5, 2-17-4, 4-17-2, 3-17-3, 1-16-6, 6-16-1, 2-16-5, 5-16-2, 3-16-4, 4-16-3, 1-15-7, 7-15-1, 2-15-6, 6-15-2, 3-15-5, 5-15-3, 4-15-4, 1-14-8, 8-14-1, 2-14-7, 7-14-2, 3-14-6, 6-14-3, 4-14-5, 5-14-4, 2-13-8, 8-13-2, 3-13-7, 7-13-3, 4-13-6, 6-13-4, 5-13-5, 1-12-10, 10-12-1, 2-12-9, 9-12-2, 3-12-8, 8-12-3, 4-12- 7, 7-12-4, 5-12-6, 6-12-5, 4-11-8, 8-11-4, 5-11-7, 7-11-5, 6-11-6, 1-22-1, 1-21-2, 2-21-1, 1-21-3, 3-20-1, 2-20-2, 1-19-4, 4-19-1, 2-19-3, 3-19-2, 1-18-5, 2-18-4, 4-18-2, 3-18-3, 1-17-6, 6-17-1, 2-17-5, 5-17-2, 3-17-4, 4-17-3, 1-16-7, 7-16-1, 2-16-6, 6-16-2, 3-16-5, 5-16-3, 4-16-4, 1-15-8, 8-15-1, 2-15-7, 7-15-2, 3-15-6, 6-15-3, 4-15-5, 5-15-4, 2-14-8, 8-14-2, 3-14-7, 7-14-3, 4-14-6, 6-14-4, 5-14-5, 3-13-8, 8-13-3, 4-13-7, 7-13-4, 5-13-6, 6-13-5, 4-12-8, 8-12-4, 5-12-7, 7-12-5, 6-12-6, 5-11-8, 8-11-5, 6-11-7, or 7-11-6. The numbers indicate the number of nucleosides in X, Y, and Z regions in the 5′-X—Y—Z-3′ gapmer.

In some embodiments, one or more nucleosides in the 5′wing region of a gapmer (X in the 5′-X—Y—Z-3′ formula) or the 3′wing region of a gapmer (Z in the 5′-X—Y—Z-3′ formula) are modified nucleotides (e.g., high-affinity modified nucleosides). In some embodiments, the modified nucleoside (e.g., high-affinity modified nucleosides) is a 2′-modified nucleoside. In some embodiments, the 2′-modified nucleoside is a 2′-4′ bicyclic nucleoside or a non-bicyclic 2′-modified nucleoside. In some embodiments, the high-affinity modified nucleoside is a 2′-4′ bicyclic nucleoside (e.g., LNA, cEt, or ENA) or a non-bicyclic 2′-modified nucleoside (e.g., 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′ DMAP), 2′-O-dimethylaminoethyloxyethyl (2′-O-DMAEOE), or 2′-O—N-methylacetamido (2′-O-NMA)).

In some embodiments, one or more nucleosides in the 5′wing region of a gapmer (X in the 5′-X—Y—Z-3′ formula) are high-affinity modified nucleosides. In some embodiments, each nucleoside in the 5′wing region of the gapmer (X in the 5′-X—Y—Z-3′ formula) is a high-affinity modified nucleoside. In some embodiments, one or more nucleosides in the 3′wing region of a gapmer (Z in the 5′-X—Y—Z-3′ formula) are high-affinity modified nucleosides. In some embodiments, each nucleoside in the 3′wing region of the gapmer (Z in the 5′-X—Y—Z-3′ formula) is a high-affinity modified nucleoside. In some embodiments, one or more nucleosides in the 5′wing region of the gapmer (X in the 5′-X—Y—Z-3′ formula) are high-affinity modified nucleosides and one or more nucleosides in the 3′wing region of the gapmer (Z in the 5′-X—Y—Z-3′ formula) are high-affinity modified nucleosides. In some embodiments, each nucleoside in the 5′wing region of the gapmer (X in the 5′-X—Y—Z-3′ formula) is a high-affinity modified nucleoside and each nucleoside in the 3′wing region of the gapmer (Z in the 5′-X—Y—Z-3′ formula) is high-affinity modified nucleoside.

In some embodiments, the 5′wing region of a gapmer (X in the 5′-X—Y—Z-3′ formula) comprises the same high affinity nucleosides as the 3′wing region of the gapmer (Z in the 5′-X—Y—Z-3′ formula). For example, the 5′wing region of the gapmer (X in the 5′-X—Y—Z-3′ formula) and the 3′wing region of the gapmer (Z in the 5′-X—Y—Z-3′ formula) may comprise one or more non-bicyclic 2′-modified nucleosides (e.g., 2′-MOE or 2′-O-Me). In another example, the 5′wing region of the gapmer (X in the 5′-X—Y—Z-3′ formula) and the 3′wing region of the gapmer (Z in the 5′-X—Y—Z-3′ formula) may comprise one or more 2′-4′ bicyclic nucleosides (e.g., LNA or cEt). In some embodiments, each nucleoside in the 5′wing region of the gapmer (X in the 5′-X—Y—Z-3′ formula) and the 3′wing region of the gapmer (Z in the 5′-X—Y—Z-3′ formula) is a non-bicyclic 2′-modified nucleosides (e.g., 2′-MOE or 2′-O-Me). In some embodiments, each nucleoside in the 5′wing region of the gapmer (X in the 5′-X—Y—Z-3′ formula) and the 3′wing region of the gapmer (Z in the 5′-X—Y—Z-3′ formula) is a 2′-4′ bicyclic nucleosides (e.g., LNA or cEt).

In some embodiments, a gapmer comprises a 5′-X—Y—Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X and Z is a non-bicyclic 2′-modified nucleosides (e.g., 2′-MOE or 2′-O-Me) and each nucleoside in Y is a 2′-deoxyribonucleoside. In some embodiments, the gapmer comprises a 5′-X—Y—Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X and Z is a 2′-4′ bicyclic nucleosides (e.g., LNA or cEt) and each nucleoside in Y is a 2′-deoxyribonucleoside. In some embodiments, the 5′wing region of the gapmer (X in the 5′-X—Y—Z-3′ formula) comprises different high affinity nucleosides as the 3′wing region of the gapmer (Z in the 5′-X—Y—Z-3′ formula). For example, the 5′wing region of the gapmer (X in the 5′-X—Y—Z-3′ formula) may comprise one or more non-bicyclic 2′-modified nucleosides (e.g., 2′-MOE or 2′-O-Me) and the 3′wing region of the gapmer (Z in the 5′-X—Y—Z-3′ formula) may comprise one or more 2′-4′ bicyclic nucleosides (e.g., LNA or cEt). In another example, the 3′wing region of the gapmer (Z in the 5′-X—Y—Z-3′ formula) may comprise one or more non-bicyclic 2′-modified nucleosides (e.g., 2′-MOE or 2′-O-Me) and the 5′wing region of the gapmer (X in the 5′-X—Y—Z-3′ formula) may comprise one or more 2′-4′ bicyclic nucleosides (e.g., LNA or cEt).

In some embodiments, a gapmer comprises a 5′-X—Y—Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X is a non-bicyclic 2′-modified nucleosides (e.g., 2′-MOE or 2′-O-Me), each nucleoside in Z is a 2′-4′ bicyclic nucleosides (e.g., LNA or cEt), and each nucleoside in Y is a 2′-deoxyribonucleoside. In some embodiments, the gapmer comprises a 5′-X—Y—Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X is a 2′-4′ bicyclic nucleosides (e.g., LNA or cEt), each nucleoside in Z is a non-bicyclic 2′-modified nucleosides (e.g., 2′-MOE or 2′-O-Me) and each nucleoside in Y is a 2′-deoxyribonucleoside.

In some embodiments, the 5′wing region of a gapmer (X in the 5′-X—Y—Z-3′ formula) comprises one or more non-bicyclic 2′-modified nucleosides (e.g., 2′-MOE or 2′-O-Me) and one or more 2′-4′ bicyclic nucleosides (e.g., LNA or cEt). In some embodiments, the 3′wing region of the gapmer (Z in the 5′-X—Y—Z-3′ formula) comprises one or more non-bicyclic 2′-modified nucleosides (e.g., 2′-MOE or 2′-O-Me) and one or more 2′-4′ bicyclic nucleosides (e.g., LNA or cEt). In some embodiments, both the 5′wing region of the gapmer (X in the 5′-X—Y—Z-3′ formula) and the 3′wing region of the gapmer (Z in the 5′-X—Y—Z-3′ formula) comprise one or more non-bicyclic 2′-modified nucleosides (e.g., 2′-MOE or 2′-O-Me) and one or more 2′-4′ bicyclic nucleosides (e.g., LNA or cEt).

In some embodiments, a gapmer comprises a 5′-X—Y—Z-3′ configuration, wherein X and Z is independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X (the 5′ most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside. In some embodiments, the gapmer comprises a 5′-X—Y—Z-3′ configuration, wherein X and Z is independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in Z (the 5′ most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside. In some embodiments, the gapmer comprises a 5′-X—Y—Z-3′ configuration, wherein X and Z is independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X and at least one of positions but not all (e.g., 1, 2, 3, 4, 5, or 6) 1, 2, 3, 4, 5, 6, or 7 in Z (the 5′ most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside.

Non-limiting examples of gapmers configurations with a mix of non-bicyclic 2′-modified nucleoside (e.g., 2′-MOE or 2′-O-Me) and 2′-4′ bicyclic nucleosides (e.g., LNA or cEt) in the 5′wing region of the gapmer (X in the 5′-X—Y—Z-3′ formula) and/or the 3′wing region of the gapmer (Z in the 5′-X—Y—Z-3′ formula) include: BBB-(D)n-BBBAA; KKK-(D)n-KKKAA; LLL-(D)n-LLLAA; BBB-(D)n-BBBEE; KKK-(D)n-KKKEE; LLL-(D)n-LLLEE; BBB-(D)n-BBBAA; KKK-(D)n-KKKAA; LLL-(D)n-LLLAA; BBB-(D)n-BBBEE; KKK-(D)n-KKKEE; LLL-(D)n-LLLEE; BBB-(D)n-BBBAAA; KKK-(D)n-KKKAAA; LLL-(D)n-LLLAAA; BBB-(D)n-BBBEEE; KKK-(D)n-KKKEEE; LLL-(D)n-LLLEEE; BBB-(D)n-BBBAAA; KKK-(D)n-KKKAAA; LLL-(D)n-LLLAAA; BBB-(D)n-BBBEEE; KKK-(D)n-KKKEEE; LLL-(D)n-LLLEEE; BABA-(D)n-ABAB; KAKA-(D)n-AKAK; LALA-(D)n-ALAL; BEBE-(D)n-EBEB; KEKE-(D)n-EKEK; LELE-(D)n-ELEL; BABA-(D)n-ABAB; KAKA-(D)n-AKAK; LALA-(D)n-ALAL; BEBE-(D)n-EBEB; KEKE-(D)n-EKEK; LELE-(D)n-ELEL; ABAB-(D)n-ABAB; AKAK-(D)n-AKAK; ALAL-(D)n-ALAL; EBEB-(D)n-EBEB; EKEK-(D)n-EKEK; ELEL-(D)n-ELEL; ABAB-(D)n-ABAB; AKAK-(D)n-AKAK; ALAL-(D)n-ALAL; EBEB-(D)n-EBEB; EKEK-(D)n-EKEK; ELEL-(D)n-ELEL; AABB-(D)n-BBAA; BBAA-(D)n-AABB; AAKK-(D)n-KKAA; AALL-(D)n-LLAA; EEBB-(D)n-BBEE; EEKK-(D)n-KKEE; EELL-(D)n-LLEE; AABB-(D)n-BBAA; AAKK-(D)n-KKAA; AALL-(D)n-LLAA; EEBB-(D)n-BBEE; EEKK-(D)n-KKEE; EELL-(D)n-LLEE; BBB-(D)n-BBA; KKK-(D)n-KKA; LLL-(D)n-LLA; BBB-(D)n-BBE; KKK-(D)n-KKE; LLL-(D)n-LLE; BBB-(D)n-BBA; KKK-(D)n-KKA; LLL-(D)n-LLA; BBB-(D)n-BBE; KKK-(D)n-KKE; LLL-(D)n-LLE; BBB-(D)n-BBA; KKK-(D)n-KKA; LLL-(D)n-LLA; BBB-(D)n-BBE; KKK-(D)n-KKE; LLL-(D)n-LLE; ABBB-(D)n-BBBA; AKKK-(D)n-KKKA; ALLL-(D)n-LLLA; EBBB-(D)n-BBBE; EKKK-(D)n-KKKE; ELLL-(D)n-LLLE; ABBB-(D)n-BBBA; AKKK-(D)n-KKKA; ALLL-(D)n-LLLA; EBBB-(D)n-BBBE; EKKK-(D)n-KKKE; ELLL-(D)n-LLLE; ABBB-(D)n-BBBAA; AKKK-(D)n-KKKAA; ALLL-(D)n-LLLAA; EBBB-(D)n-BBBEE; EKKK-(D)n-KKKEE; ELLL-(D)n-LLLEE; ABBB-(D)n-BBBAA; AKKK-(D)n-KKKAA; ALLL-(D)n-LLLAA; EBBB-(D)n-BBBEE; EKKK-(D)n-KKKEE; ELLL-(D)n-LLLEE; AABBB-(D)n-BBB; AAKKK-(D)n-KKK; AALLL-(D)n-LLL; EEBBB-(D)n-BBB; EEKKK-(D)n-KKK; EELLL-(D)n-LLL; AABBB-(D)n-BBB; AAKKK-(D)n-KKK; AALLL-(D)n-LLL; EEBBB-(D)n-BBB; EEKKK-(D)n-KKK; EELLL-(D)n-LLL; AABBB-(D)n-BBBA; AAKKK-(D)n-KKKA; AALLL-(D)n-LLLA; EEBBB-(D)n-BBBE; EEKKK-(D)n-KKKE; EELLL-(D)n-LLLE; AABBB-(D)n-BBBA; AAKKK-(D)n-KKKA; AALLL-(D)n-LLLA; EEBBB-(D)n-BBBE; EEKKK-(D)n-KKKE; EELLL-(D)n-LLLE; ABBAABB-(D)n-BB; AKKAAKK-(D)n-KK; ALLAALLL-(D)n-LL; EBBEEBB-(D)n-BB; EKKEEKK-(D)n-KK; ELLEELL-(D)n-LL; ABBAABB-(D)n-BB; AKKAAKK-(D)n-KK; ALLAALL-(D)n-LL; EBBEEBB-(D)n-BB; EKKEEKK-(D)n-KK; ELLEELL-(D)n-LL; ABBABB-(D)n-BBB; AKKAKK-(D)n-KKK; ALLALLL-(D)n-LLL; EBBEBB-(D)n-BBB; EKKEKK-(D)n-KKK; ELLELL-(D)n-LLL; ABBABB-(D)n-BBB; AKKAKK-(D)n-KKK; ALLALL-(D)n-LLL; EBBEBB-(D)n-BBB; EKKEKK-(D)n-KKK; ELLELL-(D)n-LLL; EEEK-(D)n-EEEEEEEE; EEK-(D)n-EEEEEEEEE; EK-(D)n-EEEEEEEEEE; EK-(D)n-EEEKK; K-(D)n-EEEKEKE; K-(D)n-EEEKEKEE; K-(D)n-EEKEK; EK-(D)n-EEEEKEKE; EK-(D)n-EEEKEK; EEK-(D)n-KEEKE; EK-(D)n-EEKEK; EK-(D)n-KEEK; EEK-(D)n-EEEKEK; EK-(D)n-KEEEKEE; EK-(D)n-EEKEKE; EK-(D)n-EEEKEKE; and EK-(D)n-EEEEKEK. “A” nucleosides comprise a 2′-modified nucleoside; “B” represents a 2′-4′ bicyclic nucleoside; “K” represents a constrained ethyl nucleoside (cEt); “L” represents an LNA nucleoside; and “E” represents a 2′-MOE modified ribonucleoside; “D” represents a 2′-deoxyribonucleoside; “n” represents the length of the gap segment (Y in the 5′-X—Y—Z-3′ configuration) and is an integer between 1-20.

In some embodiments, any one of the gapmers described herein comprises one or more modified nucleoside linkages (e.g., a phosphorothioate linkage) in each of the X, Y, and Z regions. In some embodiments, each internucleoside linkage in the any one of the gapmers described herein is a phosphorothioate linkage. In some embodiments, each of the X, Y, and Z regions independently comprises a mix of phosphorothioate linkages and phosphodiester linkages. In some embodiments, each internucleoside linkage in the gap region Y is a phosphorothioate linkage, the 5′wing region X comprises a mix of phosphorothioate linkages and phosphodiester linkages, and the 3′wing region Z comprises a mix of phosphorothioate linkages and phosphodiester linkages.

i. Mixmers

In some embodiments, an oligonucleotide described herein may be a mixmer or comprise a mixmer sequence pattern. In general, mixmers are oligonucleotides that comprise both naturally and non-naturally occurring nucleosides or comprise two different types of non-naturally occurring nucleosides typically in an alternating pattern. Mixmers generally have higher binding affinity than unmodified oligonucleotides and may be used to specifically bind a target molecule, e.g., to block a binding site on the target molecule. Generally, mixmers do not recruit an RNase to the target molecule and thus do not promote cleavage of the target molecule. Such oligonucleotides that are incapable of recruiting RNase H have been described, for example, see WO2007/112754 or WO2007/112753.

In some embodiments, the mixmer comprises or consists of a repeating pattern of nucleoside analogues and naturally occurring nucleosides, or one type of nucleoside analogue and a second type of nucleoside analogue. However, a mixmer need not comprise a repeating pattern and may instead comprise any arrangement of modified nucleoside s and naturally occurring nucleoside s or any arrangement of one type of modified nucleoside and a second type of modified nucleoside. The repeating pattern, may, for instance be every second or every third nucleoside is a modified nucleoside, such as LNA, and the remaining nucleoside s are naturally occurring nucleosides, such as DNA, or are a 2′ substituted nucleoside analogue such as 2′-MOE or 2′ fluoro analogues, or any other modified nucleoside described herein. It is recognized that the repeating pattern of modified nucleoside, such as LNA units, may be combined with modified nucleoside at fixed positions—e.g. at the 5′ or 3′ termini.

In some embodiments, a mixmer does not comprise a region of more than 5, more than 4, more than 3, or more than 2 consecutive naturally occurring nucleosides, such as DNA nucleosides. In some embodiments, the mixmer comprises at least a region consisting of at least two consecutive modified nucleoside, such as at least two consecutive LNAs. In some embodiments, the mixmer comprises at least a region consisting of at least three consecutive modified nucleoside units, such as at least three consecutive LNAs.

In some embodiments, the mixmer does not comprise a region of more than 7, more than 6, more than 5, more than 4, more than 3, or more than 2 consecutive nucleoside analogues, such as LNAs. In some embodiments, LNA units may be replaced with other nucleoside analogues, such as those referred to herein.

Mixmers may be designed to comprise a mixture of affinity enhancing modified nucleosides, such as in non-limiting example LNA nucleosides and 2′-O-Me nucleosides. In some embodiments, a mixmer comprises modified internucleoside linkages (e.g., phosphorothioate internucleoside linkages or other linkages) between at least two, at least three, at least four, at least five or more nucleosides.

A mixmer may be produced using any suitable method. Representative U.S. patents, U.S. patent publications, and PCT publications that teach the preparation of mixmers include U.S. patent publication Nos. US20060128646, US20090209748, US20090298916, US20110077288, and US20120322851, and U.S. Pat. No. 7,687,617.

In some embodiments, a mixmer comprises one or more morpholino nucleosides. For example, in some embodiments, a mixmer may comprise morpholino nucleosides mixed (e.g., in an alternating manner) with one or more other nucleosides (e.g., DNA, RNA nucleosides) or modified nucleosides (e.g., LNA, 2′-O-Me nucleosides).

In some embodiments, mixmers are useful for splice correcting or exon skipping, for example, as reported in Touznik A., et al., LNA/DNA mixmer-based antisense oligonucleotides correct alternative splicing of the SMN2 gene and restore SMN protein expression in type 1 SMA fibroblasts Scientific Reports, volume 7, Article number: 3672 (2017), Chen S. et al., Synthesis of a Morpholino Nucleic Acid (MNA)-Uridine Phosphoramidite, and Exon Skipping Using MNA/2′-O-Methyl Mixmer Antisense Oligonucleotide, Molecules 2016, 21, 1582, the contents of each which are incorporated herein by reference.

j. RNA Interference (RNAi)

In some embodiments, oligonucleotides provided herein may be in the form of small interfering RNAs (siRNA), also known as short interfering RNA or silencing RNA. SiRNA, is a class of double-stranded RNA molecules, typically about 20-25 base pairs in length that target nucleic acids (e.g., mRNAs) for degradation via the RNA interference (RNAi) pathway in cells. Specificity of siRNA molecules may be determined by the binding of the antisense strand of the molecule to its target RNA. Effective siRNA molecules are generally less than 30 to 35 base pairs in length to prevent the triggering of non-specific RNA interference pathways in the cell via the interferon response, although longer siRNA can also be effective. In some embodiments, the siRNA molecules are 7, 8, 9, 10, 11, 12, 13, 14, 15, 16, 17, 18, 19, 20, 21, 22, 23, 24, 25, 26, 27, 28, 29, 30, 35, 40, 45, 50, or more base pairs in length. In some embodiments, the siRNA molecules are 8 to 30 base pairs in length, 10 to 15 base pairs in length, 10 to 20 base pairs in length, 15 to 25 base pairs in length, 19 to 21 base pairs in length, 21 to 23 base pairs in length.

Following selection of an appropriate target RNA sequence, siRNA molecules that comprise a nucleotide sequence complementary to all or a portion of the target sequence, i.e. an antisense sequence, can be designed and prepared using appropriate methods (see, e.g., PCT Publication Number WO 2004/016735; and U.S. Patent Publication Nos. 2004/0077574 and 2008/0081791). The siRNA molecule can be double stranded (i.e. a dsRNA molecule comprising an antisense strand and a complementary sense strand strand that hybridizes to form the dsRNA) or single-stranded (i.e. a ssRNA molecule comprising just an antisense strand). The siRNA molecules can comprise a duplex, asymmetric duplex, hairpin or asymmetric hairpin secondary structure, having self-complementary sense and antisense strands.

In some embodiments, the antisense strand of the siRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the antisense strand is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, 21 to 23 nucleotides in lengths.

In some embodiments, the sense strand of the siRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the sense strand is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, 21 to 23 nucleotides in lengths.

In some embodiments, siRNA molecules comprise an antisense strand comprising a region of complementarity to a target region in a target mRNA. In some embodiments, the region of complementarity is at least 80%, at least 85%, at least 90%, at least 91%, at least 92%, at least 93%, at least 94%, at least 95%, at least 96%, at least 97%, at least 98%, at least 99% or 100% complementary to a target region in a target mRNA. In some embodiments, the target region is a region of consecutive nucleotides in the target mRNA. In some embodiments, a complementary nucleotide sequence need not be 100% complementary to that of its target to be specifically hybridizable or specific for a target RNA sequence.

In some embodiments, siRNA molecules comprise an antisense strand that comprises a region of complementarity to a target RNA sequence and the region of complementarity is in the range of 8 to 15, 8 to 30, 8 to 40, or 10 to 50, or 5 to 50, or 5 to 40 nucleotides in length. In some embodiments, a region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more consecutive nucleotides of a target RNA sequence. In some embodiments, siRNA molecules comprise a nucleotide sequence that contains no more than 1, 2, 3, 4, or 5 base mismatches compared to the portion of the consecutive nucleotides of target RNA sequence. In some embodiments, siRNA molecules comprise a nucleotide sequence that has up to 3 mismatches over 15 bases, or up to 2 mismatches over 10 bases.

In some embodiments, siRNA molecules comprise an antisense strand comprising a nucleotide sequence that is complementary (e.g., at least 85%, at least 90%, at least 95%, or 100%) to the target RNA sequence of the oligonculeotides provided herein. In some embodiments, siRNA molecules comprise an antisense strand comprising a nucleotide sequence that is at least 85%, at least 90%, at least 95%, or 100% identical to the oligonucleotides provided herein. In some embodiments, siRNA molecules comprise an antisense strand comprising at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more consecutive nucleotides of the oligonucleotides provided herein.

Double-stranded siRNA may comprise sense and anti-sense RNA strands that are the same length or different lengths. Double-stranded siRNA molecules can also be assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating RNAi. Small hairpin RNA (shRNA) molecules thus are also contemplated herein. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in addition or removal of one, two, three or more nucleotides from the 3′ end and/or (e.g., and) the 5′ end of either or both strands). A spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end and/or (e.g., and) the 5′ end of either or both strands). A spacer sequence is may be an unrelated nucleotide sequence that is situated between two complementary nucleotide sequence regions which, when annealed into a double-stranded nucleic acid, comprise a shRNA.

The overall length of the siRNA molecules can vary from about 14 to about 100 nucleotides depending on the type of siRNA molecule being designed. Generally between about 14 and about 50 of these nucleotides are complementary to the RNA target sequence, i.e. constitute the specific antisense sequence of the siRNA molecule. For example, when the siRNA is a double- or single-stranded siRNA, the length can vary from about 14 to about 50 nucleotides, whereas when the siRNA is a shRNA or circular molecule, the length can vary from about 40 nucleotides to about 100 nucleotides.

An siRNA molecule may comprise a 3′ overhang at one end of the molecule, The other end may be blunt-ended or have also an overhang (5′ or 3′). When the siRNA molecule comprises an overhang at both ends of the molecule, the length of the overhangs may be the same or different. In one embodiment, the siRNA molecule of the present disclosure comprises 3′ overhangs of about 1 to about 3 nucleotides on both ends of the molecule. In some embodiments, the siRNA molecule comprises 3′ overhangs of about 1 to about 3 nucleotides on the sense strand. In some embodiments, the siRNA molecule comprises 3′ overhangs of about 1 to about 3 nucleotides on the antisense strand. In some embodiments, the siRNA molecule comprises 3′ overhangs of about 1 to about 3 nucleotides on both the sense strand and the antisense strand.

In some embodiments, the siRNA molecule comprises one or more modified nucleotides (e.g. 1, 2, 3, 4, 5, 6, 7, 8, 9, 10 or more). In some embodiments, the siRNA molecule comprises one or more modified nucleotides and/or (e.g., and) one or more modified internucleotide linkages. In some embodiments, the modified nucleotide is a modified sugar moiety (e.g. a 2′ modified nucleotide). In some embodiments, the siRNA molecule comprises one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). In some embodiments, each nucleotide of the siRNA molecule is a modified nucleotide (e.g., a 2′-modified nucleotide). In some embodiments, the siRNA molecule comprises one or more phosphorodiamidate morpholinos. In some embodiments, each nucleotide of the siRNA molecule is a phosphorodiamidate morpholino.

In some embodiments, the siRNA molecule contains a phosphorothioate or other modified internucleotide linkage. In some embodiments, the siRNA molecule comprises phosphorothioate internucleoside linkages. In some embodiments, the siRNA molecule comprises phosphorothioate internucleoside linkages between at least two nucleotides. In some embodiments, the siRNA molecule comprises phosphorothioate internucleoside linkages between all nucleotides. For example, in some embodiments, the siRNA molecule comprises modified internucleotide linkages at the first, second, and/or (e.g., and) third internucleoside linkage at the 5′ or 3′ end of the siRNA molecule.

In some embodiments, the modified internucleotide linkages are phosphorus-containing linkages. In some embodiments, phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Any of the modified chemistries or formats of siRNA molecules described herein can be combined with each other. For example, one, two, three, four, five, or more different types of modifications can be included within the same siRNA molecule.

In some embodiments, the antisense strand comprises one or more modified nucleotides (e.g. 1, 2, 3, 4, 5, 6, 7, 8, 9, 10 or more). In some embodiments, the antisense strand comprises one or more modified nucleotides and/or (e.g., and) one or more modified internucleotide linkages. In some embodiments, the modified nucleotide comprises a modified sugar moiety (e.g. a 2′ modified nucleotide). In some embodiments, the antisense strand comprises one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). In some embodiments, each nucleotide of the antisense strand is a modified nucleotide (e.g., a 2′-modified nucleotide). In some embodiments, the antisense strand comprises one or more phosphorodiamidate morpholinos. In some embodiments, the antisense strand is a phosphorodiamidate morpholino oligomer (PMO).

In some embodiments, antisense strand contains a phosphorothioate or other modified internucleotide linkage. In some embodiments, the antisense strand comprises phosphorothioate internucleoside linkages. In some embodiments, the antisense strand comprises phosphorothioate internucleoside linkages between at least two nucleotides. In some embodiments, the antisense strand comprises phosphorothioate internucleoside linkages between all nucleotides. For example, in some embodiments, the antisense strand comprises modified internucleotide linkages at the first, second, and/or (e.g., and) third internucleoside linkage at the 5′ or 3′ end of the siRNA molecule. In some embodiments, the modified internucleotide linkages are phosphorus-containing linkages. In some embodiments, phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Any of the modified chemistries or formats of the antisense strand described herein can be combined with each other. For example, one, two, three, four, five, or more different types of modifications can be included within the same antisense strand.

In some embodiments, the sense strand comprises one or more modified nucleotides (e.g. 1, 2, 3, 4, 5, 6, 7, 8, 9, 10 or more). In some embodiments, the sense strand comprises one or more modified nucleotides and/or (e.g., and) one or more modified internucleotide linkages. In some embodiments, the modified nucleotide is a modified sugar moiety (e.g. a 2′ modified nucleotide). In some embodiments, the sense strand comprises one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). In some embodiments, each nucleotide of the sense strand is a modified nucleotide (e.g., a 2′-modified nucleotide). In some embodiments, the sense strand comprises one or more phosphorodiamidate morpholinos. In some embodiments, the antisense strand is a phosphorodiamidate morpholino oligomer (PMO). In some embodiments, the sense strand contains a phosphorothioate or other modified internucleotide linkage. In some embodiments, the sense strand comprises phosphorothioate internucleoside linkages. In some embodiments, the sense strand comprises phosphorothioate internucleoside linkages between at least two nucleotides. In some embodiments, the sense strand comprises phosphorothioate internucleoside linkages between all nucleotides. For example, in some embodiments, the sense strand comprises modified internucleotide linkages at the first, second, and/or (e.g., and) third internucleoside linkage at the 5′ or 3′ end of the sense strand.

In some embodiments, the modified internucleotide linkages are phosphorus-containing linkages. In some embodiments, phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Any of the modified chemistries or formats of the sense strand described herein can be combined with each other. For example, one, two, three, four, five, or more different types of modifications can be included within the same sense strand.

In some embodiments, the antisense or sense strand of the siRNA molecule comprises modifications that enhance or reduce RNA-induced silencing complex (RISC) loading. In some embodiments, the antisense strand of the siRNA molecule comprises modifications that enhance RISC loading. In some embodiments, the sense strand of the siRNA molecule comprises modifications that reduce RISC loading and reduce off-target effects. In some embodiments, the antisense strand of the siRNA molecule comprises a 2′-O-methoxyethyl (2′-MOE) modification. The addition of the 2′-O-methoxyethyl (2′-MOE) group at the cleavage site improves both the specificity and silencing activity of siRNAs by facilitating the oriented RNA-induced silencing complex (RISC) loading of the modified strand, as described in Song et al., (2017) Mol Ther Nucleic Acids 9:242-250, incorporated herein by reference in its entirety. In some embodiments, the antisense strand of the siRNA molecule comprises a 2′-OMe-phosphorodithioate modification, which increases RISC loading as described in Wu et al., (2014) Nat Commun 5:3459, incorporated herein by reference in its entirety.

In some embodiments, the sense strand of the siRNA molecule comprises a 5′-morpholino, which reduces RISC loading of the sense strand and improves antisense strand selection and RNAi activity, as described in Kumar et al., (2019) Chem Commun (Camb) 55(35):5139-5142, incorporated herein by reference in its entirety. In some embodiments, the sense strand of the siRNA molecule is modified with a synthetic RNA-like high affinity nucleotide analogue, Locked Nucleic Acid (LNA), which reduces RISC loading of the sense strand and further enhances antisense strand incorporation into RISC, as described in Elman et al., (2005) Nucleic Acids Res. 33(1): 439-447, incorporated herein by reference in its entirety. In some embodiments, the sense strand of the siRNA molecule comprises a 5′ unlocked nucleic acic (UNA) modification, which reduce RISC loading of the sense strand and improve silencing potency of the antisense strand, as described in Snead et al., (2013) Mol Ther Nucleic Acids 2(7):e103, incorporated herein by reference in its entirety. In some embodiments, the sense strand of the siRNA molecule comprises a 5-nitroindole modification, which decreased the RNAi potency of the sense strand and reduces off-target effects as described in Zhang et al., (2012) Chembiochem 13(13):1940-1945, incorporated herein by reference in its entirety. In some embodiments, the sense strand comprises a 2′-O′methyl (2′-O-Me) modification, which reduces RISC loading and the off-target effects of the sense strand, as described in Zheng et al., FASEB (2013) 27(10): 4017-4026, incorporated herein by reference in its entirety. In some embodiments, the sense strand of the siRNA molecule is fully substituted with morpholino, 2′-MOE or 2′-O-Me residues, and are not recognized by RISC as described in Kole et al., (2012) Nature reviews. Drug Discovery 11(2):125-140, incorporated herein by reference in its entirety. In some embodiments the antisense strand of the siRNA molecule comprises a 2′-MOE modification and the sense strand comprises an 2′-O-Me modification (see e.g., Song et al., (2017) Mol Ther Nucleic Acids 9:242-250),In some embodiments at least one (e.g., at least 2, at least 3, at least 4, at least 5, at least 10) siRNA molecule is linked (e.g., covalently) to a muscle-targeting agent. In some embodiments, the muscle-targeting agent may comprise, or consist of, a nucleic acid (e.g., DNA or RNA), a peptide (e.g., an antibody), a lipid (e.g., a microvesicle), or a sugar moiety (e.g., a polysaccharide). In some embodiments, the muscle-targeting agent is an antibody. In some embodiments, the muscle-targeting agent is an anti-transferrin receptor antibody (e.g., any one of the anti-TfR antibodies provided herein). In some embodiments, the muscle-targeting agent may be linked to the 5′ end of the sense strand of the siRNA molecule. In some embodiments, the muscle-targeting agent may be linked to the 3′ end of the sense strand of the siRNA molecule. In some embodiments, the muscle-targeting agent may be linked internally to the sense strand of the siRNA molecule. In some embodiments, the muscle-targeting agent may be linked to the 5′ end of the antisense strand of the siRNA molecule. In some embodiments, the muscle-targeting agent may be linked to the 3′ end of the antisense strand of the siRNA molecule. In some embodiments, the muscle-targeting agent may be linked internally to the antisense strand of the siRNA molecule.

k. microRNA (miRNAs)

In some embodiments, an oligonucleotide may be a microRNA (miRNA). MicroRNAs (referred to as “miRNAs”) are small non-coding RNAs, belonging to a class of regulatory molecules that control gene expression by binding to complementary sites on a target RNA transcript. Typically, miRNAs are generated from large RNA precursors (termed pri-miRNAs) that are processed in the nucleus into approximately 70 nucleotide pre-miRNAs, which fold into imperfect stem-loop structures. These pre-miRNAs typically undergo an additional processing step within the cytoplasm where mature miRNAs of 18-25 nucleotides in length are excised from one side of the pre-miRNA hairpin by an RNase III enzyme, Dicer.

As used herein, miRNAs including pri-miRNA, pre-miRNA, mature miRNA or fragments of variants thereof that retain the biological activity of mature miRNA. In one embodiment, the size range of the miRNA can be from 21 nucleotides to 170 nucleotides. In one embodiment the size range of the miRNA is from 70 to 170 nucleotides in length. In another embodiment, mature miRNAs of from 21 to 25 nucleotides in length can be used.

l. Aptamers

In some embodiments, oligonucleotides provided herein may be in the form of aptamers. Generally, in the context of molecular payloads, aptamer is any nucleic acid that binds specifically to a target, such as a small molecule, protein, nucleic acid in a cell. In some embodiments, the aptamer is a DNA aptamer or an RNA aptamer. In some embodiments, a nucleic acid aptamer is a single-stranded DNA or RNA (ssDNA or ssRNA). It is to be understood that a single-stranded nucleic acid aptamer may form helices and/or (e.g., and) loop structures. The nucleic acid that forms the nucleic acid aptamer may comprise naturally occurring nucleotides, modified nucleotides, naturally occurring nucleotides with hydrocarbon linkers (e.g., an alkylene) or a polyether linker (e.g., a PEG linker) inserted between one or more nucleotides, modified nucleotides with hydrocarbon or PEG linkers inserted between one or more nucleotides, or a combination of thereof. Exemplary publications and patents describing aptamers and method of producing aptamers include, e.g., Lorsch and Szostak, 1996; Jayasena, 1999; U.S. Pat. Nos. 5,270,163; 5,567,588; 5,650,275; 5,670,637; 5,683,867; 5,696,249; 5,789,157; 5,843,653; 5,864,026; 5,989,823; 6,569,630; 8,318,438 and PCT application WO 99/31275, each incorporated herein by reference.

m. Ribozymes

In some embodiments, oligonucleotides provided herein may be in the form of a ribozyme. A ribozyme (ribonucleic acid enzyme) is a molecule, typically an RNA molecule, that is capable of performing specific biochemical reactions, similar to the action of protein enzymes. Ribozymes are molecules with catalytic activities including the ability to cleave at specific phosphodiester linkages in RNA molecules to which they have hybridized, such as mRNAs, RNA-containing substrates, lncRNAs, and ribozymes, themselves.

Ribozymes may assume one of several physical structures, one of which is called a “hammerhead.” A hammerhead ribozyme is composed of a catalytic core containing nine conserved bases, a double-stranded stem and loop structure (stem-loop II), and two regions complementary to the target RNA flanking regions the catalytic core. The flanking regions enable the ribozyme to bind to the target RNA specifically by forming double-stranded stems I and III. Cleavage occurs in cis (i.e., cleavage of the same RNA molecule that contains the hammerhead motif) or in trans (cleavage of an RNA substrate other than that containing the ribozyme) next to a specific ribonucleotide triplet by a transesterification reaction from a 3′, 5′-phosphate diester to a 2′, 3′-cyclic phosphate diester. Without wishing to be bound by theory, it is believed that this catalytic activity requires the presence of specific, highly conserved sequences in the catalytic region of the ribozyme.

Modifications in ribozyme structure have also included the substitution or replacement of various non-core portions of the molecule with non-nucleotidic molecules. For example, Benseler et al. (J. Am. Chem. Soc. (1993) 115:8483-8484) disclosed hammerhead-like molecules in which two of the base pairs of stem II, and all four of the nucleotides of loop II were replaced with non-nucleoside linkers based on hexaethylene glycol, propanediol, bis(triethylene glycol) phosphate, tris(propanediol)bisphosphate, or bis(propanediol) phosphate. Ma et al. (Biochem. (1993) 32:1751-1758; Nucleic Acids Res. (1993) 21:2585-2589) replaced the six nucleotide loop of the TAR ribozyme hairpin with non-nucleotidic, ethylene glycol-related linkers. Thomson et al. (Nucleic Acids Res. (1993) 21:5600-5603) replaced loop II with linear, non-nucleotidic linkers of 13, 17, and 19 atoms in length.

Ribozyme oligonucleotides can be prepared using well known methods (see, e.g., PCT Publications WO9118624; WO9413688; WO9201806; and WO 92/07065; and U.S. Pat. Nos. 5,436,143 and 5,650,502) or can be purchased from commercial sources (e.g., US Biochemicals) and, if desired, can incorporate nucleotide analogs to increase the resistance of the oligonucleotide to degradation by nucleases in a cell. The ribozyme may be synthesized in any known manner, e.g., by use of a commercially available synthesizer produced, e.g., by Applied Biosystems, Inc. or Milligen. The ribozyme may also be produced in recombinant vectors by conventional means. See, Molecular Cloning: A Laboratory Manual, Cold Spring Harbor Laboratory (Current edition). The ribozyme RNA sequences maybe synthesized conventionally, for example, by using RNA polymerases such as T7 or SP6.

n. Guide Nucleic Acids

In some embodiments, oligonucleotides are guide nucleic acid, e.g., guide RNA (gRNA) molecules. Generally, a guide RNA is a short synthetic RNA composed of (1) a scaffold sequence that binds to a nucleic acid programmable DNA binding protein (napDNAbp), such as Cas9, and (2) a nucleotide spacer portion that defines the DNA target sequence (e.g., genomic DNA target) to which the gRNA binds in order to bring the nucleic acid programmable DNA binding protein in proximity to the DNA target sequence. In some embodiments, the napDNAbp is a nucleic acid-programmable protein that forms a complex with (e.g., binds or associates with) one or more RNA(s) that targets the nucleic acid-programmable protein to a target DNA sequence (e.g., a target genomic DNA sequence). In some embodiments, a nucleic acid-programmable nuclease, when in a complex with an RNA, may be referred to as a nuclease:RNA complex. Guide RNAs can exist as a complex of two or more RNAs, or as a single RNA molecule.

Guide RNAs (gRNAs) that exist as a single RNA molecule may be referred to as single-guide RNAs (sgRNAs), though gRNA is also used to refer to guide RNAs that exist as either single molecules or as a complex of two or more molecules. Typically, gRNAs that exist as a single RNA species comprise two domains: (1) a domain that shares homology to a target nucleic acid (i.e., directs binding of a Cas9 complex to the target); and (2) a domain that binds a Cas9 protein. In some embodiments, domain (2) corresponds to a sequence known as a tracrRNA and comprises a stem-loop structure. In some embodiments, domain (2) is identical or homologous to a tracrRNA as provided in Jinek et al., Science 337:816-821 (2012), the entire contents of which is incorporated herein by reference.

In some embodiments, a gRNA comprises two or more of domains (1) and (2), and may be referred to as an extended gRNA. For example, an extended gRNA will bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example, Cas9 (Csn1) from Streptococcus pyogenes (see, e.g., “Complete genome sequence of an M1 strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference.

o. Multimers

In some embodiments, molecular payloads may comprise multimers (e.g., concatemers) of 2 or more oligonucleotides connected by a linker. In this way, in some embodiments, the oligonucleotide loading of a complex/conjugate can be increased beyond the available linking sites on a targeting agent (e.g., available thiol sites or amine sites on an antibody) or otherwise tuned to achieve a particular payload loading content. Oligonucleotides in a multimer can be the same or different (e.g., targeting different genes or different sites on the same gene or products thereof).

In some embodiments, multimers comprise 2 or more oligonucleotides linked together by a cleavable linker. However, in some embodiments, multimers comprise 2 or more oligonucleotides linked together by a non-cleavable linker. In some embodiments, a multimer comprises 2, 3, 4, 5, 6, 7, 8, 9, 10 or more oligonucleotides linked together. In some embodiments, a multimer comprises 2 to 5, 2 to 10 or 4 to 20 oligonucleotides linked together.

In some embodiments, a multimer comprises 2 or more oligonucleotides linked end-to-end (in a linear arrangement). In some embodiments, a multimer comprises 2 or more oligonucleotides linked end-to-end via a oligonucleotide based linker (e.g., poly-dT linker, an abasic linker). In some embodiments, a multimer comprises a 5′ end of one oligonucleotide linked to a 3′ end of another oligonucleotide. In some embodiments, a multimer comprises a 3′ end of one oligonucleotide linked to a 3′ end of another oligonucleotide. In some embodiments, a multimer comprises a 5′ end of one oligonucleotide linked to a 5′ end of another oligonucleotide. Still, in some embodiments, multimers can comprise a branched structure comprising multiple oligonucleotides linked together by a branching linker.

Further examples of multimers that may be used in the complexes provided herein are disclosed, for example, in US Patent Application Number 2015/0315588 A1, entitled Methods of delivering multiple targeting oligonucleotides to a cell using cleavable linkers, which was published on Nov. 5, 2015; US Patent Application Number 2015/0247141 A1, entitled Multimeric Oligonucleotide Compounds, which was published on Sep. 3, 2015, US Patent Application Number US 2011/0158937 A1, entitled Immunostimulatory Oligonucleotide Multimers, which was published on Jun. 30, 2011; and U.S. Pat. No. 5,693,773, entitled Triplex-Forming Antisense Oligonucleotides Having Abasic Linkers Targeting Nucleic Acids Comprising Mixed Sequences Of Purines And Pyrimidines, which issued on Dec. 2, 1997, the contents of each of which are incorporated herein by reference in their entireties.

o. Splice Altering Oligonucleotides

In some embodiments, a oligonucleotide (e.g., an antisense oligonucleotide including a morpholino) of the present disclosure target splicing. In some embodiments, the oligonucleotide targets splicing by inducing exon skipping and restoring the reading frame within a gene. As a non-limiting example, the oligonucleotide may induce skipping of an exon encoding a frameshift mutation and/or (e.g., and) an exon that encodes a premature stop codon. In some embodiments, an oligonucleotide may induce exon skipping by blocking spliceosome recognition of a splice site. In some embodiments, exon skipping results in a truncated but functional protein compared to the reference protein (e.g., truncated but functional DMD protein as described below). In some embodiments, the oligonucleotide promotes inclusion of a particular exon (e.g., exon 7 of the SMN2 gene described below). In some embodiments, an oligonucleotide may induce inclusion of an exon by targeting a splice site inhibitory sequence. RNA splicing has been implicated in muscle diseases, including Duchenne muscular dystrophy (DMD) and spinal muscular atrophy (SMA).

Alterations (e.g., deletions, point mutations, and duplications) in the gene encoding dystrophin (DMD) cause DMD. These alterations can lead to frameshift mutations and/or (e.g., and) nonsense mutations. In some embodiments, an oligonucleotide of the present disclosure promotes skipping of one or more DMD exons (e.g., exon 8, exon 43, exon 44, exon 45, exon 50, exon 51, exon 52, exon 53, and/or (e.g., and) exon 55) and results in a functional truncated protein. See, e.g., U.S. Pat. No. 8,486,907 published on Jul. 16, 2013 and U.S. 20140275212 published on Sep. 18, 2014.

In SMA, there is loss of functional SMN1. Although the SMN2 gene is a paralog to SMN1, alternative splicing of the SMN2 gene predominantly leads to skipping of exon 7 and subsequent production of a truncated SMN protein that cannot compensate for SMN1 loss. In some embodiments, an oligonucleotide of the present disclosure promotes inclusion of SMN2 exon 7. In some embodiments, an oligonucleotide is an antisense oligonucleotide that targets SMN2 splice site inhibitory sequences (see, e.g., U.S. Pat. No. 7,838,657, which was published on Nov. 23, 2010).

ii. Small Molecules:

Any suitable small molecule may be used as a molecular payload, as described herein.

iii. Peptides/Proteins

Any suitable peptide or protein may be used as a molecular payload, as described herein. In some embodiments, a protein is an enzyme (e.g., an acid alpha-glucosidase, e.g., as encoded by the GAA gene). These peptides or proteins may be produced, synthesized, and/or (e.g., and) derivatized using several methodologies, e.g. phage displayed peptide libraries, one-bead one-compound peptide libraries, or positional scanning synthetic peptide combinatorial libraries. Exemplary methodologies have been characterized in the art and are incorporated by reference (Gray, B. P. and Brown, K. C. “Combinatorial Peptide Libraries: Mining for Cell-Binding Peptides” Chem Rev. 2014, 114:2, 1020-1081; Samoylova, T. I. and Smith, B. F. “Elucidation of muscle-binding peptides by phage display screening.” Muscle Nerve, 1999, 22:4. 460-6.).

iv. Nucleic Acid Constructs

Any suitable gene expression construct may be used as a molecular payload, as described herein. In some embodiments, a gene expression construct may be a vector or a cDNA fragment. In some embodiments, a gene expression construct may be messenger RNA (mRNA). In some embodiments, a mRNA used herein may be a modified mRNA, e.g., as described in U.S. Pat. No. 8,710,200, issued on Apr. 24, 2014, entitled “Engineered nucleic acids encoding a modified erythropoietin and their expression”. In some embodiments, a mRNA may comprise a 5′ methyl cap. In some embodiments, a mRNA may comprise a polyA tail, optionally of up to 160 nucleotides in length. A gene expression construct may encode a sequence of a protein that is deficient in a muscle disease. In some embodiments, the gene expression construct may be expressed, e.g., overexpressed, within the nucleus of a muscle cell. In some embodiments, the gene expression construct encodes a gene that is deficient in a muscle disease. In some embodiments, the gene expression constructs encodes a protein that comprises at least one zinc finger. In some embodiments, the gene expression construct encodes a protein that binds to a gene in Table 7. In some embodiments, the gene expression construct encodes a protein that leads to a reduction in the expression of a protein (e.g., mutant protein) encoded by a gene in Table 7. In some embodiments, the gene expression construct encodes a gene editing enzyme. Additional examples of nucleic acid constructs that may be used as molecular payloads are provided in International Patent Application Publication WO2017152149A1, published on Sep. 19, 2017, entitled, “CLOSED-ENDED LINEAR DUPLEX DNA FOR NON-VIRAL GENE TRANSFER”; U.S. Pat. No. 8,853,377B2, issued on Oct. 7, 2014, entitled, “MRNA FOR USE IN TREATMENT OF HUMAN GENETIC DISEASES”; and US Patent U.S. Pat. No. 8,822,663B2, issued on Sep. 2, 2014, ENGINEERED NUCLEIC ACIDS AND METHODS OF USE THEREOF,” the contents of each of which are incorporated herein by reference in their entireties.

v. Detectable Labels/Diagnostic Agents

Any suitable detectable label or diagnostic agent can be used as the molecular payload of the present disclosure. A “diagnostic agent” refers to an agent that is used for diagnostic purpose, e.g., by detecting another molecule in a cell or a tissue. In some embodiments, the diagnostic agent is an agent that targets (e.g., binds) a biomarker known to be associated with a disease (e.g., a nucleic acid biomarker, protein biomarker, or a metabolite biomarker) in a subject and produces a detectable signal, which can be used to determine the presence/absence of the biomarker, thus to diagnose a disease. For example, the diagnostic agent may be, without limitation, an antibody or an antisense nucleic acid.

In some embodiments, the diagnostic agent contains a detectable label. A detectable label refers to a moiety that has at least one element, isotope, or a structural or functional group incorporated that enables detection of a molecule, e.g., a protein or polypeptide, or other entity, to which the diagnostic agent binds. In some embodiments, a detectable label falls into any one (or more) of five classes: a) an agent which contains isotopic moieties, which may be radioactive or heavy isotopes, including, but not limited to, 2H, 3H, 13C, 14C, 15N, 18F, 31P, 32P, 35S, 67Ga, 76Br, 99mTc (Tc-99m), 111In, 123I, 125I, 131I, 153Gd, 169Yb, and 186Re; b) an agent which contains an immune moiety, which may be an antibody or antigen, which may be bound to an enzyme (e.g., such as horseradish peroxidase); c) an agent comprising a colored, luminescent, phosphorescent, or fluorescent moiety (e.g., such as the fluorescent label fluorescein isothiocyanate (FITC); d) an agent which has one or more photo affinity moieties; and e) an agent which is a ligand for one or more known binding partners (e.g., biotin-streptavidin, His-NiTNAFK506-FKBP). In some embodiments, a detectable label comprises a radioactive isotope. In some embodiments, a detectable label comprises a fluorescent moiety. In some embodiments, the detectable label comprises a dye, e.g., a fluorescent dye, e.g., fluorescein isothiocyanate, Texas red, rhodamine, Cy3, Cy5, Cy5.5, Alexa 647 and derivatives. In some embodiments, the detectable label comprises biotin. In some embodiments, the detectable molecule is a fluorescent polypeptide (e.g., GFP or a derivative thereof such as enhanced GFP (EGFP)) or a luciferase (e.g., a firefly, Renilla, or Gaussia luciferase). In some embodiments, a detectable label may react with a suitable substrate (e.g., a luciferin) to generate a detectable signal. Non-limiting examples of fluorescent proteins include GFP and derivatives thereof, proteins comprising chromophores that emit light of different colors such as red, yellow, and cyan fluorescent proteins, etc. Exemplary fluorescent proteins include, e.g., Sirius, Azurite, EBFP2, TagBFP, mTurquoise, ECFP, Cerulean, TagCFP, mTFP1, mUkG1, mAG1, AcGFP1, TagGFP2, EGFP, mWasabi, EmGFP, TagYPF, EYFP, Topaz, SYFP2, Venus, Citrine, mKO, mKO2, mOrange, mOrange2, TagRFP, TagRFP-T, mStrawberry, mRuby, mCherry, mRaspberry, mKate2, mPlum, mNeptune, T-Sapphire, mAmetrine, mKeima. See, e.g., Chalfie, M. and Kain, S R (eds.) Green fluorescent protein: properties, applications, and protocols (Methods of biochemical analysis, v. 47, Wiley-Interscience, and Hoboken, N.J., 2006, and/or (e.g., and) Chudakov, D M, et al., Physiol Rev. 90(3):1103-63, 2010, incorporated herein by reference, for discussion of GFP and numerous other fluorescent or luminescent proteins. In some embodiments, a detectable label comprises a dark quencher, e.g., a substance that absorbs excitation energy from a fluorophore and dissipates the energy as heat.

Further examples of complexes and molecular payloads (e.g., oligonucleotides useful for targeting muscle genes) are provided in International Patent Application Publication WO2020/028861, published on Feb. 6, 2020, entitled, “MUSCLE TARGETING COMPLEXES AND USES THEREOF FOR TREATING MYOTONIC DYSTROPHY”; International Patent Application Publication WO2020/028864, published on Feb. 6, 2020, entitled, “MUSCLE TARGETING COMPLEXES AND USES THEREOF FOR TREATING FACIOSCAPULOHUMERAL MUSCULAR DYSTROPHY”; International Patent Application Publication WO2020/028844, published on Feb. 6, 2020, entitled, “MUSCLE TARGETING COMPLEXES AND USES THEREOF FOR TREATING CENTRONUCLEAR MYOPATHY”; International Patent Application Publication WO2020/028841, published on Feb. 6, 2020, entitled, “MUSCLE TARGETING COMPLEXES AND USES THEREOF FOR TREATING POMPE DISEASE”; International Patent Application Publication WO2020/028831, published on Feb. 6, 2020, entitled, “MUSCLE TARGETING COMPLEXES AND USES THEREOF FOR TREATING FIBRODYSPLASIA OSSIFICANS PROGRESSIVA”; International Patent Application Publication WO2020/028840, published on Feb. 6, 2020, entitled, “MUSCLE TARGETING COMPLEXES AND USES THEREOF FOR TREATING FRIEDREICH'S ATAXIA”; International Patent Application Publication WO2020/028857, published on Feb. 6, 2020, entitled, “MUSCLE-TARGETING COMPLEXES AND USES THEREOF”; International Patent Application Publication WO2020/028836, published on Feb. 6, 2020, entitled, “MUSCLE-TARGETING COMPLEXES AND USES THEREOF IN TREATING MUSCLE ATROPHY”; International Patent Application Publication WO2020/028832, published on Feb. 6, 2020, entitled, “MUSCLE TARGETING COMPLEXES AND USES THEREOF FOR TREATING DYSTROPHINOPATHIES”; International Patent Application Publication WO2020/028842, published on Feb. 6, 2020, entitled, “MUSCLE TARGETING COMPLEXES AND USES THEREOF FOR TREATING HYPERTROPHIC CARDIOMYOPATHY”; the contents of each of which are incorporated herein by reference.

B. Linkers

Complexes described herein generally comprise a linker that connects any one of the anti-TfR antibodies described herein to a molecular payload. A linker comprises at least one covalent bond. In some embodiments, a linker may be a single bond, e.g., a disulfide bond or disulfide bridge, that connects an anti-TfR antibody to a molecular payload. However, in some embodiments, a linker may connect any one of the anti-TfR antibodies described herein to a molecular through multiple covalent bonds. In some embodiments, a linker may be a cleavable linker. However, in some embodiments, a linker may be a non-cleavable linker. A linker is generally stable in vitro and in vivo, and may be stable in certain cellular environments. Additionally, generally a linker does not negatively impact the functional properties of either the anti-TfR antibody or the molecular payload. Examples and methods of synthesis of linkers are known in the art (see, e.g. Kline, T. et al. “Methods to Make Homogenous Antibody Drug Conjugates.” Pharmaceutical Research, 2015, 32:11, 3480-3493; Jain, N. et al. “Current ADC Linker Chemistry” Pharm Res. 2015, 32:11, 3526-3540; McCombs, J. R. and Owen, S. C. “Antibody Drug Conjugates: Design and Selection of Linker, Payload and Conjugation Chemistry” AAPS J. 2015, 17:2, 339-351.).

A precursor to a linker typically will contain two different reactive species that allow for attachment to both the anti-TfR antibody and a molecular payload. In some embodiments, the two different reactive species may be a nucleophile and/or (e.g., and) an electrophile. In some embodiments, a linker is connected to an anti-TfR antibody via conjugation to a lysine residue or a cysteine residue of the anti-TfR antibody. In some embodiments, a linker is connected to a cysteine residue of an anti-TfR antibody via a maleimide-containing linker, wherein optionally the maleimide-containing linker comprises a maleimidocaproyl or maleimidomethyl cyclohexane-1-carboxylate group. In some embodiments, a linker is connected to a cysteine residue of an anti-TfR antibody or thiol functionalized molecular payload via a 3-arylpropionitrile functional group. In some embodiments, a linker is connected to a lysine residue of an anti-TfR antibody. In some embodiments, a linker is connected to an anti-TfR antibody and/or (e.g., and) a molecular payload via an amide bond, a carbamate bond, a hydrazide, a trizaole, a thioether, or a disulfide bond.

i. Cleavable Linkers

A cleavable linker may be a protease-sensitive linker, a pH-sensitive linker, or a glutathione-sensitive linker. These linkers are generally cleavable only intracellularly and are preferably stable in extracellular environments, e.g. extracellular to a muscle cell.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include β-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline dipeptide sequence. In some embodiments, a protease-sensitive linker can be cleaved by a lysosomal protease, e.g. cathepsin B, and/or (e.g., and) an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by an disulfide exchange reaction with a glutathione species inside a cell. In some embodiments, the disulfide moiety further comprises at least one amino acid, e.g. a cysteine residue.

In some embodiments, the linker is a Val-cit linker (e.g., as described in U.S. Pat. No. 6,214,345, incorporated herein by reference). In some embodiments, before conjugation, the val-cit linker has a structure of:

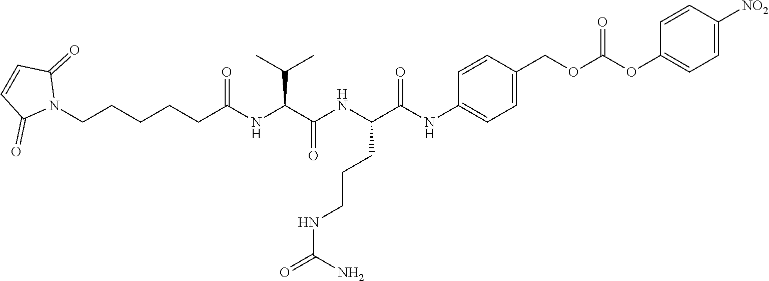 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

In some embodiments, after conjugation, the val-cit linker has a structure of:

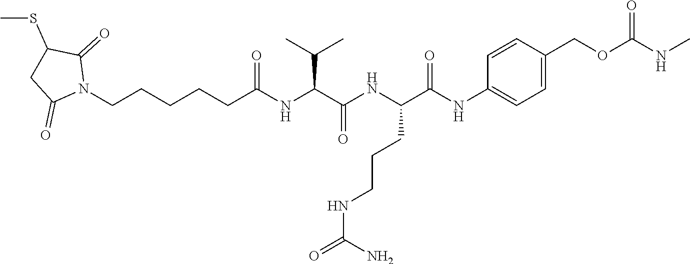

In some embodiments, the Val-cit linker is attached to a reactive chemical moiety (e.g., SPAAC for click chemistry conjugation). In some embodiments, before conjugation, the val-cit linker attached to a reactive chemical moiety (e.g., SPAAC for click chemistry conjugation) has the structure of

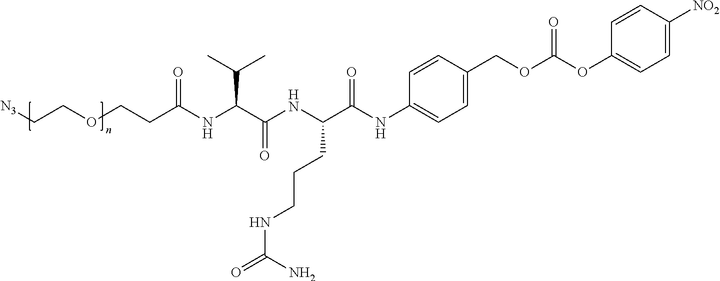

wherein n is any number from 0-10. In some embodiments, n is 3.

In some embodiments, the val-cit linker attached to a reactive chemical moiety (e.g., SPAAC for click chemistry conjugation) is conjugated (e.g., via a different chemical moiety) to a molecular payload (e.g., an oligonucleotide). In some embodiments, the val-cit linker attached to a reactive chemical moiety (e.g., SPAAC for click chemistry conjugation) and is conjugated to a molecular payload (e.g., an oligonucleotide) has the structure of (before click chemistry conjugation):

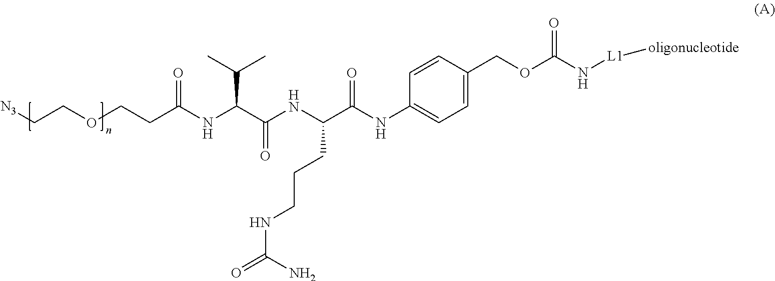

wherein n is any number from 0-10. In some embodiments, n is 3.

In some embodiments, after conjugation to a molecular payload (e.g., an oligonucleotide) and, the val-cit linker has a structure of:

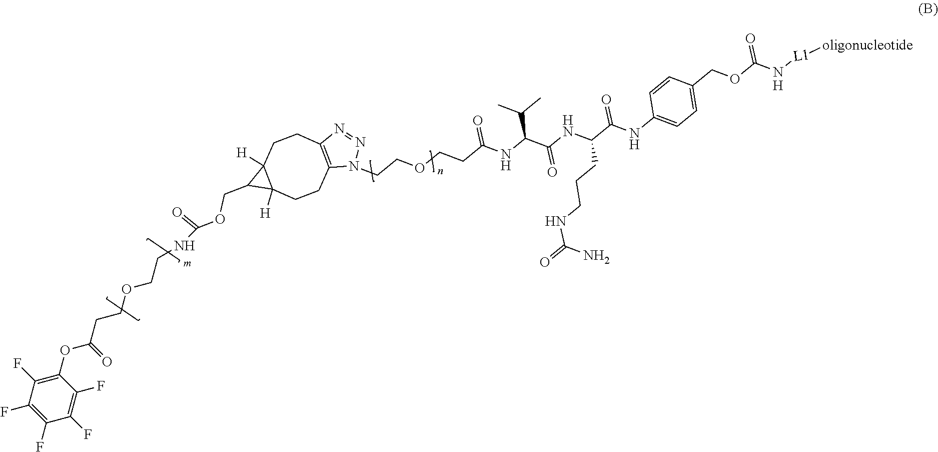

wherein n is any number from 0-10, and wherein m is any number from 0-10. In some embodiments, n is 3 and m is 4.

ii. Non-Cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyne-azide, a peptide sequence comprising a LPXTG sequence (SEQ ID NO: 235), a thioether, a biotin, a biphenyl, repeating units of polyethylene glycol or equivalent compounds, acid esters, acid amides, sulfamides, and/or (e.g., and) an alkoxy-amine linker. In some embodiments, sortase-mediated ligation will be utilized to covalently link an anti-TfR antibody comprising a LPXTG sequence (SEQ ID NO: 235) to a molecular payload comprising a (G)ₙ sequence (see, e.g. Proft T. Sortase-mediated protein ligation: an emerging biotechnology tool for protein modification and immobilization. Biotechnol Lett. 2010, 32(1):1-10.).

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S; an optionally substituted heterocyclylene further comprising at least one heteroatom selected from N, O, and S; an imino, an optionally substituted nitrogen species, an optionally substituted oxygen species O, an optionally substituted sulfur species, or a poly(alkylene oxide), e.g. polyethylene oxide or polypropylene oxide.

iii. Linker Conjugation

In some embodiments, a linker is connected to an anti-TfR antibody and/or (e.g., and) molecular payload via a phosphate, thioether, ether, carbamate, carbon-carbon, or amide bond. In some embodiments, a linker is connected to an oligonucleotide through a phosphate or phosphorothioate group, e.g. a terminal phosphate of an oligonucleotide backbone. In some embodiments, a linker is connected to an anti-TfR antibody, through a lysine or cysteine residue present on the anti-TfR antibody.

In some embodiments, a linker is connected to an anti-TfR antibody and/or (e.g., and) molecular payload by a cycloaddition reaction between an azide and an alkyne to form a triazole, wherein the azide and the alkyne may be located on the anti-TfR antibody, molecular payload, or the linker. In some embodiments, an alkyne may be a cyclic alkyne, e.g., a cyclooctyne. In some embodiments, an alkyne may be bicyclononyne (also known as bicyclo[6.1.0]nonyne or BCN) or substituted bicyclononyne. In some embodiments, a cyclooctane is as described in International Patent Application Publication WO2011136645, published on Nov. 3, 2011, entitled, “Fused Cyclooctyne Compounds And Their Use In Metal free Click Reactions”. In some embodiments, an azide may be a sugar or carbohydrate molecule that comprises an azide. In some embodiments, an azide may be 6-azido-6-deoxygalactose or 6-azido-N-acetylgalactosamine. In some embodiments, a sugar or carbohydrate molecule that comprises an azide is as described in International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”. In some embodiments, a cycloaddition reaction between an azide and an alkyne to form a triazole, wherein the azide and the alkyne may be located on the anti-TfR antibody, molecular payload, or the linker is as described in International Patent Application Publication WO2014065661, published on May 1, 2014, entitled, “Modified antibody, antibody-conjugate and process for the preparation thereof”; or International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”.

In some embodiments, a linker further comprises a spacer, e.g., a polyethylene glycol spacer or an acyl/carbomoyl sulfamide spacer, e.g., a HydraSpace™ spacer. In some embodiments, a spacer is as described in Verkade, J. M. M. et al., “A Polar Sulfamide Spacer Significantly Enhances the Manufacturability, Stability, and Therapeutic Index of Antibody-Drug Conjugates”, Antibodies, 2018, 7, 12.

In some embodiments, a linker is connected to an anti-TfR antibody and/or (e.g., and) molecular payload by the Diels-Alder reaction between a dienophile and a diene/hetero-diene, wherein the dienophile and the diene/hetero-diene may be located on the anti-TfR antibody, molecular payload, or the linker. In some embodiments a linker is connected to an anti-TfR antibody and/or (e.g., and) molecular payload by other pericyclic reactions, e.g. ene reaction. In some embodiments, a linker is connected to an anti-TfR antibody and/or (e.g., and) molecular payload by an amide, thioamide, or sulfonamide bond reaction. In some embodiments, a linker is connected to an anti-TfR antibody and/or (e.g., and) molecular payload by a condensation reaction to form an oxime, hydrazone, or semicarbazide group existing between the linker and the anti-TfR antibody and/or (e.g., and) molecular payload.

In some embodiments, a linker is connected to an anti-TfR antibody and/or (e.g., and) molecular payload by a conjugate addition reactions between a nucleophile, e.g. an amine or a hydroxyl group, and an electrophile, e.g. a carboxylic acid or an aldehyde. In some embodiments, a nucleophile may exist on a linker and an electrophile may exist on an anti-TfR antibody or molecular payload prior to a reaction between a linker and an anti-TfR antibody or molecular payload. In some embodiments, an electrophile may exist on a linker and a nucleophile may exist on an anti-TfR antibody or molecular payload prior to a reaction between a linker and an anti-TfR antibody or molecular payload. In some embodiments, an electrophile may be an azide, a pentafluorophenyl, a silicon centers, a carbonyl, a carboxylic acid, an anhydride, an isocyanate, a thioisocyanate, a succinimidyl ester, a sulfosuccinimidyl ester, a maleimide, an alkyl halide, an alkyl pseudohalide, an epoxide, an episulfide, an aziridine, an aryl, an activated phosphorus center, and/or (e.g., and) an activated sulfur center. In some embodiments, a nucleophile may be an optionally substituted alkene, an optionally substituted alkyne, an optionally substituted aryl, an optionally substituted heterocyclyl, a hydroxyl group, an amino group, an alkylamino group, an anilido group, or a thiol group.

In some embodiments, the val-cit linker attached to a reactive chemical moiety (e.g., SPAAC for click chemistry conjugation) is conjugated to the anti-TfR antibody by a structure of:

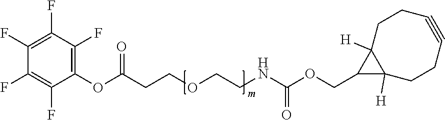

wherein m is any number from 0-10. In some embodiments, m is 4. In some embodiments, the val-cit linker attached to a reactive chemical moiety (e.g., SPAAC for click chemistry conjugation) is conjugated to an anti-TfR antibody having a structure of:

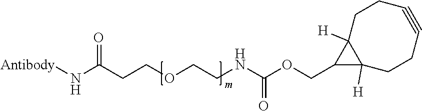

wherein m is any number from 0-10. In some embodiments, m is 4.

In some embodiments, the val-cit linker attached to a reactive chemical moiety (e.g., SPAAC for click chemistry conjugation) and is conjugated to an anti-TfR antibody has a structure of:

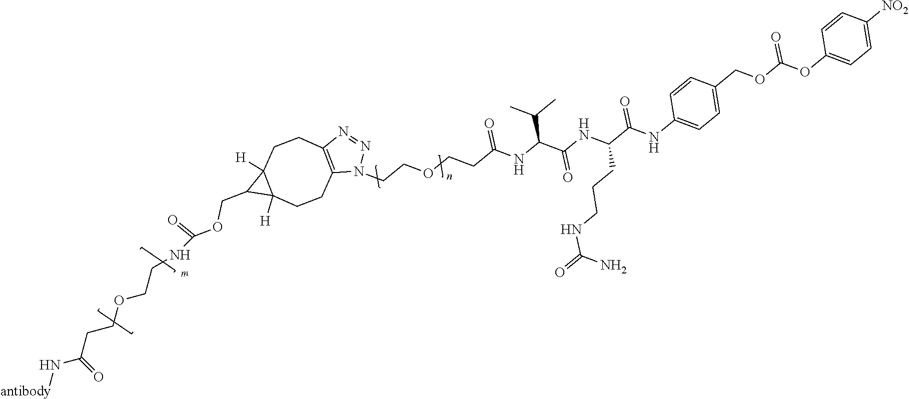

wherein n is any number from 0-10, wherein m is any number from 0-10. In some embodiments, n is 3 and/or (e.g., and) m is 4.

In some embodiments, an anti-TfR antibody and a molecular payload (e.g., an oligonucleotide) is linked via a structure of:

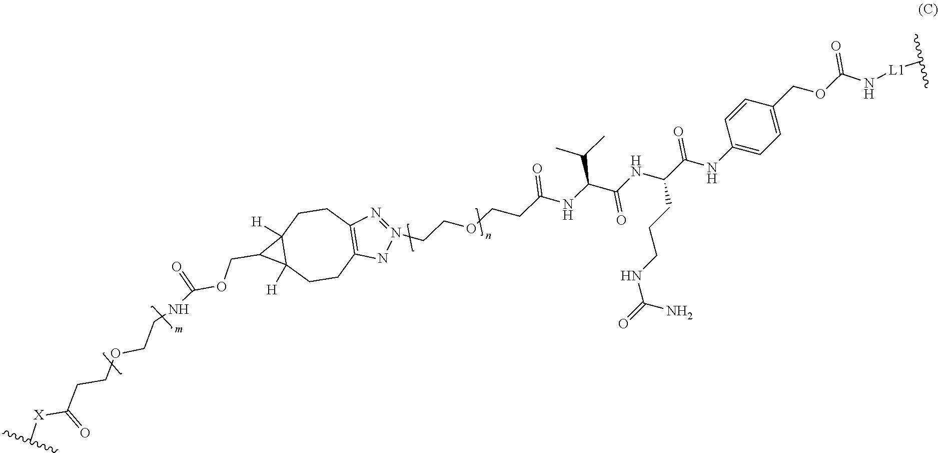

wherein n is any number from 0-10, wherein m is any number from 0-10. In some embodiments, n is 3 and/or (e.g., and) m is 4. In some embodiments, X is NH (e.g., NH from an amine group of a lysine). In some embodiments, X is S and the antibody is linked via conjugation to a cysteine of the antibody. In some embodiments, X is 0 and the antibody is linked via conjugation to a hydroxyl group of a serine, threonine, or tyrosine of the antibody.

In some embodiments, the complex described herein has a structure of:

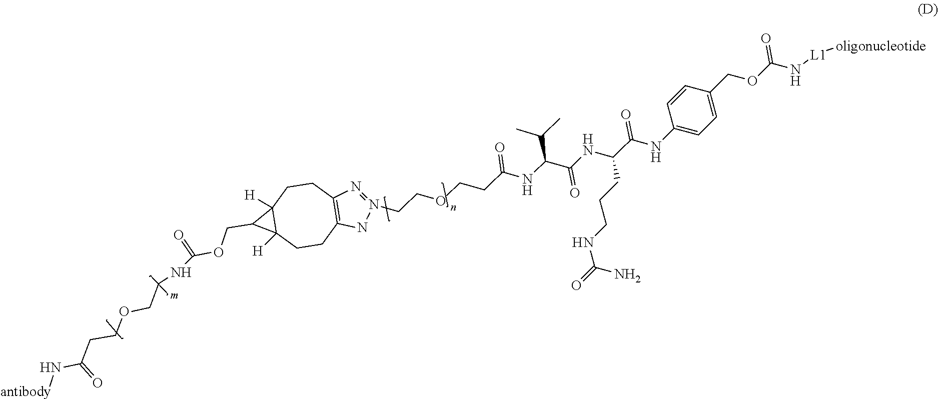

wherein n is any number from 0-10, wherein m is any number from 0-10. In some embodiments, n is 3 and/or (e.g., and) m is 4.

In structures formula (A), (B), (C), and (D), L1 is, in some embodiments, is a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof. In some embodiments, L1 is

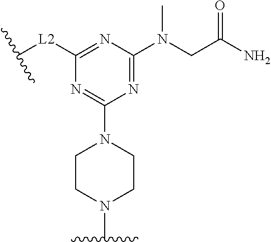

wherein the piperazine moiety links to the oligonucleotide, wherein L2 is

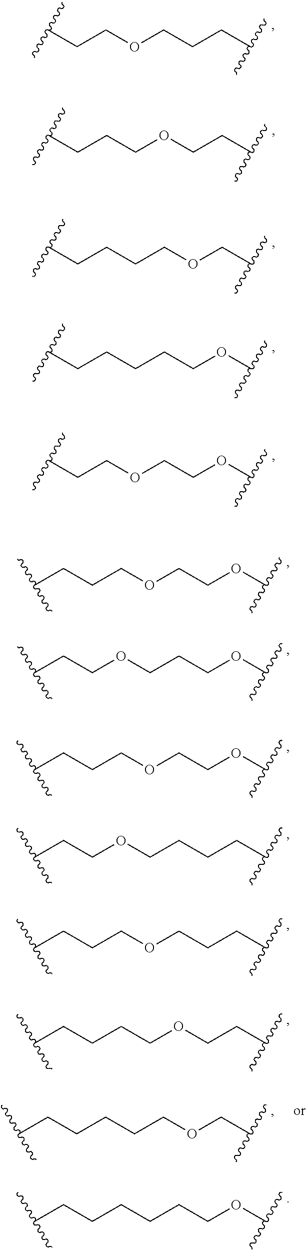

In some embodiments, L1 is:

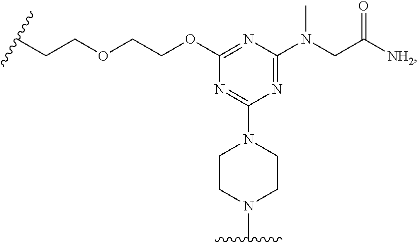 [SMILES: *CCOCCOc1nc(N(C)CC(N)=O)nc(N2CCN(*)CC2)n1]

wherein the piperazine moiety links to the oligonucleotide.

In some embodiments, L1 is

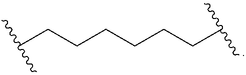 [SMILES: *CCCCCC*]

In some embodiments, L1 is linked to the 5′ phosphate of the oligonucleotide. In some embodiments, L1 is linked to the 5′ phosphorothioate of the oligonucleotide. In some embodiments, L1 is linked to the 5′ phosphonoamidate of the oligonucleotide.

In some embodiments, L1 is optional (e.g., need not be present).

C. Examples of Antibody-Molecular Payload Complexes

Further provided herein are non-limiting examples of complexes comprising any one the anti-TfR antibodies described herein covalently linked to any of the molecular payloads (e.g., an oligonucleotide) described herein. In some embodiments, the anti-TfR antibody (e.g., any one of the anti-TfR antibody provided in Table 1) is covalently linked to a molecular payload (e.g., an oligonucleotide) via a linker. Any of the linkers described herein may be used. In some embodiments, if the molecular payload is an oligonucleotide, the linker is linked to the 5′ end, the 3′ end, or internally of the oligonucleotide. In some embodiments, the linker is linked to the anti-TfR antibody via a thiol-reactive linkage (e.g., via a cysteine in the anti-TfR antibody). In some embodiments, the linker (e.g., a Val-cit linker) is linked to the antibody (e.g., an anti-TfR antibody described herein) via a n amine group (e.g., via a lysine in the antibody).

An example of a structure of a complex comprising an anti-TfR antibody covalently linked to a molecular payload via a Val-cit linker is provided below:

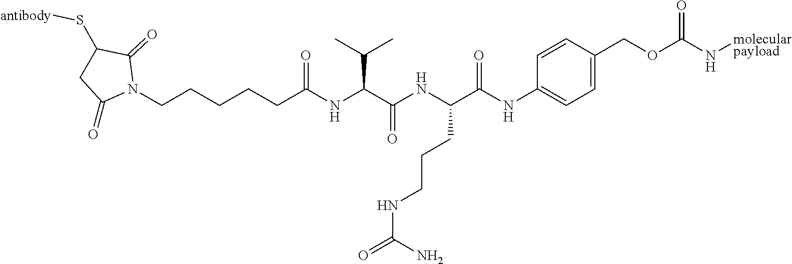

wherein the linker is linked to the antibody via a thiol-reactive linkage (e.g., via a cysteine in the antibody).

Another example of a structure of a complex comprising an anti-TfR antibody covalently linked to a molecular payload via a Val-cit linker is provided below:

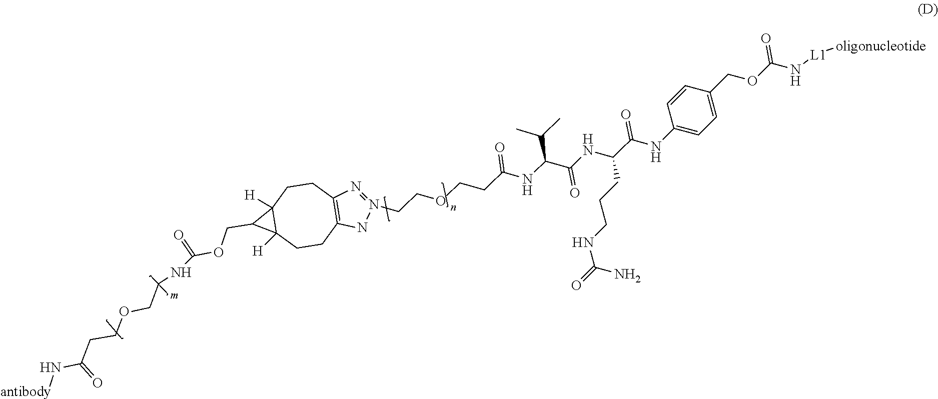

wherein n is a number between 0-10, wherein m is a number between 0-10, wherein the linker is linked to the antibody via an amine group (e.g., on a lysine residue), and/or (e.g., and) wherein the linker is linked to the oligonucleotide (e.g., at the 5′ end, 3′ end, or internally). In some embodiments, the linker is linked to the antibody via a lysine, the linker is linked to the oligonucleotide at the 5′ end, n is 3, and m is 4. In some embodiments, the molecular payload is an oligonucleotide comprising a sense strand and an antisense strand, and, the linker is linked to the sense strand or the antisense strand at the 5′ end or the 3′ end.

It should be appreciated that antibodies can be linked to molecular payloads with different stochiometries, a property that may be referred to as a drug to antibody ratios (DAR) with the “drug” being the molecular payload. In some embodiments, one molecular payload is linked to an antibody (DAR=1). In some embodiments, two molecular payloads are linked to an antibody (DAR=2). In some embodiments, three molecular payloads are linked to an antibody (DAR=3). In some embodiments, four molecular payloads are linked to an antibody (DAR=4). In some embodiments, a mixture of different complexes, each having a different DAR, is provided. In some embodiments, an average DAR of complexes in such a mixture may be in a range of 1 to 3, 1 to 4, 1 to 5 or more. DAR may be increased by conjugating molecular payloads to different sites on an antibody and/or (e.g., and) by conjugating multimers to one or more sites on antibody. For example, a DAR of 2 may be achieved by conjugating a single molecular payload to two different sites on an antibody or by conjugating a dimer molecular payload to a single site of an antibody.

In some embodiments, the complex described herein comprises an anti-TfR antibody described herein (e.g., the 3-A4, 3-M12, and 5-H12 antibodies provided in Table 1 in a IgG or FAB form) covalently linked to a molecular payload. In some embodiments, the complex described herein comprises an anti-TfR antibody described herein (e.g., the 3-A4, 3-M12, and 5-H12 antibodies provided in Table 1 in a IgG or FAB form) covalently linked to molecular payload via a linker (e.g., a Val-cit linker). In some embodiments, the linker (e.g., a Val-cit linker) is linked to the antibody (e.g., an anti-TfR antibody described herein) via a thiol-reactive linkage (e.g., via a cysteine in the antibody). In some embodiments, the linker (e.g., a Val-cit linker) is linked to the antibody (e.g., an anti-TfR antibody described herein) via a n amine group (e.g., via a lysine in the antibody).

In some embodiments, the complex described herein comprises an anti-TfR antibody covalently linked to a molecular payload, wherein the anti-TfR antibody comprises a CDR-H1, a CDR-H2, and a CDR-H3 that are the same as the CDR-H1, CDR-H2, and CDR-H3 shown in Table 3; and a CDR-L1, a CDR-L2, and a CDR-L3 that are the same as the CDR-L1, CDR-L2, and CDR-L3 shown in Table 3.

In some embodiments, the complex described herein comprises an anti-TfR antibody covalently linked to a molecular payload, wherein the anti-TfR antibody comprises a VH having the amino acid sequence of SEQ ID NO: 7 and a VL having the amino acid sequence of SEQ ID NO: 8. In some embodiments, the molecular payload is an oligonucleotide.

In some embodiments, the complex described herein comprises an anti-TfR antibody covalently linked to a molecular payload, wherein the anti-TfR antibody comprises a VH having the amino acid sequence of SEQ ID NO: 7 with the N at position 55 being substituted with T or S, and a VL having the amino acid sequence of SEQ ID NO: 8. In some embodiments, the molecular payload is an oligonucleotide.

In some embodiments, the complex described herein comprises an anti-TfR antibody covalently linked to a molecular payload, wherein the anti-TfR antibody comprises a VH having the amino acid sequence of SEQ ID NO: 15 and a VL having the amino acid sequence of SEQ ID NO: 16. In some embodiments, the molecular payload is an oligonucleotide.

In some embodiments, the complex described herein comprises an anti-TfR antibody covalently linked to a molecular payload, wherein the anti-TfR antibody comprises a VH having the amino acid sequence of SEQ ID NO: 23 and a VL having the amino acid sequence of SEQ ID NO: 24. In some embodiments, the molecular payload is an oligonucleotide.

In some embodiments, the complex described herein comprises an anti-TfR antibody covalently linked to a molecular payload, wherein the anti-TfR antibody comprises a VH having the amino acid sequence of SEQ ID NO: 23 with the C at position 33 being substituted with Y or D, and a VL having the amino acid sequence of SEQ ID NO: 24. In some embodiments, the molecular payload is an oligonucleotide.

In some embodiments, the complex described herein comprises an anti-TfR antibody covalently linked to a molecular payload, wherein the anti-TfR antibody comprises a heavy chain having the amino acid sequence of SEQ ID NO: 178, SEQ ID NO: 185, SEQ ID NO: 269, SEQ ID NO: 270, SEQ ID NO: 273, or SEQ ID NO: 274, and a light chain having the amino acid sequence of SEQ ID NO: 179. In some embodiments, the molecular payload is an oligonucleotide.

In some embodiments, the complex described herein comprises an anti-TfR antibody covalently linked to a molecular payload, wherein the anti-TfR antibody comprises a heavy chain having the amino acid sequence of SEQ ID NO: 180 or SEQ ID NO: 186, and a light chain having the amino acid sequence of SEQ ID NO: 181. In some embodiments, the molecular payload is an oligonucleotide.

In some embodiments, the complex described herein comprises an anti-TfR antibody covalently linked to a molecular payload, wherein the anti-TfR antibody comprises a heavy chain having the amino acid sequence of SEQ ID NO: 182, SEQ ID NO: 187, SEQ ID NO: 271, SEQ ID NO: 272, SEQ ID NO: 275, or SEQ ID NO: 276, and a light chain having the amino acid sequence of SEQ ID NO: 183. In some embodiments, the molecular payload is an oligonucleotide.

In some embodiments, the complex described herein comprises a humanized anti-TfR antibody described herein covalently linked to a molecular payload, wherein the anti-TfR antibody comprises a CDR-H1, a CDR-H2, and a CDR-H3 that are the same as the CDR-H1, CDR-H2, and CDR-H3 shown in Table 3; and a CDR-L1, a CDR-L2, and a CDR-L3 that are the same as the CDR-L1, CDR-L2, and CDR-L3 shown in Table 3, and comprises a humanized VH and a humanized VL. In some embodiments, the molecular payload is an oligonucleotide.

In some embodiments, the complex described herein comprises an anti-TfR antibody covalently linked to a molecular payload, wherein the antibody is a humanized antibody that comprises a VH that contains human framework regions with the CDR-H1, CDR-H2, and CDR-H3 of a murine antibody listed in Table 1 or Table 3 (e.g., 3A4, 3M12, or 5H12), and a VL that contains human framework regions with the CDR-L1, CDR-L2, and CDR-L3 of a murine antibody listed in Table 1 or Table 3 (e.g., 3A4, 3M12, or 5H12). In some embodiments, the molecular payload is an oligonucleotide.

In some embodiments, the complex described herein comprises an anti-TfR antibody covalently linked to a molecular payload, wherein the antibody comprises a VH that contains human framework regions with the CDR-H1, CDR-H2, and CDR-H3 of a VH as set forth in SEQ ID NO: 7, and a VL that contains human framework regions with the CDR-L1, CDR-L2, and CDR-L3 of a VL as forth in SEQ ID NO: 8. In some embodiments, the molecular payload is an oligonucleotide.

In some embodiments, the complex described herein comprises an anti-TfR antibody covalently linked to a molecular payload, wherein the antibody comprises a VH that contains human framework regions with the CDR-H1, CDR-H2, and CDR-H3 of a VH as set forth in SEQ ID NO: 15, and a VL that contains human framework regions with the CDR-L1, CDR-L2, and CDR-L3 of a VL as forth in SEQ ID NO: 16. In some embodiments, the molecular payload is an oligonucleotide.

In some embodiments, the complex described herein comprises an anti-TfR antibody covalently linked to a molecular payload, wherein the antibody comprises a VH that contains human framework regions with the CDR-H1, CDR-H2, and CDR-H3 of a VH as set forth in SEQ ID NO: 23, and a VL that contains human framework regions with the CDR-L1, CDR-L2, and CDR-L3 of a VL as forth in SEQ ID NO: 24. In some embodiments, the molecular payload is an oligonucleotide.

In some embodiments, the complex described herein comprises an anti-TfR antibody covalently linked to a molecular payload, wherein the antibody is an IgG1 kappa that comprises human framework regions with the CDRs of a murine antibody listed in Table 1 or Table 3 (e.g., 3A4, 3M12, or 5H12). In some embodiments, the molecular payload is an oligonucleotide.

In some embodiments, the complex described herein comprises an anti-TfR antibody covalently linked to a molecular payload, wherein the antibody is a Fab′ fragment of an IgG1 kappa that comprises human framework regions with the CDRs of a murine antibody listed in Table 1 or Table 3 (e.g., 3A4, 3M12, or 5H12). In some embodiments, the molecular payload is an oligonucleotide.

In some embodiments, the complex described herein comprises an anti-TfR antibody covalently linked to a molecular payload, wherein the antibody is a Fab′ fragment of an IgG1 kappa that comprises human framework regions with the CDRs of a murine antibody listed in Table 1 or Table 3 (e.g., 3A4, 3M12, or 5H12). In some embodiments, the molecular payload is an oligonucleotide.

In some embodiments, the complex described herein comprises an anti-TfR antibody covalently linked via a lysine to the 5′ end of an oligonucleotide, wherein the antibody is a Fab′ fragment of an IgG1 kappa that comprises human framework regions with the CDRs of a murine antibody listed in Table 1 or Table 3 (e.g., 3A4, 3M12, or 5H12), wherein the complex has the structure of:

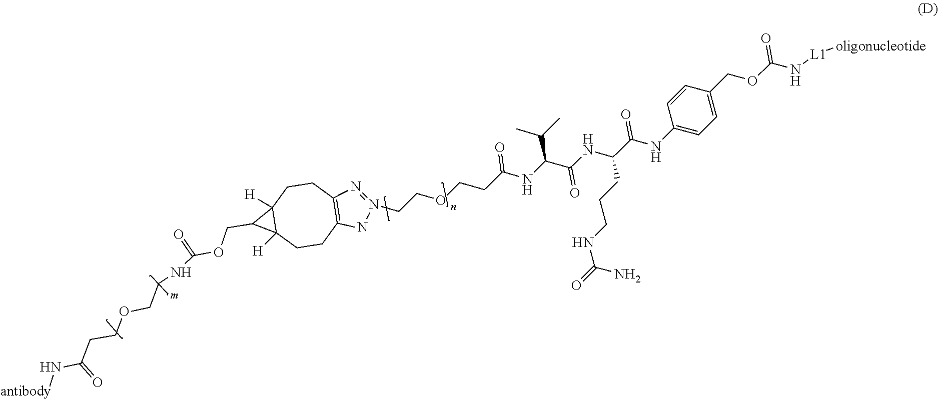

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 1, a CDR-H2 as set forth in SEQ ID NO: 2, a CDR-H3 as set forth in SEQ ID NO: 3, a CDR-L1 as set forth in SEQ ID NO: 4, a CDR-L2 as set forth in SEQ ID NO: 5, and a CDR-L3 as set forth in SEQ ID NO: 6; wherein the complex has the structure of:

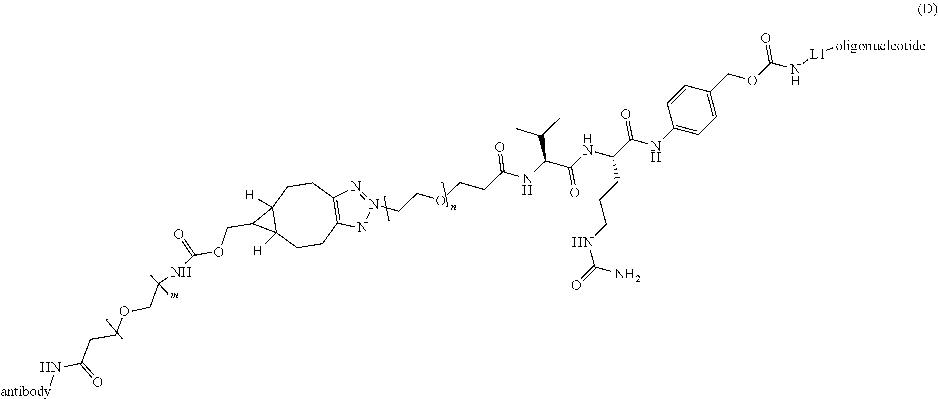

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 1, a CDR-H2 as set forth in SEQ ID NO: 233, a CDR-H3 as set forth in SEQ ID NO: 3, a CDR-L1 as set forth in SEQ ID NO: 4, a CDR-L2 as set forth in SEQ ID NO: 5, and a CDR-L3 as set forth in SEQ ID NO: 6; wherein the complex has the structure of:

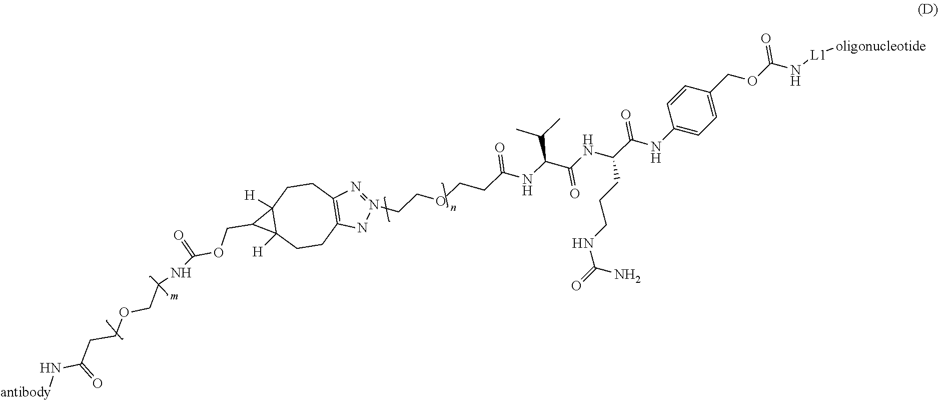

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 1, a CDR-H2 as set forth in SEQ ID NO: 80, a CDR-H3 as set forth in SEQ ID NO: 3, a CDR-L1 as set forth in SEQ ID NO: 4, a CDR-L2 as set forth in SEQ ID NO: 5, and a CDR-L3 as set forth in SEQ ID NO: 6; wherein the complex has the structure of:

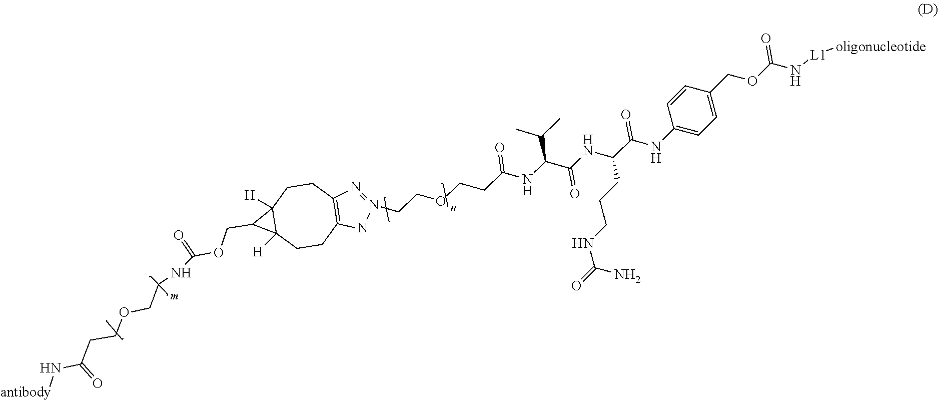

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 9, a CDR-H2 as set forth in SEQ ID NO: 10, a CDR-H3 as set forth in SEQ ID NO: 11, a CDR-L1 as set forth in SEQ ID NO: 12, a CDR-L2 as set forth in SEQ ID NO: 13, and a CDR-L3 as set forth in SEQ ID NO: 14; wherein the complex has the structure of:

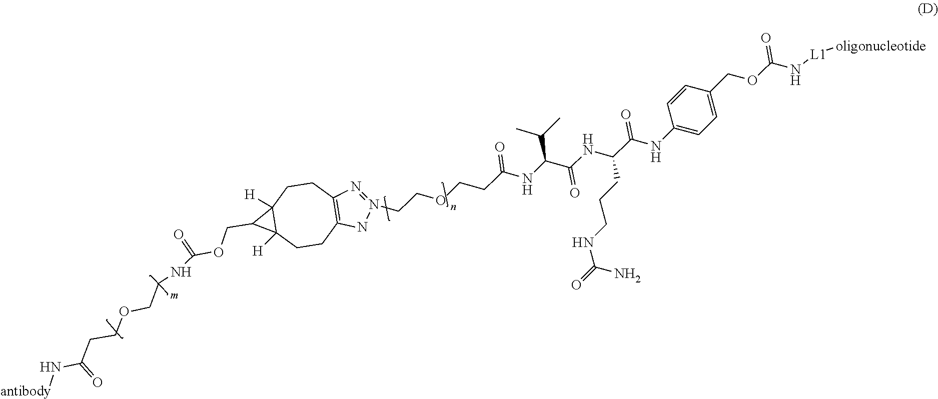

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 17, a CDR-H2 as set forth in SEQ ID NO: 18, a CDR-H3 as set forth in SEQ ID NO: 19, a CDR-L1 as set forth in SEQ ID NO: 20, a CDR-L2 as set forth in SEQ ID NO: 21, and a CDR-L3 as set forth in SEQ ID NO: 22; wherein the complex has the structure of:

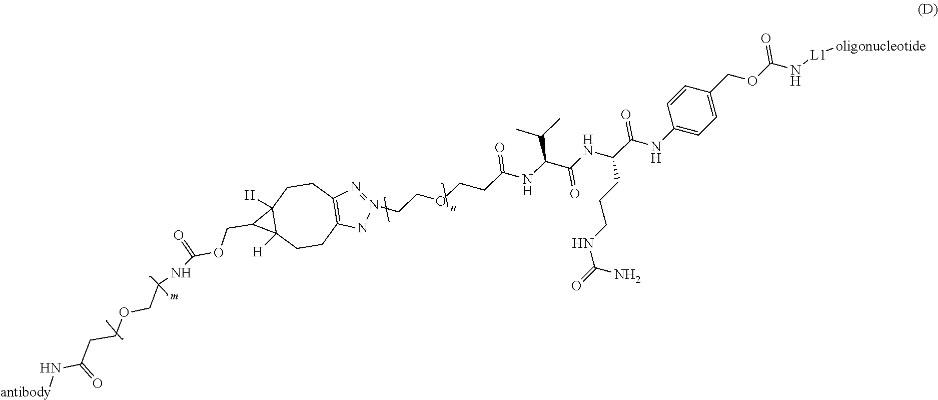

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 237, a CDR-H2 as set forth in SEQ ID NO: 18, a CDR-H3 as set forth in SEQ ID NO: 19, a CDR-L1 as set forth in SEQ ID NO: 20, a CDR-L2 as set forth in SEQ ID NO: 21, and a CDR-L3 as set forth in SEQ ID NO: 22; wherein the complex has the structure of:

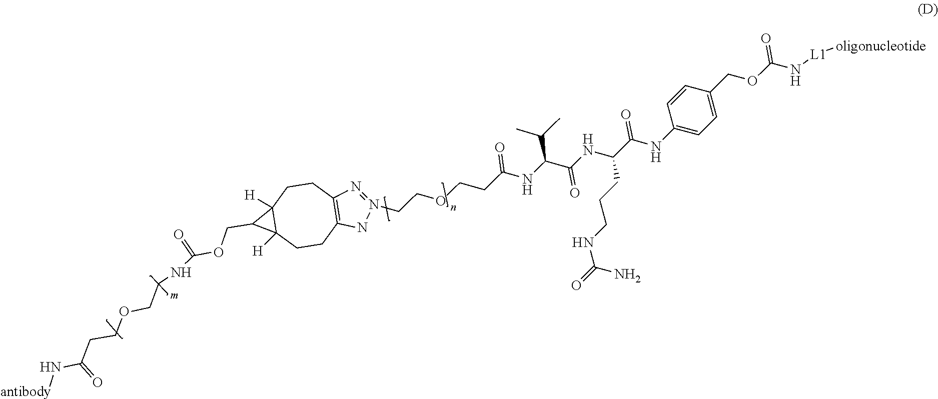

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 239, a CDR-H2 as set forth in SEQ ID NO: 18, a CDR-H3 as set forth in SEQ ID NO: 19, a CDR-L1 as set forth in SEQ ID NO: 20, a CDR-L2 as set forth in SEQ ID NO: 21, and a CDR-L3 as set forth in SEQ ID NO: 22; wherein the complex has the structure of:

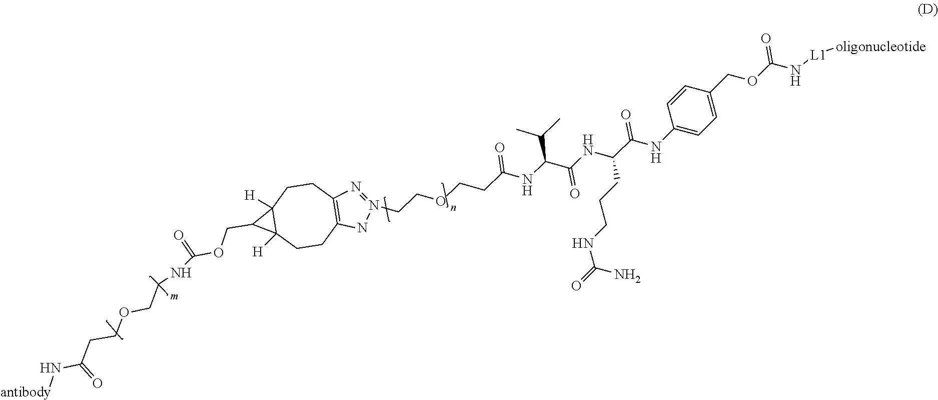

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 145, a CDR-H2 as set forth in SEQ ID NO: 146, a CDR-H3 as set forth in SEQ ID NO: 147, a CDR-L1 as set forth in SEQ ID NO: 148, a CDR-L2 as set forth in SEQ ID NO: 149, and a CDR-L3 as set forth in SEQ ID NO: 6; wherein the complex has the structure of:

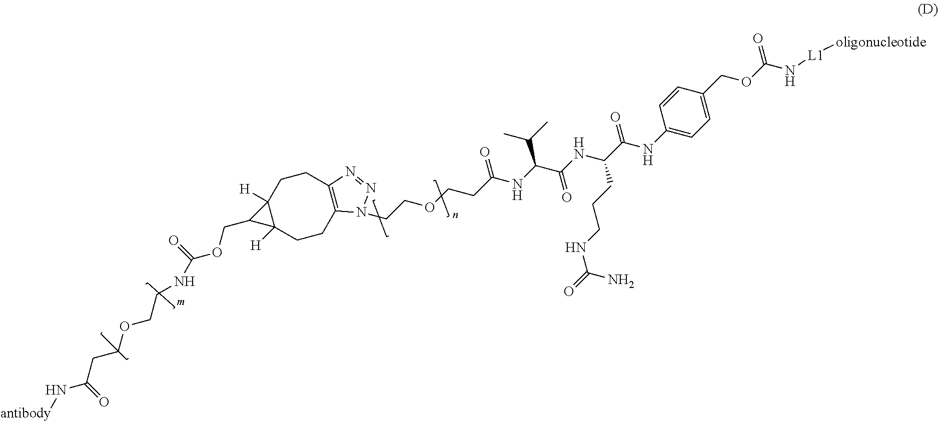

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 145, a CDR-H2 as set forth in SEQ ID NO: 234, a CDR-H3 as set forth in SEQ ID NO: 147, a CDR-L1 as set forth in SEQ ID NO: 148, a CDR-L2 as set forth in SEQ ID NO: 149, and a CDR-L3 as set forth in SEQ ID NO: 6; wherein the complex has the structure of:

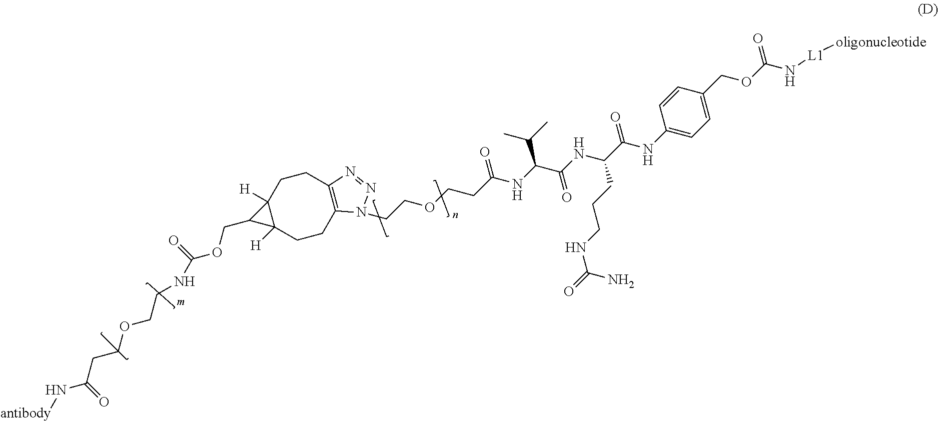

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 145, a CDR-H2 as set forth in SEQ ID NO: 236, a CDR-H3 as set forth in SEQ ID NO: 147, a CDR-L1 as set forth in SEQ ID NO: 148, a CDR-L2 as set forth in SEQ ID NO: 149, and a CDR-L3 as set forth in SEQ ID NO: 6; wherein the complex has the structure of:

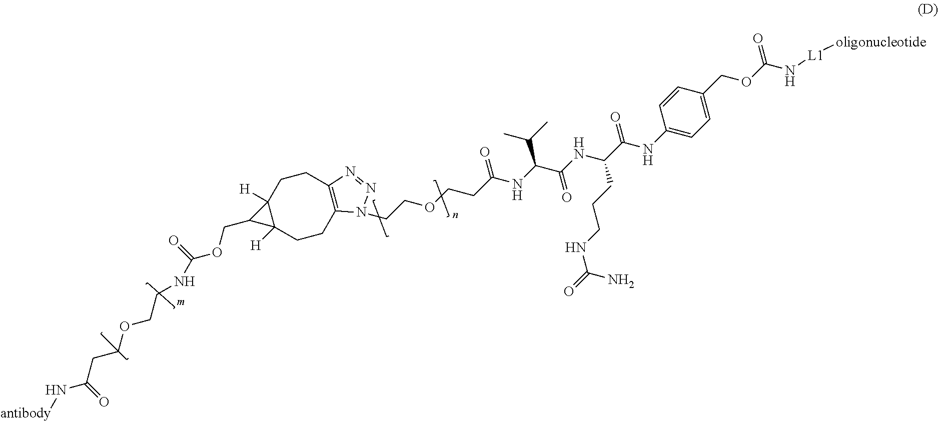

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 155, a CDR-H2 as set forth in SEQ ID NO: 156, a CDR-H3 as set forth in SEQ ID NO: 157, a CDR-L1 as set forth in SEQ ID NO: 158, a CDR-L2 as set forth in SEQ ID NO: 159, and a CDR-L3 as set forth in SEQ ID NO: 14; wherein the complex has the structure of:

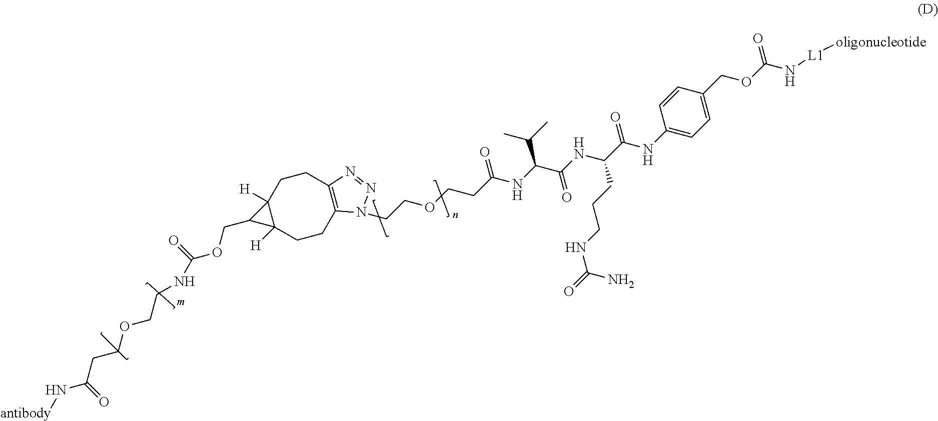

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 165, a CDR-H2 as set forth in SEQ ID NO: 166, a CDR-H3 as set forth in SEQ ID NO: 167, a CDR-L1 as set forth in SEQ ID NO: 168, a CDR-L2 as set forth in SEQ ID NO: 169, and a CDR-L3 as set forth in SEQ ID NO: 22; wherein the complex has the structure of:

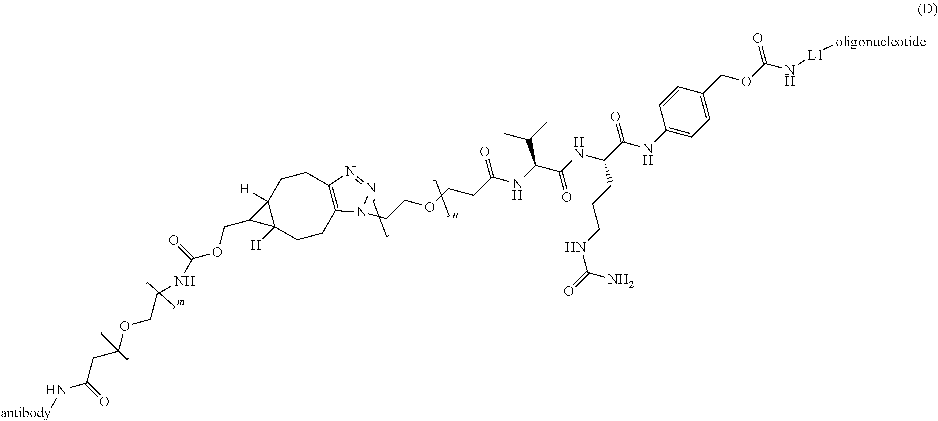

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 238, a CDR-H2 as set forth in SEQ ID NO: 166, a CDR-H3 as set forth in SEQ ID NO: 167, a CDR-L1 as set forth in SEQ ID NO: 168, a CDR-L2 as set forth in SEQ ID NO: 169, and a CDR-L3 as set forth in SEQ ID NO: 22; wherein the complex has the structure of:

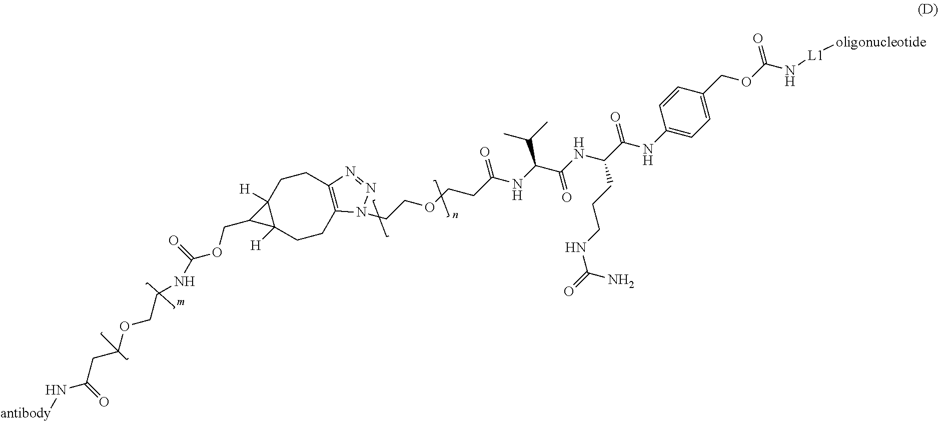

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 240, a CDR-H2 as set forth in SEQ ID NO: 166, a CDR-H3 as set forth in SEQ ID NO: 167, a CDR-L1 as set forth in SEQ ID NO: 168, a CDR-L2 as set forth in SEQ ID NO: 169, and a CDR-L3 as set forth in SEQ ID NO: 22; wherein the complex has the structure of:

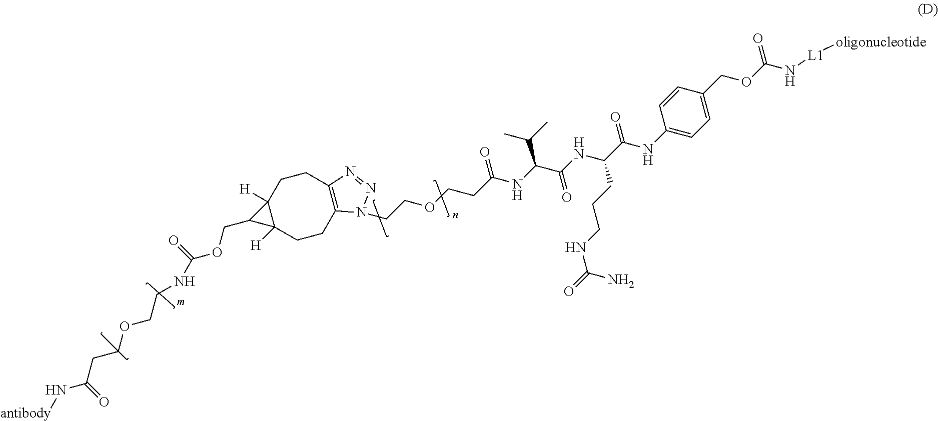

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 150, a CDR-H2 as set forth in SEQ ID NO: 151, a CDR-H3 as set forth in SEQ ID NO: 152, a CDR-L1 as set forth in SEQ ID NO: 153, a CDR-L2 as set forth in SEQ ID NO: 5, and a CDR-L3 as set forth in SEQ ID NO: 154; wherein the complex has the structure of:

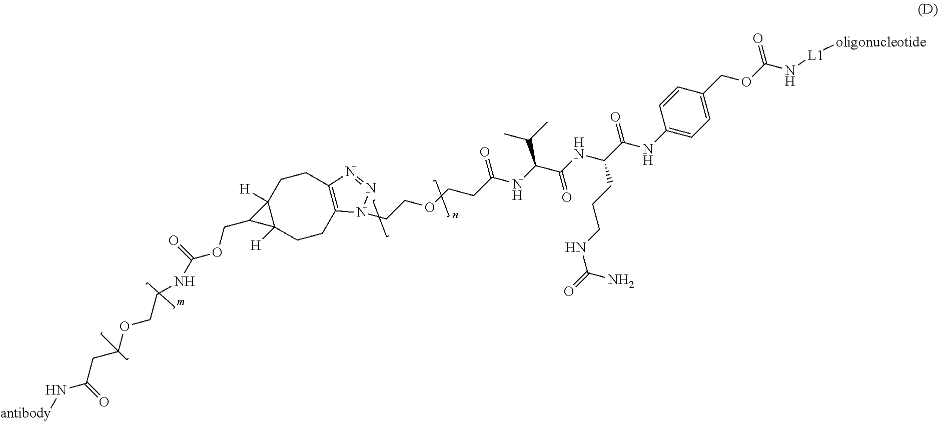

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 150, a CDR-H2 as set forth in SEQ ID NO: 277, a CDR-H3 as set forth in SEQ ID NO: 152, a CDR-L1 as set forth in SEQ ID NO: 153, a CDR-L2 as set forth in SEQ ID NO: 5, and a CDR-L3 as set forth in SEQ ID NO: 154; wherein the complex has the structure of:

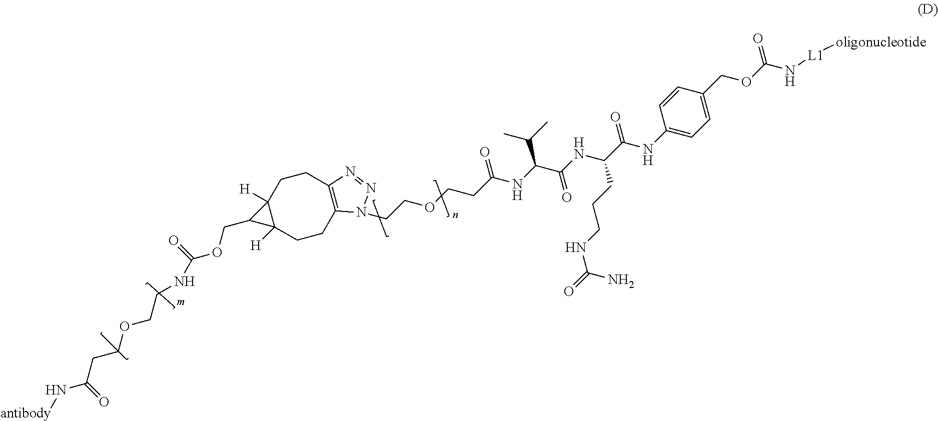

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 150, a CDR-H2 as set forth in SEQ ID NO: 278, a CDR-H3 as set forth in SEQ ID NO: 152, a CDR-L1 as set forth in SEQ ID NO: 153, a CDR-L2 as set forth in SEQ ID NO: 5, and a CDR-L3 as set forth in SEQ ID NO: 154; wherein the complex has the structure of:

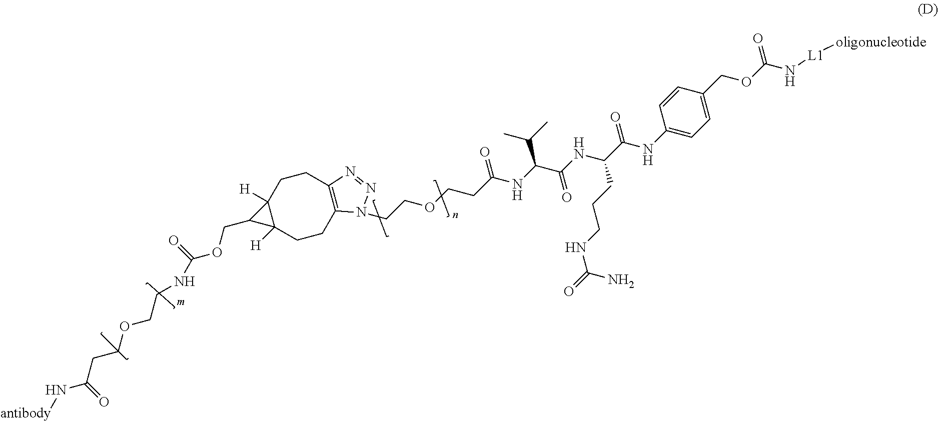

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 160, a CDR-H2 as set forth in SEQ ID NO: 161, a CDR-H3 as set forth in SEQ ID NO: 162, a CDR-L1 as set forth in SEQ ID NO: 163, a CDR-L2 as set forth in SEQ ID NO: 13, and a CDR-L3 as set forth in SEQ ID NO: 164; wherein the complex has the structure of:

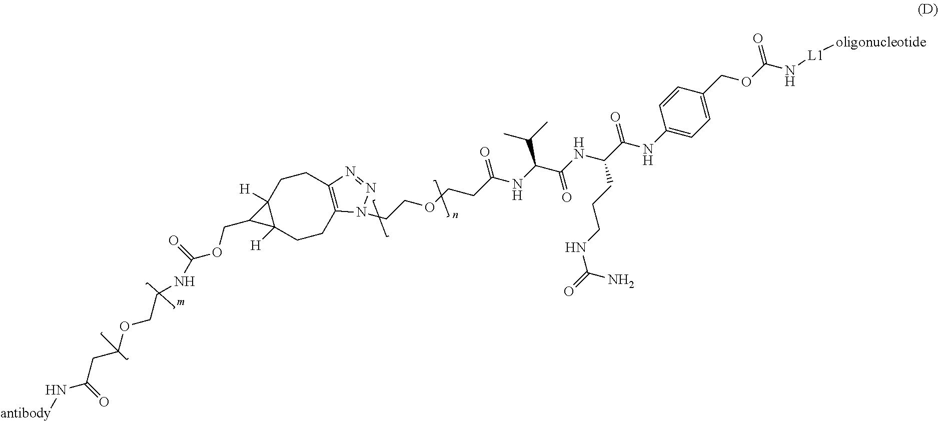

wherein n is 3 and m is 4.

In some embodiments, the complex described herein comprises an anti-TfR Fab covalently linked via a lysine to the 5′ end of an oligonucleotide, wherein the anti-TfR Fab comprises a CDR-H1 as set forth in SEQ ID NO: 170, a CDR-H2 as set forth in SEQ ID NO: 171, a CDR-H3 as set forth in SEQ ID NO: 172, a CDR-L1 as set forth in SEQ ID NO: 173, a CDR-L2 as set forth in SEQ ID NO: 21, and a CDR-L3 as set forth in SEQ ID NO: 174; wherein the complex has the structure of:

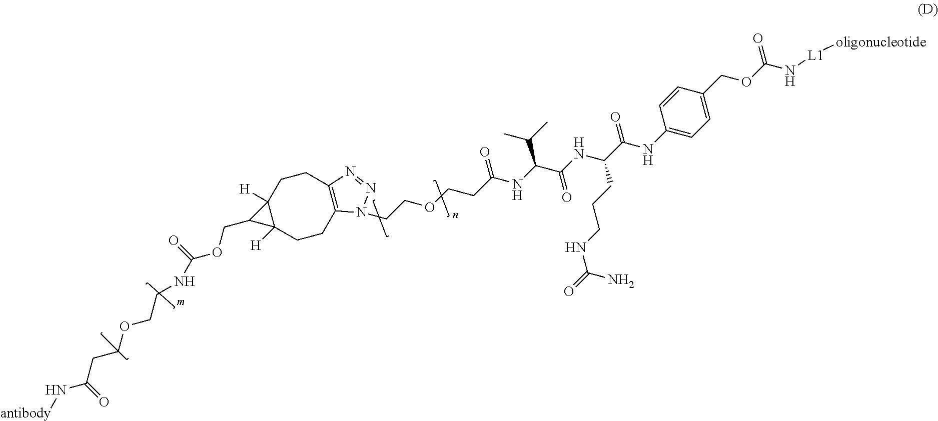

wherein n is 3 and m is 4.

In some embodiments, in any one of the examples of complexes described herein, L1 is any one of the spacers described herein.

In some embodiments, L1 is:

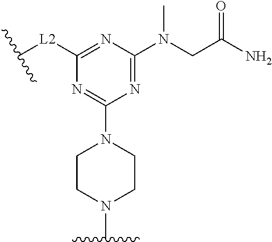

wherein the piperazine moiety links to the oligonucleotide, wherein L2 is

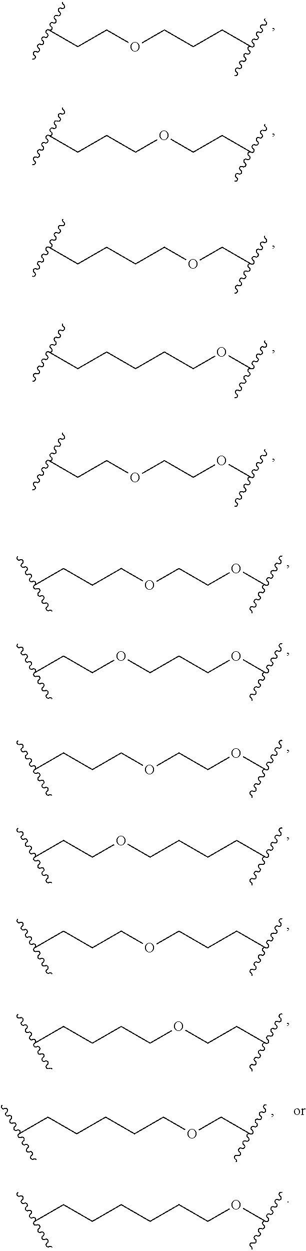

In some embodiments, L1 is:

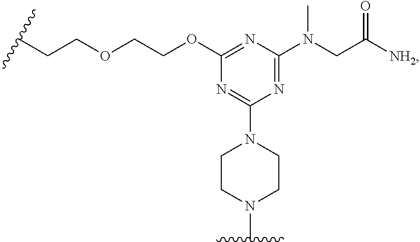 [SMILES: *CCOCCOc1nc(N(C)CC(N)=O)nc(N2CCN(*)CC2)n1]

wherein the piperazine moiety links to the oligonucleotide.

In some embodiments, L1 is

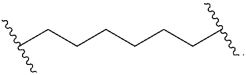 [SMILES: *CCCCCC*]

In some embodiments, L1 is linked to the 5′ phosphate of the oligonucleotide. In some embodiments, L1 is linked to the 5′ phosphorothioate of the oligonucleotide. In some embodiments, L1 is linked to the 5′ phosphonoamidate of the oligonucleotide.

In some embodiments, L1 is optional (e.g., need not be present).

### IV. FORMULATIONS

The anti-TfR antibodies or complexes provided herein may be formulated in any suitable manner. Generally, the antibodies or complexes provided herein are formulated in a manner suitable for pharmaceutical use. For example, the antibodies or complexes can be delivered to a subject using a formulation that minimizes degradation, facilitates delivery and/or (e.g., and) uptake, or provides another beneficial property to the complexes in the formulation. In some embodiments, provided herein are compositions comprising the antibodies or complexes and pharmaceutically acceptable carriers. Such compositions can be suitably formulated such that when administered to a subject, either into the immediate environment of a target cell or systemically, a sufficient amount of the complexes enter target muscle cells. In some embodiments, antibodies or complexes are formulated in buffer solutions such as phosphate-buffered saline solutions, liposomes, micellar structures, and capsids.

It should be appreciated that, in some embodiments, compositions may include separately one or more components of complexes provided herein (e.g., anti-TfR antibodies, linkers, molecular payloads, or precursor molecules of any one of them).

In some embodiments, antibodies or complexes are formulated in water or in an aqueous solution (e.g., water with pH adjustments). In some embodiments, antibodies or complexes are formulated in basic buffered aqueous solutions (e.g., PBS). In some embodiments, formulations as disclosed herein comprise an excipient. In some embodiments, an excipient confers to a composition improved stability, improved absorption, improved solubility and/or (e.g., and) therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil).

In some embodiments, a complex or component thereof (e.g., oligonucleotide or antibody) is lyophilized for extending its shelf-life and then made into a solution before use (e.g., administration to a subject). Accordingly, an excipient in a composition comprising a complex, or component thereof, described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, administration. Typically, the route of administration is intravenous or subcutaneous.

Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), and suitable mixtures thereof. In some embodiments, formulations include isotonic agents, for example, sugars, polyalcohols such as mannitol, sorbitol, and sodium chloride in the composition. Sterile injectable solutions can be prepared by incorporating the complexes in a required amount in a selected solvent with one or a combination of ingredients enumerated above, as required, followed by filtered sterilization.

In some embodiments, a composition may contain at least about 0.1% of the a complex, or component thereof, or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### V. METHODS OF USE

Some aspects of the present disclosure provide various uses of the anti-TfR antibodies, antibody fragments or variants, nucleic acids encoding such, and complexes described herein, including in research, diagnostic methods, detection methods, and therapeutic methods. In some embodiments, the anti-TfR antibodies described herein is used for delivering a molecular payload (e.g., a diagnostic or therapeutic agent) to a target cell or tissue that expresses a transferrin receptor. In some embodiments, the target cell is a muscle cell. In some embodiments, the target tissue is muscle. In some embodiments, the target tissue is brain. For delivering the molecular payload, the anti-TfR antibody may be conjugated (e.g., covalently conjugated) to the molecular payload to form a complex.

a. Diagnostic and Detection Methods

Also provided herein are the use of any one of the above described antibodies, antigen-binding fragments, polynucleotides, vectors or cells and optionally suitable means in diagnostic and/or (e.g., and) detection methods. The antibodies or antigen-binding fragments are, for example, suited for use in immunoassays in which they can be utilized in liquid phase or bound to a solid phase carrier. Examples of immunoassays which can utilize the antibody or antigen-binding fragments are competitive and non-competitive immunoassays in either a direct or indirect format. Examples of such immunoassays are the Enzyme Linked Immunoassay (ELISA), radioimmunoassay (RIA), the sandwich (immunometric assay), flow cytometry, the western blot assay, immunoprecipitation assays, immunohistochemistry, immuno-microscopy, lateral flow immuno-chromatographic assays, and proteomics arrays. The antigens and antibodies or antigen-binding fragments can be bound to many different solid supports (e.g., carriers, membrane, columns, proteomics array, etc.). Examples of well known solid support materials include glass, polystyrene, polyvinyl chloride, polyvinylidene difluoride, polypropylene, polyethylene, polycarbonate, dextran, nylon, amyloses, natural and modified celluloses, such as nitrocellulose, polyacrylamides, agaroses, and magnetite. The nature of the support can be either fixed or suspended in a solution (e.g., beads).

In some embodiments, any one of the anti-TfR antibodies provided herein is useful for detecting the presence of transferrin receptor in a biological sample. The term “detecting” as used herein encompasses quantitative or qualitative detection. In certain embodiments, a biological sample comprises a cell or tissue, such as blood, CSF, and BBB-containing tissue. The biological sample can be in vitro (e.g., cultured) or in vivo (e.g., in a subject). The present disclosure also contemplates the use of any one of the anti-TfR antibodies described herein in research use (e.g., as a reagent for immuno assays such as western blotting, immunostaining, ELISA, and/or (e.g., and) FACS).

In some embodiments, an anti-TfR antibody for use in a method of diagnosis or detection is provided. In some aspects, a method of detecting the presence of transferrin receptor in a biological sample is provided. In certain embodiments, the method comprises contacting the biological sample with an anti-TfR antibody as described herein under conditions permissive for binding of the anti-TfR antibody to the transferrin receptor, and detecting whether a complex is formed between the anti-TfR antibody and the transferrin receptor. Such method may be an in vitro or in vivo method. In some embodiments, an anti-TfR antibody is used to select subjects eligible for therapy with an anti-TfR antibody, e.g. where transferrin receptor is a biomarker for selection of patients.

Exemplary disorders that may be diagnosed using an anti-TfR antibody described herein include disorders involving immature red blood cells, due to the fact that transferrin receptor is expressed in reticulocytes and is therefore detectable by any of the antibodies of the invention. Such disorders include anemia and other disorders arising from reduced levels of reticulocytes, or congenital polycythemia or neoplastic polycythemia vera, where raised red blood cell counts due to hyperproliferation of, e.g., reticulocytes, results in thickening of blood and concomitant physiological symptoms.

In some embodiments, to detect the presence/level of transferrin receptor in a biological sample, labeled anti-TfR antibodies are used. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes 32P, 14C, 125I, 3H, and 131, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like. In some embodiments, the detectable label is an agent suitable for detecting transferrin receptor in a cell in vitro, which can be a radioactive molecule, a radiopharmaceutical, or an iron oxide particle. Radioactive molecules suitable for in vivo imaging include, but are not limited to, ¹²²I, ¹²³I, ¹²⁴I, ¹²⁵I, ¹³¹I, ¹⁸F, ⁷⁵Br, ⁷⁶Br, ⁷⁷Br, ²¹¹At, ²²⁵Ac, ¹⁷⁷Lu, ¹⁵³Sm, ¹⁸⁶Re, ¹⁸⁸Re, ⁶⁷Cu, ²¹³Bi, ²¹²Bi, ²¹²Pb, and ⁶⁷Ga. Exemplary radiopharmaceuticals suitable for in vivo imaging include ¹¹¹In Oxyquinoline, ¹³¹I Sodium iodide, ⁹⁹mTc Mebrofenin, and ⁹⁹mTc Red Blood Cells, ¹²³I Sodium iodide, ⁹⁹mTc Exametazime, ⁹⁹mTc Macroaggregate Albumin, ⁹⁹mTc Medronate, ⁹⁹mTc Mertiatide, ⁹⁹mTc Oxidronate, ⁹⁹mTc Pentetate, ⁹⁹mTc Pertechnetate, ⁹⁹mTc Sestamibi, ⁹⁹mTc Sulfur Colloid, ⁹⁹mTc Tetrofosmin, Thallium-201, or Xenon-133.

In certain embodiments, the anti-TfR antibody described herein can be used to deliver a detectable label to a target cell or tissue (e.g., muscle cell or across the blood brain barrier to the brain) for visualization of the cell or tissue (e.g., by fluorescent microscopy or by magnetic resonance imaging (MRI). Any of the detectable labels described herein can be used for this purpose.

In some embodiments, the anti-TfR antibody used in a diagnostic or detection method lacks effector function or has reduced effector function. In some embodiments, the anti-TfR antibody used in a diagnostic/detection method is engineered to have no or reduced effector function (e.g., by using a Fab, modifying the Ig backbone, introducing one or more Fc mutations reducing or eliminating effector function, and/or (e.g., and) modifying the glycosylation state of the antibody).

Various techniques are available for determining binding of the antibody to the transferrin receptor. One such assay is an enzyme linked immunosorbent assay (ELISA) for confirming an ability to bind to human transferrin receptor (and brain antigen). According to this assay, plates coated with antigen (e.g. recombinant transferrin receptor) are incubated with a sample comprising the anti-TfR antibody and binding of the antibody to the antigen of interest is determined.

To perform a diagnostic assay in vivo, a suitable amount of anti-TfR antibodies, conjugated with a label (e.g., an imaging agent or a contrast agent), can be administered to a subject in need of the examination. Presence of the labeled antibody can be detected based on the signal released from the label by routine methods. Assays for evaluating uptake of systemically administered antibody and other biological activity of the antibody are known to those skilled in the art.

To perform scientific research assays, an anti-TfR antibody can be used to study bioactivity of transferrin receptor and/or (e.g., and) detect the presence of transferrin receptor intracellularly. For example, a suitable amount of anti-TfR antibody can be brought in contact with a sample (e.g. a new cell type that is not previously identified as transferrin receptor producing cells) suspected of producing transferrin receptor. The antibody and the sample may be incubated under suitable conditions for a suitable period to allow for binding of the antibody to the transferrin receptor antigen. Such an interaction can then be detected via routine methods, e.g., ELISA, histological staining or FACS.

b. Treatment Methods

The anti-TfR antibodies described herein can be used for delivering molecular payloads that are therapeutic agents (e.g., oligonucleotides, peptides/proteins, nucleic acid constructs, etc.). In some aspects, the present disclosure also provide complexes comprising the anti-TfR antibodies covalently conjugated to a molecular payload for use in treating diseases.

In some aspects, complexes comprising an anti-TfR antibody covalently linked to a molecular payload as described herein are effective in treating a muscle disease (e.g., a rare muscle disease or muscle atrophy). In some embodiments, complexes are effective in treating a rare muscle disease provided in Table 7. In some embodiments, a muscle disease is associated with a disease allele, for example, a disease allele for a particular muscle disease may comprise a genetic alteration in a corresponding gene listed in Table 7.

In some embodiments, complexes are effective in treating muscle atrophy associated with the activity of one or more genes listed in Table 7 under the “Muscle Atrophy Gene Targets” section. In some embodiments, muscle atrophy is due to a chronic illness, including AIDS, congestive heart failure, cancer, chronic obstructive pulmonary disease, and renal failure, or muscle disuse.

In other aspects, complexes comprising an anti-TfR antibody covalently linked to a molecular payload as described herein are effective in treating a neurological disease. In some embodiments, the neurological disease include, but are not limited to, neuropathy, amyloidosis, cancer, an ocular disease or disorder, viral or microbial infection, inflammation, ischemia, neurodegenerative disease, seizure, behavioral disorders, and a lysosomal storage disease. For the purposes of this application, the CNS will be understood to include the eye, which is normally sequestered from the rest of the body by the blood-retina barrier. Specific examples of neurological disorders include, but are not limited to, neurodegenerative diseases (including, but not limited to, Lewy body disease, postpoliomyelitis syndrome, Shy-Draeger syndrome, olivopontocerebellar atrophy, Parkinson's disease, multiple system atrophy, striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g. of the CNS, including brain metastases resulting from cancer elsewhere in the body). In some embodiments, for treating a neurological disease, the complex comprises an anti-TfR antibody described herein conjugated to a drug for treating a neurological disease (e.g., the drugs listed in Table 8).

In some embodiments, a subject may be a human subject, a non-human primate subject, a rodent subject, or any suitable mammalian subject. In some embodiments, a subject may have a muscle disease provided in Table 7. In some embodiments, a subject may have muscle atrophy, or be at risk of developing muscle atrophy.

An aspect of the disclosure includes a methods involving administering to a subject an effective amount of a complex as described herein. In some embodiments, an effective amount of a pharmaceutical composition that comprises a complex comprising an anti-TfR antibody covalently linked to a molecular payload can be administered to a subject in need of treatment. In some embodiments, a pharmaceutical composition comprising a complex as described herein may be administered by a suitable route, which may include intravenous administration, e.g., as a bolus or by continuous infusion over a period of time. In some embodiments, intravenous administration may be performed by intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

Compositions for intravenous administration may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, a pharmaceutical composition that comprises a complex comprising an anti-TfR antibody covalently linked to a molecular payload is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising an anti-TfR antibody covalently linked to a molecular payload is administered at an effective concentration that confers therapeutic effect on a subject. Effective amounts vary, as recognized by those skilled in the art, depending on the severity of the disease, unique characteristics of the subject being treated, e.g. age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g. the half-life of the complex in a subject, generally will contribute to determination of the concentration of pharmaceutical composition that is used for treatment. The frequency of administration may be empirically determined and adjusted to maximize the efficacy of the treatment.

Generally, for administration of any of the complexes described herein, an initial candidate dosage may be about 1 to 100 mg/kg, or more, depending on the factors described above, e.g. safety or efficacy. In some embodiments, a treatment will be administered once. In some embodiments, a treatment will be administered daily, biweekly, weekly, bimonthly, monthly, or at any time interval that provide maximum efficacy while minimizing safety risks to the subject. Generally, the efficacy and the treatment and safety risks may be monitored throughout the course of treatment

The efficacy of treatment may be assessed using any suitable methods. In some embodiments, the efficacy of treatment may be assessed by evaluation of observation of symptoms associated with a muscle disease and/or (e.g., and) muscle atrophy.

In some embodiments, a pharmaceutical composition that comprises a complex comprising an anti-TfR antibody covalently linked to a molecular payload described herein is administered to a subject at an effective concentration sufficient to inhibit activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

In some embodiments, a single dose or administration of a pharmaceutical composition that comprises a complex comprising an anti-TfR antibody covalently linked to a molecular payload described herein to a subject is sufficient to inhibit activity or expression of a target gene for at least 1-5, 1-10, 5-15, 10-20, 15-30, 20-40, 25-50, or more days. In some embodiments, a single dose or administration of a pharmaceutical composition that comprises a complex comprising an anti-TfR antibody covalently linked to a molecular payload described herein to a subject is sufficient to inhibit activity or expression of a target gene for at least 1, 2, 3, 4, 5, 6, 7, 8, 9, 10, 11, or 12 weeks. In some embodiments, a single dose or administration of a pharmaceutical composition that comprises a complex comprising an anti-TfR antibody covalently linked to a molecular payload described herein to a subject is sufficient to inhibit activity or expression of a target gene for at least 1, 2, 3, 4, 5, or 6 months.

In some embodiments, a pharmaceutical composition may comprises more than one complex comprising an anti-TfR antibody covalently linked to a molecular payload. In some embodiments, a pharmaceutical composition may further comprise any other suitable therapeutic agent for treatment of a subject, e.g. a human subject having a muscle disease (e.g., a muscle disease provided in Table 7). In some embodiments, the other therapeutic agents may enhance or supplement the effectiveness of the complexes described herein. In some embodiments, the other therapeutic agents may function to treat a different symptom or disease than the complexes described herein.

c. Kits for Therapeutic and Diagnostic Applications

The present disclosure also provides kits for the therapeutic or diagnostic applications as disclosed herein. Such kits can include one or more containers comprising an anti-TfR antibody, e.g., any of those described herein.

In some embodiments, the kit can comprise instructions for use in accordance with any of the methods described herein. The included instructions can comprise a description of administration of the anti-TfR antibody to treat, delay the onset, or alleviate a target disease as those described herein. The kit may further comprise a description of selecting an individual suitable for treatment based on identifying whether that individual has the target disease. In still other embodiments, the instructions comprise a description of administering an antibody to an individual at risk of the target disease.

The instructions relating to the use of an anti-TfR antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or (e.g., and) alleviating a disease or disorder treatable by modulating immune responses, such as autoimmune diseases. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like.

Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-TfR antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

Also provided herein are kits for use in detecting transferrin receptor in a sample. Such a kit may comprise any of the anti-TfR antibodies described herein. In some instances, the anti-TfR antibody can be conjugated with a detectable label as those described herein. As used herein, “conjugated” or “attached” means two entities are associated, preferably with sufficient affinity that the therapeutic/diagnostic benefit of the association between the two entities is realized. The association between the two entities can be either direct or via a linker, such as a polymer linker. Conjugated or attached can include covalent or noncovalent bonding as well as other forms of association, such as entrapment, e.g., of one entity on or within the other, or of either or both entities on or within a third entity, such as a micelle.

Alternatively or in addition (e.g., in addition), the kit may comprise a secondary antibody capable of binding to anti-TfR antibody. The kit may further comprise instructions for using the anti-TfR antibody for detecting transferrin receptor.

### EXAMPLES

### Example 1: Screening of Anti-TfR Antibodies

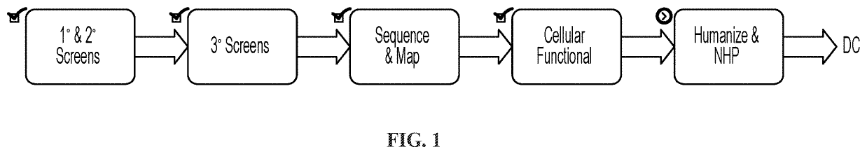

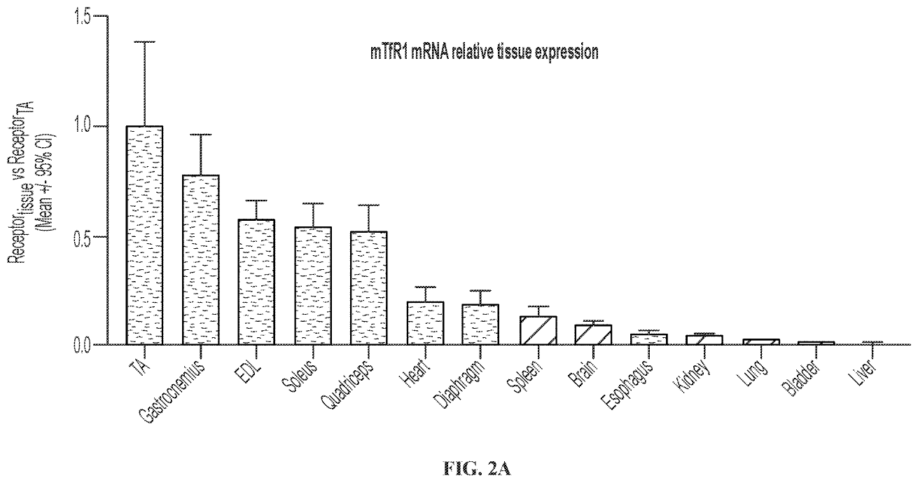

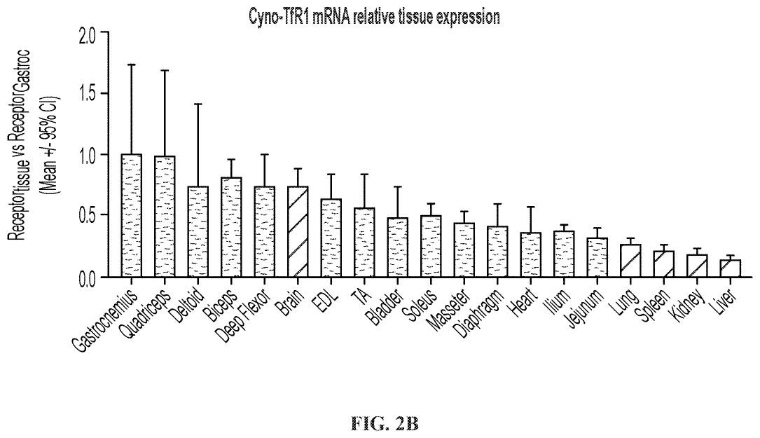

From mice immunized with the ECD domain of human TfR1, a total of 347 positive hybridoma clones were identified as being able to bind to huTfR1 by ELISA. The 347 antibodies were then tested in several assays for selecting the top candidates (e.g., see FIG. 1). For example, in an ELISA assay that tested binding to cynoTfR1, antibodies that are cynoTfR1 non-binders were removed from the candidate pool. Subsequently, antibodies that binned with known anti-TfR antibodies were removed from the candidate pool. Next, the remaining antibodies were tested in an ELISA assay for their ability to bind huTfR2 and antibodies that bind to huTfR2 binders were removed from the candidate pool. Further, in a FACS assay that tested binding to HepG2 and CHO cells, antibodies that non-specifically bound to CHO cells were removed from the candidate pool. Finally, in a competitive ELISA assay that tested competition between the anti-TfR antibodies and transferrin, antibodies that compete with and inhibited transferrin binding to TfR were removed from the candidate pool.

Exemplary selection criteria include:
K_{D} of about 60 pM-about 20 nM to human TfR1
K_{D} to human TfR1 and cyno TfR1 within 1 log
K_{off} rate within similar K_{D} range
Minimal potential deamidation, isomerization, or oxidation sites

These filters selectively narrowed down the 347 positive hybridoma clones to about 90 clones. Examples of heavy chain variable domains (VHs) and light chain variable domains (VLs) were sequenced and the sequences are provided in Table 1. In silico sequence analysis was performed to identify potential modification sites or liable residues. Carterra kinetic binding data was obtained to determine Kd, Kₒₙ, and K_{off} rates. Cross-binning was performed for the selected clones against each other, to place groups of antibodies into bins based on their ability to compete with one another. Linear epitope mapping was performed to identify any consensus binding sites.

All of this data was combined to choose 20 antibodies spanning a wide-range of affinities, a diverse sequence repertoire, and desirable antibody characteristics were selected for further testing and conjugation with molecular payloads.

The binding epitopes on human TfR1 for three selected antibodies (3-A4, 3-M12, and 5-H12) were also mapped. Antibody 3-A4 binds to an epitope KGVEPKT, corresponding to amino acids 90-96 of the human TfR1 as set forth in SEQ ID NO: 228. Based on the binding epitope, the 3-A4 antibody is expected to bind to TfR1 even when the extracellular domain of TfR1 is cleaved. No linear binding epitopes antibodies 3-M12 or 5-H12 was identified, indicating that the binding may be conformational.

### Example 2: Binding Affinity of Anti-TfR1 Antibodies to Human TfR1

Selected anti-TfR1 antibodies were tested for their binding affinity to human TfR1 for measurement of Ka (association rate constant), Kd (dissociation rate constant), and K_{D} (affinity). Two known anti-TfR1 antibodies were used as control, 15G11 and OKT9. The binding experiment was performed on Carterra LSA at 25 C. An anti-mouse IgG and anti-human IgG antibody “lawn” was prepared on a HC30M chip by amine coupling. 59 IgGs (58 mouse mAbs and 1 human mAb) were captured on the chip. Dilution series of hTfR1, cyTfR1, and hTfR2 were injected to the chip for binding (starting from 1000 nM, 1:3 dilution, 8 concentrations).

Binding data were referenced by subtracting the responses from a buffer analyte injection and globally fitting to a 1:1 Langmuir binding model for estimate of Ka (association rate constant), Kd (dissociation rate constant), and K_{D} (affinity) using the Carterra™ Kinetics software. 5-6 concentrations were used for curve fitting.

The result showed that 54 out of 56 mouse mAbs demonstrated binding to hTfR1 with K_{D} values ranging from 13 pM to 50 nM. A majority of the mouse mAbs had K_{D} values in the single digit nanomolar to sub-nanomolar range. 49 out of 56 mouse mAbs showed cross-reactive binding to cyTfR1 with K_{D} values ranging from 16 pM to 22 nM.

Ka, Kd, and K_{D} values of anti-TfR1 antibodies are provided in Table 9.

TABLE 9
Ka, Kd, and K_{D} values of anti-TfR1 antibodies

| Name | K_{D} (M) | Kₐ (M) | K_{d} (M) |
|---|---|---|---|
| ctrl-15G11 | 2.83E−10 | 3.70E+05 | 1.04E−04 |
| ctrl-OKT9 mIgG | 5.36E−10 | 7.74E+05 | 4.15E−04 |
| 3-A04 | 4.36E−10 | 4.47E+05 | 1.95E−04 |
| 3-M12 | 7.68E−10 | 1.66E+05 | 1.27E−04 |
| 5-H12 | 2.08E−07 | 6.67E+04 | 1.39E−02 |
| 10-H02 | 2.72E−09 | 1.26E+05 | 3.42E−04 |
| 10-P05 | 1.63E−09 | 1.70E+05 | 2.78E−04 |
| 2-H19 | 2.06E−09 | 2.22E+05 | 4.56E−04 |
| 3-E05 | 4.55E−10 | 2.20E+04 | 1.00E−05 |
| 3-F03 | 2.23E−09 | 1.38E+05 | 3.09E−04 |
| 3-M09 | 2.54E−09 | 1.50E+05 | 3.82E−04 |
| 3-P24 | 9.70E−10 | 6.72E+04 | 6.52E−05 |
| 4-C05 | 1.61E−09 | 3.01E+04 | 4.85E−05 |
| 4-H04 | 1.39E−08 | 6.17E+04 | 8.57E−04 |
| 4-O12 | 1.80E−09 | 7.98E+04 | 1.43E−04 |
| 6-D03 | 9.86E−10 | 1.08E+05 | 1.07E−04 |
| 8-D15 | 8.22E−09 | 3.13E+04 | 2.57E−04 |
| 8-K06 | 6.94E−11 | 1.44E+05 | 1.00E−05 |
| 8-O17 | 1.83E−09 | 4.99E+04 | 9.12E−05 |
| 9-C04 | 1.41E−08 | 4.10E+04 | 5.75E−04 |
| 9-D04 | 5.86E−09 | 4.20E+04 | 2.46E−04 |
| 9-K23 | 4.01E−10 | 5.40E+04 | 2.17E−05 |

### Example 3: Conjugation of Anti-TfR1 Antibodies with Oligonucleotides

Complexes containing an anti-TfR1 antibody covalently conjugated to an antisense oligonucleotide (ASO) targeting DMPK (control DMPK-ASO) were generated. First, Fab′ fragments of anti-TfR antibody clones 3-A4, 3-M12, 5-H12, 8-K6, 9-K23, 3-E5, 6-D3, 4-O12, 4-C5, 10-P5, 2-H19, 3-F3, 8-O17, 3-M9, 10-H2, 4-J22, 9-D4, 8-D15, 4-H4, and 9-C4 were prepared by cutting the mouse monoclonal antibodies with an enzyme in or below the hinge region of the full IgG followed by partial reduction. The Fab's were confirmed to be comparable to mAbs in avidity or affinity.

Muscle-targeting complexes was generated by covalently linking the anti-TfR mAbs to an ASO targeting DMPK via a cathepsin cleavable linker. Briefly, a Bicyclo[6.1.0]nonyne-PEG3-L-valine-L-citrulline-pentafluorophenyl ester (BCN-PEG3-Val-Cit-PFP) linker molecule was coupled to DMPK-targeting ASO through a carbamate bond. Excess linker and organic solvents were removed by tangential flow filtration (TFF). The purified Val-Cit-linker-ASO was then coupled to an azide functionalized anti-transferrin receptor antibody generated through modifying ε-amine on lysine with Azide-PEG4-PFP. A positive control muscle-targeting complex was also generated using 15G11.

The product of the antibody coupling reaction was then subjected to two purification methods to remove free antibody and free payload: 1) hydrophobic interaction chromatography (HIC-HPLC), and 2) Size exclusion chromatography (SEC). The HIC column utilized a decreasing salt gradient to separate free antibody from conjugate. During SEC, fractionation was performed based on A260/A280 traces to specifically collect conjugated material. Concentrations of the conjugates were determined by either Nanodrop A280 or BCA protein assay (for antibody) and Quant-It Ribogreen assay (for payload). Corresponding drug-antibody ratios (DARs) were calculated. DARs ranged between 0.8 and 2.0, and were standardized so that all samples receive equal amounts of payload.

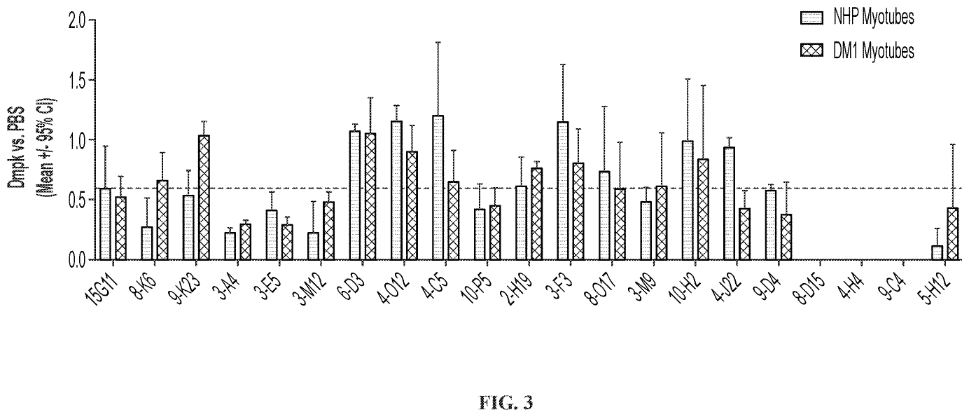

The purified complexes were then tested for cellular internalization and inhibition of DMPK. Non-human primate (NHP) or DM1 (donated by DM1 patients) cells were grown in 96-well plates and differentiated into myotubes for 7 days. Cells were then treated with escalating concentrations (0.5 nM, 5 nM, 50 nM) of each complex for 72 hours. Cells were harvested, RNA was isolated, and reverse transcription was performed to generate cDNA. qPCR was performed using TaqMan kits specific for Ppib (control) and DMPK on the QuantStudio 7. The relative amounts of remaining DMPK transcript in treated vs non-treated cells was were calculated and the results are shown in Table 10 and FIG. 3.

The results demonstrated that the anti-TfR1 antibodies are able to target muscle cells, be internalized by the muscle cells with the molecular payload (DMPK ASO), and that the molecular payload (DMPK ASO) are able to target and knockdown the target gene (DMPK).

TABLE 10
Binding Affinity of anti-TfR1 Antibodies and Efficacy of Conjugates

| | | | % knockdown | % knockdown |
|---|---|---|---|---|
| | | | of DMPK in | of DMPK in |
| | | | NHP cells | cells from |
| | huTfR1 | cyTfR1 | using | human DM1 |
| | Avg K_{D} | Avg | Antibody- | patients using |
| | (M) | K_{D} (M) | DMPK | Antibody- |
| Clone | (antibody | (antibody | ASO | DMPK |
| Name | alone) | alone) | conjugate | ASO conjugate |
| 15G11 (control) | 8.0E−10 | 1.0E−09 | 36 | 46 |
| 3-A4 | 4.36E−10 | 2.32E−09 | 77 | 70 |
| 3-M12 | 7.68E−10 | 5.18E−09 | 77 | 52 |
| 5-H12 | 2.02316E−07 | 1.20E−08 | 88 | 57 |
| 8-K6 | 6.78121E−11 | 3.76E−10 | 73 | 34 |
| 9-K23 | 3.19783E−10 | 6.62E−10 | 47 | −4 |
| 3-E5 | 4.55E−10 | 6.71E−10 | 59 | 71 |
| 6-D3 | 9.86E−10 | 7.78E−10 | −8 | −5 |
| 4-O12 | 1.27416E−09 | 2.70E−07 | −16 | 10 |
| 4-C5 | 1.38324E−09 | 1.36E−08 | −20 | 35 |
| 10-P5 | 1.63E−09 | 1.10E−08 | 58 | 55 |
| 2-H19 | 2.06E−09 | 5.75E−09 | 39 | 24 |
| 3-F3 | 2.23E−09 | 1.84E−08 | −15 | 20 |
| 8-O17 | 2.24245E−09 | 1.10E−09 | 26 | 41 |
| 3-M9 | 2.50135E−09 | 4.37E−09 | 52 | 39 |
| 10-H2 | 2.72E−09 | 1.24E−08 | 2 | 16 |
| 4-J22 | 3.41E−09 | 1.37E−09 | 7 | 57 |
| 9-D4 | 5.79556E−09 | 8.68E−10 | 42 | 62 |
| 8-D15 | 9.15057E−09 | 1.11E−08 | * | * |
| 4-H4 | 1.39E−08 | 2.18E−08 | * | * |
| 9-C4 | 1.47657E−08 | 1.20E−08 | * | * |

* very low yield from expression/conjugation

Interestingly, the DMPK knockdown results showed a lack of correlation between the binding affinity of the anti-TfR to transferrin receptor and efficacy in delivering a DMPK ASO to cells for DMPK knockdown. Surprisingly, the anti-TfR antibodies provided herein (e.g., at least 3-A4, 3-M12, 5-H12, 8-K6, 3-E5, 10-P5, 3-M9, and 9-D4) demonstrated superior activity in delivering a payload (e.g., DMPK ASO) to the target cells and achieving the biological effect of the molecular payload (e.g., DMPK knockdown) in either cyno cells or human DM1 patient cells, compared to the control antibody 15G11, despite the comparable binding affinity (or, in certain instances, such as 5-H12, lower binding affinity) to human or cyno transferrin receptor between these antibodies and the control antibody 15G11.

Top attributes such as high huTfR1 affinity, >50% knockdown of DMPK in NHP and DM1 patient cell line, identified epitope binding with 3 unique sequences, low/no predicted PTM sites, and good expression and conjugation efficiency were considered for the selection of clones for humanization.

### Example 4: Structure-Activity Relationship (SAR) of the Anti-TfR Antibodies

Structure-Activity Relationship (SAR) analysis was performed to determine consensus sequences in the CDR and/or (e.g., and) framework region. The analysis shows that 3-A4 has the same heavy chain and light chain CDRs as 9-B22, and differs from 9-B22 by one amino acid in the heavy chain framework region at amino acid 64 (V in 3-A4, and M in 9-B22).

For antibody 3-M12, antibodies having similar heavy chain and/or (e.g., and) light chain CDR sequences were identified, including 10C21, 10-K10, 1-102, 6-B10, and 9-13. The respective CDRs are shown in Table 11 below. 5-H12 has unique heavy chain and light CDR sequences and no other antibodies that share similar sequences were identified in the analysis.

TABLE 11
SAR Analysis of 3-M12

| | Affinity | | | | | | |
|---|---|---|---|---|---|---|---|
| | to | | | | CDR- | CDR- | CDR- |
| Clone | huTfR1 | CDR-H1 | CDR-H2 | CDR-H3 | L1 | L2 | L3 |
| 3M12 | 768 pM | GYSIT | ITFDGAN | TRSSYDY | QDISNF | YTS | QQGHTLPYT |
| | | SGYY | (SEQ ID | DVLDY | (SEQ ID | (SEQ | (SEQ ID NO: |
| | | (SEQ ID | NO: 10) | (SEQ ID | NO: 12) | ID NO: | 14) |
| | | NO: 9) | | NO: 11) | | 13) | |
| 10C21 | NA | GYTFTDYG | IYPSSGN | ARSNYYG | QDISNC | YTS | QQGNTLPWT |
| | | (SEQ ID | T (SEQ ID | SPIDF | (SEQ ID | (SEQ | (SEQ ID NO: |
| | | NO: 261) | NO: 263) | (SEQ ID | NO: 278) | ID NO: | 282) |
| | | | | NO: 266) | | 13) | |
| 10K10 | 21 nM | GYSITSGY | INFDGNN | ARFYYD | QDISNC | YTS | QQGNTLPWT |
| | | Y (SEQ ID | (SEQ ID | YDYFDY | (SEQ ID | (SEQ | (SEQ ID NO: |
| | | NO: 9) | NO: 264) | (SEQ ID | NO: 278) | ID NO: | 282) |
| | | | | NO: 267) | | 13) | |
| 1102 | 67 pM | GYSITSGY | INFDGNN | ARIYYD | QDISNS | YTS | QQGNTLPFT |
| | | Y (SEQ ID | (SEQ ID | YDYFDY | (SEQ ID | (SEQ | (SEQ ID NO: |
| | | NO: 9) | NO: 264) | (SEQ ID | NO: 279) | ID NO: | 283) |
| | | | | NO: 268) | | 13) | |
| 6B10 | 287 pM | GYSITSGY | INFDGNS | TRSSYD | QDITNF | YTS | QQGNTLPYT |
| | | Y (SEQ ID | (SEQ ID | YDVLDY | (SEQ ID | (SEQ | (SEQ ID NO: |
| | | NO: 9) | NO: 265) | (SEQ ID | NO: 280) | ID NO: | 284) |
| | | | | NO: 11) | | 13) | |
| 913 | 2.4 nM | GFSIT | INFD | ARSFYD | QDISNY | YTS | QQGHAYPYT |
| | | SGYY | GNN | YDVLDY | (SEQ ID | (SEQ | (SEQ ID |
| | | (SEQ ID | (SEQ ID | (SEQ ID | NO: 281) | ID NO: | NO: 285) |
| | | NO: 262) | NO: 264) | NO: 277) | | 13) | |

### Example 3. Serum Stability of the Linker Linking the Anti-TfR Antibody and the Molecular Payload

Oligonucleotides which were linked to antibodies in examples were conjugated via a cleavable linker shown in Formula (C). It is important that the linker maintain stability in serum and provide release kinetics that favor sufficient payload accumulation in the targeted muscle cell. This serum stability is important for systemic intravenous administration, stability of the conjugated oligonucleotide in the bloodstream, delivery to muscle tissue and internalization of the therapeutic payload in the muscle cells. The linker has been confirmed to facilitate precise conjugation of multiple types of payloads (including ASOs, siRNAs and PMOs) to Fabs. This flexibility enabled rational selection of the appropriate type of payload to address the genetic basis of each muscle disease. Additionally, the linker and conjugation chemistry allowed the optimization of the ratio of payload molecules attached to each Fab for each type of payload, and enabled rapid design, production and screening of molecules to enable use in various muscle disease applications.

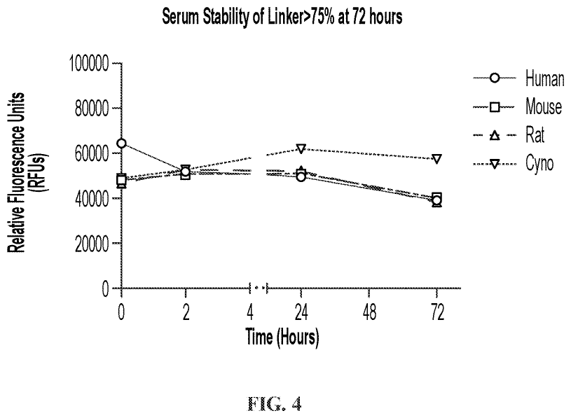

FIG. 4 shows serum stability of the linker in vivo, which was comparable across multiple species over the course of 72 hours after intravenous dosing. At least 75% stability was measured in each case at 72 hours after dosing.

### Materials and Methods

### Hu/cynoTfR1 ELISA Protocol

A 20 μg vial of recombinant huTfR1 or cynoTFR1 was diluted into 10 mLs of PBS. A high binding, black, flat bottom, 96 well-plate (Corning #3925) was coated with 100 μL/well of the recombinant huTfR1 or cynoTfR1 at 1 μg/mL in PBS and left overnight at 4° C. After the coating, liquid was flicked out and the kim-wipe was gently tapped on to remove residual liquid. Lyophilized BSA was dissolved at 10 mg/mL to make a 1% w/v solution and then 200 μL of the 1% BSA (w/w) in PBS was added to each well with multichannel. Blocking was allowed to proceed for 2 hours at RT on shaker, 300 rpm. 20×TBST solution (Thermo #J77500-K2) was diluted in nuclease free water. After blocking, the liquid was flicked out and the plate was washed 3× with 300 μL of TBST. A serial dilution of anti-TfR1 antibodies in 0.5% BSA/TBST was added to row A in triplicate with 8 point serial dilution in column format. Positive control and isotype controls were included on the plate. The dilution range was 5 μg/mL to 5 ng/mL. The plate was placed at room temperature on an orbital shaker for 60 minutes, at 300 rpm. Then liquid was flicked out and the plate was washed 3× with 300 μL of TBST. Anti-(H+L)IgG-A488 (1:500) (Invitrogen #A11013) were diluted in 0.5% BSA in TBST, and 100 μL were added per well. The plate was then incubated for 60 minutes at room temperature, at 300 rpm on an orbital shaker. Liquid was then flicked out and the plate was washed 4× with 300 μL of TBST. The plate was read on a spectramax plate reader at 495 nm excitation, 520 nm emission (15 nm bandwidth). The data was recorded as an excel file and analyzed in Prism for EC50 calculation and log curves.

### TFN Competition Assays

ELISA Assay of Ab Blocking of TfN or HFE Binding to TfR1 were performed using two methods. The buffers used in both methods include a Wash Buffer: containing 1×PBS supplemented with 0.05% Tween-20 and a Blocking Buffer: containing 1×PBS supplemented with 3% BSA.

### Method 1

Recombinant purified TfR1-HIS protein was dilute to 1 μg/mL with 1×PBS and aliquot 10 μL into each well. The plates were then covered incubated at 4° C. overnight (12 to 20 hours) such that the plates are coated with TfR1 protein. After overnight coating, the plates were washed plates 2× with Wash Buffer. The remaining Wash Buffer was discarded. The wells were incubated with Blocking Buffer (55 μL) at room temperature (RT) for 1 hour. At the end of the block, the block solution was discarded. Plates were washed 2× with Wash Buffer. The antibodies were then prewashed with protein-binding partners. Premix A: control antibodies (20m/mL, 2×) were mixed with either 200 nM Tfn (2×) or 200 nM HFE-HIS (2×). Premix B: saturated supernatants were mixed with either 200 nM Tfn (2×) or 200 nM HFE-HIS (2×). Premix A or Premix B (20 μL) were added to wells, which were then incubated for 1 hour at room temperature.

After incubation, the supernatant was discarded and plates were washed 4× with Wash Buffer. The wells were incubated with either anti-mouse IgG-HRP or anti-human IgG-HRP (1:7000 dilution; 20 μL/well) at room temperature for 45 minutes.

The SuperSignal ELISA Pico Chemiluminescence Substrate solution was prepared by pre-mixing Peroxidase Solution with Luminol Enhancer Solution as a 1:1 solution. The mixture was diluted to 50% with 1×PBS.

After the 45 minute incubation, each plate was washed 4× using 1× wash buffer. The plate was rotated 180 degrees and washed an additional 4×. The pre-mixed SuperSignal ELISA Pico substrate solution (20 μL) was added to each well. The plate was read on MolecuLar Devices SpectraMax M3 luminometer and Softmax Pro Version 6.2 within 15 minutes of adding the substrate.

### Method 2

Tfn stock protein was thawed and diluted to 2 μg/mL with 1×PBS. 10 μL was distributed into each well. Each plate was covered and incubated at 4° C. overnight (12 to 20 hours).

After overnight coating, the plates were washed 2× with Wash Buffer. The remaining Wash Buffer was discarded. The wells were incubated with Blocking Buffer (55 μL). and incubated at room temperature (RT) for 1 hour. At the end of the block, the block solution was discarded. The plates were washed 2× with Wash Buffer. The antibodies were premixed with protein-binding partners. Premix C: control antibodies (20m/mL, 2×) were mixed with 30.8 μg/mL TfR1 (2×). Premix D: saturated supernatants were mixed with 30.8 μg/mL TfR1 (2×). Add Premix C or Premix D (20 μL) was to wells, which were then incubated for 1 hour at room temperature.

The supernatant was discarded. The plates were washed 4× with Wash Buffer. The wells were incubated with 1 μg/mL Biotin-MD10 (20 μl/well) at room temperature for 45 minutes. The plates were washed 4× with Wash Buffer. The wells were incubated with 1 μg/mL Streptavidin-HRP (20 μl/well) at room temperature for 45 minutes.

The SuperSignal ELISA Pico Chemiluminescence Substrate solution was prepared by pre-mixing Peroxidase Solution with Luminol Enhancer Solution as a 1:1 solution. The mixture was diluted to 50% with 1×PBS.

After the 45 minute incubation, wash each plate was washed 4× using 1× wash buffer. The plate was rotated 180 degrees and washed an additional 4×. The pre-mixed SuperSignal ELISA Pico substrate solution (20 μL) was added to each well. The plate was read on MolecuLar Devices SpectraMax M3 luminometer and Softmax Pro Version 6.2 within 15 minutes of adding the substrate.

Reverse Transcription and qPCR

Procedure (SuperScript IV First Strand Synthesis):

1. Thaw all required kit reagents on ice.

Note: Do not remove SuperScript IV RT from −20 until required.

2. Combine the components of Master Mix 1 into a nuclease free tube/well:

| | # of Rxns | |
|---|---|---|
| Component | 1x (uL) | 70 |
| 50 ng/uL random hexamers | 0.5 | 38.5 |
| 10 mM dNTP mix (10 mM each) | 0.5 | 38.5 |
| Template RNA | Up to 6.5 uL | N/A |
| DEPC-treated water | final volume of | N/A |
| | 6.5 uL | |

3. Mix briefly using by pipetting then centrifuge to collect contents at bottom of the wells.
4. Heat the Master Mix 1:RNA solution at 65° C. for 5 minutes, then incubate on ice for at least 1.5 minutes.
5. Vortex and spin down the 5×SSIV Buffer.
6. Combine the components of Master Mix 2 into a nuclease free tube/well:

| Component | 1x (uL) | 70 |
|---|---|---|
| 5x SSIV Buffer | 2 | 154 |
| 100 mM DTT | 0.5 | 38.5 |
| Ribonuclease Inhibitor | 0.5 | 38.5 |
| SuperScript IV RT (200 U/uL) | 0.5 | 38.5 |

7. Mix by pipetting.
8. Add 7 uL of Master Mix 2 to the annealed RNA reaction.
9. Start the thermal cycler and run the RT program:

| Step | Temp (° C.) | Time (min) |
|---|---|---|
| Reverse Transcription (hold) | 23 | 10 |
| RT inactivation (hold) | 55 | 10 |
| Storage | 80 | 10 |

10. Store cDNA until ready for qPCR.
Procedure (qPCR):
1. Determine number of reactions required for each TaqMan kit:

| Kit | # of samples |
|---|---|
| Ppib | 75 |
| Dmpk | 75 |

2. Create master mixes for the reactions:

Ppib

| | Volume | |
|---|---|---|
| | (4 rxn plus | Volume |
| Component | overage) | Required |
| TaqMan Gene Expression | 50 | 3750 |
| Master Mix (2x) | | |
| TaqMan Gene Expression | 1.7 | 127.5 |
| Assay (60x) | | |
| TaqMan Gene Expression | 1.7 | 127.5 |
| Assay (60x) | | |
| cDNA Template | 5 | |
| H2O | 41.6 | 3120 |

3. Aliquot Master Mixes into wells of 96 well plate as outlined by the plate map.
4. Add cDNA/H2O to wells as outlined by the plate map.
5. Seal 96 well plate with adhesive cover
6. Mix cDNA and Master Mixes by vortexing
7. Briefly spin 96-well plate to ensure all liquid is at the bottom of each well.
8. Aliquot contents of 96-well plate to 384-well plate (20 uL) using 12-channel micropipette.
9. Run qPCR analysis using the QuantStudio 7 (reservation required).

### ADDITIONAL EMBODIMENTS

1. An antibody that binds to human transferrin receptor (TfR), wherein the antibody comprises:

(i) a heavy chain complementary determining region 1 (CDR-H1), a heavy chain complementary determining region 2 (CDR-H2), and a heavy chain complementary determining region 3 (CDR-H3) of a heavy chain variable region (VH) comprising the amino acid sequence of SEQ ID NO: 7; and/or

(ii) a light chain complementary determining region 1 (CDR-L1), a light chain complementary determining region 2 (CDR-L2), and a light chain complementary determining region 3 (CDR-L3) of a light chain variable region (VL) comprising the amino acid sequence of SEQ ID NO: 8.

2. The antibody of embodiment 1, wherein the antibody comprises:

(i) a CDR-H1 as set forth in SEQ ID NO: 1, a CDR-H2 as set forth in SEQ ID NO: 2, a CDR-H3 as set forth in SEQ ID NO: 3; and/or a CDR-L1 as set forth in SEQ ID NO: 4, a CDR-L2 as set forth in SEQ ID NO: 5, and a CDR-L3 as set forth in SEQ ID NO: 6;

(ii) a CDR-H1 as set forth in SEQ ID NO: 145, a CDR-H2 as set forth in SEQ ID NO: 146, a CDR-H3 as set forth in SEQ ID NO: 147; and/or a CDR-L1 as set forth in SEQ ID NO: 148, a CDR-L2 as set forth in SEQ ID NO: 149, and a CDR-L3 as set forth in SEQ ID NO: 6; or

(iii) a CDR-H1 as set forth in SEQ ID NO: 150, a CDR-H2 as set forth in SEQ ID NO: 151, a CDR-H3 as set forth in SEQ ID NO: 152; and/or a CDR-L1 as set forth in SEQ ID NO: 153, a CDR-L2 as set forth in SEQ ID NO: 5, and a CDR-L3 as set forth in SEQ ID NO: 154.

3. An antibody that binds to human transferrin receptor (TfR), wherein the antibody comprises:

(i) a CDR-H1 as set forth in SEQ ID NO: 1, a CDR-H2 as set forth in SEQ ID NO: 233 or SEQ ID NO: 80, a CDR-H3 as set forth in SEQ ID NO: 3; and/or a CDR-L1 as set forth in SEQ ID NO: 4, a CDR-L2 as set forth in SEQ ID NO: 5, and a CDR-L3 as set forth in SEQ ID NO: 6;

(ii) a CDR-H1 as set forth in SEQ ID NO: 145, a CDR-H2 as set forth in SEQ ID NO: 234 or SEQ ID NO: 236, a CDR-H3 as set forth in SEQ ID NO: 147; and/or a CDR-L1 as set forth in SEQ ID NO: 148, a CDR-L2 as set forth in SEQ ID NO: 149, and a CDR-L3 as set forth in SEQ ID NO: 6; or

(iii) a CDR-H1 as set forth in SEQ ID NO: 150, a CDR-H2 as set forth in SEQ ID NO: 277 or SEQ ID NO: 278, a CDR-H3 as set forth in SEQ ID NO: 152; and/or a CDR-L1 as set forth in SEQ ID NO: 153, a CDR-L2 as set forth in SEQ ID NO: 5, and a CDR-L3 as set forth in SEQ ID NO: 154.

4. The antibody of any one of embodiments 1-3, wherein the antibody comprises a VH comprising an amino acid sequence at least 85% identical to SEQ ID NO: 7, and/or a VL comprising an amino acid sequence at least 85% identical to SEQ ID NO: 8.
5. An antibody that binds to human transferrin receptor (TfR), wherein the antibody comprises:

(i) a heavy chain complementary determining region 1 (CDR-H1), a heavy chain complementary determining region 2 (CDR-H2), and a heavy chain complementary determining region 3 (CDR-H3) of a heavy chain variable region (VH) comprising the amino acid sequence of SEQ ID NO: 15; and/or

(ii) a light chain complementary determining region 1 (CDR-L1), a light chain complementary determining region 2 (CDR-L2), and a light chain complementary determining region 3 (CDR-L3) of a light chain variable region (VL) comprising the amino acid sequence of SEQ ID NO: 16.

6. The antibody of embodiment 5, wherein the antibody comprises:

(i) a CDR-H1 as set forth in SEQ ID NO: 9, a CDR-H2 as set forth in SEQ ID NO: 10, a CDR-H3 as set forth in SEQ ID NO: 11; and/or a CDR-L1 as set forth in SEQ ID NO: 12, a CDR-L2 as set forth in SEQ ID NO: 13, and a CDR-L3 as set forth in SEQ ID NO: 14;

(ii) a CDR-H1 as set forth in SEQ ID NO: 155, a CDR-H2 as set forth in SEQ ID NO: 156, a CDR-H3 as set forth in SEQ ID NO: 157; and/or a CDR-L1 as set forth in SEQ ID NO: 158, a CDR-L2 as set forth in SEQ ID NO: 159, and a CDR-L3 as set forth in SEQ ID NO: 14; or

(iii) a CDR-H1 as set forth in SEQ ID NO: 160, a CDR-H2 as set forth in SEQ ID NO: 161, a CDR-H3 as set forth in SEQ ID NO: 162; and/or a CDR-L1 as set forth in SEQ ID NO: 163, a CDR-L2 as set forth in SEQ ID NO: 13, and a CDR-L3 as set forth in SEQ ID NO: 164.

7. The antibody of embodiment 5 or embodiment 6, wherein the antibody comprises a VH comprising an amino acid sequence at least 85% identical to SEQ ID NO: 15, and/or a VL comprising an amino acid sequence at least 85% identical to SEQ ID NO: 16.
8. An antibody that binds to human transferrin receptor (TfR), wherein the antibody comprises:

(i) a heavy chain complementary determining region 1 (CDR-H1), a heavy chain complementary determining region 2 (CDR-H2), and a heavy chain complementary determining region 3 (CDR-H3) of a heavy chain variable region (VH) comprising the amino acid sequence of SEQ ID NO: 23; and/or

(ii) a light chain complementary determining region 1 (CDR-L1), a light chain complementary determining region 2 (CDR-L2), and a light chain complementary determining region 3 (CDR-L3) of a light chain variable region (VL) comprising the amino acid sequence of SEQ ID NO: 24.

9. The antibody of embodiment 8, wherein the antibody comprises:

(i) a CDR-H1 as set forth in SEQ ID NO: 17, a CDR-H2 as set forth in SEQ ID NO: 18, a CDR-H3 as set forth in SEQ ID NO: 19; and/or a CDR-L1 as set forth in SEQ ID NO: 20, a CDR-L2 as set forth in SEQ ID NO: 21, and a CDR-L3 as set forth in SEQ ID NO: 22;

(ii) a CDR-H1 as set forth in SEQ ID NO: 165, a CDR-H2 as set forth in SEQ ID NO: 166, a CDR-H3 as set forth in SEQ ID NO: 167; and/or a CDR-L1 as set forth in SEQ ID NO: 168, a CDR-L2 as set forth in SEQ ID NO: 169, and a CDR-L3 as set forth in SEQ ID NO: 22; or

(iii) a CDR-H1 as set forth in SEQ ID NO: 170, a CDR-H2 as set forth in SEQ ID NO: 171, a CDR-H3 as set forth in SEQ ID NO: 172; and/or a CDR-L1 as set forth in SEQ ID NO: 173, a CDR-L2 as set forth in SEQ ID NO: 21, and a CDR-L3 as set forth in SEQ ID NO: 174.

10. An antibody that binds to human transferrin receptor (TfR), wherein the antibody comprises:

(i) a CDR-H1 as set forth in SEQ ID NO: 237 or SEQ ID NO: 239, a CDR-H2 as set forth in SEQ ID NO: 18, a CDR-H3 as set forth in SEQ ID NO: 19; and/or a CDR-L1 as set forth in SEQ ID NO: 20, a CDR-L2 as set forth in SEQ ID NO: 21, and a CDR-L3 as set forth in SEQ ID NO: 22; or

(ii) a CDR-H1 as set forth in SEQ ID NO: 238 or SEQ ID NO: 240, a CDR-H2 as set forth in SEQ ID NO: 166, a CDR-H3 as set forth in SEQ ID NO: 167; and/or a CDR-L1 as set forth in SEQ ID NO: 168, a CDR-L2 as set forth in SEQ ID NO: 169, and a CDR-L3 as set forth in SEQ ID NO: 22.

11. The antibody of any one of embodiments 8-10, wherein the antibody comprises a VH comprising an amino acid sequence at least 85% identical to SEQ ID NO: 23, and/or a VL comprising an amino acid sequence at least 85% identical to SEQ ID NO: 24.
12. The antibody of any one of embodiments 1-11, wherein the antibody is a humanized antibody.
13. The antibody of embodiment 10, wherein the humanized antibody comprises a humanized VH and/or a humanized VL.
14. The antibody of any one of embodiments 1-13, wherein the antibody is selected from the group consisting of a full-length IgG, a Fab fragment, a F(ab′) fragment, a F(ab′)2 fragment, a scFv, and a Fv.
15. The antibody of embodiment 14, wherein the antibody is a full-length IgG.
16. The antibody of embodiment 15, wherein the antibody comprises a heavy chain constant region of the isotype IgG1, IgG2, IgG3, or IgG4.
17. The antibody of embodiment 16, wherein the antibody comprises a heavy chain constant region of the isotype IgG1 set forth in SEQ ID NO: 175 or SEQ ID NO: 176.
18. The antibody of any one of embodiments 15 to 17, wherein the antibody comprises:

(i) a heavy chain comprising an amino acid sequence at least 85% identical to SEQ ID NO: 178, and/or a light chain comprising an amino acid sequence at least 85% identical to SEQ ID NO: 179;

(ii) a heavy chain comprising an amino acid sequence at least 85% identical to SEQ ID NO: 180, and/or a light chain comprising an amino acid sequence at least 85% identical to SEQ ID NO: 181; or

(iii) a heavy chain comprising an amino acid sequence at least 85% identical to SEQ ID NO: 182, and/or a light chain comprising an amino acid sequence at least 85% identical to SEQ ID NO: 183.

19. The antibody of embodiment 14, wherein the antibody is a F(ab′) fragment.
20. The antibody of embodiment 19, wherein the antibody comprises:

(i) a heavy chain comprising an amino acid sequence at least 85% identical to SEQ ID NO: 185, and/or a light chain comprising an amino acid sequence at least 85% identical to SEQ ID NO: 179;

(ii) a heavy chain comprising an amino acid sequence at least 85% identical to SEQ ID NO: 186, and/or a light chain comprising an amino acid sequence at least 85% identical to SEQ ID NO: 181; or

(iii) a heavy chain comprising an amino acid sequence at least 85% identical to SEQ ID NO: 187, and/or a light chain comprising an amino acid sequence at least 85% identical to SEQ ID NO: 183.

21. An antibody that binds to human transferrin receptor (TfR), wherein the antibody comprises: a CDR-H1, a CDR-H2, and a CDR-H3, which collectively contains no more than 10 amino acid variations, preferably no more than 8 amino acid variations, and more preferably no more than 5 amino acid variations, as compared with the CDR-H1, a CDR-H2, and a CDR-H3 of any one of the antibodies listed in Table 1; and/or wherein the antibody comprises a CDR-L1, a CDR-L2, and a CDR-L3, which collectively contains no more than 10 amino acid variations, preferably no more than 8 amino acids variations, as compared with the a CDR-L1, a CDR-L2, and a CDR-L3 of any one of the antibodies listed in Table 1 or Table 3.
22. An antibody that binds to human transferrin receptor (TfR), wherein the antibody comprises: a CDR-H1, a CDR-H2, and a CDR-H3 of any one of the antibodies listed in Table 1; and/or wherein the antibody comprises a CDR-L1, a CDR-L2, and a CDR-L3, of any one of the antibodies listed in Table 1 or Table 3.
23. An antibody that binds to human transferrin receptor (TfR), wherein the antibody comprises:

a CDR-H1 set forth as GYSITSGYX₁ (SEQ ID NO: 286), in which X₁ can be Y or G;

a CDR-H2 set forth as IX₂FDGX₃X₄ (SEQ ID NO: 287), in which X₂ can be T or N, X₃ can be A or N, and X₄ can be N, T, or S;

a CDR-H3 set forth as X₅RX₆X₇YDYDX₈X₉DX₁₀ (SEQ ID NO: 288), wherein X₅ is T or A, X₆ is S, F, or I, X₇ is S, N, or Y, X₈ is P, Y, or V, X₉ is I, F, or L, and X₁₀ is Y or F; and/or

a CDR-L1 set forth as QDIX₁₁NX₁₂ (SEQ ID NO: 289), in which X₁₁ is S or T and X₁₂ is F, C, S, or Y;

a CDR-L2 set forth as YTS (SEQ ID NO: 13);

and a CDR-L3 set forth as QQGX₁₃X₁₄X₁₅PX₁₆T (SEQ ID NO: 290), in which X₁₃ is H or N, X₁₄ is T or A, X₁₅ is L or Y, and X₁₆ is Y, W, or F.

24. The antibody of any one of embodiments 1-21, wherein the antibody binds transferrin receptor 1 (TfR1) with a K_{D} of less than 10⁻⁹M.
25. A nucleic acid encoding the antibody in any one of embodiments 1-24.
26. A vector comprising the nucleic acid of embodiment 25.
27. A cell comprising the vector of embodiment 26.
28. A method producing an anti-TfR1 antibody, comprising culturing the cell of embodiment 27 under conditions suitable for the expression of the antibody.
29. A complex comprising the antibody of any one of embodiments 1-23 covalently linked to a molecular payload.
30. The complex of embodiment 29, wherein the molecular payload is a diagnostic agent or a therapeutic agent.
31. The complex of embodiment 29, wherein the molecular payload is an oligonucleotide, a polypeptide, or a small molecule.
32. The complex of any one of embodiments 29-31, wherein the antibody and the molecular payload are linked via a linker.
33. The complex of embodiment 32, wherein the linker is a reversible linker.
34. The complex of embodiment 33, wherein the linker is a val-Cit linker.
35. A composition comprising the antibody of any one of embodiments 1-23, the nucleic acid of embodiment 25, the vector of embodiment 26, or the complex of any one of embodiments 26-31.
36. The composition of embodiment 32, further comprising a pharmaceutically acceptable carrier.
37. A method of detecting a transferrin receptor in a biological sample, comprising contacting the antibody of any one of embodiments 1-23 with the biological sample and measuring binding of the antibody to the biological sample.
38. The method of embodiment 37, wherein the antibody is covalently linked to a diagnostic agent.
39. The method of embodiment 38, wherein the biological sample is obtained from a human subject suspected of having or at risk for a disease associated with transferrin receptor.
40. The method of embodiment 39, wherein the contacting step is performed by administering the subject an effective amount of the anti-TfR antibody.
41. A method of delivering a molecular payload to a cell, comprising contacting the complex of any one of embodiments 29-34 with the cell.
42. The method of embodiment 41, wherein the cell is a muscle cell.
43. The method of embodiment 41 or embodiment 42, wherein the cell is in vitro.
44. The method of embodiment 43, wherein the cell is in a subject.
45. The method of embodiment 44, wherein the subject is human.
46. A method of delivering a molecular payload to the brain or the muscle of a subject, comprising administering to the subject an effective amount of the complex of any one of embodiments 29-34.
47. The method of embodiment 46, wherein the administration is intravenous.
48. A method of treating a disease, comprising administering to the subject an effective amount of the complex of any one of embodiments 29-34, wherein the molecular payload is a therapeutic agent.
49. The method of embodiment 48, wherein the disease is a neurological disease and the molecular payload is a drug for treating a neurological disease.
50. The method of embodiment 48, wherein the disease is a muscle disease and the molecular payload is a drug for treating a muscle disease.
51. The method of embodiment 50, wherein the muscle disease is a rare muscle disease or muscle atrophy.

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or (e.g., and) one or more modified nucleotides and/or (e.g., and) one or more modified internucleotide linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

1. An antibody that binds to human transferrin receptor (TfR), wherein the antibody comprises the antibody comprises a heavy chain complementarity determining region 1 (CDR-H1), a heavy chain complementarity determining region 2 (CDR-H2), a heavy chain complementarity determining region 3 (CDR-H3) of a heavy chain variable region (VH) comprising the amino acid sequence of SEQ ID NO: 54, and a light chain complementarity determining region 1 (CDR-L1), a light chain complementarity determining region 2 (CDR-L2), a light chain complementarity determining region 3 (CDR-L3) of a light chain variable region (VL) comprising the amino acid sequence of SEQ ID NO: 55.

2. The antibody of claim 1, wherein the antibody comprises a CDR-H1 of SEQ ID NO: 49, a CDR-H2 of SEQ ID NO: 50, a CDR-H3 of SEQ ID NO: 51, a CDR-L1 of SEQ ID NO: 52, a CDR-L2 of SEQ ID NO: 29, and a CDR-L3 of SEQ ID NO: 53.

3. The antibody of claim 1, wherein the antibody comprises human or humanized framework regions with the CDR-H1, the CDR-H2, the CDR-H3 of a VH as set forth in SEQ ID NO: 54, and the CDR-L1, the CDR-L2, the CDR-L3 of a VL as set forth in SEQ ID NO: 55.

4. The antibody of claim 1, wherein the antibody comprises a VH comprising an amino acid sequence at least 80% identical to SEQ ID NO: 54, and a VL comprising an amino acid sequence at least 80% identical to SEQ ID NO: 55.

5. The antibody of claim 1, wherein the antibody is selected from the group consisting of a full-length IgG, a Fab fragment, a F(ab′) fragment, a F(ab′)2 fragment, a scFv, and a Fv.

6. The antibody of claim 5, wherein the antibody is a full-length IgG.

7. The antibody of claim 6, wherein the antibody comprises a heavy chain constant region of the isotype IgG1 set forth in SEQ ID NO: 175 or SEQ ID NO: 176.

8. The antibody of claim 6, wherein the antibody comprises:
(i) a heavy chain comprising an amino acid sequence at least 80% identical to SEQ ID NO: 180, and a light chain comprising an amino acid sequence at least 80% identical to SEQ ID NO: 181;
(ii) a heavy chain comprising an amino acid sequence at least 80% identical to SEQ ID NO: 178, and a light chain comprising an amino acid sequence at least 80% identical to SEQ ID NO: 179; or
(iii) a heavy chain comprising an amino acid sequence at least 80% identical to SEQ ID NO: 182, and a light chain comprising an amino acid sequence at least 80% identical to SEQ ID NO: 183.

9. The antibody of claim 5, wherein the antibody is a F(ab′) fragment.

10. The antibody of claim 9, wherein the antibody comprises:
(i) a heavy chain comprising an amino acid sequence at least 85% identical to SEQ ID NO: 186, and a light chain comprising an amino acid sequence at least 85% identical to SEQ ID NO: 181;
(ii) a heavy chain comprising an amino acid sequence at least 85% identical to SEQ ID NO: 185, and a light chain comprising an amino acid sequence at least 85% identical to SEQ ID NO: 179; or
(iii) a heavy chain comprising an amino acid sequence at least 85% identical to SEQ ID NO: 187, and a light chain comprising an amino acid sequence at least 85% identical to SEQ ID NO: 183.

11. The antibody of claim 1, wherein the antibody binds transferrin receptor 1 (TfR1) with a K_{D} of less than 10⁻⁸ M.

12. A nucleic acid encoding the antibody of claim 1.

13. A method of producing an anti-TfR1 antibody, comprising culturing cells comprising the nucleic acid of claim 12 under conditions suitable for the expression of the antibody.

14. A complex comprising the antibody of claim 1 covalently linked to a molecular payload.

15. A method of detecting a transferrin receptor in a biological sample, comprising contacting the antibody of claim 1 with the biological sample and measuring binding of the antibody to the biological sample.

16. A method of delivering a molecular payload to a cell, comprising contacting the cell with the complex of claim 14.

17. The method of claim 16, wherein the cell is a muscle cell.

18. A method of delivering a molecular payload to the brain or the muscle of a subject, comprising administering to the subject an effective amount of the complex of claim 14.

19. A method of treating a disease, comprising administering to the subject an effective amount of the complex of claim 14, wherein the molecular payload is a therapeutic agent.

20. The method of claim 19, wherein the disease is a neurological disease and the molecular payload is a drug for treating a neurological disease, or wherein the disease is a muscle disease and the molecular payload is a drug for treating a muscle disease.
